(19) United States
(12) Patent Application Publication
(10) Pub. No.: US 2024/0018123 A1
(43) Pub. Date: Jan. 18, 2024

(54) COMPOUND FOR INHIBITING OR DISINTEGRATING ANDROGEN RECEPTOR, AND PHARMACEUTICAL USE THEREOF

(71) Applicants: KOREA RESEARCH INSTITUTE OF CHEMICAL TECHNOLOGY, Daejeon (KR); KOREA RESEARCH INSTITUTE OF BIOSCIENCE AND BIOTECHNOLOGY, Daejeon (KR); UBIX THERAPEUTICS, INC., Seoul (KR)

(72) Inventors: Jong Yeon HWANG, Daejeon (KR); Jae Du HA, Daejeon (KR); Hyun Jin KIM, Daejeon (KR); Sung Yun CHO, Daejeon (KR); Pilho KIM, Daejeon (KR); Chong Ock LEE, Daejeon (KR); Jeong Hoon KIM, Daejeon (KR); Byoung Chul PARK, Daejeon (KR); Sung Goo PARK, Daejeon (KR); Sunhong KIM, Seoul (KR); Yuri CHOI, Incheon (KR); Yaejin WOO, Gyeonggi-do (KR); Song Hee LEE, Seoul (KR); Je Ho RYU, Gyeonggi-do (KR); Jungmin AHN, Incheon (KR); Ji Youn PARK, Gyeonggi-do (KR); Onnuri BAE, Incheon (KR); Hanwool KIM, Gyeonggi-do (KR)

(21) Appl. No.: 18/017,997

(22) PCT Filed: Jul. 28, 2021
(86) PCT No.: PCT/KR2021/009832

§ 371 Date: Jan. 25, 2023

(30) Foreign Application Priority Data
Jul. 29, 2020 (KR) 10-2020-0094789

(51) Int. Cl. C07D 401/14; C07D 295/155; C07D 211/26

(52) U.S. Cl. CPC C07D 401/14; C07D 211/26; C07D 295/155

(57) ABSTRACT

The present disclosure provides a compound of a chemical structure having the ability of inhibiting or disintegrating an androgen receptor (AR), or a pharmaceutically acceptable salt of the compound. The present disclosure also provides a composition comprising such a compound or a pharmaceutically acceptable salt thereof. The present disclosure provides a pharmaceutical use of a compound according to the present disclosure, a salt thereof, and a composition comprising same for the treatment or prevention of AR-related diseases. The present disclosure also provides a method for treating or preventing AR-related diseases, comprising administering to a subject in need of treatment an effective amount of a compound according to the present disclosure, a salt thereof, or a composition comprising same.

### TECHNICAL FIELD

This application claims priority to Korean Patent Application No. 10-2020-0094789 filed on Jul. 29, 2020, and all contents disclosed in the specification and drawings of the application are incorporated into this application.

The present invention relates to a group of compounds that have the activity of inhibiting or degrading the androgen receptor. The present invention also relates to pharmaceutical compositions comprising such compounds. The present invention also relates to useful methods of treating androgen receptor related diseases using such compounds. That is, the present invention relates to the medical use of the compounds according to the present disclosure for treating or preventing androgen receptor related diseases.

### BACKGROUND ART

Androgen hormone receptor (AR) is a transcription factor that belongs to the nuclear hormone receptor (NR). It binds to the hormone dihydrotestosterone (DHT), translocates to the nucleus, and consequently activates the transcription of target genes. In the absence of androgen, AR binds to Heat Shock Protein 90 (Hsp90) in the cytosol, and when androgen binds to AR, Hsp90 and AR are separated, and nuclear localization signal (NLS) is changed to expose.

AR contributes to the development of masculinity, but it is also a well-known oncogene in certain forms of cancer, including prostate cancer (Endocr. Rev. 2004, 25(2), 276-308). Current treatment regimens for androgen-related prostate cancer can be divided into two main categories. The first approach is to control androgen levels by removing androgens or preventing their translocation into the nucleus by interfering with the binding of its ligand, DHT. The second strategy aims to inhibit AR function by targeting AR (Nature Reviews Drug Discovery, 2013, 12, 823-824). That is, an alternative approach to treatment of prostate cancer involves deleting the AR protein. AR is an important driver of tumorigenesis in many forms of prostate cancer.

This AR can also be a major target for the treatment of acne, alopecia (especially androgenetic alopecia), cutaneous wounds, hirsutism, etc. (Arch Dermatol Res. 2012 September; 304-(7): 499-510, Biomedicine & Pharmacotherapy 137 (2021) 111247), and it has been found that the expression and activation of AR also play an important role in breast cancer (especially androgen receptor-positive triple-negative breast cancer (AR+TNBC)) (npj Breast Cancer (2020) 6:47).

Representative anti-androgen receptor drugs include enzalutamide and bicalutamide, and apalutamide has recently been approved. However, about 15-25% of prostate cancer patients do not respond to anti-androgen drugs, and the approved drug shows excellent anticancer effects in the initial stage of administration, but drug resistance develops due to continuous use, making it difficult to use them any longer. Therefore, there is an urgent need to develop new therapeutic agents.

### DISCLOSURE

### Technical Problem

Accordingly, the problem to be solved by the present disclosure is to provide a compound having activity for inhibiting or degrading androgen receptor (AR), pharmaceutical compositions comprising the same as an active ingredient, and medical uses for treating or preventing AR-related diseases.

Another problem to be solved by the present disclosure is to provide a method for treating or alleviating AR-related diseases, characterized in that it inhibits AR activity, and it comprises administering to a patient in need of treatment, improvement or prevention of AR related diseases the compound according to the present invention.

### Technical Solution

### Compounds of the Present Invention

In order to solve the above problem, one embodiment of the present invention provides a compound represented by the following Chemical Formula 1 or a pharmaceutically acceptable salt thereof.

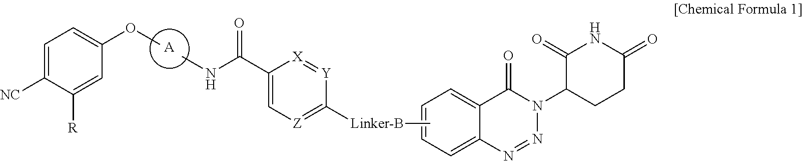

In the Chemical Formula 1,

R is H, C₁₋₆alkyl, halogen, or haloC₁₋₆alkyl,

A is C₃₋10cycloalkyl or heterocycle, wherein these optionally have one or more hydrogens replaced by C₁₋₆alkyl, C₁₋₆alkoxy, halogen, haloC₁₋₆alkyl, ═O, or ═S (preferably, replaced by C₁₋₆alkyl, C₁₋₆alkoxy, or haloC₁₋₆alkyl; more preferably, replaced by C₁₋₆alkyl; most preferably, replaced by methyl),

X, Y, and Z are each independently CH or N,

Linker-B is a linker that connects the moieties on both sides of Linker-B.

The present inventors tried to develop a new compound with excellent AR inhibition and/or degrdation activity, (metabolism) stability, etc., and excellent physicochemical properties (c Log P value, water solubility, cell membrane permeability) as an active ingredient by combining a CRBN ligand (right moiety relative to the linker) with a specific structure that binds to E3 ubiquitin ligase and a moiety that binds to AR (left moiety relative to the linker). To this end, various AR binding moiety structures such as enzalutamide were used, but contrary to expectations, the desired degree of activity or physical properties could not be achieved, and only specific combinations met the various purposes of the present invention.

As used herein, the terms “substituent”, “radical”, “group”, “moiety”, and “fragment” may be used interchangeably.

If a substituent is described as “optionally substituted”, the substituent may be (1) unsubstituted or (2) substituted with one or more of the defined substituents. If the substitutable position is unsubstituted, the default substituent is hydride radical.

As used herein, the term “alkyl” means a saturated straight chain or branched non-cyclic hydrocarbon, unless the context clearly dictates otherwise, having from 1 to 10 carbon atoms. “Lower alkyl” means alkyl having from 1 to 4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl, while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkoxy” means —O-(alkyl) including —OCH₃, —OCH₂CH₃, —O(CH₂)₂CH₃, —O(CH₂)₃CH₃, —O(CH₂)₄CH₃, —O(CH₂)₅CH₃, and the like, wherein alkyl is as defined above.

As used herein, the term “alkenyl” means a saturated straight-chain or branched non-cyclic hydrocarbon containing from 2 to 10 carbon atoms and at least one carbon-carbon double bond. Representative straight and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, —3-hexeenyl, -1-heptenyl, -2-heptenyl, -3-heptenyl, -1-octenyl, -2-octenyl, -3-octenyl, -1-nonenyl, -2-none yl, -3-nonenyl, -1-disenyl, -2-disenyl, and -3-disenyl. These alkenyl groups may be optionally substituted.

As used herein, the term “alkynyl” means a straight-chain or branched non-cyclic hydrocarbon having 2 to 10 carbon atoms and containing at least one carbon-carbon triple bond. Representative straight or branched (C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, and -9-decynyl. These alkynyl groups may be optionally substituted.

As used herein, if the term “C₁₋₆”, “C1-6”, or “C1-C6” is used, it means the number of carbon atoms is from 1 to 6. For example, C₁₋₆alkyl means an alkyl which carbon number is any integer of from 1 to 6.

As used herein, the terms “halogen” and “halo” mean fluorine, chlorine, bromine or iodine. In a preferred embodiment of the present invention, the halogen is chlorine.

As used herein, the term “haloalkyl”, “haloalkoxy”, “haloalkenyl” or “haloalkynyl” means an alkyl, alkoxy, alkenyl, or alkynyl group, respectively, wherein one or more hydrogen atoms are substituted with halogen atoms. For example, the haloalkyl includes —CF₃, —CHF₂, —CH₂F, —CBr₃, —CHBr₂, —CH₂Br, —CC1₃, —CHC1₂, —CH₂Cl, —Cl₃, —CHI₂, —CH₂I, —CH₂—CF₃, —CH₂—CHF₂, —CH₂—CH₂F, —CH₂—CBr₃, —CH₂—CHBr₂, —CH₂—CH₂Br, —CH₂—CC1₃, —CH₂—CHC1₂, —CH₂—CH₂Cl, —CH₂—Cl₃, —CH₂—CHI₂, —CH₂—CH₂I, and the like, wherein alkyl and halogen are as described above. In a preferred embodiment of the present invention, haloalkyl is —CF₃.

As used herein, the term “cycloalkyl” means a monocyclic or polycyclic saturated ring having carbon and hydrogen atoms and having no carbon-carbon multiple bonds. Examples of cycloalkyl groups include, but are not limited to, (C3-C7)cycloalkyl groups, including cyclopropyl, cyclobutyl, cyclopentyl, cyclohexyl, and cycloheptyl. A cycloalkyl group can be unsubstituted or optionally substituted. In one embodiment, the cycloalkyl group is a monocyclic or bicyclic ring.

As used herein, the term “heterocycle (heteroring)” or “heterocycloalkyl” means a 5- to 7-membered monocyclic or 7- to 12-membered bicyclic, saturated or unsaturated heterocyclic ring which contains from 1 to 4 heteroatoms independently selected from nitrogen, oxygen and sulfur, and wherein the nitrogen and sulfur heteroatoms can be optionally oxidized, and the nitrogen heteroatom can be optionally quaternized. The term includes bicyclic rings wherein some of the heterocycle are fused to a benzene ring. Heterocycles may be attached by heteroatoms or carbon atoms. Heterocycles include heteroaryls as defined below. Representative heterocycles include morpholinyl, pyrrolidinonyl, pyrrolidinyl, piperidinyl, hydantoinyl, valerolactamyl, oxiranil, oxetanil, tetrahydrofuranyl, tetrahydropyranyl, tetrahydropyridinyl, tetrahydropyrimidinyl, tetrahydrothiophenyl, tetrahydrothiopyranyl, tetrahydropyrimidinyl, tetrahydrothiophenyl, and tetrahydrothiopyranyl.

As used herein, the term “aryl” means a carbocyclic aromatic group containing from 5 to 10 ring atoms. Representative examples include, but are not limited to, phenyl, tolyl, xylyl, naphthyl, tetrahydronaphthyl, anthracenyl, fluorenyl, indenyl, and azulenyl. A carbocyclic aromatic group can be unsubstituted or optionally substituted.

As used herein, the term “heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, oxetanyl, azepinyl, piperazinyl, morpholinyl, dioxanyl, thietanyl and oxazolyl.

Although various aspects may be affected by the linker described below, in a preferred embodiment of the present invention, the Chemical Formula 1 has the structure of Chemical Formula 1a below.

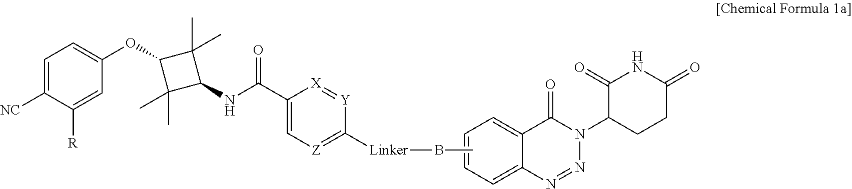

In the Chemical Formula 1a,

R is H, C₁₋₆alkyl, halogen, or haloC₁₋₆alkyl (preferably, halogen (more preferably, Cl) or CF₃),

X, Y, and Z are each independently CH or N,

Linker-B is a linker that connects the moieties on both sides of Linker-B.

In the Chemical Formula 1a, the AR binding moiety is more preferable for various purposes of the present invention compared to many known AR binding substances such as enzalutamide in relation to the CRBN ligand of a specific structure used in the present invention. In addition, the AR binding moiety of Chemical Formula 1a was more preferred than AR binding moieties of other structures included within the scope of this present disclosure (e.g., the unsubstituted cyclohexyl-containing moiety of Example 164).

In one embodiment of the present invention, the linker of Chemical Formula 1 and/or 1a is a linker that connects the AR binding ligand moiety and the CRBN ligand moiety according to the present invention. These linkers can be connected through an alkyl bond through chloride, bromide, iodide, or tosylate; an amide bond through acid; or an amide bond through amine. As such a linker, for example, linkers disclosed in prior patents US20180353501 A1, WO2019199816 A1, WO2019023553 A1, US20180125821 A1, US20190192668 A1, WO2017197056 A1, WO2019186358 A1, and/or WO2018089736 A1 may be used. The contents described in the prior patent application publication are incorporated herein in their entirety by this reference.

In one exemplary embodiment, the linker has a chain (alkane, alkene, or alkyne) of 2 to 14, 15, 16, 17, 18, or 20 or more carbon atoms, at least one of which may be replaced by a heteroatom such as O, N, S or P. In certain embodiments, the chain has 2, 3, 4, 5, 6, 7, 8, 9, 10, 11, 12, 13, 14, 15, 16, 17, 18, 19 or 20 contiguous atoms in the chain. For example, the chain may contain one or more ethylene glycol units (e.g., 2, 3, 4, 5, 6, 7, 8, 9, 10, 11 or 12 ethylene glycol units) which may be continuous, partially continuous or discontinuous. In certain embodiments, the chain may have at least 1, 2, 3, 4, 5, 6, 7 or 8 of continuous chains which can have branch(es) that may independently be alkyl, heteroalkyl, aryl, heteroaryl, alkenyl or alkynyl, aliphatic, heteroaliphatic, cycloalkyl or heterocycloalkyl substituents.

In other embodiments, the linker may comprise or consist of one or more units of ethylene glycol, propylene glycol, lactic acid and/or glycolic acid. Generally, propylene glycol adds hydrophobicity, while ethylene glycol adds hydrophilicity. The lactic acid segment has a longer half-life than the glycolic acid segment. Block and random lactic acid-glycolic acid moieties, as well as ethylene glycol and propylene glycol, are known in the art as pharmaceutically acceptable and can be modified or arranged to obtain desired half-life and hydrophilicity. In certain aspects, these units are flanked by other moieties, including alkyl, heteroaliphatic, aryl, heteroaryl, heterocyclic, cycloalkyl, etc., or may be interspersed as necessary to achieve appropriate drug properties.

In a preferred embodiment of the present invention, the linker of Chemical Formula 1 and/or 1a has the structure of Chemical Formula 2 below.

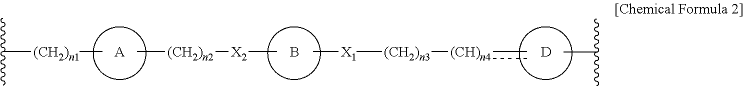

In the Chemical Formula 2,

A is any one of the following structures:

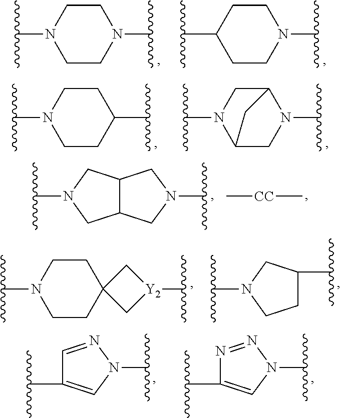

B is direct bond,

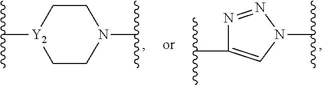

X₁, and X₂ are each independently direct bond, O, or C(O),

D is direct bond, NH, or any one of the following structures:

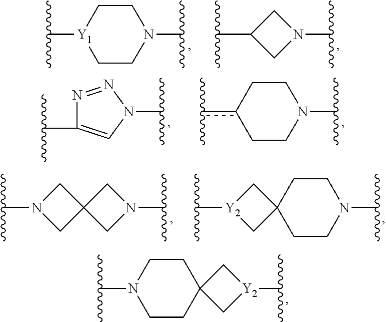

Y₁ and Y₂ are each independently N, CH, C(OH), or CF,

n₁, n₂, and n₃ are each independently an integer of from 0 to 5,

n₄ is 0 or 1,

[symbol] is single bond or double bond.

In a more preferred embodiment of the present invention, the linker of Chemical Formula 1 and/or 1a has any one of the following structures:

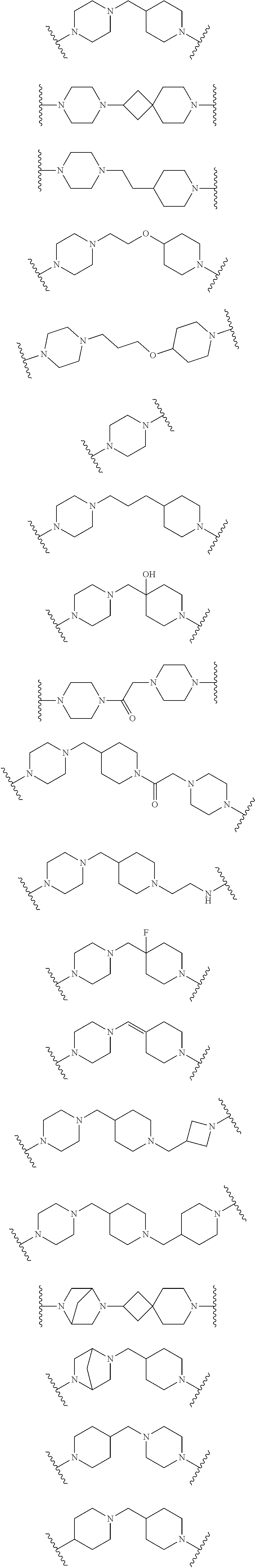

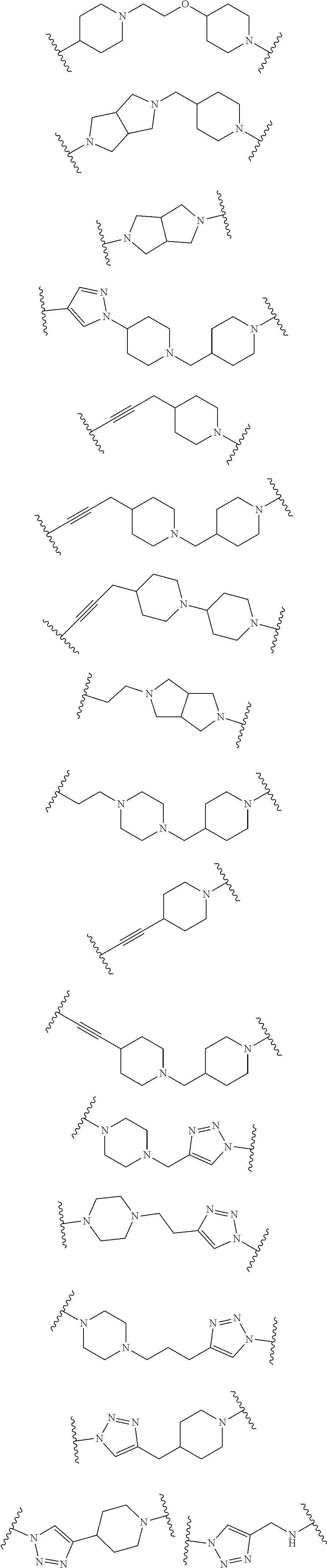

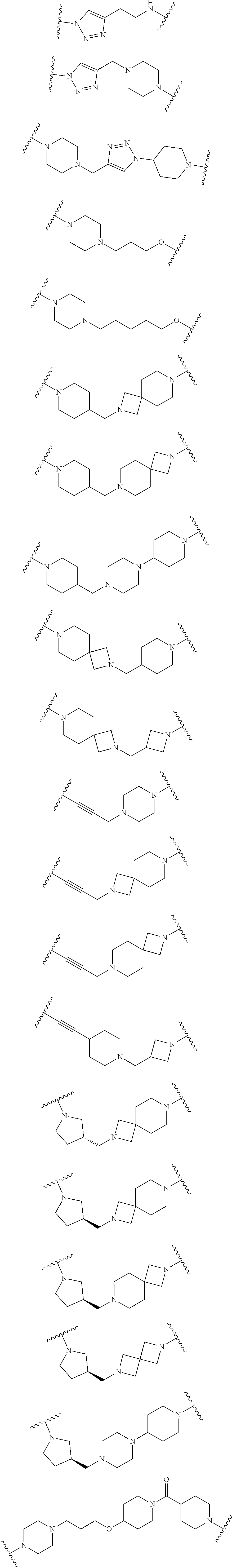

In various evaluation experiments by the present inventors, linkers of the above specific structures were more preferable in various aspects of the present invention, such as activity and (metabolic) stability. Linkers having (poly)ethylene structure(s) were less preferred in terms of metabolic stability, and somewhat rigidified linkers were preferred. In addition, it is considered to be excellent for various purposes of the present invention, such as activity, when the length of the linker is appropriate (not too long or short), but the present invention is not limited to these theoretical assumptions.

As a result of various experiments according to the object of the present invention, among the linkers, the linker having the following structure is preferred.

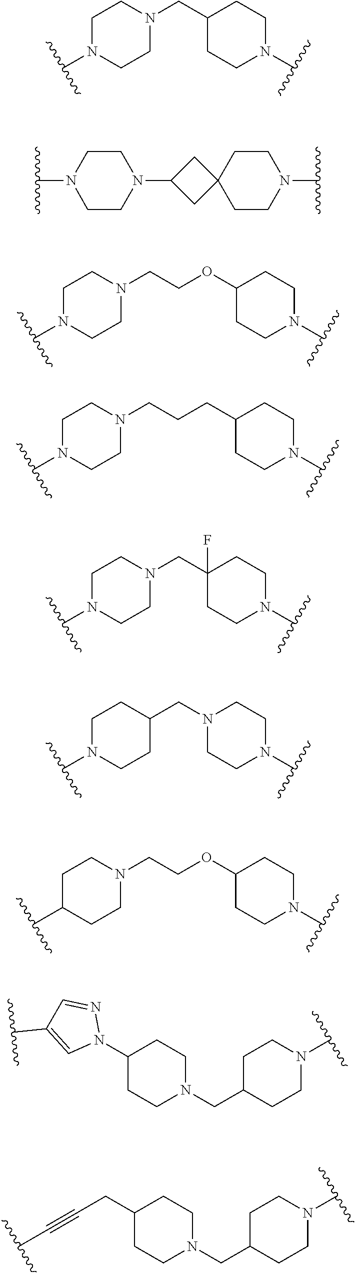

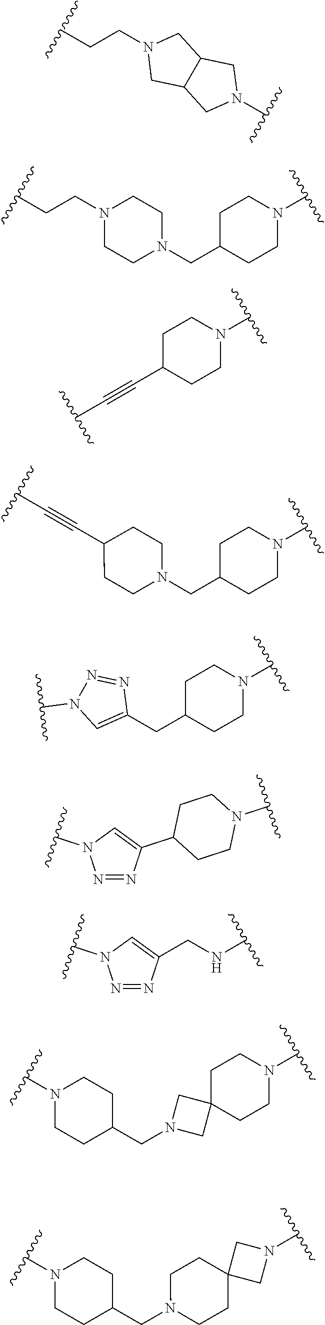

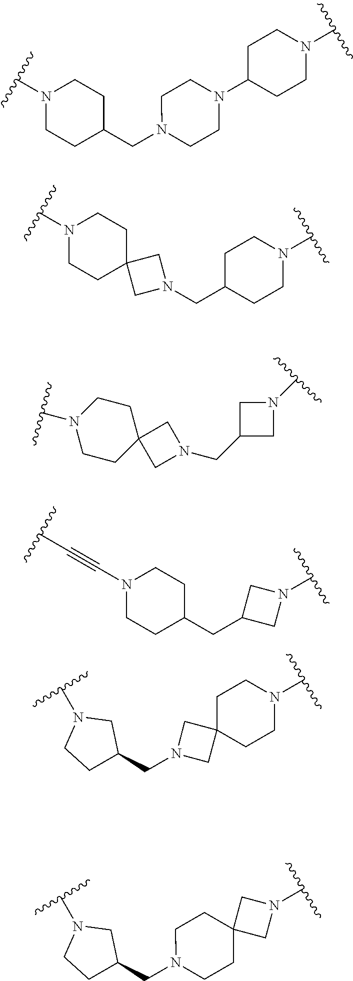 [SMILES: *N1CCC(CN2CCN(C3CCN(*)CC3)CC2)CC1 ; *N1CCC(CN2CC3(CCN(*)CC3)C2)CC1 ; *N1CCC2(CC1)CN(CC1CN(*)C1)C2 ; *C#CN1CCC(CC2CN(*)C2)CC1 ; *N1CCC2(CC1)CN(C[C@H]1CCN(*)C1)C2 ; *N1CC[C@H](CN2CCC3(CC2)CN(*)C3)C1]

For example, the linkers used in Examples 59 and 69 were generally less preferred compared to the linker having the above preferred structure, despite the similar structure.

In one embodiment of the present invention, B of Chemical Formula 1 or 1a is direct bond (that is, it does not exist), *—C(O)—CH₂—O*, *—C(O)—CH₂—NH—*,

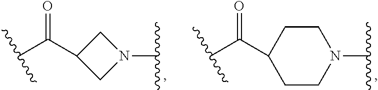 [SMILES: *C(=O)C1CN(*)C1 ; *C(=O)C1CCN(*)CC1]

O, or NH. In a preferred embodiment of the present invention, Bof Chemical Formula 1 or 1a is direct bond, *—C(O)—CH₂—O*, *—C(O)—CH₂—NH—*,

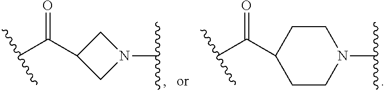 [SMILES: *C(=O)C1CN(*)C1 ; *C(=O)C1CCN(*)CC1]

In a preferred embodiment of the present invention, the linker-B (linker when B is direct bond) binds at the same position as

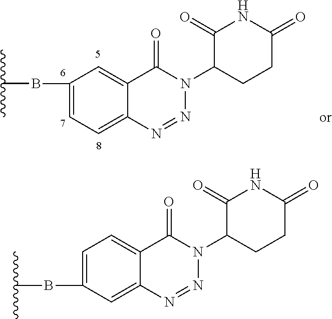

when binding to the CRBN moiety. Linker-B or linker is more preferable in terms of activity such as AR degradation activity and cancer cell line cytotoxicity when bound at position 6 or 7 rather than position 5 and 8, and is also preferred in terms of other physical properties as an active ingredient. For example, in the case of Examples 8 and 23 coupled to the 6^{th} position rather than Examples 7 and 22 coupled to the 5^{th} position, the effect was good even though the other structures were almost the same.

In this specification, *, [symbol] , or L means that it is connected to another moiety.

In the linker and linker-B, the left * means that they are linked to the AR binder moiety, and the right * means that they are linked to the CRBN ligand moiety.

Non-limiting examples of compounds of Chemical Formula 1 according to the present invention are shown in Table 1 below.

TABLE 1

| Example | IUPAC name |
|---|---|
| Example | N-((1r,3r)-3-(3-chloro-4-cyanophenoxy)-2,2,4,4-tetramethylcyclobutyl)-4-((17-((3-(2,6- |
| 1 | dioxopiperidin-3-yl)-4-oxo-3,4-dihydrobenzo[d][1,2,3]triazin-5-yl)amino)-3,6,9,12,15- |
| | pentaoxaheptadecyl)oxy)benzamide |
| Example | N-((1r,3r)-3-(3-chloro-4-cyanophenoxy)-2,2,4,4-tetramethylcyclobutyl)-4-((14-((3-(2,6- |
| 2 | dioxopiperidin-3-yl)-4-oxo-3,4-dihydrobenzo[d][1,2,3]triazin-5-yl)amino)-3,6,9,12- |
| | tetraoxatetradecyl)oxy)benzamide |
| Example | N-((1r,3r)-3-(3-chloro-4-cyanophenoxy)-2,2,4,4-tetramethylcyclobutyl)-4-((18-((3-(2,6- |
| 3 | dioxopiperidin-3-yl)-4-oxo-3,4-dihydrobenzo[d][1,2,3]triazin-5-yl)amino)-3,6,9,12,15- |
| | pentaoxaoctadecyl)oxy)benzamide |
| Example | N-((1r,3r)-3-(3-chloro-4-cyanophenoxy)-2,2,4,4-tetramethylcyclobutyl)-4-((18-((3-(2,6- |
| 4 | dioxopiperidin-3-yl)-4-oxo-3,4-dihydrobenzo[d][1,2,3]triazin-5-yl)amino)-3,6,9,12,15- |
| | pentaoxaoctadecyl)oxy)benzamide |
| Example | N-((1r,3r)-3-(3-chloro-4-cyanophenoxy)-2,2,4,4-tetramethylcyclobutyl)-4-((19-((3-(2,6- |
| 5 | dioxopiperidin-3-yl)-4-oxo-3,4-dihydrobenzo[d][1,2,3]triazin-5-yl)amino)-3,6,9,12,15- |
| | pentaoxanonadecyl)oxy)benzamide |
| Example | N-((1r,3r)-3-(3-chloro-4-cyanophenoxy)-2,2,4,4-tetramethylcyclobutyl)-4-((22-((3-(2,6- |
| 6 | dioxopiperidin-3-yl)-4-oxo-3,4-dihydrobenzo[d][1,2,3]triazin-5-yl)amino)-3,6,9,12,15,18- |
| | hexaoxadocosyl)oxy)benzamide |
| Example | N-((1r,3r)-3-(3-chloro-4-cyanophenoxy)-2,2,4,4-tetramethylcyclobutyl)-4-(4-((1-(3-(2,6- |
| 7 | dioxopiperidin-3-yl)-4-oxo-3,4-dihydrobenzo[d][1,2,3]triazin-5-yl)piperidin-4- |
| | yl)methyl)piperazin-1-yl)benzamide |
| Example | N-((1r,3r)-3-(3-chloro-4-cyanophenoxy)-2,2,4,4-tetramethylcyclobutyl)-4-(4-((1-(3-(2,6- |
| 8 | dioxopiperidin-3-yl)-4-oxo-3,4-dihydrobenzo[d][1,2,3]triazin-6-yl)piperidin-4- |
| | yl)methyl)piperazin-1-yl)benzamide |
| Example | N-((1r,3r)-3-(3-chloro-4-cyanophenoxy)-2,2,4,4-tetramethylcyclobutyl)-4-(4-((1-((3-(2,6- |
| 9 | dioxopiperidin-3-yl)-4-oxo-3,4-dihydrobenzo[d][1,2,3]triazin-6-yl)glycyl)piperidin-4- |
| | yl)methyl)piperazin-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 10 | dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 11 | dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-(3-(2,6- |
| 12 | dioxopiperidin-3-yl)-4-oxo-3,4-dihydrobenzo[d][1,2,3]triazin-7-yl)piperidin-4- |
| | yl)methyl)piperazin-1-yl)benzamide |
| Example | N-((1r,3r)-3-(3-chloro-4-cyanophenoxy)-2,2,4,4-tetramethylcyclobutyl)-4-(4-((1-(2-((3-(2,6- |
| 13 | dioxopiperidin-3-yl)-4-oxo-3,4-dihydrobenzo[d][1,2,3]triazin-6-yl)oxy)acetyl)piperidin-4- |
| | yl)methyl)piperazin-1-yl)benzamide |
| Example | N-((1r,3r)-3-(3-chloro-4-cyanophenoxy)-2,2,4,4-tetramethylcyclobutyl)-4-((22-((3-(2,6- |
| 14 | dioxopiperidin-3-yl)-4-oxo-3,4-dihydrobenzo[d][1,2,3]triazin-5-yl)amino)-21-oxo-3,6,9,12,15- |
| | pentaoxa-20-azadocosyl)oxy)benzamide |
| Example | N-(1-(4-(((1r,3r)-3-(3-chloro-4-cyanophenoxy)-2,2,4,4- |
| 15 | tetramethylcyclobutyl)carbamoyl)phenoxy)-3,6,9,12,15-pentaoxanonadecan-19-yl)-1-(3-(2,6- |
| | dioxopiperidin-3-yl)-4-oxo-3,4-dihydrobenzo[d][1,2,3]triazin-6-yl)piperidine-4-carboxamide |
| Example | N-(1-(4-(((1r,3r)-3-(3-chloro-4-cyanophenoxy)-2,2,4,4- |
| 16 | tetramethylcyclobutyl)carbamoyl)phenoxy)-3,6,9,12,15-pentaoxanonadecan-19-yl)-1-(3-(2,6- |
| | dioxopiperidin-3-yl)-4-oxo-3,4-dihydrobenzo[d][1,2,3]triazin-6-yl)azetidine-3-carboxamide |
| Example | N-((1r,3r)-3-(3-chloro-4-cyanophenoxy)-2,2,4,4-tetramethylcyclobutyl)-4-((19-((3-(2,6- |
| 17 | dioxopiperidin-3-yl)-4-oxo-3,4-dihydrobenzo[d][1,2,3]triazin-6-yl)amino)-3,6,9,12,15- |
| | pentaoxanonadecyl)oxy)benzamide |
| Example | N-((1r,3r)-3-(3-chloro-4-cyanophenoxy)-2,2,4,4-tetramethylcyclobutyl)-4-(4-(7-(2-((3-(2,6- |
| 18 | dioxopiperidin-3-yl)-4-oxo-3,4-dihydrobenzo[d][1,2,3]triazin-6-yl)oxy)acetyl)-7- |
| | azaspiro[3.5]nonan-2-yl)piperazin-1-yl)benzamide |
| Example | N-((1r,3r)-3-(3-chloro-4-cyanophenoxy)-2,2,4,4-tetramethylcyclobutyl)-4-(4-(7-((3-(2,6- |
| 19 | dioxopiperidin-3-yl)-4-oxo-3,4-dihydrobenzo[d][1,2,3]triazin-6-yl)glycyl)-7-azaspiro[3.5]nonan- |
| | 2-yl)piperazin-1-yl)benzamide |
| Example | N-((1r,3r)-3-(3-chloro-4-cyanophenoxy)-2,2,4,4-tetramethylcyclobutyl)-4-(4-(7-(1-(3-(2,6- |
| 20 | dioxopiperidin-3-yl)-4-oxo-3,4-dihydrobenzo[d][1,2,3]triazin-6-yl)piperidine-4-carbonyl)-7- |
| | azaspiro[3.5]nonan-2-yl)piperazin-1-yl)benzamide |
| Example | N-((1r,3r)-3-(3-chloro-4-cyanophenoxy)-2,2,4,4-tetramethylcyclobutyl)-4-(4-(7-(1-(3-(2,6- |
| 21 | dioxopiperidin-3-yl)-4-oxo-3,4-dihydrobenzo[d][1,2,3]triazin-6-yl)azetidine-3-carbonyl)-7- |
| | azaspiro[3.5]nonan-2-yl)piperazin-1-yl)benzamide |
| Example | N-((1r,3r)-3-(3-chloro-4-cyanophenoxy)-2,2,4,4-tetramethylcyclobutyl)-4-(4-(7-(3-(2,6- |
| 22 | dioxopiperidin-3-yl)-4-oxo-3,4-dihydrobenzo[d][1,2,3]triazin-5-yl)-7-azaspiro[3.5]nonan-2- |
| | yl)piperazin-1-yl)benzamide |
| Example | N-((1r,3r)-3-(3-chloro-4-cyanophenoxy)-2,2,4,4-tetramethylcyclobutyl)-4-(4-(7-(3-(2,6- |
| 23 | dioxopiperidin-3-yl)-4-oxo-3,4-dihydrobenzo[d][1,2,3]triazin-6-yl)-7-azaspiro[3.5]nonan-2- |
| | yl)piperazin-1-yl)benzamide |
| Example | N-((1r,3r)-3-(3-chloro-4-cyanophenoxy)-2,2,4,4-tetramethylcyclobutyl)-4-(4-(2-(1-(2-((3-(2,6- |
| 24 | dioxopiperidin-3-yl)-4-oxo-3,4-dihydrobenzo[d][1,2,3]triazin-6-yl)oxy)acetyl)piperidin-4- |
| | yl)ethyl)piperazin-1-yl)benzamide |
| Example | N-((1r,3r)-3-(3-chloro-4-cyanophenoxy)-2,2,4,4-tetramethylcyclobutyl)-4-(4-(2-(1-((3-(2,6- |
| 25 | dioxopiperidin-3-yl)-4-oxo-3,4-dihydrobenzo[d][1,2,3]triazin-6-yl)glycyl)piperidin-4- |
| | yl)ethyl)piperazin-1-yl)benzamide |
| Example | N-((1r,3r)-3-(3-chloro-4-cyanophenoxy)-2,2,4,4-tetramethylcyclobutyl)-4-(4-(2-(1-(1-(3-(2,6- |
| 26 | dioxopiperidin-3-yl)-4-oxo-3,4-dihydrobenzo[d][1,2,3]triazin-6-yl)piperidine-4- |
| | carbonyl)piperidin-4-yl)ethyl)piperazin-1-yl)benzamide |
| Example | N-((1r,3r)-3-(3-chloro-4-cyanophenoxy)-2,2,4,4-tetramethylcyclobutyl)-4-(4-(2-(1-(1-(3-(2,6- |
| 27 | dioxopiperidin-3-yl)-4-oxo-3,4-dihydrobenzo[d][1,2,3]triazin-6-yl)azetidine-3- |
| | carbonyl)piperidin-4-yl)ethyl)piperazin-1-yl)benzamide |
| Example | N-((1r,3r)-3-(3-chloro-4-cyanophenoxy)-2,2,4,4-tetramethylcyclobutyl)-4-(4-(2-(1-(3-(2,6- |
| 28 | dioxopiperidin-3-yl)-4-oxo-3,4-dihydrobenzo[d][1,2,3]triazin-5-yl)piperidin-4-yl)ethyl)piperazin- |
| | 1-yl)benzamide |
| Example | N-((1r,3r)-3-(3-chloro-4-cyanophenoxy)-2,2,4,4-tetramethylcyclobutyl)-4-(4-(2-(1-(3-(2,6- |
| 29 | dioxopiperidin-3-yl)-4-oxo-3,4-dihydrobenzo[d][1,2,3]triazin-6-yl)piperidin-4-yl)ethyl)piperazin- |
| | 1-yl)benzamide |
| Example | N-((1r,3r)-3-(3-chloro-4-cyanophenoxy)-2,2,4,4-tetramethylcyclobutyl)-4-(4-(2-(1-(3-(2,6- |
| 30 | dioxopiperidin-3-yl)-4-oxo-3,4-dihydrobenzo[d][1,2,3]triazin-6-yl)piperidin-4-yl)ethyl)piperazin- |
| | 1-yl)benzamide |
| Example | N-((1r,3r)-3-(3-chloro-4-cyanophenoxy)-2,2,4,4-tetramethylcyclobutyl)-4-(4-(2-((1-((3-(2,6- |
| 31 | dioxopiperidin-3-yl)-4-oxo-3,4-dihydrobenzo[d][1,2,3]triazin-6-yl)glycyl)piperidin-4- |
| | yl)oxy)ethyl)piperazin-1-yl)benzamide |
| Example | N-((1r,3r)-3-(3-chloro-4-cyanophenoxy)-2,2,4,4-tetramethylcyclobutyl)-4-(4-(2-((1-(2-((3-(2,6- |
| 32 | dioxopiperidin-3-yl)-4-oxo-3,4-dihydrobenzo[d][1,2,3]triazin-6-yl)oxy)acetyl)piperidin-4- |
| | yl)oxy)ethyl)piperazin-1-yl)benzamide |
| Example | N-((1r,3r)-3-(3-chloro-4-cyanophenoxy)-2,2,4,4-tetramethylcyclobutyl)-4-(4-(2-((1-(2-((3-(2,6- |
| 33 | dioxopiperidin-3-yl)-4-oxo-3,4-dihydrobenzo[d][1,2,3]triazin-6-yl)oxy)acetyl)piperidin-4- |
| | yl)oxy)ethyl)piperazin-1-yl)benzamide |
| Example | N-((1r,3r)-3-(3-chloro-4-cyanophenoxy)-2,2,4,4-tetramethylcyclobutyl)-4-(4-(2-((1-(3-(2,6- |
| 34 | dioxopiperidin-3-yl)-4-oxo-3,4-dihydrobenzo[d][1,2,3]triazin-6-yl)piperidin-4- |
| | yl)oxy)ethyl)piperazin-1-yl)benzamide |
| Example | N-((1r,3r)-3-(3-chloro-4-cyanophenoxy)-2,2,4,4-tetramethylcyclobutyl)-4-(4-(3-((1-(1-(3-(2,6- |
| 35 | dioxopiperidin-3-yl)-4-oxo-3,4-dihydrobenzo[d][1,2,3]triazin-6-yl)piperidine-4- |
| | carbonyl)piperidin-4-yl)oxy)propyl)piperazin-1-yl)benzamide |
| Example | N-((1r,3r)-3-(3-chloro-4-cyanophenoxy)-2,2,4,4-tetramethylcyclobutyl)-4-(4-(3-((1-(1-(3-(2,6- |
| 36 | dioxopiperidin-3-yl)-4-oxo-3,4-dihydrobenzo[d][1,2,3]triazin-6-yl)azetidine-3- |
| | carbonyl)piperidin-4-yl)oxy)propyl)piperazin-1-yl)benzamide |
| Example | N-((1r,3r)-3-(3-chloro-4-cyanophenoxy)-2,2,4,4-tetramethylcyclobutyl)-4-(4-(3-((1-((3-(2,6- |
| 37 | dioxopiperidin-3-yl)-4-oxo-3,4-dihydrobenzo[d][1,2,3]triazin-6-yl)glycyl)piperidin-4- |
| | yl)oxy)propyl)piperazin-1-yl)benzamide |
| Example | N-((1r,3r)-3-(3-chloro-4-cyanophenoxy)-2,2,4,4-tetramethylcyclobutyl)-4-(4-(3-((1-(2-((3-(2,6- |
| 38 | dioxopiperidin-3-yl)-4-oxo-3,4-dihydrobenzo[d][1,2,3]triazin-6-yl)oxy)acetyl)piperidin-4- |
| | yl)oxy)propyl)piperazin-1-yl)benzamide |
| Example | N-((1r,3r)-3-(3-chloro-4-cyanophenoxy)-2,2,4,4-tetramethylcyclobutyl)-4-(4-(3-((1-(3-(2,6- |
| 39 | dioxopiperidin-3-yl)-4-oxo-3,4-dihydrobenzo[d][1,2,3]triazin-6-yl)piperidin-4- |
| | yl)oxy)propyl)piperazin-1-yl)benzamide |
| Example | N-((1r,3r)-3-(3-chloro-4-cyanophenoxy)-2,2,4,4-tetramethylcyclobutyl)-4-(4-((3-(2,6- |
| 40 | dioxopiperidin-3-yl)-4-oxo-3,4-dihydrobenzo[d][1,2,3]triazin-6-yl)glycyl)piperazin-1- |
| | yl)benzamide |
| Example | N-((1r,3r)-3-(3-chloro-4-cyanophenoxy)-2,2,4,4-tetramethylcyclobutyl)-4-(4-(2-((3-(2,6- |
| 41 | dioxopiperidin-3-yl)-4-oxo-3,4-dihydrobenzo[d][1,2,3]triazin-6-yl)oxy)acetyl)piperazin-1- |
| | yl)benzamide |
| Example | N-((1r,3r)-3-(3-chloro-4-cyanophenoxy)-2,2,4,4-tetramethylcyclobutyl)-4-(4-(1-(3-(2,6- |
| 42 | dioxopiperidin-3-yl)-4-oxo-3,4-dihydrobenzo[d][1,2,3]triazin-6-yl)piperidine-4- |
| | carbonyl)piperazin-1-yl)benzamide |
| Example | N-((1r,3r)-3-(3-chloro-4-cyanophenoxy)-2,2,4,4-tetramethylcyclobutyl)-4-(4-(1-(3-(2,6- |
| 43 | dioxopiperidin-3-yl)-4-oxo-3,4-dihydrobenzo[d][1,2,3]triazin-6-yl)azetidine-3- |
| | carbonyl)piperazin-1-yl)benzamide |
| Example | N-((1r,3r)-3-(3-chloro-4-cyanophenoxy)-2,2,4,4-tetramethylcyclobutyl)-4-(4-(1-(3-(2,6- |
| 44 | dioxopiperidin-3-yl)-4-oxo-3,4-dihydrobenzo[d][1,2,3]triazin-6-yl)azetidine-3- |
| | carbonyl)piperazin-1-yl)benzamide |
| Example | N-((1r,3r)-3-(3-chloro-4-cyanophenoxy)-2,2,4,4-tetramethylcyclobutyl)-4-(4-(3-(1-(2-((3-(2,6- |
| 45 | dioxopiperidin-3-yl)-4-oxo-3,4-dihydrobenzo[d][1,2,3]triazin-6-yl)oxy)acetyl)piperidin-4- |
| | yl)propyl)piperazin-1-yl)benzamide |
| Example | N-((1r,3r)-3-(3-chloro-4-cyanophenoxy)-2,2,4,4-tetramethylcyclobutyl)-4-(4-(3-(1-(1-(3-(2,6- |
| 46 | dioxopiperidin-3-yl)-4-oxo-3,4-dihydrobenzo[d][1,2,3]triazin-6-yl)piperidine-4- |
| | carbonyl)piperidin-4-yl)propyl)piperazin-1-yl)benzamide |
| Example | N-((1r,3r)-3-(3-chloro-4-cyanophenoxy)-2,2,4,4-tetramethylcyclobutyl)-4-(4-(3-(1-(1-(3-(2,6- |
| 47 | dioxopiperidin-3-yl)-4-oxo-3,4-dihydrobenzo[d][1,2,3]triazin-6-yl)azetidine-3- |
| | carbonyl)piperidin-4-yl)propyl)piperazin-1-yl)benzamide |
| Example | N-((1r,3r)-3-(3-chloro-4-cyanophenoxy)-2,2,4,4-tetramethylcyclobutyl)-4-(4-(3-(1-(3-(2,6- |
| 48 | dioxopiperidin-3-yl)-4-oxo-3,4-dihydrobenzo[d][1,2,3]triazin-6-yl)piperidin-4- |
| | yl)propyl)piperazin-1-yl)benzamide |
| Example | N-((1r,3r)-3-(3-chloro-4-cyanophenoxy)-2,2,4,4-tetramethylcyclobutyl)-4-(4-((4-(1-(3-(2,6- |
| 49 | dioxopiperidin-3-yl)-4-oxo-3,4-dihydrobenzo[d][1,2,3]triazin-6-yl)piperidine-4- |
| | carbonyl)piperazin-1-yl)methyl)piperidin-1-yl)benzamide |
| Example | N-((1r,3r)-3-(3-chloro-4-cyanophenoxy)-2,2,4,4-tetramethylcyclobutyl)-4-(1-((1-(3-(2,6- |
| 50 | dioxopiperidin-3-yl)-4-oxo-3,4-dihydrobenzo[d][1,2,3]triazin-6-yl)piperidin-4- |
| | yl)methyl)piperidin-4-yl)benzamide |
| Example | N-((1r,3r)-3-(3-chloro-4-cyanophenoxy)-2,2,4,4-tetramethylcyclobutyl)-4-(1-((1-(1-(3-(2,6- |
| 51 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)benzamide |
| Example | N-((1r,3r)-3-(3-chloro-4-cyanophenoxy)-2,2,4,4-tetramethylcyclobutyl)-4-(1-((1-(1-(3-(2,6- |
| 52 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)benzamide |
| Example | N-((1r,3r)-3-(3-chloro-4-cyanophenoxy)-2,2,4,4-tetramethylcyclobutyl)-4-(4-((1-((3-(2,6- |
| 53 | dioxopiperidin-3-yl)-4-oxo-3,4-dihydrobenzo[d][1,2,3]triazin-6-yl)glycyl)-4-hydroxypiperidin-4- |
| | yl)methyl)piperazin-1-yl)benzamide |
| Example | N-((1r,3r)-3-(3-chloro-4-cyanophenoxy)-2,2,4,4-tetramethylcyclobutyl)-4-(4-((1-(2-((3-(2,6- |
| 54 | dioxopiperidin-3-yl)-4-oxo-3,4-dihydrobenzo[d][1,2,3]triazin-6-yl)oxy)acetyl)-4- |
| | hydroxypiperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 55 | dioxopiperidin-3-yl)-4-oxo-3,4-dihydrobenzo[d][1,2,3]triazin-6-yl)piperidine-4-carbonyl)-4- |
| | hydroxypiperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 56 | dioxopiperidin-3-yl)-4-oxo-3,4-dihydrobenzo[d][1,2,3]triazin-6-yl)azetidine-3-carbonyl)-4- |
| | hydroxypiperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-(2-(4-(3-(2,6- |
| 57 | dioxopiperidin-3-yl)-4-oxo-3,4-dihydrobenzo[d][1,2,3]triazin-6-yl)piperazin-1- |
| | yl)acetyl)piperazin-1-yl)benzamide |
| Example | N-((1r,3r)-3-(3-chloro-4-cyanophenoxy)-2,2,4,4-tetramethylcyclobutyl)-4-(4-((1-(2-(4-(3-(2,6- |
| 58 | dioxopiperidin-3-yl)-4-oxo-3,4-dihydrobenzo[d][1,2,3]triazin-6-yl)piperazin-1- |
| | yl)acetyl)piperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-(2-(2-((3-(2,6- |
| 59 | dioxopiperidin-3-yl)-4-oxo-3,4-dihydrobenzo[d][1,2,3]triazin-6- |
| | yl)amino)acetamido)ethyl)piperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-(2-(2-((3-(2,6- |
| 60 | dioxopiperidin-3-yl)-4-oxo-3,4-dihydrobenzo[d][1,2,3]triazin-6- |
| | yl)oxy)acetamido)ethyl)piperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-(3-(2,6- |
| 61 | dioxopiperidin-3-yl)-4-oxo-3,4-dihydrobenzo[d][1,2,3]triazin-6-yl)-4-fluoropiperidin-4- |
| | yl)methyl)piperazin-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 62 | dioxopiperidin-3-yl)-4-oxo-3,4-dihydrobenzo[d][1,2,3]triazin-6-yl)piperidine-4-carbonyl)-4- |
| | fluoropiperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 63 | dioxopiperidin-3-yl)-4-oxo-3,4-dihydrobenzo[d][1,2,3]triazin-6-yl)azetidine-3-carbonyl)-4- |
| | fluoropiperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-(3-(2,6- |
| 64 | dioxopiperidin-3-yl)-4-oxo-3,4-dihydrobenzo[d][1,2,3]triazin-6-yl)piperidin-4- |
| | ylidene)methyl)piperazin-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 65 | dioxopiperidin-3-yl)-4-oxo-3,4-dihydrobenzo[d][1,2,3]triazin-6-yl)piperidine-4- |
| | carbonyl)piperidin-4-ylidene)methyl)piperazin-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 66 | dioxopiperidin-3-yl)-4-oxo-3,4-dihydrobenzo[d][1,2,3]triazin-6-yl)azetidine-3- |
| | carbonyl)piperidin-4-ylidene)methyl)piperazin-1-yl)benzamide |
| Example | N-((1r,3r)-3-(3-chloro-4-cyanophenoxy)-2,2,4,4-tetramethylcyclobutyl)-4-(4-((1-((1-(3-(2,6- |
| 67 | dioxopiperidin-3-yl)-4-oxo-3,4-dihydrobenzo[d][1,2,3]triazin-6-yl)azetidin-3-yl)methyl)piperidin- |
| | 4-yl)methyl)piperazin-1-yl)benzamide |
| Example | N-(2-(4-((4-(4-(((1r,3r)-3-(3-chloro-4-cyanophenoxy)-2,2,4,4- |
| 68 | tetramethylcyclobutyl)carbamoyl)phenyl)piperazin-1-yl)methyl)piperidin-1-yl)ethyl)-1-(3-(2,6- |
| | dioxopiperidin-3-yl)-4-oxo-3,4-dihydrobenzo[d][1,2,3]triazin-6-yl)piperidine-4-carboxamide |
| Example | N-(2-(4-((4-(4-(((1r,3r)-3-(3-chloro-4-cyanophenoxy)-2,2,4,4- |
| 69 | tetramethylcyclobutyl)carbamoyl)phenyl)piperazin-1-yl)methyl)piperidin-1-yl)ethyl)-1-(3-(2,6- |
| | dioxopiperidin-3-yl)-4-oxo-3,4-dihydrobenzo[d][1,2,3]triazin-6-yl)azetidine-3-carboxamide |
| Example | N-((1r,3r)-3-(3-chloro-4-cyanophenoxy)-2,2,4,4-tetramethylcyclobutyl)-4-(4-((1-((1-(3-(2,6- |
| 70 | dioxopiperidin-3-yl)-4-oxo-3,4-dihydrobenzo[d][1,2,3]triazin-6-yl)piperidin-4- |
| | yl)methyl)piperidin-4-yl)methyl)piperazin-1-yl)benzamide |
| Example | N-((1r,3r)-3-(3-chloro-4-cyanophenoxy)-2,2,4,4-tetramethylcyclobutyl)-4-(5-(7-(1-(3-(2,6- |
| 71 | dioxopiperidin-3-yl)-4-oxo-3,4-dihydrobenzo[d][1,2,3]triazin-6-yl)piperidine-4-carbonyl)-7- |
| | azaspiro[3.5]nonan-2-yl)-2,5-diazabicyclo[2.2.1]heptan-2-yl)benzamide |
| Example | N-((1r,3r)-3-(3-chloro-4-cyanophenoxy)-2,2,4,4-tetramethylcyclobutyl)-4-(5-((1-(3-(2,6- |
| 72 | dioxopiperidin-3-yl)-4-oxo-3,4-dihydrobenzo[d][1,2,3]triazin-6-yl)piperidin-4-yl)methyl)-2,5- |
| | diazabicyclo[2.2.1]heptan-2-yl)benzamide |
| Example | N-((1r,3r)-3-(3-chloro-4-cyanophenoxy)-2,2,4,4-tetramethylcyclobutyl)-4-(5-((1-(1-(3-(2,6- |
| 73 | dioxopiperidin-3-yl)-4-oxo-3,4-dihydrobenzo[d][1,2,3]triazin-6-yl)azetidine-3- |
| | carbonyl)piperidin-4-yl)methyl)-2,5-diazabicyclo[2.2.1]heptan-2-yl)benzamide |
| Example | N-((1r,3r)-3-(3-chloro-4-cyanophenoxy)-2,2,4,4-tetramethylcyclobutyl)-4-(1-(2-((1-(1-(3-(2,6- |
| 74 | dioxopiperidin-3-yl)-4-oxo-3,4-dihydrobenzo[d][1,2,3]triazin-6-yl)piperidine-4- |
| | carbonyl)piperidin-4-yl)oxy)ethyl)piperidin-4-yl)benzamide |
| Example | N-((1r,3r)-3-(3-chloro-4-cyanophenoxy)-2,2,4,4-tetramethylcyclobutyl)-4-(1-(2-((1-(1-(3-(2,6- |
| 75 | dioxopiperidin-3-yl)-4-oxo-3,4-dihydrobenzo[d][1,2,3]triazin-6-yl)azetidine-3- |
| | carbonyl)piperidin-4-yl)oxy)ethyl)piperidin-4-yl)benzamide |
| Example | N-((1r,3r)-3-(3-chloro-4-cyanophenoxy)-2,2,4,4-tetramethylcyclobutyl)-4-(1-(2-((1-(3-(2,6- |
| 76 | dioxopiperidin-3-yl)-4-oxo-3,4-dihydrobenzo[d][1,2,3]triazin-6-yl)piperidin-4- |
| | yl)oxy)ethyl)piperidin-4-yl)benzamide |
| Example | N-((1r,3r)-3-(3-chloro-4-isocyanophenoxy)-2,2,4,4-tetramethylcyclobutyl)-4-(4-(2-(1-(3-(2,6- |
| 77 | dioxopiperidin-3-yl)-4-oxo-3,4-dihydrobenzo[d][1,2,3]triazin-6-yl)-1H-1,2,3-triazol-4- |
| | yl)ethyl)piperazin-1-yl)benzamide |
| Example | N-((1r,3r)-3-(3-chloro-4-cyanophenoxy)-2,2,4,4-tetramethylcyclobutyl)-6-(4-((1-(3-(2,6- |
| 78 | dioxopiperidin-3-yl)-4-oxo-3,4-dihydrobenzo[d][1,2,3]triazin-6-yl)piperidin-4- |
| | yl)methyl)piperazin-1-yl)nicotinamide |
| Example | N-((1r,3r)-3-(3-chloro-4-cyanophenoxy)-2,2,4,4-tetramethylcyclobutyl)-5-(4-((1-(3-(2,6- |
| 79 | dioxopiperidin-3-yl)-4-oxo-3,4-dihydrobenzo[d][1,2,3]triazin-6-yl)piperidin-4- |
| | yl)methyl)piperazin-1-yl)pyrazine-2-carboxamide |
| Example | N-((1r,3r)-3-(3-chloro-4-cyanophenoxy)-2,2,4,4-tetramethylcyclobutyl)-6-(4-((1-(1-(3-(2,6- |
| 80 | dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)nicotinamide |
| Example | N-((1r,3r)-3-(3-chloro-4-cyanophenoxy)-2,2,4,4-tetramethylcyclobutyl)-6-(4-((1-(1-(3-(2,6- |
| 81 | dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)nicotinamide |
| Example | N-((1r,3r)-3-(3-chloro-4-cyanophenoxy)-2,2,4,4-tetramethylcyclobutyl)-4-(1-(1-((1-(1-(3-(2,6- |
| 82 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)benzamide |
| Example | N-((1r,3r)-3-(3-chloro-4-cyanophenoxy)-2,2,4,4-tetramethylcyclobutyl)-4-(1-(1-((1-(1-(3-(2,6- |
| 83 | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)benzamide |
| Example | N-((1r,3r)-3-(3-chloro-4-cyanophenoxy)-2,2,4,4-tetramethylcyclobutyl)-4-(1-(1-((1-(3-(2,6- |
| 84 | dioxopiperidin-3-yl)-4-oxo-3,4-dihydrobenzo[d][1,2,3]triazin-6-yl)piperidin-4- |
| | yl)methyl)piperidin-4-yl)-1H-pyrazol-4-yl)benzamide |
| Example | N-((1r,3r)-3-(3-chloro-4-cyanophenoxy)-2,2,4,4-tetramethylcyclobutyl)-4-(3-(1-(3-(2,6- |
| 85 | dioxopiperidin-3-yl)-4-oxo-3,4-dihydrobenzo[d][1,2,3]triazin-6-yl)piperidin-4-yl)prop-1-yn-1- |
| | yl)benzamide |
| Example | N-((1r,3r)-3-(3-chloro-4-cyanophenoxy)-2,2,4,4-tetramethylcyclobutyl)-4-(3-(1-(1-(3-(2,6- |
| 86 | dioxopiperidin-3-yl)-4-oxo-3,4-dihydrobenzo[d][1,2,3]triazin-6-yl)piperidine-4- |
| | carbonyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(3-(1-(1-(3-(2,6- |
| 87 | dioxopiperidin-3-yl)-4-oxo-3,4-dihydrobenzo[d][1,2,3]triazin-6-yl)azetidine-3- |
| | carbonyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(3-(1-((1-(3-(2,6- |
| 88 | dioxopiperidin-3-yl)-4-oxo-3,4-dihydrobenzo[d][1,2,3]triazin-6-yl)piperidin-4- |
| | yl)methyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(3-(1 [symbol] (3-(2,6- |
| 89 | dioxopiperidin-3-yl)-4-oxo-3,4-dihydrobenzo[d][1,2,3]triazin-6-yl)-[1,4 [symbol] bipiperdin]-4-yl)prop-1- |
| | yn-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 90 | dioxopiperidin-3-yl)-4-oxo-3,4-dihydrobenzo[d][1,2,3]triazin-6-yl)azetidine-3- |
| | carbonyl)piperidin-4-yl)methyl)-1H-1,2,3-triazol-1-yl)benzamide |
| Example | N-((1r,3r)-3-(3-chloro-4-cyanophenoxy)-2,2,4,4-tetramethylcyclobutyl)-4-(4-((1-(3-(2,6- |
| 91 | dioxopiperidin-3-yl)-4-oxo-3,4-dihydrobenzo[d][1,2,3]triazin-6-yl)-1H-1,2,3-triazol-4- |
| | yl)methyl)piperazin-1-yl)benzamide |
| Example | N-((1r,3r)-3-(3-chloro-4-cyanophenoxy)-2,2,4,4-tetramethylcyclobutyl)-4-(2-(5-(3-(2,6- |
| 92 | dioxopiperidin-3-yl)-4-oxo-3,4-dihydrobenzo[d][1,2,3]triazin-6-yl)hexahydropyrrolo[3,4- |
| | c]pyrrol-2(1H)-yl)ethyl)benzamide |
| Example | N-((1r,3r)-3-(3-chloro-4-cyanophenoxy)-2,2,4,4-tetramethylcyclobutyl)-4-(2-(5-(1-(3-(2,6- |
| 93 | dioxopiperidin-3-yl)-4-oxo-3,4-dihydrobenzo[d][1,2,3]triazin-6-yl)piperidine-4- |
| | carbonyl)hexahydropyrrolo[3,4-c]pyrrol-2(1H)-yl)ethyl)benzamide |
| Example | N-((1r,3r)-3-(3-chloro-4-cyanophenoxy)-2,2,4,4-tetramethylcyclobutyl)-4-(2-(5-(1-(3-(2,6- |
| 94 | dioxopiperidin-3-yl)-4-oxo-3,4-dihydrobenzo[d][1,2,3]triazin-6-yl)azetidine-3- |
| | carbonyl)hexahydropyrrolo[3,4-c]pyrrol-2(1H)-yl)ethyl)benzamide |
| Example | N-((1r,3r)-3-(3-chloro-4-cyanophenoxy)-2,2,4,4-tetramethylcyclobutyl)-4-((1-(1-(3-(2,6- |
| 95 | dioxopiperidin-3-yl)-4-oxo-3,4-dihydrobenzo[d][1,2,3]triazin-6-yl)azetidine-3- |
| | carbonyl)piperidin-4-yl)ethynyl)benzamide |
| Example | N-((1r,3r)-3-(3-chloro-4-cyanophenoxy)-2,2,4,4-tetramethylcyclobutyl)-4-((1-((1-(1-(3-(2,6- |
| 96 | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)ethynyl)benzamide |
| Example | N-((1r,3r)-3-(3-chloro-4-cyanophenoxy)-2,2,4,4-tetramethylcyclobutyl)-4-((1-((1-(1-(3-(2,6- |
| 97 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)benzamide |
| Example | N-((1r,3r)-3-(3-chloro-4-cyanophenoxy)-2,2,4,4-tetramethylcyclobutyl)-4-((1-((1-((3-(2,6- |
| 98 | dioxopiperidin-3-yl)-4-oxo-3,4-dihydrobenzo[d][1,2,3]triazin-6-yl)glycyl)piperidin-4- |
| | yl)methyl)piperidin-4-yl)ethynyl)benzamide |
| Example | N-((1r,3r)-3-(3-chloro-4-cyanophenoxy)-2,2,4,4-tetramethylcyclobutyl)-4-((1-((1-(3-(2,6- |
| 99 | dioxopiperidin-3-yl)-4-oxo-3,4-dihydrobenzo[d][1,2,3]triazin-6-yl)piperidin-4- |
| | yl)methyl)piperidin-4-yl)ethynyl)benzamide |
| Example | N-((1r,3r)-3-(3-chloro-4-cyanophenoxy)-2,2,4,4-tetramethylcyclobutyl)-2-(4-((1-(3-(2,6- |
| 100 | dioxopiperidin-3-yl)-4-oxo-3,4-dihydrobenzo[d][1,2,3]triazin-6-yl)piperidin-4- |
| | yl)methyl)piperazin-1-yl)pyrimidine-5-carboxamide |
| Example | N-((1r,3r)-3-(3-chloro-4-cyanophenoxy)-2,2,4,4-tetramethylcyclobutyl)-4-(4-(1-(1-(3-(2,6- |
| 101 | dioxopiperidin-3-yl)-4-oxo-3,4-dihydrobenzo[d][1,2,3]triazin-6-yl)azetidine-3- |
| | carbonyl)piperidin-4-yl)-1H-1,2,3-triazol-1-yl)benzamide |
| Example | N-((1r,3r)-3-(3-chloro-4-cyanophenoxy)-2,2,4,4-tetramethylcyclobutyl)-4-(4-(1-(1-(3-(2,6- |
| 102 | dioxopiperidin-3-yl)-4-oxo-3,4-dihydrobenzo[d][1,2,3]triazin-6-yl)piperidine-4- |
| | carbonyl)piperidin-4-yl)-1H-1,2,3-triazol-1-yl)benzamide |
| Example | N-((1r,3r)-3-(3-chloro-4-cyanophenoxy)-2,2,4,4-tetramethylcyclobutyl)-4-(4-((1-(3-(2,6- |
| 103 | dioxopiperidin-3-yl)-4-oxo-3,4-dihydrobenzo[d][1,2,3]triazin-6-yl)piperidin-4-yl)methyl)-1H- |
| | 1,2,3-triazol-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 104 | dioxopiperidin-3-yl)-4-oxo-3,4-dihydrobenzo[d][1,2,3]triazin-6-yl)piperidine-4- |
| | carbonyl)piperidin-4-yl)methyl)-1H-1,2,3-triazol-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 105 | dioxopiperidin-3-yl)-4-oxo-3,4-dihydrobenzo[d][1,2,3]triazin-6-yl)piperidin-4-yl)-1H-1,2,3- |
| | triazol-4-yl)methyl)piperazin-1-yl)benzamide |
| Example | N-((1r,3r)-3-(3-chloro-4-cyanophenoxy)-2,2,4,4-tetramethylcyclobutyl)-4-(4-((1-((3-(2,6- |
| 106 | dioxopiperidin-3-yl)-4-oxo-3,4-dihydrobenzo[d][1,2,3]triazin-6-yl)glycyl)piperidin-4-yl)methyl)- |
| | 1H-1,2,3-triazol-1-yl)benzamide |
| Example | N-((1r,3r)-3-(3-chloro-4-cyanophenoxy)-2,2,4,4-tetramethylcyclobutyl)-4-(4-(1-((3-(2,6- |
| 107 | dioxopiperidin-3-yl)-4-oxo-3,4-dihydrobenzo[d][1,2,3]triazin-6-yl)glycyl)piperidin-4-yl)-1H- |
| | 1,2,3-triazol-1-yl)benzamide |
| Example | N-((1r,3r)-3-(3-chloro-4-cyanophenoxy)-2,2,4,4-tetramethylcyclobutyl)-4-(2-(4-((1-(1-(3-(2,6- |
| 108 | dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)ethyl)benzamide |
| Example | N-((1r,3r)-3-(3-chloro-4-cyanophenoxy)-2,2,4,4-tetramethylcyclobutyl)-4-(2-(4-((1-(1-(3-(2,6- |
| 109 | dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)ethyl)benzamide |
| Example | N-((1r,3r)-3-(3-chloro-4-cyanophenoxy)-2,2,4,4-tetramethylcyclobutyl)-4-(2-(4-((1-((3-(2,6- |
| 110 | dioxopiperidin-3-yl)-4-oxo-3,4-dihydrobenzo[d][1,2,3]triazin-6-yl)glycyl)piperidin-4- |
| | yl)methyl)piperazin-1-yl)ethyl)benzamide |
| Example | N-((1r,3r)-3-(3-chloro-4-cyanophenoxy)-2,2,4,4-tetramethylcyclobutyl)-4-(2-(4-((1-(3-(2,6- |
| 111 | dioxopiperidin-3-yl)-4-oxo-3,4-dihydrobenzo[d][1,2,3]triazin-6-yl)piperidin-4- |
| | yl)methyl)piperazin-1-yl)ethyl)benzamide |
| Example | N-((1r,3r)-3-(3-chloro-4-cyanophenoxy)-2,2,4,4-tetramethylcyclobutyl)-4-(1-(1-((1-((3-(2,6- |
| 112 | dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)benzamide |
| Example | N-((1r,3r)-3-(3-chloro-4-cyanophenoxy)-2,2,4,4-tetramethylcyclobutyl)-4-(1-(1-((1-(2-((3-(2,6- |
| 113 | dioxopiperidin-3-yl)-4-oxo-3,4-dihydrobenzo[d][1,2,3]triazin-6-yl)oxy)acetyl)piperidin-4- |
| | yl)methyl)piperidin-4-yl)-1H-pyrazol-4-yl)benzamide |
| Example | N-((1r,3r)-3-(3-chloro-4-cyanophenoxy)-2,2,4,4-tetramethylcyclobutyl)-6-(1-(1-((1-(1-(3-(2,6- |
| 114 | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)nicotinamide |
| Example | N-((1r,3r)-3-(3-chloro-4-cyanophenoxy)-2,2,4,4-tetramethylcyclobutyl)-6-(1-(1-((1-(3-(2,6- |
| 115 | dioxopiperidin-3-yl)-4-oxo-3,4-dihydrobenzo[d][1,2,3]triazin-6-yl)piperidin-4- |
| | yl)methyl)piperidin-4-yl)-1H-pyrazol-4-yl)nicotinamide |
| Example | N-((1r,3r)-3-(3-chloro-4-cyanophenoxy)-2,2,4,4-tetramethylcyclobutyl)-4-(3-(1-((1-(1-(3-(2,6- |
| 116 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(3-(1-((1-(1-(3-(2,6- |
| 117 | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(3-(1-((1-((3-(2,6- |
| 118 | dioxopiperidin-3-yl)-4-oxo-3,4-dihydrobenzo[d][1,2,3]triazin-6-yl)glycyl)piperidin-4- |
| | yl)methyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(3-(1-((1-(2-((3-(2,6- |
| 119 | dioxopiperidin-3-yl)-4-oxo-3,4-dihydrobenzo[d][1,2,3]triazin-6-yl)oxy)acetyl)piperidin-4- |
| | yl)methyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(4-((4-(3-(2,6- |
| 120 | dioxopiperidin-3-yl)-4-oxo-3,4-dihydrobenzo[d][1,2,3]triazin-6-yl)piperazin-1-yl)methyl)-1H- |
| | 1,2,3-triazol-1-yl)benzamide |
| Example | N-((1r,3r)-3-(3-chloro-4-cyanophenoxy)-2,2,4,4-tetramethylcyclobutyl)-4-(4-((4-(1-(3-(2,6- |
| 121 | dioxopiperidin-3-yl)-4-oxo-3,4-dihydrobenzo[d][1,2,3]triazin-6-yl)piperidine-4- |
| | carbonyl)piperazin-1-yl)methyl)-1H-1,2,3-triazol-1-yl)benzamide |
| Example | N-((1r,3r)-3-(3-chloro-4-cyanophenoxy)-2,2,4,4-tetramethylcyclobutyl)-4-(4-((4-(1-(3-(2,6- |
| 122 | dioxopiperidin-3-yl)-4-oxo-3,4-dihydrobenzo[d][1,2,3]triazin-6-yl)azetidine-3- |
| | carbonyl)piperazin-1-yl)methyl)-1H-1,2,3-triazol-1-yl)benzamide |
| Example | N-((1r,3r)-3-(3-chloro-4-cyanophenoxy)-2,2,4,4-tetramethylcyclobutyl)-4-(4-((4-((3-(2,6- |
| 123 | dioxopiperidin-3-yl)-4-oxo-3,4-dihydrobenzo[d][1,2,3]triazin-6-yl)glycyl)piperazin-1-yl)methyl)- |
| | 1H-1,2,3-triazol-1-yl)benzamide |
| Example | N-((1r,3r)-3-(3-chloro-4-cyanophenoxy)-2,2,4,4-tetramethylcyclobutyl)-6-(1-(1-((1-(1-(3-(2,6- |
| 124 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)nicotinamide |
| Example | N-((1r,3r)-3-(3-chloro-4-cyanophenoxy)-2,2,4,4-tetramethylcyclobutyl)-6-(1-(1-((1-((3-(2,6- |
| 125 | dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)nicotinamide |
| Example | N-((1r,3r)-3-(3-chloro-4-cyanophenoxy)-2,2,4,4-tetramethylcyclobutyl)-6-(3-(1-((1-(1-(3-(2,6- |
| 126 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)prop-1-yn-1-yl)nicotinamide |
| Example | N-((1r,3r)-3-(3-chloro-4-cyanophenoxy)-2,2,4,4-tetramethylcyclobutyl)-6-(3-(1-((1-(1-(3-(2,6- |
| 127 | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)prop-1-yn-1-yl)nicotinamide |
| Example | N-((1r,3r)-3-(3-chloro-4-cyanophenoxy)-2,2,4,4-tetramethylcyclobutyl)-6-(3-(1-((1-((3-(2,6- |
| 128 | dioxopiperidin-3-yl)-4-oxo-3,4-dihydrobenzo[d][1,2,3]triazin-6-yl)glycyl)piperidin-4- |
| | yl)methyl)piperidin-4-yl)prop-1-yn-1-yl)nicotinamide |
| Example | N-((1r,3r)-3-(3-chloro-4-cyanophenoxy)-2,2,4,4-tetramethylcyclobutyl)-6-(3-(1-((1-(3-(2,6- |
| 129 | dioxopiperidin-3-yl)-4-oxo-3,4-dihydrobenzo[d][1,2,3]triazin-6-yl)piperidin-4- |
| | yl)methyl)piperidin-4-yl)prop-1-yn-1-yl)nicotinamide |
| Example | N-((1r,3r)-3-(3-chloro-4-cyanophenoxy)-2,2,4,4-tetramethylcyclobutyl)-6-((1-((1-(1-(3-(2,6- |
| 130 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)nicotinamide |
| Example | N-((1r,3r)-3-(3-chloro-4-cyanophenoxy)-2,2,4,4-tetramethylcyclobutyl)-6-((1-((1-(1-(3-(2,6- |
| 131 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)nicotinamide |
| Example | N-((1r,3r)-3-(3-chloro-4-cyanophenoxy)-2,2,4,4-tetramethylcyclobutyl)-6-((1-((1-((3-(2,6- |
| 132 | dioxopiperidin-3-yl)-4-oxo-3,4-dihydrobenzo[d][1,2,3]triazin-6-yl)glycyl)piperidin-4- |
| | yl)methyl)piperidin-4-yl)ethynyl)nicotinamide |
| Example | N-((1r,3r)-3-(3-chloro-4-cyanophenoxy)-2,2,4,4-tetramethylcyclobutyl)-6-((1-((1-(2-((3-(2,6- |
| 133 | dioxopiperidin-3-yl)-4-oxo-3,4-dihydrobenzo[d][1,2,3]triazin-6-yl)oxy)acetyl)piperidin-4- |
| | yl)methyl)piperidin-4-yl)ethynyl)nicotinamide |
| Example | N-((1r,3r)-3-(3-chloro-4-cyanophenoxy)-2,2,4,4-tetramethylcyclobutyl)-4-(4-(3-(1-(3-(2,6- |
| 134 | dioxopiperidin-3-yl)-4-oxo-3,4-dihydrobenzo[d][1,2,3]triazin-6-yl)-1H-1,2,3-triazol-4- |
| | yl)propyl)piperazin-1-yl)benzamide |
| Example | N-((1r,3r)-3-(3-chloro-4-cyanophenoxy)-2,2,4,4-tetramethylcyclobutyl)-4-(5-(2-((3-(2,6- |
| 135 | dioxopiperidin-3-yl)-4-oxo-3,4-dihydrobenzo[d][1,2,3]triazin-6- |
| | yl)oxy)acetyl)hexahydropyrrolo[3,4-c]pyrrol-2(1H)-yl)benzamide |
| Example | N-((1r,3r)-3-(3-chloro-4-cyanophenoxy)-2,2,4,4-tetramethylcyclobutyl)-4-(5-((3-(2,6- |
| 136 | dioxopiperidin-3-yl)-4-oxo-3,4-dihydrobenzo[d][1,2,3]triazin-6-yl)glycyl)hexahydropyrrolo[3,4- |
| | c]pyrrol-2(1H)-yl)benzamide |
| Example | N-((1r,3r)-3-(3-chloro-4-cyanophenoxy)-2,2,4,4-tetramethylcyclobutyl)-4-(5-((1-(2-((3-(2,6- |
| 137 | dioxopiperidin-3-yl)-4-oxo-3,4-dihydrobenzo[d][1,2,3]triazin-6-yl)oxy)acetyl)piperidin-4- |
| | yl)methyl)hexahydropyrrolo[3,4-c]pyrrol-2(1H)-yl)benzamide |
| Example | N-((1r,3r)-3-(3-chloro-4-cyanophenoxy)-2,2,4,4-tetramethylcyclobutyl)-4-(5-((1-((3-(2,6- |
| 138 | dioxopiperidin-3-yl)-4-oxo-3,4-dihydrobenzo[d][1,2,3]triazin-6-yl)glycyl)piperidin-4- |
| | yl)methyl)hexahydropyrrolo[3,4-c]pyrrol-2(1H)-yl)benzamide |
| Example | N-((1r,3r)-3-(3-chloro-4-cyanophenoxy)-2,2,4,4-tetramethylcyclobutyl)-4-(5-((1-(1-(3-(2,6- |
| 139 | dioxopiperidin-3-yl)-4-oxo-3,4-dihydrobenzo[d][1,2,3]triazin-6-yl)piperidine-4- |
| | carbonyl)piperidin-4-yl)methyl)hexahydropyrrolo[3,4-c]pyrrol-2(1H)-yl)benzamide |
| Example | N-((1r,3r)-3-(3-chloro-4-cyanophenoxy)-2,2,4,4-tetramethylcyclobutyl)-4-(5-((1-(1-(3-(2,6- |
| 140 | dioxopiperidin-3-yl)-4-oxo-3,4-dihydrobenzo[d][1,2,3]triazin-6-yl)azetidine-3- |
| | carbonyl)piperidin-4-yl)methyl)hexahydropyrrolo[3,4-c]pyrrol-2(1H)-yl)benzamide |
| Example | N-((1r,3r)-3-(3-chloro-4-cyanophenoxy)-2,2,4,4-tetramethylcyclobutyl)-4-((1-(1-(3-(2,6- |
| 141 | dioxopiperidin-3-yl)-4-oxo-3,4-dihydrobenzo[d][1,2,3]triazin-6-yl)piperidine-4- |
| | carbonyl)piperidin-4-yl)ethynyl)benzamide |
| Example | N-((1r,3r)-3-(3-chloro-4-cyanophenoxy)-2,2,4,4-tetramethylcyclobutyl)-4-((1-(3-(2,6- |
| 142 | dioxopiperidin-3-yl)-4-oxo-3,4-dihydrobenzo[d][1,2,3]triazin-6-yl)piperidin-4- |
| | yl)ethynyl)benzamide |
| Example | N-((1r,3r)-3-(3-chloro-4-cyanophenoxy)-2,2,4,4-tetramethylcyclobutyl)-4-((1-((3-(2,6- |
| 143 | dioxopiperidin-3-yl)-4-oxo-3,4-dihydrobenzo[d][1,2,3]triazin-6-yl)glycyl)piperidin-4- |
| | yl)ethynyl)benzamide |
| Example | N-((1r,3r)-3-(3-chloro-4-cyanophenoxy)-2,2,4,4-tetramethylcyclobutyl)-4-((1-(2-((3-(2,6- |
| 144 | dioxopiperidin-3-yl)-4-oxo-3,4-dihydrobenzo[d][1,2,3]triazin-6-yl)oxy)acetyl)piperidin-4- |
| | yl)ethynyl)benzamide |
| Example | N-((1r,3r)-3-(3-chloro-4-cyanophenoxy)-2,2,4,4-tetramethylcyclobutyl)-2-(4-((1-(1-(3-(2,6- |
| 145 | dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)pyrimidine-5-carboxamide |
| Example | N-((1r,3r)-3-(3-chloro-4-cyanophenoxy)-2,2,4,4-tetramethylcyclobutyl)-2-(4-((1-(1-(3-(2,6- |
| 146 | dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)pyrimidine-5-carboxamide |
| Example | N-((1r,3r)-3-(3-chloro-4-cyanophenoxy)-2,2,4,4-tetramethylcyclobutyl)-2-(4-((1-((3-(2,6- |
| 147 | dioxopiperidin-3-yl)-4-oxo-3,4-dihydrobenzo[d][1,2,3]triazin-6-yl)glycyl)piperidin-4- |
| | yl)methyl)piperazin-1-yl)pyrimidine-5-carboxamide |
| Example | N-((1r,3r)-3-(3-chloro-4-cyanophenoxy)-2,2,4,4-tetramethylcyclobutyl)-2-(4-((1-(2-((3-(2,6- |
| 148 | dioxopiperidin-3-yl)-4-oxo-3,4-dihydrobenzo[d][1,2,3]triazin-6-yl)oxy)acetyl)piperidin-4- |
| | yl)methyl)piperazin-1-yl)pyrimidine-5-carboxamide |
| Example | N-((1r,3r)-3-(3-chloro-4-cyanophenoxy)-2,2,4,4-tetramethylcyclobutyl)-4-(4-(((3-(2,6- |
| 149 | dioxopiperidin-3-yl)-4-oxo-3,4-dihydrobenzo[d][1,2,3]triazin-6-yl)amino)methyl)-1H-1,2,3- |
| | triazol-1-yl)benzamide |
| Example | N-((1-(4-(((1r,3r)-3-(3-chloro-4-cyanophenoxy)-2,2,4,4- |
| 150 | tetramethylcyclobutyl)carbamoyl)phenyl)-1H-1,2,3-triazol-4-yl)methyl)-1-(3-(2,6- |
| | dioxopiperidin-3-yl)-4-oxo-3,4-dihydrobenzo[d][1,2,3]triazin-6-yl)piperidine-4-carboxamide |
| Example | N-((1-(4-(((1r,3r)-3-(3-chloro-4-cyanophenoxy)-2,2,4,4- |
| 151 | tetramethylcyclobutyl)carbamoyl)phenyl)-1H-1,2,3-triazol-4-yl)methyl)-1-(3-(2,6- |
| | dioxopiperidin-3-yl)-4-oxo-3,4-dihydrobenzo[d][1,2,3]triazin-6-yl)azetidine-3-carboxamide |
| Example | N-((1r,3r)-3-(3-chloro-4-cyanophenoxy)-2,2,4,4-tetramethylcyclobutyl)-4-(4-((2-((3-(2,6- |
| 152 | dioxopiperidin-3-yl)-4-oxo-3,4-dihydrobenzo[d][1,2,3]triazin-6-yl)amino)acetamido)methyl)- |
| | 1H-1,2,3-triazol-1-yl)benzamide |
| Example | N-((1r,3r)-3-(3-chloro-4-cyanophenoxy)-2,2,4,4-tetramethylcyclobutyl)-4-(4-(2-((3-(2,6- |
| 153 | dioxopiperidin-3-yl)-4-oxo-3,4-dihydrobenzo[d][1,2,3]triazin-6-yl)amino)ethyl)-1H-1,2,3-triazol- |
| | 1-yl)benzamide |
| Example | N-(2-(1-(4-(((1r,3r)-3-(3-chloro-4-cyanophenoxy)-2,2,4,4- |
| 154 | tetramethylcyclobutyl)carbamoyl)phenyl)-1H-1,2,3-triazol-4-yl)ethyl)-1-(3-(2,6-dioxopiperidin- |
| | 3-yl)-4-oxo-3,4-dihydrobenzo[d][1,2,3]triazin-6-yl)piperidine-4-carboxamide |
| Example | N-(2-(1-(4-(((1r,3r)-3-(3-chloro-4-cyanophenoxy)-2,2,4,4- |
| 155 | tetramethylcyclobutyl)carbamoyl)phenyl)-1H-1,2,3-triazol-4-yl)ethyl)-1-(3-(2,6-dioxopiperidin- |
| | 3-yl)-4-oxo-3,4-dihydrobenzo[d][1,2,3]triazin-6-yl)azetidine-3-carboxamide |
| Example | N-((1r,3r)-3-(3-chloro-4-cyanophenoxy)-2,2,4,4-tetramethylcyclobutyl)-4-(4-(2-(2-((3-(2,6- |
| 156 | dioxopiperidin-3-yl)-4-oxo-3,4-dihydrobenzo[d][1,2,3]triazin-6-yl)amino)acetamido)ethyl)-1H- |
| | 1,2,3-triazol-1-yl)benzamide |
| Example | N-(2-(2-(2-(1-(4-(((1r,3r)-3-(3-chloro-4-cyanophenoxy)-2,2,4,4- |
| 157 | tetramethylcyclobutyl)carbamoyl)phenyl)-1H-1,2,3-triazol-4-yl)ethoxy)ethoxy)ethyl)-1-(3-(2,6- |
| | dioxopiperidin-3-yl)-4-oxo-3,4-dihydrobenzo[d][1,2,3]triazin-6-yl)azetidine-3-carboxamide |
| Example | N-(2-(2-(2-(1-(4-(((1r,3r)-3-(3-chloro-4-cyanophenoxy)-2,2,4,4- |
| 158 | tetramethylcyclobutyl)carbamoyl)phenyl)-1H-1,2,3-triazol-4-yl)ethoxy)ethoxy)ethyl)-1-(3-(2,6- |
| | dioxopiperidin-3-yl)-4-oxo-3,4-dihydrobenzo[d][1,2,3]triazin-6-yl)piperidine-4-carboxamide |
| Example | N-((1r,3r)-3-(3-chloro-4-cyanophenoxy)-2,2,4,4-tetramethylcyclobutyl)-4-(4-(2-(2-(2-(2-((3- |
| 159 | (2,6-dioxopiperidin-3-yl)-4-oxo-3,4-dihydrobenzo[d][1,2,3]triazin-6- |
| | yl)amino)acetamido)ethoxy)ethoxy)ethyl)-1H-1,2,3-triazol-1-yl)benzamide |
| Example | N-((1r,4r)-4-(3-chloro-4-cyanophenoxy)cyclohexyl)-6-(4-((4-(3-(2,6-dioxopiperidin-3-yl)-4-oxo- |
| 160 | 3,4-dihydrobenzo[d][1,2,3]triazin-6-yl)piperazin-1-yl)methyl)piperidin-1-yl)nicotinamide |
| Example | N-((1r,4r)-4-(3-chloro-4-cyanophenoxy)cyclohexyl)-6-(4-(3-((3-(2,6-dioxopiperidin-3-yl)-4-oxo- |
| 161 | 3,4-dihydrobenzo[d][1,2,3]triazin-6-yl)oxy)propyl)piperazin-1-yl)nicotinamide |
| Example | N-((1r,3r)-3-(3-chloro-4-cyanophenoxy)-2,2,4,4-tetramethylcyclobutyl)-6-(4-((4-(3-(2,6- |
| 162 | dioxopiperidin-3-yl)-4-oxo-3,4-dihydrobenzo[d][1,2,3]triazin-6-yl)piperazin-1- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(4-(5-((3-(2,6- |
| 163 | dioxopiperidin-3-yl)-4-oxo-3,4-dihydrobenzo[d][1,2,3]triazin-6-yl)oxy)pentyl)piperazin-1- |
| | yl)nicotinamide |
| Example | N-((1r,4r)-4-(3-chloro-4-cyanophenoxy)cyclohexyl)-6-(4-((4-(3-(2,6-dioxopiperidin-3-yl)-4-oxo- |
| 164 | 3,4-dihydrobenzo[d][1,2,3]triazin-7-yl)piperazin-1-yl)methyl)piperidin-1-yl)nicotinamide |
| Example | N-((1r,3r)-3-(3-chloro-4-cyanophenoxy)-2,2,4,4-tetramethylcyclobutyl)-4-(4-((4-(3-(2,6- |
| 165 | dioxopiperidin-3-yl)-4-oxo-3,4-dihydrobenzo[d][1,2,3]triazin-7-yl)piperazin-1- |
| | yl)methyl)piperidin-1-yl)benzamide |
| Example | N-((1r,3r)-3-(3-chloro-4-cyanophenoxy)-2,2,4,4-tetramethylcyclobutyl)-4-(4-((4-(3-(2,6- |
| 166 | dioxopiperidin-3-yl)-4-oxo-3,4-dihydrobenzo[d][1,2,3]triazin-6-yl)piperazin-1- |
| | yl)methyl)piperidin-1-yl)benzamide |
| Example | N-((1r,4r)-4-(3-chloro-4-cyanophenoxy)cyclohexyl)-6-(4-((7-(3-(2,6-dioxopiperidin-3-yl)-4-oxo- |
| 167 | 3,4-dihydrobenzo[d][1,2,3]triazin-7-yl)-2,7-diazaspiro[3.5]nonan-2-yl)methyl)piperidin-1- |
| | yl)nicotinamide |
| Example | N-((1r,4r)-4-(3-chloro-4-cyanophenoxy)cyclohexyl)-6-(4-((7-(3-(2,6-dioxopiperidin-3-yl)-4-oxo- |
| 168 | 3,4-dihydrobenzo[d][1,2,3]triazin-6-yl)-2,7-diazaspiro[3.5]nonan-2-yl)methyl)piperidin-1- |
| | yl)nicotinamide |
| Example | N-((1r,3r)-3-(3-chloro-4-cyanophenoxy)-2,2,4,4-tetramethylcyclobutyl)-6-(4-((7-(3-(2,6- |
| 169 | dioxopiperidin-3-yl)-4-oxo-3,4-dihydrobenzo[d][1,2,3]triazin-7-yl)-2,7-diazaspiro[3.5]nonan-2- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(4-((7-(3-(2,6- |
| 170 | dioxopiperidin-3-yl)-4-oxo-3,4-dihydrobenzo[d][1,2,3]triazin-6-yl)-2,7-diazaspiro[3.5]nonan-2- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(4-((2-(3-(2,6- |
| 171 | dioxopiperidin-3-yl)-4-oxo-3,4-dihydrobenzo[d][1,2,3]triazin-7-yl)-2,7-diazaspiro[3.5]nonan-7- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(4-((2-(3-(2,6- |
| 172 | dioxopiperidin-3-yl)-4-oxo-3,4-dihydrobenzo[d][1,2,3]triazin-6-yl)-2,7-diazaspiro[3.5]nonan-7- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(2-((1-(3-(2,6- |
| 173 | dioxopiperidin-3-yl)-4-oxo-3,4-dihydrobenzo[d][1,2,3]triazin-7-yl)piperidin-4-yl)methyl)-2,7- |
| | diazaspiro[3.5]nonan-7-yl)nicotinamide |
| Example | N-((1r,3r)-3-(3-chloro-4-cyanophenoxy)-2,2,4,4-tetramethylcyclobutyl)-6-(2-((1-(3-(2,6- |
| 174 | dioxopiperidin-3-yl)-4-oxo-3,4-dihydrobenzo[d][1,2,3]triazin-6-yl)piperidin-4-yl)methyl)-2,7- |
| | diazaspiro[3.5]nonan-7-yl)nicotinamide |
| Example | N-((1r,3r)-3-(3-chloro-4-cyanophenoxy)-2,2,4,4-tetramethylcyclobutyl)-6-(2-((1-(3-(2,6- |
| 175 | dioxopiperidin-3-yl)-4-oxo-3,4-dihydrobenzo[d][1,2,3]triazin-7-yl)azetidin-3-yl)methyl)-2,7- |
| | diazaspiro[3.5]nonan-7-yl)nicotinamide |
| Example | N-((1r,3r)-3-(3-chloro-4-cyanophenoxy)-2,2,4,4-tetramethylcyclobutyl)-6-(2-((1-(3-(2,6- |
| 176 | dioxopiperidin-3-yl)-4-oxo-3,4-dihydrobenzo[d][1,2,3]triazin-6-yl)azetidin-3-yl)methyl)-2,7- |
| | diazaspiro[3.5]nonan-7-yl)nicotinamide |
| Example | N-((1r,3r)-3-(3-chloro-4-cyanophenoxy)-2,2,4,4-tetramethylcyclobutyl)-6-(3-(4-(3-(2,6- |
| 177 | dioxopiperidin-3-yl)-4-oxo-3,4-dihydrobenzo[d][1,2,3]triazin-7-yl)piperazin-1-yl)prop-1-yn-1- |
| | yl)nicotinamide |
| Example | N-((1r,3r)-3-(3-chloro-4-cyanophenoxy)-2,2,4,4-tetramethylcyclobutyl)-6-(3-(4-(3-(2,6- |
| 178 | dioxopiperidin-3-yl)-4-oxo-3,4-dihydrobenzo[d][1,2,3]triazin-6-yl)piperazin-1-yl)prop-1-yn-1- |
| | yl)nicotinamide |
| Example | N-((1r,3r)-3-(3-chloro-4-cyanophenoxy)-2,2,4,4-tetramethylcyclobutyl)-6-(3-(7-(3-(2,6- |
| 179 | dioxopiperidin-3-yl)-4-oxo-3,4-dihydrobenzo[d][1,2,3]triazin-7-yl)-2,7-diazaspiro[3.5]nonan-2- |
| | yl)prop-1-yn-1-yl)nicotinamide |
| Example | N-((1r,3r)-3-(3-chloro-4-cyanophenoxy)-2,2,4,4-tetramethylcyclobutyl)-6-(3-(7-(3-(2,6- |
| 180 | dioxopiperidin-3-yl)-4-oxo-3,4-dihydrobenzo[d][1,2,3]triazin-6-yl)-2,7-diazaspiro[3.5]nonan-2- |
| | yl)prop-1-yn-1-yl)nicotinamide |
| Example | N-((1r,3r)-3-(3-chloro-4-cyanophenoxy)-2,2,4,4-tetramethylcyclobutyl)-6-(3-(2-(3-(2,6- |
| 181 | dioxopiperidin-3-yl)-4-oxo-3,4-dihydrobenzo[d][1,2,3]triazin-7-yl)-2,7-diazaspiro[3.5]nonan-7- |
| | yl)prop-1-yn-1-yl)nicotinamide |
| Example | N-((1r,3r)-3-(3-chloro-4-cyanophenoxy)-2,2,4,4-tetramethylcyclobutyl)-6-(3-(2-(3-(2,6- |
| 182 | dioxopiperidin-3-yl)-4-oxo-3,4-dihydrobenzo[d][1,2,3]triazin-6-yl)-2,7-diazaspiro[3.5]nonan-7- |
| | yl)prop-1-yn-1-yl)nicotinamide |
| Example | N-((1r,3r)-3-(3-chloro-4-cyanophenoxy)-2,2,4,4-tetramethylcyclobutyl)-6-((1-((1-(3-(2,6- |
| 183 | dioxopiperidin-3-yl)-4-oxo-3,4-dihydrobenzo[d][1,2,3]triazin-7-yl)azetidin-3-yl)methyl)piperidin- |
| | 4-yl)ethynyl)nicotinamide |
| Example | N-((1r,3r)-3-(3-chloro-4-cyanophenoxy)-2,2,4,4-tetramethylcyclobutyl)-6-((1-((1-(3-(2,6- |
| 184 | dioxopiperidin-3-yl)-4-oxo-3,4-dihydrobenzo[d][1,2,3]triazin-6-yl)azetidin-3-yl)methyl)piperidin- |
| | 4-yl)ethynyl)nicotinamide |
| Example | N-((1r,3R)-3-(3-chloro-4-cyanophenoxy)-2,2,4,4-tetramethylcyclobutyl)-6-((3R)-3-((7-(3-(2,6- |
| 185 | dioxopiperidin-3-yl)-4-oxo-3,4-dihydrobenzo[d][1,2,3]triazin-7-yl)-2,7-diazaspiro[3.5]nonan-2- |
| | yl)methyl)pyrrolidin-1-yl)nicotinamide |
| Example | N-((1r,3R)-3-(3-chloro-4-cyanophenoxy)-2,2,4,4-tetramethylcyclobutyl)-6-((3R)-3-((7-(3-(2,6- |
| 186 | dioxopiperidin-3-yl)-4-oxo-3,4-dihydrobenzo[d][1,2,3]triazin-6-yl)-2,7-diazaspiro[3.5]nonan-2- |
| | yl)methyl)pyrrolidin-1-yl)nicotinamide |
| Example | N-((1r,3S)-3-(3-chloro-4-cyanophenoxy)-2,2,4,4-tetramethylcyclobutyl)-6-((3S)-3-((7-(3-(2,6- |
| 187 | dioxopiperidin-3-yl)-4-oxo-3,4-dihydrobenzo[d][1,2,3]triazin-7-yl)-2,7-diazaspiro[3.5]nonan-2- |
| | yl)methyl)pyrrolidin-1-yl)nicotinamide |
| Example | N-((1r,3S)-3-(3-chloro-4-cyanophenoxy)-2,2,4,4-tetramethylcyclobutyl)-6-((3S)-3-((7-(3-(2,6- |
| 188 | dioxopiperidin-3-yl)-4-oxo-3,4-dihydrobenzo[d][1,2,3]triazin-6-yl)-2,7-diazaspiro[3.5]nonan-2- |
| | yl)methyl)pyrrolidin-1-yl)nicotinamide |
| Example | N-((1r,3R)-3-(3-chloro-4-cyanophenoxy)-2,2,4,4-tetramethylcyclobutyl)-6-((3R)-3-((2-(3-(2,6- |
| 189 | dioxopiperidin-3-yl)-4-oxo-3,4-dihydrobenzo[d][1,2,3]triazin-7-yl)-2,7-diazaspiro[3.5]nonan-7- |
| | yl)methyl)pyrrolidin-1-yl)nicotinamide |
| Example | N-((1r,3R)-3-(3-chloro-4-cyanophenoxy)-2,2,4,4-tetramethylcyclobutyl)-6-((3R)-3-((6-(3-(2,6- |
| 190 | dioxopiperidin-3-yl)-4-oxo-3,4-dihydrobenzo[d][1,2,3]triazin-7-yl)-2,6-diazaspiro[3.3]heptan-2- |
| | yl)methyl)pyrrolidin-1-yl)nicotinamide |
| Example | N-((1r,3R)-3-(3-chloro-4-cyanophenoxy)-2,2,4,4-tetramethylcyclobutyl)-6-((3R)-3-((4-(1-(3- |
| 191 | (2,6-dioxopiperidin-3-yl)-4-oxo-3,4-dihydrobenzo[d][1,2,3]triazin-7-yl)piperidin-4-yl)piperazin- |
| | 1-yl)methyl)pyrrolidin-1-yl)nicotinamide |
| Example | N-((1r,3R)-3-(3-chloro-4-cyanophenoxy)-2,2,4,4-tetramethylcyclobutyl)-6-((3R)-3-((4-(1-(3- |
| 192 | (2,6-dioxopiperidin-3-yl)-4-oxo-3,4-dihydrobenzo[d][1,2,3]triazin-6-yl)piperidin-4-yl)piperazin- |
| | 1-yl)methyl)pyrrolidin-1-yl)nicotinamide |
| Example | N-((1r,3r)-3-(3-chloro-4-cyanophenoxy)-2,2,4,4-tetramethylcyclobutyl)-6-(4-((4-(1-(3-(2,6- |
| 193 | dioxopiperidin-3-yl)-4-oxo-3,4-dihydrobenzo[d][1,2,3]triazin-7-yl)piperidin-4-yl)piperazin-1- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(4-((4-(1-(3-(2,6- |
| 194 | dioxopiperidin-3-yl)-4-oxo-3,4-dihydrobenzo[d][1,2,3]triazin-6-yl)piperidin-4-yl)piperazin-1- |
| | yl)methyl)piperidin-1-yl)nicotinamide |
| Example | N-((1r,3r)-3-(3-chloro-4-cyanophenoxy)-2,2,4,4-tetramethylcyclobutyl)-6-((1-((1-(3-(2,6- |
| 195 | dioxopiperidin-3-yl)-4-oxo-3,4-dihydrobenzo[d][1,2,3]triazin-7-yl)piperidin-4- |
| | yl)methyl)piperidin-4-yl)ethynyl)nicotinamide |
| Example | N-((1r,3r)-3-(3-chloro-4-cyanophenoxy)-2,2,4,4-tetramethylcyclobutyl)-6-((1-((1-(3-(2,6- |
| 196 | dioxopiperidin-3-yl)-4-oxo-3,4-dihydrobenzo[d][1,2,3]triazin-6-yl)piperidin-4- |
| | yl)methyl)piperidin-4-yl)ethynyl)nicotinamide |
| Example | N-((1r,3r)-3-(3-chloro-4-cyanophenoxy)-2,2,4,4-tetramethylcyclobutyl)-6-(4-(2-(4-(3-(2,6- |
| 197 | dioxopiperidin-3-yl)-4-oxo-3,4-dihydrobenzo[d][1,2,3]triazin-7-yl)piperazin-1-yl)ethyl)piperidin- |
| | 1-yl)nicotinamide |
| Example | N-((1r,3r)-3-(3-chloro-4-cyanophenoxy)-2,2,4,4-tetramethylcyclobutyl)-6-(4-(2-(4-(3-(2,6- |
| 198 | dioxopiperidin-3-yl)-4-oxo-3,4-dihydrobenzo[d][1,2,3]triazin-6-yl)piperazin-1-yl)ethyl)piperidin- |
| | 1-yl)nicotinamide |

Among the above compounds, the compounds in Table 2 below were particularly preferred in various aspects such as AR degradation activity, cancer cell line cytotoxicity, (metabolism) stability, and physicochemical properties.

TABLE 2

| Example | IUPAC name |
|---|---|
| Example | N-((1r,3r)-3-(3-chloro-4-cyanophenoxy)-2,2,4,4-tetramethylcyclobutyl)-4-(4-((1-(1-(3-(2,6- |
| 11 | dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)benzamide |
| Example | N-((1r,3r)-3-(3-chloro-4-cyanophenoxy)-2,2,4,4-tetramethylcyclobutyl)-4-(4-(7-(1-(3-(2,6- |
| 20 | dioxopiperidin-3-yl)-4-oxo-3,4-dihydrobenzo[d][1,2,3]triazin-6-yl)piperidine-4-carbonyl)-7- |
| | azaspiro[3.5]nonan-2-yl)piperazin-1-yl)benzamide |
| Example | N-((1r,3r)-3-(3-chloro-4-cyanophenoxy)-2,2,4,4-tetramethylcyclobutyl)-4-(4-(2-((1-(2-((3-(2,6- |
| 33 | dioxopiperidin-3-yl)-4-oxo-3,4-dihydrobenzo[d][1,2,3]triazin-6-yl)oxy)acetyl)piperidin-4- |
| | yl)oxy)ethyl)piperazin-1-yl)benzamide |
| Example | N-((1r,3r)-3-(3-chloro-4-cyanophenoxy)-2,2,4,4-tetramethylcyclobutyl)-4-(4-(2-((1-(3-(2,6- |
| 34 | dioxopiperidin-3-yl)-4-oxo-3,4-dihydrobenzo[d][1,2,3]triazin-6-yl)piperidin-4- |
| | yl)oxy)ethyl)piperazin-1-yl)benzamide |
| Example | N-((1r,3r)-3-(3-chloro-4-cyanophenoxy)-2,2,4,4-tetramethylcyclobutyl)-4-(4-(1-(3-(2,6- |
| 44 | dioxopiperidin-3-yl)-4-oxo-3,4-dihydrobenzo[d][1,2,3]triazin-6-yl)azetidine-3- |
| | carbonyl)piperazin-1-yl)benzamide |
| Example | N-((1r,3r)-3-(3-chloro-4-cyanophenoxy)-2,2,4,4-tetramethylcyclobutyl)-4-(4-(3-(1-(1-(3-(2,6- |
| 47 | dioxopiperidin-3-yl)-4-oxo-3,4-dihydrobenzo[d][1,2,3]triazin-6-yl)azetidine-3- |
| | carbonyl)piperidin-4-yl)propyl)piperazin-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 63 | dioxopiperidin-3-yl)-4-oxo-3,4-dihydrobenzo[d][1,2,3]triazin-6-yl)azetidine-3-carbonyl)-4- |
| | fluoropiperidin-4-yl)methyl)piperazin-1-yl)benzamide |
| Example | N-((1r,3r)-3-(3-chloro-4-cyanophenoxy)-2,2,4,4-tetramethylcyclobutyl)-4-(1-(2-((1-(1-(3-(2,6- |
| 75 | dioxopiperidin-3-yl)-4-oxo-3,4-dihydrobenzo[d][1,2,3]triazin-6-yl)azetidine-3- |
| | carbonyl)piperidin-4-yl)oxy)ethyl)piperidin-4-yl)benzamide |
| Example | N-((1r,3r)-3-(3-chloro-4-cyanophenoxy)-2,2,4,4-tetramethylcyclobutyl)-4-(1-(2-((1-(3-(2,6- |
| 76 | dioxopiperidin-3-yl)-4-oxo-3,4-dihydrobenzo[d][1,2,3]triazin-6-yl)piperidin-4- |
| | yl)oxy)ethyl)piperidin-4-yl)benzamide |
| Example | N-((1r,3r)-3-(3-chloro-4-cyanophenoxy)-2,2,4,4-tetramethylcyclobutyl)-6-(4-((1-(3-(2,6- |
| 78 | dioxopiperidin-3-yl)-4-oxo-3,4-dihydrobenzo[d][1,2,3]triazin-6-yl)piperidin-4- |
| | yl)methyl)piperazin-1-yl)nicotinamide |
| Example | N-((1r,3r)-3-(3-chloro-4-cyanophenoxy)-2,2,4,4-tetramethylcyclobutyl)-4-(1-(1-((1-(1-(3-(2,6- |
| 82 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)benzamide |
| Example | N-((1r,3r)-3-(3-chloro-4-cyanophenoxy)-2,2,4,4-tetramethylcyclobutyl)-4-(1-(1-((1-(1-(3-(2,6- |
| 83 | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)benzamide |
| Example | N-((1r,3r)-3-(3-chloro-4-cyanophenoxy)-2,2,4,4-tetramethylcyclobutyl)-4-(1-(1-((1-(3-(2,6- |
| 84 | dioxopiperidin-3-yl)-4-oxo-3,4-dihydrobenzo[d][1,2,3]triazin-6-yl)piperidin-4- |
| | yl)methyl)piperidin-4-yl)-1H-pyrazol-4-yl)benzamide |
| Example | N-((1r,3r)-3-(3-chloro-4-cyanophenoxy)-2,2,4,4-tetramethylcyclobutyl)-4-(3-(1-((1-(3-(2,6- |
| 88 | dioxopiperidin-3-yl)-4-oxo-3,4-dihydrobenzo[d][1,2,3]triazin-6-yl)piperidin-4- |
| | yl)methyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 90 | dioxopiperidin-3-yl)-4-oxo-3,4-dihydrobenzo[d][1,2,3]triazin-6-yl)azetidine-3- |
| | carbonyl)piperidin-4-yl)methyl)-1H-1,2,3-triazol-1-yl)benzamide |
| Example | N-((1r,3r)-3-(3-chloro-4-cyanophenoxy)-2,2,4,4-tetramethylcyclobutyl)-4-(2-(5-(1-(3-(2,6- |
| 94 | dioxopiperidin-3-yl)-4-oxo-3,4-dihydrobenzo[d][1,2,3]triazin-6-yl)azetidine-3- |
| | carbonyl)hexahydropyrrolo[3,4-c]pyrrol-2(1H)-yl)ethyl)benzamide |
| Example | N-((1r,3r)-3-(3-chloro-4-cyanophenoxy)-2,2,4,4-tetramethylcyclobutyl)-4-((1-(1-(3-(2,6- |
| 95 | dioxopiperidin-3-yl)-4-oxo-3,4-dihydrobenzo[d][1,2,3]triazin-6-yl)azetidine-3- |
| | carbonyl)piperidin-4-yl)ethynyl)benzamide |
| Example | N-((1r,3r)-3-(3-chloro-4-cyanophenoxy)-2,2,4,4-tetramethylcyclobutyl)-4-((1-((1-(1-(3-(2,6- |
| 96 | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)ethynyl)benzamide |
| Example | N-((1r,3r)-3-(3-chloro-4-cyanophenoxy)-2,2,4,4-tetramethylcyclobutyl)-4-((1-((1-(1-(3-(2,6- |
| 97 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)benzamide |
| Example | N-((1r,3r)-3-(3-chloro-4-cyanophenoxy)-2,2,4,4-tetramethylcyclobutyl)-4-((1-((1-((3-(2,6- |
| 98 | dioxopiperidin-3-yl)-4-oxo-3,4-dihydrobenzo[d][1,2,3]triazin-6-yl)glycyl)piperidin-4- |
| | yl)methyl)piperidin-4-yl)ethynyl)benzamide |
| Example | N-((1r,3r)-3-(3-chloro-4-cyanophenoxy)-2,2,4,4-tetramethylcyclobutyl)-4-((1-((1-(3-(2,6- |
| 99 | dioxopiperidin-3-yl)-4-oxo-3,4-dihydrobenzo[d][1,2,3]triazin-6-yl)piperidin-4- |
| | yl)methyl)piperidin-4-yl)ethynyl)benzamide |
| Example | N-((1r,3r)-3-(3-chloro-4-cyanophenoxy)-2,2,4,4-tetramethylcyclobutyl)-2-(4-((1-(3-(2,6- |
| 100 | dioxopiperidin-3-yl)-4-oxo-3,4-dihydrobenzo[d][1,2,3]triazin-6-yl)piperidin-4- |
| | yl)methyl)piperazin-1-yl)pyrimidine-5-carboxamide |
| Example | N-((1r,3r)-3-(3-chloro-4-cyanophenoxy)-2,2,4,4-tetramethylcyclobutyl)-4-(4-(1-(1-(3-(2,6- |
| 101 | dioxopiperidin-3-yl)-4-oxo-3,4-dihydrobenzo[d][1,2,3]triazin-6-yl)azetidine-3- |
| | carbonyl)piperidin-4-yl)-1H-1,2,3-triazol-1-yl)benzamide |
| Example | N-((1r,3r)-3-(3-chloro-4-cyanophenoxy)-2,2,4,4-tetramethylcyclobutyl)-4-(4-(1-(1-(3-(2,6- |
| 102 | dioxopiperidin-3-yl)-4-oxo-3,4-dihydrobenzo[d][1,2,3]triazin-6-yl)piperidine-4- |
| | carbonyl)piperidin-4-yl)-1H-1,2,3-triazol-1-yl)benzamide |
| Example | N-((1r,3r)-3-(3-chloro-4-cyanophenoxy)-2,2,4,4-tetramethylcyclobutyl)-4-(4-((1-(3-(2,6- |
| 103 | dioxopiperidin-3-yl)-4-oxo-3,4-dihydrobenzo[d][1,2,3]triazin-6-yl)piperidin-4-yl)methyl)-1H- |
| | 1,2,3-triazol-1-yl)benzamide |
| Example | N-((1r,3r)-3-(3-chloro-4-cyanophenoxy)-2,2,4,4-tetramethylcyclobutyl)-4-(4-((1-(1-(3-(2,6- |
| 104 | dioxopiperidin-3-yl)-4-oxo-3,4-dihydrobenzo[d][1,2,3]triazin-6-yl)piperidine-4- |
| | carbonyl)piperidin-4-yl)methyl)-1H-1,2,3-triazol-1-yl)benzamide |
| Example | N-((1r,3r)-3-(3-chloro-4-cyanophenoxy)-2,2,4,4-tetramethylcyclobutyl)-4-(4-((1-((3-(2,6- |
| 106 | dioxopiperidin-3-yl)-4-oxo-3,4-dihydrobenzo[d][1,2,3]triazin-6-yl)glycyl)piperidin-4-yl)methyl)- |
| | 1H-1,2,3-triazol-1-yl)benzamide |
| Example | N-((1r,3r)-3-(3-chloro-4-cyanophenoxy)-2,2,4,4-tetramethylcyclobutyl)-4-(4-(1-((3-(2,6- |
| 107 | dioxopiperidin-3-yl)-4-oxo-3,4-dihydrobenzo[d][1,2,3]triazin-6-yl)glycyl)piperidin-4-yl)-1H- |
| | 1,2,3-triazol-1-yl)benzamide |
| Example | N-((1r,3r)-3-(3-chloro-4-cyanophenoxy)-2,2,4,4-tetramethylcyclobutyl)-4-(2-(4-((1-(1-(3-(2,6- |
| 108 | dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)ethyl)benzamide |
| Example | N-((1r,3r)-3-(3-chloro-4-cyanophenoxy)-2,2,4,4-tetramethylcyclobutyl)-4-(2-(4-((1-((3-(2,6- |
| 110 | dioxopiperidin-3-yl)-4-oxo-3,4-dihydrobenzo[d][1,2,3]triazin-6-yl)glycyl)piperidin-4- |
| | yl)methyl)piperazin-1-yl)ethyl)benzamide |
| Example | N-((1r,3r)-3-(3-chloro-4-cyanophenoxy)-2,2,4,4-tetramethylcyclobutyl)-4-(2-(4-((1-(3-(2,6- |
| 111 | dioxopiperidin-3-yl)-4-oxo-3,4-dihydrobenzo[d][1,2,3]triazin-6-yl)piperidin-4- |
| | yl)methyl)piperazin-1-yl)ethyl)benzamide |
| Example | N-((1r,3r)-3-(3-chloro-4-cyanophenoxy)-2,2,4,4-tetramethylcyclobutyl)-4-(1-(1-((1-((3-(2,6- |
| 112 | dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)benzamide |
| Example | N-((1r,3r)-3-(3-chloro-4-cyanophenoxy)-2,2,4,4-tetramethylcyclobutyl)-6-(1-(1-((1-(1-(3-(2,6- |
| 114 | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)nicotinamide |
| Example | N-((1r,3r)-3-(3-chloro-4-cyanophenoxy)-2,2,4,4-tetramethylcyclobutyl)-4-(3-(1-((1-(1-(3-(2,6- |
| 116 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(3-(1-((1-(1-(3-(2,6- |
| 117 | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(3-(1-((1-((3-(2,6- |
| 118 | dioxopiperidin-3-yl)-4-oxo-3,4-dihydrobenzo[d][1,2,3]triazin-6-yl)glycyl)piperidin-4- |
| | yl)methyl)piperidin-4-yl)prop-1-yn-1-yl)benzamide |
| Example | N-((1r,3r)-3-(3-chloro-4-cyanophenoxy)-2,2,4,4-tetramethylcyclobutyl)-4-(4-((4-(1-(3-(2,6- |
| 121 | dioxopiperidin-3-yl)-4-oxo-3,4-dihydrobenzo[d][1,2,3]triazin-6-yl)piperidine-4- |
| | carbonyl)piperazin-1-yl)methyl)-1H-1,2,3-triazol-1-yl)benzamide |
| Example | N-((1r,3r)-3-(3-chloro-4-cyanophenoxy)-2,2,4,4-tetramethylcyclobutyl)-4-(4-((4-(1-(3-(2,6- |
| 122 | dioxopiperidin-3-yl)-4-oxo-3,4-dihydrobenzo[d][1,2,3]triazin-6-yl)azetidine-3- |
| | carbonyl)piperazin-1-yl)methyl)-1H-1,2,3-triazol-1-yl)benzamide |
| Example | N-((1r,3r)-3-(3-chloro-4-cyanophenoxy)-2,2,4,4-tetramethylcyclobutyl)-6-(1-(1-((1-(1-(3-(2,6- |
| 124 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)nicotinamide |
| Example | N-((1r,3r)-3-(3-chloro-4-cyanophenoxy)-2,2,4,4-tetramethylcyclobutyl)-6-(1-(1-((1-((3-(2,6- |
| 125 | dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)nicotinamide |
| Example | N-((1r,3r)-3-(3-chloro-4-cyanophenoxy)-2,2,4,4-tetramethylcyclobutyl)-6-(3-(1-((1-((3-(2,6- |
| 128 | dioxopiperidin-3-yl)-4-oxo-3,4-dihydrobenzo[d][1,2,3]triazin-6-yl)glycyl)piperidin-4- |
| | yl)methyl)piperidin-4-yl)prop-1-yn-1-yl)nicotinamide |
| Example | N-((1r,3r)-3-(3-chloro-4-cyanophenoxy)-2,2,4,4-tetramethylcyclobutyl)-6-(3-(1-((1-(3-(2,6- |
| 129 | dioxopiperidin-3-yl)-4-oxo-3,4-dihydrobenzo[d][1,2,3]triazin-6-yl)piperidin-4- |
| | yl)methyl)piperidin-4-yl)prop-1-yn-1-yl)nicotinamide |
| Example | N-((1r,3r)-3-(3-chloro-4-cyanophenoxy)-2,2,4,4-tetramethylcyclobutyl)-6-((1-((1-(1-(3-(2,6- |
| 130 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)nicotinamide |
| Example | N-((1r,3r)-3-(3-chloro-4-cyanophenoxy)-2,2,4,4-tetramethylcyclobutyl)-6-((1-((1-(1-(3-(2,6- |
| 131 | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)nicotinamide |
| Example | N-((1r,3r)-3-(3-chloro-4-cyanophenoxy)-2,2,4,4-tetramethylcyclobutyl)-6-((1-((1-((3-(2,6- |
| 132 | dioxopiperidin-3-yl)-4-oxo-3,4-dihydrobenzo[d][1,2,3]triazin-6-yl)glycyl)piperidin-4- |
| | yl)methyl)piperidin-4-yl)ethynyl)nicotinamide |
| Example | N-((1r,3r)-3-(3-chloro-4-cyanophenoxy)-2,2,4,4-tetramethylcyclobutyl)-6-((1-((1-(2-((3-(2,6- |
| 133 | dioxopiperidin-3-yl)-4-oxo-3,4-dihydrobenzo[d][1,2,3]triazin-6-yl)oxy)acetyl)piperidin-4- |
| | yl)methyl)piperidin-4-yl)ethynyl)nicotinamide |
| Example | N-((1r,3r)-3-(3-chloro-4-cyanophenoxy)-2,2,4,4-tetramethylcyclobutyl)-4-((1-(3-(2,6- |
| 142 | dioxopiperidin-3-yl)-4-oxo-3,4-dihydrobenzo[d][1,2,3]triazin-6-yl)piperidin-4- |
| | yl)ethynyl)benzamide |
| Example | N-((1r,3r)-3-(3-chloro-4-cyanophenoxy)-2,2,4,4-tetramethylcyclobutyl)-4-((1-((3-(2,6- |
| 143 | dioxopiperidin-3-yl)-4-oxo-3,4-dihydrobenzo[d][1,2,3]triazin-6-yl)glycyl)piperidin-4- |
| | yl)ethynyl)benzamide |
| Example | N-((1-(4-(((1r,3r)-3-(3-chloro-4-cyanophenoxy)-2,2,4,4- |
| 150 | tetramethylcyclobutyl)carbamoyl)phenyl)-1H-1,2,3-triazol-4-yl)methyl)-1-(3-(2,6- |
| | dioxopiperidin-3-yl)-4-oxo-3,4-dihydrobenzo[d][1,2,3]triazin-6-yl)piperidine-4-carboxamide |
| Example | N-((1r,3r)-3-(3-chloro-4-cyanophenoxy)-2,2,4,4-tetramethylcyclobutyl)-4-(4-((4-(3-(2,6- |
| 165 | dioxopiperidin-3-yl)-4-oxo-3,4-dihydrobenzo[d][1,2,3]triazin-7-yl)piperazin-1- |
| | yl)methyl)piperidin-1-yl)benzamide |
| Example | N-((1r,3r)-3-(3-chloro-4-cyanophenoxy)-2,2,4,4-tetramethylcyclobutyl)-4-(4-((4-(3-(2,6- |
| 166 | dioxopiperidin-3-yl)-4-oxo-3,4-dihydrobenzo[d][1,2,3]triazin-6-yl)piperazin-1- |
| | yl)methyl)piperidin-1-yl)benzamide |
| Example | N-((1r,3r)-3-(3-chloro-4-cyanophenoxy)-2,2,4,4-tetramethylcyclobutyl)-6-(4-((7-(3-(2,6- |
| 169 | dioxopiperidin-3-yl)-4-oxo-3,4-dihydrobenzo[d][1,2,3]triazin-7-yl)-2,7-diazaspiro[3.5]nonan-2- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(4-((7-(3-(2,6- |
| 170 | dioxopiperidin-3-yl)-4-oxo-3,4-dihydrobenzo[d][1,2,3]triazin-6-yl)-2,7-diazaspiro[3.5]nonan-2- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(4-((2-(3-(2,6- |
| 171 | dioxopiperidin-3-yl)-4-oxo-3,4-dihydrobenzo[d][1,2,3]triazin-7-yl)-2,7-diazaspiro[3.5]nonan-7- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(4-((2-(3-(2,6- |
| 172 | dioxopiperidin-3-yl)-4-oxo-3,4-dihydrobenzo[d][1,2,3]triazin-6-yl)-2,7-diazaspiro[3.5]nonan-7- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(2-((1-(3-(2,6- |
| 173 | dioxopiperidin-3-yl)-4-oxo-3,4-dihydrobenzo[d][1,2,3]triazin-7-yl)piperidin-4-yl)methyl)-2,7- |
| | diazaspiro[3.5]nonan-7-yl)nicotinamide |
| Example | N-((1r,3r)-3-(3-chloro-4-cyanophenoxy)-2,2,4,4-tetramethylcyclobutyl)-6-(2-((1-(3-(2,6- |
| 174 | dioxopiperidin-3-yl)-4-oxo-3,4-dihydrobenzo[d][1,2,3]triazin-6-yl)piperidin-4-yl)methyl)-2,7- |
| | diazaspiro[3.5]nonan-7-yl)nicotinamide |
| Example | N-((1r,3r)-3-(3-chloro-4-cyanophenoxy)-2,2,4,4-tetramethylcyclobutyl)-6-(2-((1-(3-(2,6- |
| 175 | dioxopiperidin-3-yl)-4-oxo-3,4-dihydrobenzo[d][1,2,3]triazin-7-yl)azetidin-3-yl)methyl)-2,7- |
| | diazaspiro[3.5]nonan-7-yl)nicotinamide |
| Example | N-((1r,3r)-3-(3-chloro-4-cyanophenoxy)-2,2,4,4-tetramethylcyclobutyl)-6-(2-((1-(3-(2,6- |
| 176 | dioxopiperidin-3-yl)-4-oxo-3,4-dihydrobenzo[d][1,2,3]triazin-6-yl)azetidin-3-yl)methyl)-2,7- |
| | diazaspiro[3.5]nonan-7-yl)nicotinamide |
| Example | N-((1r,3r)-3-(3-chloro-4-cyanophenoxy)-2,2,4,4-tetramethylcyclobutyl)-6-((1-((1-(3-(2,6- |
| 183 | dioxopiperidin-3-yl)-4-oxo-3,4-dihydrobenzo[d][1,2,3]triazin-7-yl)azetidin-3-yl)methyl)piperidin- |
| | 4-yl)ethynyl)nicotinamide |
| Example | N-((1r,3r)-3-(3-chloro-4-cyanophenoxy)-2,2,4,4-tetramethylcyclobutyl)-6-((1-((1-(3-(2,6- |
| 184 | dioxopiperidin-3-yl)-4-oxo-3,4-dihydrobenzo[d][1,2,3]triazin-6-yl)azetidin-3-yl)methyl)piperidin- |
| | 4-yl)ethynyl)nicotinamide |
| Example | N-((1r,3R)-3-(3-chloro-4-cyanophenoxy)-2,2,4,4-tetramethylcyclobutyl)-6-((3R)-3-((7-(3-(2,6- |
| 185 | dioxopiperidin-3-yl)-4-oxo-3,4-dihydrobenzo[d][1,2,3]triazin-7-yl)-2,7-diazaspiro[3.5]nonan-2- |
| | yl)methyl)pyrrolidin-1-yl)nicotinamide |
| Example | N-((1r,3R)-3-(3-chloro-4-cyanophenoxy)-2,2,4,4-tetramethylcyclobutyl)-6-((3R)-3-((7-(3-(2,6- |
| 186 | dioxopiperidin-3-yl)-4-oxo-3,4-dihydrobenzo[d][1,2,3]triazin-6-yl)-2,7-diazaspiro[3.5]nonan-2- |
| | yl)methyl)pyrrolidin-1-yl)nicotinamide |
| Example | N-((1r,3r)-3-(3-chloro-4-cyanophenoxy)-2,2,4,4-tetramethylcyclobutyl)-6-(4-((4-(1-(3-(2,6- |
| 193 | dioxopiperidin-3-yl)-4-oxo-3,4-dihydrobenzo[d][1,2,3]triazin-7-yl)piperidin-4-yl)piperazin-1- |
| | yl)methyl)piperidin-1-yl)nicotinamide |
| Example | N-((1r,3r)-3-(3-chloro-4-cyanophenoxy)-2,2,4,4-tetramethylcyclobutyl)-6-(4-((4-(1-(3-(2,6- |
| 194 | dioxopiperidin-3-yl)-4-oxo-3,4-dihydrobenzo[d][1,2,3]triazin-6-yl)piperidin-4-yl)piperazin-1- |
| | yl)methyl)piperidin-1-yl)nicotinamide |
| Example | N-((1r,3r)-3-(3-chloro-4-cyanophenoxy)-2,2,4,4-tetramethylcyclobutyl)-6-((1-((1-(3-(2,6- |
| 195 | dioxopiperidin-3-yl)-4-oxo-3,4-dihydrobenzo[d][1,2,3]triazin-7-yl)piperidin-4- |
| | yl)methyl)piperidin-4-yl)ethynyl)nicotinamide |
| Example | N-((1r,3r)-3-(3-chloro-4-cyanophenoxy)-2,2,4,4-tetramethylcyclobutyl)-6-((1-((1-(3-(2,6- |
| 196 | dioxopiperidin-3-yl)-4-oxo-3,4-dihydrobenzo[d][1,2,3]triazin-6-yl)piperidin-4- |
| | yl)methyl)piperidin-4-yl)ethynyl)nicotinamide |

As used herein, the term “pharmaceutically acceptable salt(s)” refers to a salt prepared from active compounds according to the present disclosure with relatively non-toxic acids or bases, depending on the particular substituents of those compounds. When the compounds have a relatively acidic group, base-added salts can be obtained by contacting the neutral compounds with a sufficient amount of the desired base and a pure or inert solvent. Suitable pharmaceutically acceptable base addition salts include, but are not limited to sodium, potassium, calcium, aluminum, organic amino, magnesium salts and the like. When the compounds have a relatively basic group, acid-added salts can be obtained by contacting the neutral compounds with a sufficient amount of the desired acid and pure or inert solvent. Suitable pharmaceutically acceptable acid addition salts include salts derived from non-toxic organic acids including, but are not limited to, acetic acid, propionic acid, isobutyl acid, oxalic acid, maleic acid, malonic acid, benzoic acid, succinic acid, suberic acid, fumaric acid, mandelic acid, phthalic acid, benzenesulfonic acid, p-tolylsulfonic acid, citric acid, tartaric acid, methanesulfonic acid, and the like, and non-toxic inorganic acids including, but are not limited to, hydrochloric acid, hydrobromic acid, nitric acid, carbonic acid, monohydrogencarbonic acid, phosphoric acid, monohydrogenphosphoric acid, dihydrogenphosphoric acid, sulfuric acid, monohydrogensulfuric acid, hydrogen iodide, phosphorous acid and the like. Also it includes a salt of amino acid such as arginate or its analogues, and it also includes analogues of organic acid such as glucuronic or galacturonic acid. Some specific compounds of this disclosure have both basic and acidic functionality for the conversion of compounds with a basic or acidic portion (addition) salts.

As used herein, the phrase “compound(s) of this/the invention” includes any compound(s) of Chemical Formula 1 or 1a, as well as clathrates, hydrates, solvates, or polymorphs thereof. And, even if the term “compound(s) of the invention” does not mention its pharmaceutically acceptable sat, the term includes salts thereof. In one embodiment, the compounds of this disclosure include stereo-chemically pure compounds, e.g., those substantially free (e.g., greater than 85% ee, greater than 90% ee, greater than 95% ee, greater than 97% ee, or greater than 99% ee) of other stereoisomers. That is, if the compounds of Chemical Formula 1 or 1a according to the present disclosure or salts thereof are tautomeric isomers and/or stereoisomers (e.g., geometrical isomers and conformational isomers), such isolated isomers and their mixtures also are included in the scope of this disclosure. If the compounds of the present disclosure or salts thereof have an asymmetric carbon in their structures, their active optical isomers and their racemic mixtures also are included in the scope of this disclosure.

As used herein, the term “polymorph” refers to solid crystalline forms of a compound of this disclosure or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “solvate” means a compound or its salt according to this disclosure that further includes a stoichiometric or non-stoichiometric amount of a solvent bound by non-covalent intermolecular forces. Preferred solvents are volatile, non-toxic, and acceptable for administration to humans in trace amounts.

As used herein, the term “hydrate” means a compound or its salt according to this disclosure that further includes a stoichiometric or non-stoichiometric amount of water bound by non-covalent intermolecular forces.

As used herein, the term “clathrate” means a compound or its salt in the form of a crystal lattice that contains spaces (e.g., channels) that have a guest molecule (e.g., a solvent or water) trapped within.

As used herein, the term “purified” means that when isolated, the isolate is greater than 90% pure, in one embodiment greater than 95% pure, in another embodiment greater than 99% pure and in another embodiment greater than 99.9% pure.

### Medical Uses and Methods of Treatment of the Compounds According to the Present Invention

The present invention further provides methods for treating a disease or condition in a subject having or susceptible to having such a disease or condition, by administering to the subject a therapeutically-effective amount of one or more compounds as described above. In one embodiment, the treatment is preventative treatment. In another embodiment, the treatment is palliative treatment. In another embodiment, the treatment is restorative treatment.

1. Diseases or Conditions

The compounds of the present invention for inhibiting AR activity are useful for various therapeutic or prophylactic uses (e.g., cancer, prostate cancer, Kennedy disease). These compounds can be used to inhibit or suppress AR activity, and can also be used to treat AR-related diseases or to prevent exacerbation of these diseases. Accordingly, the present invention provides a method for inhibiting or suppressing AR activity, or degrading AR in a cell. In this method the cells are contacted with an effective amount of a compound of the invention. In one embodiment, the cell is present in a subject. The method of the present invention comprises administering to a subject in need of treatment or prevention a pharmaceutical composition comprising a therapeutically or prophylactically effective amount of a compound according to the present invention.

In one embodiment, the present invention provides a method of inhibiting or suppressing AR activity, or degrading AR in a cell of an AR-associated disease. For example, the present invention can be used to inhibit AR activity in cells of a subject having an AR-related disease, which will be described later, and consequently lower AR activity. In another embodiment of the present invention, the present invention can be used to inhibit AR activity in cells of cancer, in particular prostate cancer.

In another embodiment, the present invention provides a method of treating an AR-related disease, comprising administering to a subject a therapeutically effective amount of a compound of Chemical Formula 1 or 1a or a pharmaceutically acceptable salt thereof. Such a method comprises administering to a subject in need of treatment an amount of a compound of the invention sufficient to inhibit AR activity, i.e., a therapeutically effective amount. In such a method, a compound of the present invention may be administered to the subject in the form of a pharmaceutical composition described herein.

In the present invention, AR-related diseases include, but are not limited to, asthma (multiple sclerosis, cancer (especially prostate cancer, breast cancer (especially androgen receptor positive triple negative breast cancer (AR+TNBC))), Kennedy® disease, acne, alopecia (especially androgenetic alopecia), cutaneous wound, Hirsutism, ciliopathy, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, cystic fibrosis, Duchenne muscular dystrophy, haemochromatosis, haemophilia, Klinefelter® syndrome, neurofibromatosis, phenylketonuria, polycystic kidney disease, Prader-Willi syndrome, sickle-cell disease, Tay-Sachs disease, Turner syndrome. In a preferred embodiment of the present invention, the AR-related disease is cancer, more preferably prostate cancer.

That is, the present invention provides a medical use of the compound of Chemical Formula 1 or 1a or a pharmaceutically acceptable salt thereof for treating or preventing the above diseases.

2. Subjects

Suitable subjects to be treated according to the present invention include mammalian subjects. Mammals according to the present disclosure include, but are not limited to, human, canine, feline, bovine, caprine, equine, ovine, porcine, rodents, lagomorphs, primates, and the like, and encompass mammals in utero.

In one embodiment, the suitable subject to be treated according to the present invention is human.

3. Administration and Dosing

The compounds of the present invention are generally administered in a therapeutically effective amount.

As used herein, “effective amount” refers to an amount of a compound of the invention sufficient to slow or minimize the progression of a AR-related disease or to provide a therapeutic benefit in the treatment or management of a AR-related disease. “Effective amount” also refers to an amount sufficient to inhibit or reduce AR activity, either in vitro or in vivo.

The compounds of the present invention can be administered by any suitable route in the form of a pharmaceutical composition adapted to such a route, and in a dose effective for the treatment intended. An effective dosage is typically in the range of about 0.001 to about 100 mg per kg body weight per day, preferably about 0.01 to about 50 mg/kg/day, in single or divided doses. Depending on age, species and disease or condition being treated, dosage levels below the lower limit of this range may be suitable. In other cases, still larger doses may be used without harmful side effects. Larger doses may also be divided into several smaller doses, for administration throughout the day.

### Pharmaceutical Compositions of the Compounds of the Present Invention

In another embodiment, the present invention provides a pharmaceutical composition comprising the compound of Chemical Formula 1 or 1a or a pharmaceutically acceptable salt thereof, and a pharmaceutically acceptable carrier or excipient. In one embodiment of the present invention, the pharmaceutical composition is used for the treatment or prevention of AR-related diseases, preferably prostate cancer, which is described above.

The term “pharmaceutically acceptable” means suitable for use as a pharmaceutical preparation, and generally considered safe for such use. The term also means that it has been officially approved by the governing body of a country for this use, or is listed in the Korean Pharmacopoeia or the United States Pharmacopoeia.

### Pharmaceutical Compositions, Dosage Forms and Administration Routes

For the treatment of the diseases or conditions referred to above, the compounds described herein or pharmaceutically acceptable salts thereof can be administered as follows:

Oral Administration

The compounds of the present invention may be administered orally, including by swallowing, so that the compound enters the gastrointestinal tract, or absorbed into the blood stream directly from the mouth (e.g., buccal or sublingual administration).

Suitable compositions for oral administration include solid, liquid, gel or powder formulations, and have a dosage form such as tablet, lozenge, capsule, granule or powder.

Compositions for oral administration may optionally be enteric coated and may exhibit delayed or sustained release through the enteric coating. That is, the composition for oral administration according to the present invention may be a formulation having an immediate or modified release pattern.

Liquid formulations can include solutions, syrups and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

In a tablet dosage form the amount of drug, active ingredient, present may be from about 0.05% to about 95% by weight, more typically from about 2% to about 50% by weight of the dosage form. In addition, tablets may contain a disintegrant, comprising from about 0.5% to about 35% by weight, more typically from about 2% to about 25% of the dosage form. Examples of disintegrants include, but are not limited to, lactose, starch, sodium starch glycolate, crospovidone, croscarmellose sodium, maltodextrin, or mixtures thereof.

Suitable lubricants, for use in a tablet, may be present in amounts from about 0.1% to about 5% by weight, and include, but are not limited to, talc, silicon dioxide, stearic acid, calcium, zinc or magnesium stearate, sodium stearyl fumarate and the like.

Suitable binders, for use in a tablet, include, but are not limited to, gelatin, polyethylene glycol, sugars, gums, starch, polyvinyl pyrrolidone, hydroxypropyl cellulose, hydroxypropylmethyl cellulose and the like. Suitable diluents, for use in a tablet, include, but are not limited to, mannitol, xylitol, lactose, dextrose, sucrose, sorbitol, microcrystalline cellulose and starch.

Suitable solubilizers, for use in a tablet, may be present in amounts from about 0.1% to about 3% by weight, and include, but are not limited to, polysorbates, sodium lauryl sulfate, sodium dodecyl sulfate, propylene carbonate, diethyleneglycol monoethyl ether, dimethyl isosorbide, polyethylene glycol (natural or hydrogenated) castor oil, HCOR™ (Nikkol), oleyl ester, Gelucire™, caprylic/caprylic acid mono/diglyceride, sorbitan fatty acid esters, and Solutol HS™.

Parenteral Administration

Compounds of the present disclosure may be administered directly into the blood stream, muscle, or internal organs. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods.

Compositions for parenteral administration may be formulated as immediate or modified release, including delayed or sustained release.

Most parenteral formulations are liquid compositions, and the liquid composition is an aqueous solution containing the active ingredient according to the present invention, a salt, a buffering agent, an isotonic agent, and the like.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

Topical Administration

Compounds of the present invention may be administered topically to the skin or transdermally. Formulations for this topical administration can include lotions, solutions, creams, gels, hydrogels, ointments, foams, implants, patches and the like. Pharmaceutically acceptable carriers for topical administration formulations can include water, alcohol, mineral oil, glycerin, polyethylene glycol and the like. Topical administration can also be performed by electroporation, iontophoresis, phonophoresis and the like.

Compositions for topical administration may be formulated as immediate or modified release, including delayed or sustained release.

### Advantageous Effects

The present invention provides compounds capable of exhibiting various pharmacological activities by inhibiting or suppressing AR activity, or degrading AR, pharmaceutical compositions comprising the compound as an active ingredient, their medical uses (especially prostate cancer), and methods of treatment or prevention comprising administering the compound to a subject in need of such treatment or prevention. The compound according to the present invention or a pharmaceutically acceptable salt thereof is excellent in various aspects such as efficacy, (metabolism) stability, physicochemical properties, etc.

### MODE FOR INVENTION

Hereinafter, the present invention is described in considerable detail with examples to help those skilled in the art understand the present invention. However, the following examples are offered by way of illustration and are not intended to limit the scope of the invention. It is apparent that various changes may be made without departing from the spirit and scope of the invention or sacrificing all of its material advantages.

### Preparation of Compounds of the Present Invention

Hereinafter, the synthesis process of some compounds of the present invention will be described, and the other compounds not mentioned below can be prepared by substituting starting materials, intermediates and/or reactants in a similar manner.

### Synthesis of N-((1r,3r)-3-(3-chloro-4-cyanophenoxy)-2,2,4,4-tetramethylcyclobutyl)-4-(piperazin-1-yl)benzamide

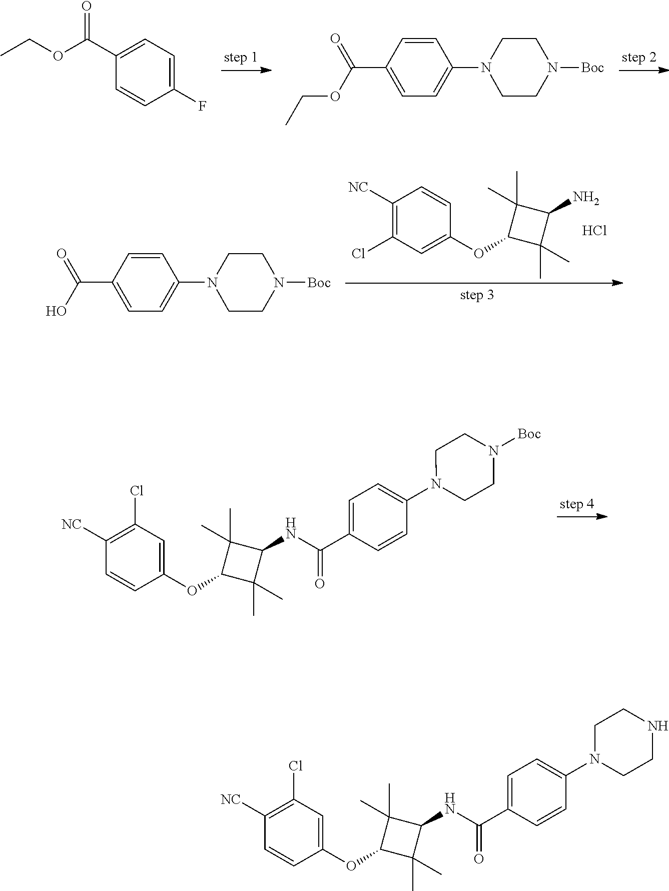

### Synthesis of tert-butyl 4-(4-(ethoxycarbonyl)phenyl)piperazine-1-carboxylate (Step 1)

Ethyl 4-fluorobenzoate (5.0 g, 29.7 mmol) and tert-butyl piperazine-1-carboxylate (6.7 g, 35.64 mmol) were dissolved in DMSO (40 mL), and DIPEA (15.5 mL, 89.1 mmol) was added and stirred at 130° C. for 24 hours. After cooling the reactant to room temperature, it was extracted with water and ethyl acetate. The organic layer was washed with brine, the residue was removed with anhydrous magnesium sulfate, and concentrated. The mixture was subjected to column chromatography to obtain the desired compound as a white solid (5.5 g, 55%).

¹H NMR (300 MHz, CDCl₃) δ 7.97-7.88 (m, 2H), 6.89-6.82 (m, 2H), 4.33 (q, J=7.1 Hz, 2H), 3.58 (m, 2H), 3.30 (m, 2H), 1.49 (s, 9H), 1.37 (t, J=7.1 Hz, 3H).

### Synthesis of 4-(4-(2-(1-(tert-butoxycarbonyl)piperidin-4-yl)ethyl)piperazin-1-yl)benzoic Acid (Step 2)

Tert-butyl 4-(4-(ethoxycarbonyl)phenyl)piperazine-1-carboxylate (5.5 g, 16.44 mmol) was dissolved in the mixture of MeOH (10 mL), H₂O (10 mL) and THF (40 mL), and LiOH·H₂O (1 g, 24.66 mmol) was added and stirred at 40° C. for 12 hours. After cooling to room temperature, extraction was performed with water and ethyl acetate. The aqueous layer was neutralized with 1 N HCl solution, and the solid was filtered, washed with water, and dried to obtain the target compound as a white solid.

¹H NMR (500 MHz, CDCl₃) δ 8.20-7.68 (m, 2H), 7.02-6.73 (m, 2H), 3.59 (t, J=5.2 Hz, 4H), 3.34 (t, J=5.2 Hz, 4H), 1.49 (s, 9H).

### Synthesis of tert-butyl 4-(4-(((1r,3r)-3-(3-chloro-4-cyanophenoxy)-2,2,4,4-tetramethylcyclobutyl)carbamoyl)phenyl)piperazine-1-carboxylate (Step 3)

4-(4-(2-(1-(tert-butoxycarbonyl)piperidin-4-yl)ethyl)piperazin-1-yl)benzoic acid (1.5 g, 4.75 mmol), 4-((1r,3r)-3-amino-2,2,4,4-tetramethylcyclobutoxy)-2-chlorobenzonitrile hydrochloride (2.9 g, 9.51 mmol), EDCl·HCl (1.0 g, 5.22 mmol), and HOBt (0.705 g, 5.22 mmol) were dissolved in DMF (20 mL), and DIPEA (3.3 mL, 19.0 mmol) was added and stirred at room temperature for 12 hours. The reactant was extracted with water and ethyl acetate. The organic layer was washed with brine, the residue was removed with anhydrous magnesium sulfate, and concentrated. Then, the desired compound (2.2 g, 81%) was obtained as a white solid by column chromatography.

¹H NMR (300 MHz, CDCl₃) δ 7.78-7.64 (m, 2H), 7.57 (d, J=8.7 Hz, 1H), 6.97 (d, J=2.4 Hz, 1H), 6.94-6.89 (m, 2H), 6.81 (dd, J=8.7, 2.5 Hz, 1H), 6.14 (d, J=8.1 Hz, 1H), 4.21-4.10 (m, 1H), 4.05 (s, 1H), 3.65-3.56 (m, 4H), 3.27 (m, 4H), 1.49 (s, 9H), 1.27 (s, 6H), 1.22 (s, 6H).

### Synthesis of N-((1r,3r)-3-(3-chloro-4-cyanophenoxy)-2,2,4,4-tetramethylcyclobutyl)-4-(piperazin-1-yl)benzamide (Step 4)

Tert-butyl 4-(4-(((1r,3r)-3-(3-chloro-4-cyanophenoxy)-2,2,4,4-tetramethylcyclobutyl)carbamoyl)phenyl)piperazine-1-carboxylate (1.5 g, 2.64 mmol) was dissolved in DCM (10 mL), and 4.0 N HCl 1,4-dioxane (1.0 mL) was added and stirred at room temperature for 3 hours. The reaction solution was concentrated under reduced pressure, neutralized with 5% K₂CO₃ aqueous solution, and extracted with ethyl acetate. The organic layer was washed with brine, the residue was removed with anhydrous magnesium sulfate, and concentrated. The mixture was subjected to column chromatography to obtain the desired compound (1.0 g, 83%) as a white solid.

¹H NMR (300 MHz, CDCl₃) δ 7.70 (m, 2H), 7.57 (d, J=8.7 Hz, 1H), 6.97 (d, J z=2.4 Hz, 1H), 6.94-6.88 (m, 2H), 6.81 (dd, J=8.7, 2.4 Hz, 1H), 6.11 (d, J=8.2 Hz, 1H), 4.15 (d, J=8.1 Hz, 1H), 4.04 (s, 1H), 3.40-3.19 (m, 4H), 3.13-2.97 (m, 4H), 1.26 (m, 7H), 1.22 (s, 6H).

### Synthesis of N-((1r,3r)-3-(3-chloro-4-cyanophenoxy)-2,2,4,4-tetramethylcyclobutyl)-4-(4-(piperidin-4-ylmthyl) piperazin-1-yl)benzamide hydrochloride

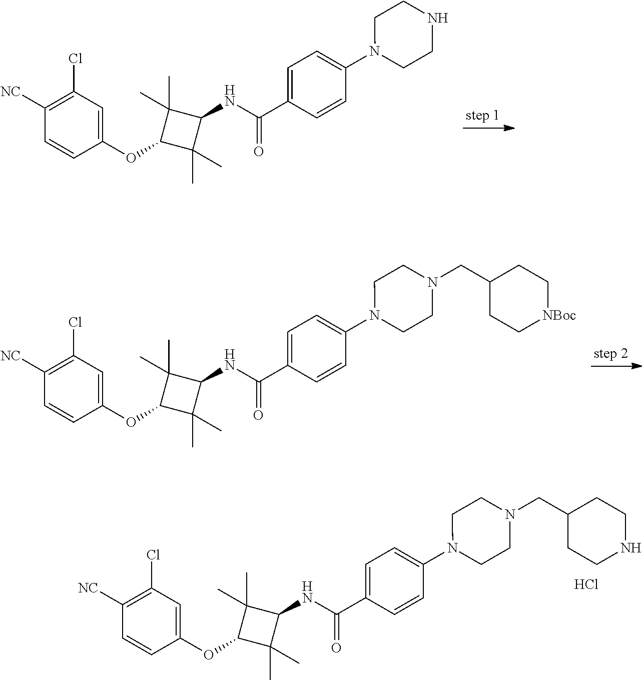 [SMILES: CC1(C)[C@H](NC(=O)c2ccc(N3CCNCC3)cc2)C(C)(C)[C@H]1Oc1ccc(C#N)c(Cl)c1 ; CC(C)(C)OC(=O)N1CCC(CN2CCN(c3ccc(C(=O)N[C@H]4C(C)(C)[C@H](Oc5ccc(C#N)c(Cl)c5)C4(C)C)cc3)CC2)CC1 ; CC1(C)[C@H](NC(=O)c2ccc(N3CCN(CC4CCNCC4)CC3)cc2)C(C)(C)[C@H]1Oc1ccc(C#N)c(Cl)c1 ; Cl]

### Synthesis of 4 tert-butyl 4-((4-(4-(((1R,3R)-3-(3-chloro-4-cyanophenoxy)-2,2,4,4-tetramethylcyclobutyl) carbamoyl)phenyl)piperazin-1-yl)methyl)piperidine-1-carboxylate (Step 1)

N-((1r,3r)-3-(3-chloro-4-cyanophenoxy)-2,2,4,4-tetramethylcyclobutyl)-4-(piperazin-1-yl)benzamide (700 mg, 1.49 mmol) was dissolved in MeOH (15 mL), and tert-butyl 4-formylpiperidine-1-carboxylate (1.3 g, 5.99 mmol), AcOH (1.0 mL) was added and stirred at room temperature for 1 hour. NaBH₃CN (561 mg, 8.94 mmol) was slowly added to the reaction mixture and stirred at room temperature for 12 hours. The reactants were concentrated, neutralized with 5% K₂CO₃ aqueous solution, and then extracted with dichloromethane. The organic layer was washed with brine, the residue was removed with anhydrous magnesium sulfate, and concentrated. The mixture was subjected to column chromatography to obtain the desired compound (700 mg, 70%) as a white solid.

¹H NMR (400 MHz, CDCl₃) δ 7.73-7.65 (m, 2H), 7.56 (d, J=8.7 Hz, 1H), 6.97 (d, J=2.4 Hz, 1H), 6.94-6.89 (m, 2H), 6.81 (dd, J=8.7, 2.4 Hz, 1H), 6.11 (d, J=8.2 Hz, 1H), 4.14 (m, 2H), 4.04 (m, 2H), 3.29 (t, J=5.0 Hz, 4H), 2.71 (t, J=12.6 Hz, 2H), 2.56 (t, J=5.1 Hz, 4H), 2.23 (d, J=7.0 Hz, 2H), 1.75 (d, J=13.5 Hz, 2H), 1.67 (m, 1H), 1.46 (s, 9H), 1.26 (s, 6H), 1.22 (s, 6H), 1.16-1.05 (m, 2H); LC/MS (M+H)⁺ (m/z) 664.1

### Synthesis of N-((1r,3r)-3-(3-chloro-4-cyanophenoxy)-2,2,4,4-tetramethylcyclobutyl)-4-(4-(piperidin-4-ylmthyl) piperazin-1-yl)benzamide hydrochloride (Step 2)

4 tert-butyl 4-((4-(4-(((1R,3R)-3-(3-chloro-4-cyanophenoxy)-2,2,4,4-tetramethylcyclobutyl) carbamoyl)phenyl)piperazin-1-yl)methyl)piperidine-1-carboxylate (700 mg, 1.053 mmol) was dissolved in DCM (10 mL), and 4.0 N HCl 1,4-dioxane (2 mL) was added and stirred at room temperature for 2 hours. The reaction product was concentrated under reduced pressure to obtain the desired compound (600 mg, 94%) as a white solid.

¹H NMR (500 MHz, MeOH-d4) δ 7.81 (d, J=8.3 Hz, 2H), 7.72 (d, J=8.7 Hz, 1H), 7.16-7.05 (m, 4H), 6.98 (d, J=8.8 Hz, 1H), 4.02 (d, J=13.7 Hz, 3H), 3.76 (t, J=11.3 Hz, 3H), 3.65 (d, J=1.5 Hz, 3H), 3.46 (d, J=13.1 Hz, 2H), 3.37 (t, J=12.7 Hz, 2H), 3.23 (d, J=6.8 Hz, 2H), 3.09 (t, J=12.9 Hz, 2H), 2.15 (d, J=14.5 Hz, 2H), 1.59 (q, J=12.8 Hz, 2H), 1.28 (s, 6H), 1.22 (s, 6H); LC/MS (M+H)⁺ (m/z) 564.1

### Synthesis of N-((1R,3R)-3-(3-chloro-4-cyanophenoxy)-2,2,4,4-tetramethylcyclobutyl)-4-(4-((1-(3-(2,6-dioxopiperidin-3-yl)-4-oxo-3,4-dihydrobenzo[d][1,2,3]triazin-6-yl)piperidin-4-yl)methyl)piperazin-1-yl)benzamide (Example 8)

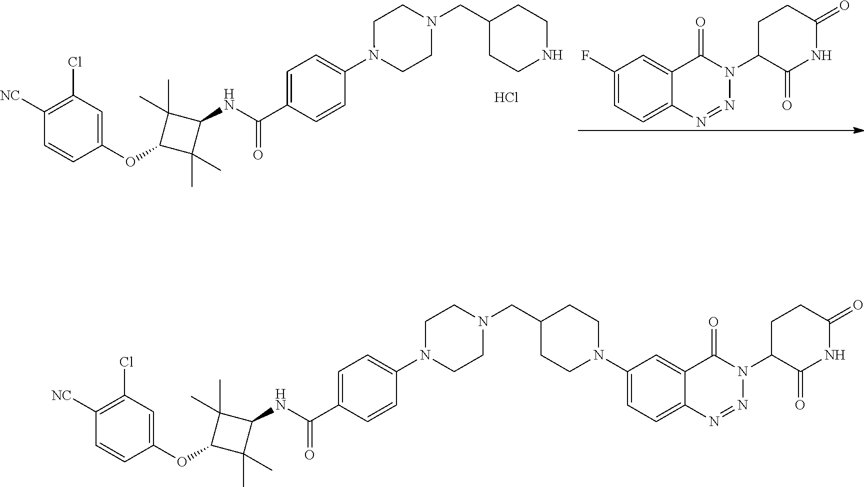

N-((1r,3r)-3-(3-chloro-4-cyanophenoxy)-2,2,4,4-tetramethylcyclobutyl)-4-(4-(piperidin-4-ylmthyl) piperazin-1-yl)benzamide hydrochloride (80 mg, 0.133 mmol), and 3-(6-fluoro-4-oxobenzo[d][1,2,3]triazin-3(4H)-yl)piperidine-2,6-dione (44 mg, 0.159 mmol) were dissolved in DMF (10 mL), and DIPEA (0.092 mL, 0.532 mmol) was added and stirred at 90° C. for 12 hours. The reactant was diluted with water and extracted with ethyl acetate. The organic layer was washed with brine, the residue was removed with anhydrous magnesium sulfate, and concentrated. The mixture was subjected to column chromatography to obtain the desired compound (45 mg, 41%) as a yellow solid.

¹H NMR (500 MHz, CDCl₃) δ 8.36 (s, 1H), 7.98 (d, J=9.1 Hz, 1H), 7.70 (d, J=8.4 Hz, 2H), 7.57 (d, J=8.7 Hz, 1H), 7.52 (d, J=2.8 Hz, 1H), 7.45 (dd, J=9.2, 2.9 Hz, 1H), 6.99-6.94 (m, 1H), 6.92 (d, J=8.5 Hz, 2H), 6.81 (dd, J=8.2, 2.3 Hz, 1H), 6.14 (d, J=8.1 Hz, 1H), 5.77 (dd, J=11.6, 5.4 Hz, 1H), 4.15 (d, J=8.1 Hz, 1H), 4.07 (m, 1H), 4.05 (s, 2H), 3.32 (t, J=5.0 Hz, 4H), 3.02 (t, J=12.5 Hz, 2H), 2.98-2.81 (m, 3H), 2.59 (t, J=5.0 Hz, 4H), 2.40-2.34 (m, 1H), 2.28 (d, J=7.1 Hz, 2H), 1.99-1.92 (m, 2H), 1.86 (m, 1H), 1.33 (m, 2H), 1.27 (s, 6H), 1.22 (s, 6H); ¹³C NMR (101 MHz, CDCl₃) δ 171.03, 168.09, 167.13, 162.66, 156.09, 153.51, 153.29, 138.27, 136.37, 135.08, 130.34, 128.28, 123.93, 121.73, 121.51, 116.77, 116.37, 114.28, 114.19, 105.33, 105.01, 84.90, 64.23, 58.46, 58.12, 53.37, 47.79, 40.32, 33.30, 31.05, 30.13, 23.61, 23.52, 23.27; LC/MS (M+H)⁺ (m/z) 846.1, (M+H)⁻ (m/z) 844.0.

### Synthesis of N-((1r,3r)-3-(3-chloro-4-cyanophenoxy)-2,2,4,4-tetramethylcyclobutyl)-4-(4-((1-(2-((3-(2,6-dioxopiperidin-3-yl)-4-oxo-3,4-dihydrobenzo[d][1,2,3]triazin-6-yl)oxy)acetyl)piperidin-4-yl)methylpiperazin-1-yl)benzamide (Example 13)

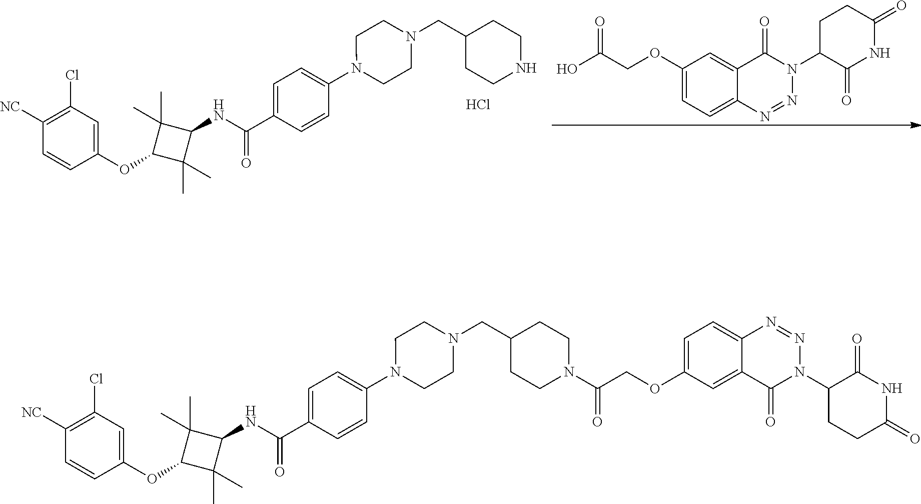 [SMILES: CC1(C)[C@H](NC(=O)c2ccc(N3CCN(CC4CCNCC4)CC3)cc2)C(C)(C)[C@H]1Oc1ccc(C#N)c(Cl)c1 ; Cl ; O=C(O)COc1ccc2nnn(C3CCC(=O)NC3=O)c(=O)c2c1 ; CC1(C)[C@H](NC(=O)c2ccc(N3CCN(CC4CCN(C(=O)COc5ccc6nnn(C7CCC(=O)NC7=O)c(=O)c6c5)CC4)CC3)cc2)C(C)(C)[C@H]1Oc1ccc(C#N)c(Cl)c1]

N-((1r,3r)-3-(3-chloro-4-cyanophenoxy)-2,2,4,4-tetramethylcyclobutyl)-4-(4-(piperidin-4-ylmthyl) piperazin-1-yl)benzamide hydrochloride (15 mg, 0.0249 mmol), 2-((3-(2,6-dioxopiperidin-3-yl)-4-oxo-3,4-dihydrobenzo[d][1,2,3]triazin-6-yl)oxy)acetic acid (15 mg, 0.0249 mmol), EDCl HCl (5.2 mg, 0.0273 mmol), and HOBt H₂O (4 mg, 0.0273 mmol) were dissolved in DMF (2 mL), and DIPEA (0.022 mL, 0.124 mmol) was added and stirred at room temperature for 12 hours. The reactant was diluted with water and then extracted with ethyl acetate. The organic layer was washed with brine, the residue was removed with anhydrous magnesium sulfate, and concentrated. The mixture was subjected to column chromatography to obtain the desired compound (8 mg, 34%) as a white solid.

¹H NMR (500 MHz, CDCl₃) δ 8.15 (d, J=8.1 Hz, 2H), 7.69 (d, J=8.4 Hz, 2H), 7.64 (d, J=7.3 Hz, 2H), 7.56 (d, J=8.7 Hz, 1H), 6.96 (s, 1H), 6.91 (d, J=8.5 Hz, 2H), 6.81 (d, J=8.8 Hz, 1H), 6.11 (d, J=8.1 Hz, 1H), 5.86-5.72 (m, 1H), 4.90 (s, 2H), 4.58 (d, J=13.2 Hz, 1H), 4.15 (d, J=8.1 Hz, 1H), 4.04 (s, 1H), 3.87-3.78 (m, 1H), 3.48 (q, J=6.9 Hz, 1H), 3.31 (m, 4H), 3.13 (t, J=12.9 Hz, 1H), 3.03-2.91 (m, 2H), 2.89-2.79 (m, 1H), 2.68 (t, J=13.0 Hz, 1H), 2.58 (m, 4H), 2.39 (m, 1H), 2.27 (m, 2H), 1.95 (d, J=13.2 Hz, 1H), 1.86 (d, J=14.0 Hz, 2H), 1.26 (s, 6H), 1.22 (m, 8H).

### Synthesis of N-((1r,3r)-3-(3-chloro-4-cyanophenoxy)-2,2,4,4-tetramethylcyclobutyl)-4-(4-(2-(piperidin-4-yloxy)ethyl)piperazin-1-yl)benzamide hydrogen chloride

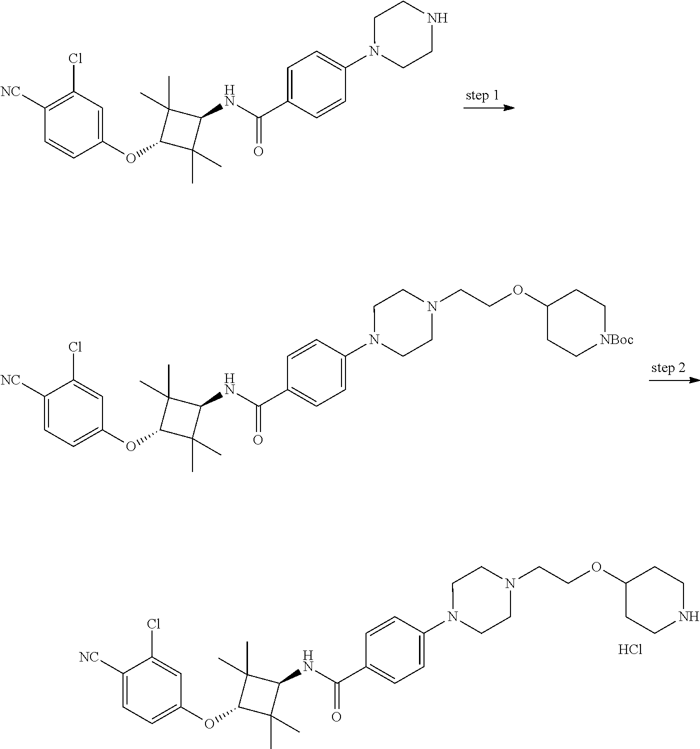 [SMILES: CC1(C)[C@H](NC(=O)c2ccc(N3CCNCC3)cc2)C(C)(C)[C@H]1Oc1ccc(C#N)c(Cl)c1 ; CC(C)(C)OC(=O)N1CCC(OCCN2CCN(c3ccc(C(=O)N[C@H]4C(C)(C)[C@H](Oc5ccc(C#N)c(Cl)c5)C4(C)C)cc3)CC2)CC1 ; CC1(C)[C@H](NC(=O)c2ccc(N3CCN(CCOC4CCNCC4)CC3)cc2)C(C)(C)[C@H]1Oc1ccc(C#N)c(Cl)c1 ; Cl]

### Synthesis of tert-butyl 4-(2-(4-(4-(((1r,3r)-3-(3-chloro-4-cyanophenoxy)-2,2,4,4-tetramethylcyclobutyl)carbamoyl)phenyl)piperazin-1-yl)ethoxy)piperidine-1-carboxylate (Step 1)

N-((1r,3r)-3-(3-chloro-4-cyanophenoxy)-2,2,4,4-tetramethylcyclobutyl)-4-(piperazin-1-yl)benzamide (200 mg, 0.428 mmol) was dissolved in DMF, and tert-butyl 4-(2-iodoethoxy)piperidine-1-carboxylate (228 mg, 0.642 mmol) and K₂CO₃ (118 mg, 0.856 mmol) were added and stirred overnight at room temperature. The reaction product was extracted with water and ethyl acetate, and the organic layer was washed with brine, dried over anhydrous magnesium sulfate, filtered and concentrated. The mixture was subjected to column chromatography to obtain the desired compound (237 mg, 0.341 mmol, 72%) as a white solid.

¹H NMR (300 MHz, CDCl₃) δ 7.75-7.68 (m, 2H), 7.58 (d, J=8.7 Hz, 1H), 6.98 (d, J=2.4 Hz, 1H), 6.96-6.89 (m, 2H), 6.83 (dd, J=8.7, 2.4 Hz, 1H), 6.13 (d, J=8.1 Hz, 1H), 4.17 (d, J=8.1 Hz, 1H), 4.06 (s, 1H), 3.86-3.72 (m, 2H), 3.67 (t, J=5.8 Hz, 2H), 3.54-3.43 (m, 1H), 3.40-3.27 (m, 4H), 3.18-3.03 (m, 2H), 2.79-2.60 (m, 6H), 1.92-1.78 (m, 2H), 1.62-1.50 (m, 2H), 1.48 (s, 9H), 1.28 (s, 6H), 1.24 (s, 6H).

### Synthesis of N-((1r,3r)-3-(3-chloro-4-cyanophenoxy)-2,2,4,4-tetramethylcyclobutyl)-4-(4-(2-(piperidin-4-yloxy)ethyl)piperazin-1-yl)benzamide hydrogen chloride (Step 2)

Tert-butyl 4-(2-(4-(4-(((1r,3r)-3-(3-chloro-4-cyanophenoxy)-2,2,4,4-tetramethylcyclobutyl)carbamoyl)phenyl)piperazin-1-yl)ethoxy)piperidine-1-carboxylate (237 mg, 0.341 mmol) were dissolved in DCM (4 mL), and 4.0 N HCl 1,4-dioxane (2 mL) was added and stirred at room temperature for 2 hours. The reaction product was concentrated under reduced pressure to obtain the desired compound (220 mg) as a white solid.

¹H NMR (300 MHz, DMSO-d₆) δ 9.09 (s, 2H), 7.91 (d, J=8.7 Hz, 1H), 7.82 (d, J=8.5 Hz, 2H), 7.64 (d, J=9.2 Hz, 1H), 7.21 (d, J=2.4 Hz, 1H), 7.14-6.96 (m, 3H), 4.35 (s, 1H), 4.07 (d, J=9.2 Hz, 1H), 4.02-3.84 (m, 4H), 3.76-3.55 (m, 3H), 3.43-3.29 (m, 4H), 3.28-3.20 (m, 2H), 3.02-2.86 (m, 2H), 2.08-1.90 (m, 2H), 1.85-1.69 (m, 2H), 1.23 (s, 6H), 1.13 (s, 6H).

### Synthesis of N-((1r,3r)-3-(3-chloro-4-cyanophenoxy)-2,2,4,4-tetramethylcyclobutyl)-4-(4-(2-((1-(1-(3-(2,6-dioxopiperidin-3-yl)-4-oxo-3,4-dihydrobenzo[d][1,2,3]triazin-6-yl)piperidine-4-carbonyl piperidin-4-yl)oxy)ethyl)piperazin-1-yl)benzamide (Example 30)

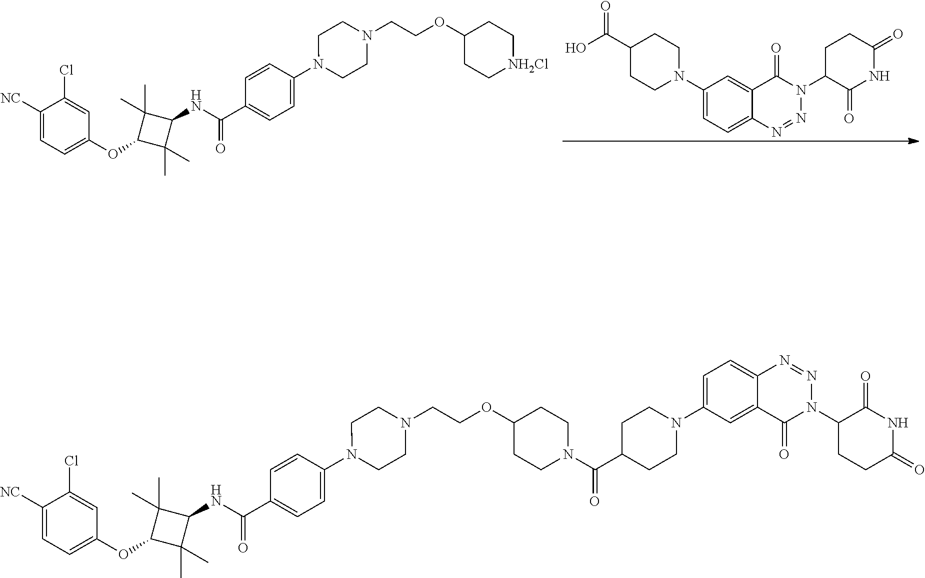

1-(3-(2,6-dioxopiperidin-3-yl)-4-oxo-3,4-dihydrobenzo[d][1,2,3]triazin-6-yl)piperidine-4-carboxylic acid (15 mg, 0.039 mmol), N-((1r,3r)-3-(3-chloro-4-cyanophenoxy)-2,2,4,4-tetramethylcyclobutyl)-4-(4-(2-(piperidin-4-yloxy)ethyl)piperazin-1-yl)benzamide hydrogen chloride (25 mg, 0.039 mmol), EDCl HCl (8 mg, 0.043 mmol), HOBt H₂O (6 mg, 0.043 mmol), and DIPEA (0.027 mL, 0.158 mmol) were dissolved in DMF and stirred overnight at room temperature. The reaction product was extracted with water and ethyl acetate, and the organic layer was washed with brine, dried over anhydrous magnesium sulfate, filtered and concentrated. The mixture was subjected to column chromatography to obtain the desired compound (21 mg, 0.022 mmol, 56%) as an ivory solid.

¹H NMR (500 MHz, CDCl₃) δ 8.36 (s, 1H), 7.99 (d, J=9.1 Hz, 1H), 7.73-7.66 (m, 2H), 7.57 (d, J=8.7 Hz, 1H), 7.52 (d, J=2.8 Hz, 1H), 7.45 (dd, J=9.1, 2.8 Hz, 1H), 6.97 (d, J=2.4 Hz, 1H), 6.94-6.88 (m, 2H), 6.81 (dd, J=8.7, 2.4 Hz, 1H), 6.13 (d, J=8.1 Hz, 1H), 5.81-5.73 (m, 1H), 4.15 (d, J=8.1 Hz, 1H), 4.10-4.02 (m, 3H), 3.98-3.89 (m, 1H), 3.81-3.72 (m, 1H), 3.72-3.63 (m, 2H), 3.63-3.56 (m, 1H), 3.41-3.28 (m, 6H), 3.15-3.05 (m, 2H), 3.00-2.88 (m, 2H), 2.88-2.77 (m, 2H), 2.76-2.64 (m, 6H), 2.42-2.33 (m, 1H), 2.01-1.80 (m, 6H), 1.70-1.55 (m, 2H), 1.26 (s, 6H), 1.22 (s, 6H); LC/MS (ESI) m/z 961.0 [M+H]⁺

### Synthesis of N-((1r,3r)-3-(3-chloro-4-cyanophenoxy)-2,2,4,4-tetramethylcyclobutyl)-4-(4-(2-((1-((3-2,6-dioxopiperidin-3-yl)-4-oxo-3,4-dihydrobenzo[d][1,2,3]triazin-6-yl)glycyl)piperidin-4-yl)oxy)ethyl)piperazin-1-yl)benzamide (Example 31)

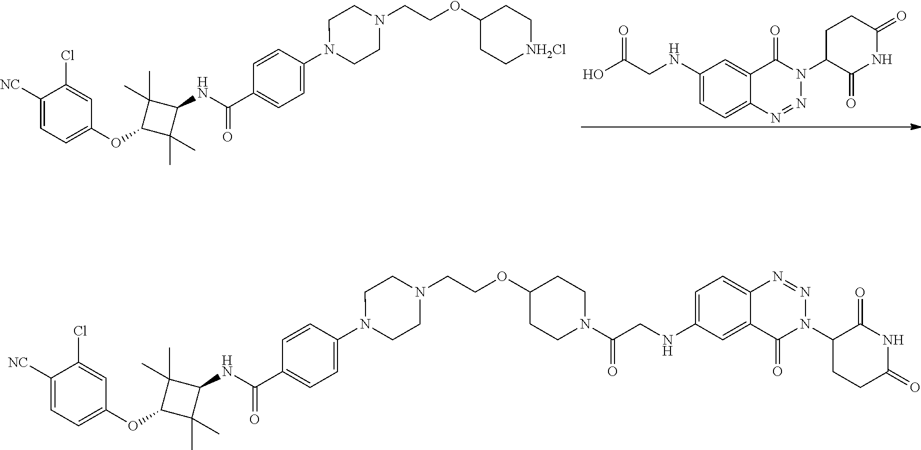

(3-(2,6-dioxopiperidin-3-yl)-4-oxo-3,4-dihydrobenzo[d][1,2,3]triazin-6-yl)glycine (13 mg, 0.039 mmol), N-((1r,3r)-3-(3-chloro-4-cyanophenoxy)-2,2,4,4-tetramethylcyclobutyl)-4-(4-(2-(piperidin-4-yloxy)ethyl)piperazin-1-yl)benzamide hydrogen chloride (25 mg, 0.039 mmol), EDCl HCl (8 mg, 0.043 mmol), HOBt H₂O (6 mg, 0.043 mmol), and DIPEA (0.027 mL, 0.158 mmol) were dissolved in DMF and stirred overnight at room temperature. The reaction product was extracted with water and ethyl acetate, and the organic layer was washed with brine, dried over anhydrous magnesium sulfate, filtered and concentrated. The mixture was subjected to column chromatography to obtain the desired compound (26 mg, 0.027 mmol, 70%) as an ivory solid.

¹H NMR (500 MHz, CDCl₃) δ 8.61-8.50 (m, 1H), 7.94 (d, J=8.9 Hz, 1H), 7.74-7.65 (m, 2H), 7.56 (d, J=8.7 Hz, 1H), 7.25 (dd, J=8.9, 2.7 Hz, 1H), 7.13 (d, J=2.7 Hz, 1H), 6.96 (d, J=2.4 Hz, 1H), 6.94-6.85 (m, 2H), 6.81 (dd, J=8.7, 2.4 Hz, 1H), 6.12 (d, J=8.1 Hz, 1H), 6.03 (t, J=3.9 Hz, 1H), 5.84-5.74 (m, 1H), 4.15 (d, J=8.1 Hz, 1H), 4.04 (s, 1H), 4.03-3.98 (m, 2H), 3.94-3.85 (m, 1H), 3.72-3.59 (m, 4H), 3.58-3.51 (m, 1H), 3.37-3.27 (m, 5H), 3.01-2.79 (m, 3H), 2.77-2.65 (m, 6H), 2.42-2.33 (m, 1H), 1.95-1.82 (m, 2H), 1.79-1.64 (m, 2H), 1.26 (s, 6H), 1.22 (s, 6H). ; LC/MS (ESI) m/z 907.0 [M+H]⁺

### Synthesis of N-((1r,3r)-3-(3-chloro-4-cyanophenoxy)-2,2,4,4-tetramethylcyclobutyl-4-(4-(2-((1-(2-((3-2,6-dioxopiperidin-3-yl)-4-oxo-3,4-dihydrobenzo[d][1,2,3]triazin-6-yl)oxyl)acetyl)piperidin-4-yl)oxy)ethyl)piperazin-1-yl)benzamide (Example 32)

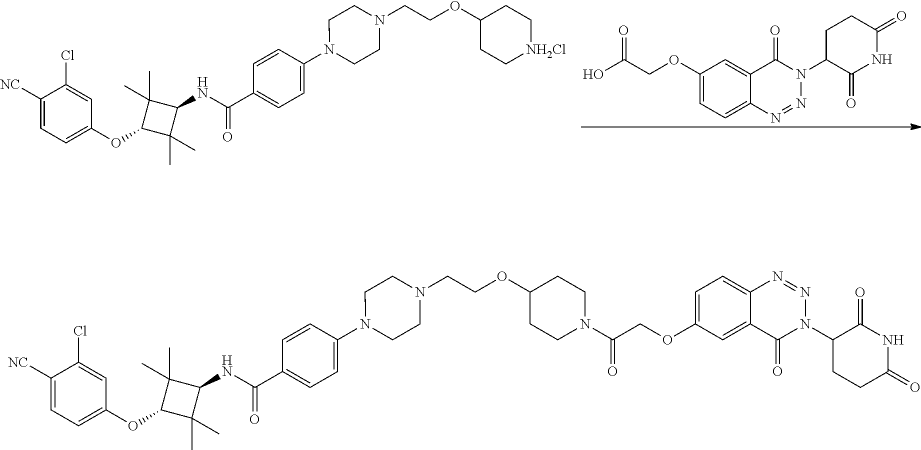

2-((3-(2,6-dioxopiperidin-3-yl)-4-oxo-3,4-dihydrobenzo[d][1,2,3]triazin-6-yl)oxy)acetic acid (11 mg, 0.032 mmo), N-((1r,3r)-3-(3-chloro-4-cyanophenoxy)-2,2,4,4-tetramethylcyclobutyl)-4-(4-(2-(piperidin-4-yloxy)ethyl)piperazin-1-yl)benzamide hydrogen chloride (25 mg, 0.039 mmol), EDCl HCl (7 mg, 0.035 mmol), HOBt H₂O (5 mg, 0.035 mmol), and DIPEA (0.022 mL, 0.128 mmol) were dissolved in DMF and stirred overnight at room temperature. The reaction product was extracted with water and ethyl acetate, and the organic layer was washed with brine, dried over anhydrous magnesium sulfate, filtered and concentrated. The mixture was subjected to column chromatography to obtain the desired compound (7 mg, 0.007 mmol, 21%) as a white solid.

¹H NMR (500 MHz, CDCl₃) δ 8.30 (s, 0.5H), 8.26 (s, 0.5H), 8.14 (d, J=8.6 Hz, 1H), 7.70 (d, J=8.4 Hz, 2H), 7.67-7.60 (m, 2H), 7.57 (d, J=8.7 Hz, 1H), 6.97 (d, J=2.3 Hz, 1H), 6.92 (d, J=8.5 Hz, 2H), 6.81 (dd, J=8.7, 2.3 Hz, 1H), 6.13 (d, J=8.1 Hz, 1H), 5.82-5.73 (m, 1H), 4.91 (s, 2H), 4.15 (d, J=8.0 Hz, 1H), 4.05 (s, 1H), 3.94-3.58 (m, 5H), 3.53-3.37 (m, 4H), 3.37-3.27 (m, 2H), 3.03-2.72 (m, 8H), 2.43-2.35 (m, 1H), 1.98-1.83 (m, 2H), 1.77-1.58 (m, 4H), 1.26 (s, 6H), 1.22 (s, 6H). ; LC/MS (ESI) m/z 908.6 [M+H]⁺

### Synthesis of N-((1r,3r)-3-(3-chloro-4-cyanophenoxy)-2,2,4,4-tetramethylcyclobutyl)-4-(4-(2-((1-(1-(3-(2,6-dioxopiperidin-3-yl)-4-oxo-3,4-dihydrobenzo[d][1,2,3]triazin-6-yl)azetidine-3-carbonyl)piperidin-4-yl)oxy)ethyl)piperazin-1-yl)benzamide (Example 33)

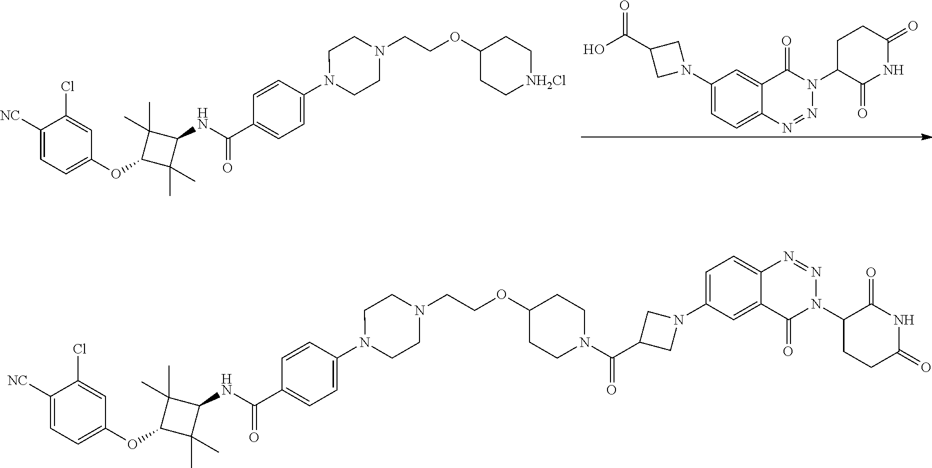

1-(3-(2,6-dioxopiperidin-3-yl)-4-oxo-3,4-dihydrobenzo[d][1,2,3]triazin-6-yl)azetidine-3-carboxylic acid (11 mg, 0.032 mmo), N-((1r,3r)-3-(3-chloro-4-cyanophenoxy)-2,2,4,4-tetramethylcyclobutyl)-4-(4-(2-(piperidin-4-yloxy)ethyl)piperazin-1-yl)benzamide hydrogen chloride (20 mg, 0.032 mmol), EDCl HCl (7 mg, 0.035 mmol), HOBt H₂O (5 mg, 0.035 mmol), and DIPEA (0.022 mL, 0.128 mmol) were dissolved in DMF and stirred overnight at room temperature. The reaction product was extracted with water and ethyl acetate, and the organic layer was washed with brine, dried over anhydrous magnesium sulfate, filtered and concentrated. The mixture was subjected to column chromatography to obtain the desired compound (13 mg, 0.014 mmol, 43%) as a white solid.

¹H NMR (500 MHz, CDCl₃) δ 8.24 (d, J=5.2 Hz, 1H), 7.97 (d, J=8.8 Hz, 1H), 7.73-7.66 (m, 2H), 7.57 (d, J=8.7 Hz, 1H), 7.06-7.02 (m, 1H), 6.96 (d, J=2.4 Hz, 1H), 6.96-6.93 (m, 1H), 6.93-6.89 (m, 2H), 6.81 (dd, J=8.7, 2.4 Hz, 1H), 6.12 (d, J=8.1 Hz, 1H), 5.80-5.72 (m, 1H), 4.32 (t, J=7.1 Hz, 2H), 4.30-4.24 (m, 2H), 4.15 (d, J=8.1 Hz, 1H), 4.04 (s, 1H), 3.94-3.84 (m, 1H), 3.84-3.74 (m, 1H), 3.73-3.63 (m, 2H), 3.64-3.57 (m, 1H), 3.57-3.49 (m, 1H), 3.50-3.42 (m, 1H), 3.38-3.27 (m, 4H), 3.23-3.14 (m, 1H), 3.00-2.78 (m, 3H), 2.76-2.62 (m, 6H), 2.42-2.33 (m, 1H), 1.92-1.80 (m, 2H), 1.71-1.59 (m, 2H), 1.26 (s, 6H), 1.22 (s, 6H).

### Synthesis of N-((1r,3r)-3-(3-chloro-4-cyanophenoxy)-2,2,4,4-tetramethylcyclobutyl)-4-(4-(2-((1-(3-(2,6-dioxopiperidin-3-yl)-4-oxo-3,4-dihydrobenzo[d][1,2,3]triazin-6-yl)piperidin-4-yl)oxy)ethyl piperazin-1-yl)benzamide (Example 34)

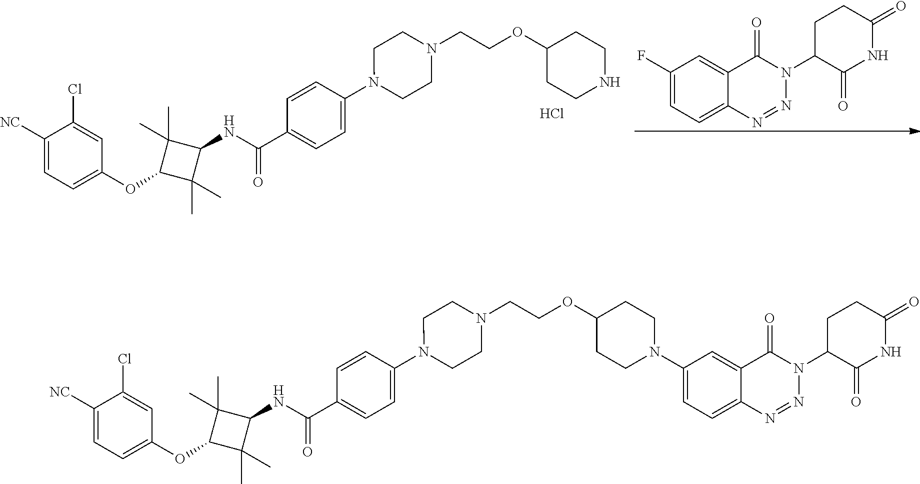

N-((1r,3r)-3-(3-chloro-4-cyanophenoxy)-2,2,4,4-tetramethylcyclobutyl)-4-(4-(2-(piperidin-4-yloxy)ethyl)piperazin-1-yl)benzamide hydrogen chloride (20 mg, 0.032 mmol), and 3-(6-fluoro-4-oxobenzo[d][1,2,3]triazin-3(4H)-yl)piperidine-2,6-dione (9 mg, 0.032 mmol) were dissolved in DMF (10 mL), and DIPEA (0.016 mL, 0.096 mmol) was added and stirred at 90° C. for 12 hours. The reactant was diluted with water and extracted with ethyl acetate. The organic layer was washed with brine, the residue was removed with anhydrous magnesium sulfate, and concentrated. The mixture was subjected to column chromatography to obtain the desired compound (17 mg, 0.019 mmol, 62%) as a yellow solid.

¹H NMR (500 MHz, CDCl₃) δ 8.33 (s, 1H), 7.99 (d, J=9.2 Hz, 1H), 7.69 (d, J=8.4 Hz, 2H), 7.56 (d, J=8.7 Hz, 1H), 7.55-7.51 (m, 1H), 7.44 (dd, J=9.2, 2.8 Hz, 1H), 6.99-6.95 (m, 1H), 6.91 (d, J=8.5 Hz, 2H), 6.84-6.77 (m, 1H), 6.12 (d, J=8.1 Hz, 1H), 5.81-5.73 (m, 1H), 4.15 (d, J=8.1 Hz, 1H), 4.04 (s, 1H), 3.83-3.74 (m, 2H), 3.70 (t, J=5.8 Hz, 2H), 3.66-3.59 (m, 1H), 3.38-3.34 (m, 1H), 3.34-3.26 (m, 5H), 3.00-2.78 (m, 3H), 2.75-2.64 (m, 6H), 2.42-2.33 (m, 1H), 2.04-1.94 (m, 2H), 1.80-1.71 (m, 2H), 1.26 (s, 6H), 1.22 (s, 6H).

### Synthesis of N-((1r,3r)-3-(3-chloro-4-cyanophenoxy)-2,2,4,4-tetramethylcyclobutyl-4-(4-(piperazin-1-ylmethyl)piperidin-1-yl)benzamide

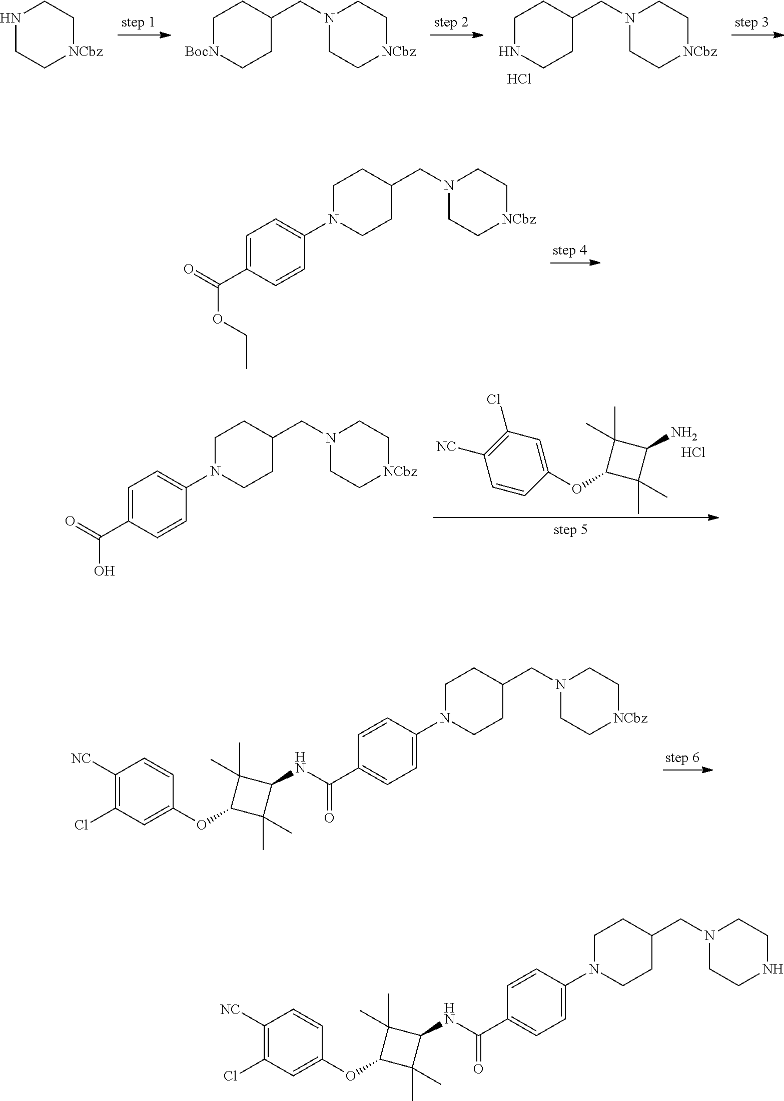

### Synthesis of benzyl 4-((1-tert-butoxycarbonyl)piperidin-4-yl)methyl)piperazine-1-carboxylate (Step 1)

Benzyl piperazine-1-carboxylate (1.29 g, 5.86 mmol), tert-butyl-4-formylpiperidine-1-carboxylate (1.50 g, 7.03 mmol), and AcOH (2 ml) were dissolved in MeOH (20 mL) and stirred at room temperature for 1 hour. NaBH₃CN (1.47 g, 23.4 mmol) was slowly added thereto and stirred at room temperature for 1 hour. When the reaction was complete, the reactant was concentrated, neutralized with 5% K₂CO₃ aqueous solution, and then extracted with dichloromethane. The organic layer was washed with brine, the residue was removed with anhydrous magnesium sulfate, and concentrated. The mixture was subjected to column chromatography to obtain the desired compound (1.25 g, 2.99 mmol, 51%) as a clear oil.

¹H NMR (300 MHz, CDCl₃) δ 7.42-7.28 (m, 5H), 5.13 (s, 2H), 4.21-3.97 (m, 2H), 3.58-3.40 (m, 4H), 2.68 (t, J=12.7 Hz, 2H), 2.48-2.27 (m, 4H), 2.17 (d, J=7.0 Hz, 2H), 1.78-1.61 (m, 3H), 1.45 (s, 9H), 1.18-0.95 (m, 2H).

### Synthesis of benzyl 4-(piperidin-4-ylmethyl)piperazine-1-carboxylate hydrochloride (Step 2)

Benzyl 4-((1-(tert-butoxycarbonyl)piperidin-4-yl)methyl)piperazine-1-carboxylate (1.25 g, 2.99 mmol) was dissolved in DCM (12 mL), and 4 M HCl in dioxane (6 ml) was added and stirred at room temperature for 1 hour. The reactant was concentrated to give the desired compound (1.1 g) as a white solid.

### Synthesis of benzyl 4-((1-(4-(ethoxycarbonyl)phenyl)piperidin-4-yl)methyl)piperazine-1-carboxylate (Step 3)

Ethyl 4-fluorobenzoate (419 mg, 2.49 mmol) was dissolved in DMSO, and benzyl 4-(piperidin-4-ylmethyl)piperazine-1-carboxylate hydrochloride (1.05 g, 2.99 mmol), and DIPEA (4.33 mL, 24.9 mmol) were added and stirred overnight at 130° C. The reaction product was diluted with water (20 ml) and extracted with ethyl acetate. The organic layer was washed with brine, the residue was removed with anhydrous magnesium sulfate, and concentrated. The mixture was subjected to column chromatography to obtain the desired compound (250 mg, 0.537 mmol, 21%) as a yellow oil.

¹H NMR (300 MHz, CDCl₃) δ 7.95-7.86 (m, 2H), 7.41-7.28 (m, 5H), 6.91-6.81 (m, 2H), 5.14 (s, 2H), 4.32 (q, J=7.1 Hz, 2H), 3.97-3.78 (m, 2H), 3.60-3.43 (m, 4H), 2.92-2.73 (m, 2H), 2.50-2.28 (m, 4H), 2.21 (d, J=7.1 Hz, 2H), 1.92-1.80 (m, 2H), 1.80-1.66 (m, 1H), 1.36 (t, J=7.1 Hz, 3H), 1.31-1.24 (m, 2H).

### Synthesis of 4-(4-((4-((benzyloxy)carbonyl))piperazin-1-yl)methyl)piperidin-1-yl)benzoic Acid (Step 4)

Benzyl 4-((1-(4-(ethoxycarbonyl)phenyl)piperidin-4-yl)methyl)piperazine-1-carboxylate (250 mg, 0.537 mmol) was dissolved in the mixture of MeOH (1 ml), water (1 mL), and THF (3 ml), and LiOH·H₂O (56 mg, 1.34 mmol) was added and stirred overnight at 40° C. The reactant was neutralized to pH 6 with 1 N HCl aqueous solution. The reaction product was extracted with ethyl acetate, the organic layer was washed with brine, the residue was removed with anhydrous magnesium sulfate, and concentrated. The mixture was subjected to column chromatography to obtain the desired compound (220 mg, 0.503 mmol, 93%) as a beige solid.

¹H NMR (300 MHz, DMSO-d₆) δ 7.79-7.70 (m, 2H), 7.44-7.28 (m, 5H), 6.97-6.88 (m, 2H), 5.08 (s, 2H), 3.97-3.80 (m, 2H), 2.89-2.70 (m, 2H), 2.39-2.26 (m, 4H), 2.16 (d, J=6.7 Hz, 2H), 1.85-1.66 (m, 3H), 1.21-1.06 (m, 2H).

### Synthesis of 4-((1-(4-(((1r,3r)-3-(3-chloro-4-cyanophenoxy)-2,2,4,4-tetramethylcyclobutyl)carbamoyl)phenyl)piperidin-4-yl)methyl)piperazine-1-carboxylate (Step 5)

4-((1r,3r)-3-amino-2,2,4,4-tetramethylcyclobutoxy)-2-chlorobenzonitrile hydrochloride (158 mg, 0.503 mmol) was dissolved in DMF, and 4-(4-((4-((benzyloxy)carbonyl)piperazin-1-yl)methyl)piperidin-1-yl)benzoic acid (220 mg, 0.503 mmol), EDCl HCl (106 mg, 0.553 mmol), HOBt H₂O (84 mg, 0.553 mmol), and DIPEA (0.350 mL, 2.01 mmol) were added and stirred overnight at room temperature. The reactant was extracted with water and ethyl acetate. The organic layer was washed with brine, the residue was removed with anhydrous magnesium sulfate, and concentrated. The mixture was subjected to column chromatography to obtain the desired compound (50 mg, 0.071 mmol, 14%) as a beige solid.

¹H NMR (500 MHz, CDCl₃) δ 7.73-7.64 (m, 2H), 7.62-7.52 (m, 1H), 7.42-7.29 (m, 5H), 6.97 (d, J=2.4 Hz, 1H), 6.95-6.87 (m, 2H), 6.81 (dd, J=8.7, 2.4 Hz, 1H), 6.10 (d, J=8.1 Hz, 1H), 5.14 (s, 2H), 4.15 (d, J=8.2 Hz, 1H), 4.08-4.03 (m, 1H), 3.93-3.76 (m, 2H), 3.60-3.44 (m, 4H), 2.88-2.73 (m, 2H), 2.48-2.30 (m, 4H), 2.22 (d, J=7.1 Hz, 2H), 1.94-1.82 (m, 2H), 1.80-1.67 (m, 1H), 1.26 (s, 6H), 1.22 (s, 6H), 1.20-1.14 (m, 2H).

### Synthesis of N-((1r,3r)-3-(3-chloro-4-cyanophenoxy)-2,2,4,4-tetramethylcyclobutyl)-4-(4-(piperazin-1-ylmethyl)piperidin-1-yl))benzamide (Step 6)

4-((1-(4-(((1r,3r)-3-(3-chloro-4-cyanophenoxy)-2,2,4,4-tetramethylcyclobutyl)carbamoyl)phenyl)piperidin-4-yl)methyl)piperazine-1-carboxylate (50 mg, 0.071 mmol) was dissolved in MeOH (3 ml). After adding 10% Pd/C (5 mg), the mixture was stirred at room temperature for 5 hours under hydrogen gas. The reaction solution was filtered through celite and concentrated under reduced pressure to obtain the desired compound (43 mg) as a beige solid.

### Synthesis of N-((1r,3r)-3-(3-chloro-4-cyanophenoxy)-2,2,4,4-tetramethylcyclobutyl)-4-(4-((4-(1-(3-(2,6-dioxopiperidin-3-yl)-4-oxo-3,4-dihydrobenzo[d][1,2,3]triazin-6-yl)piperidine-4-carbonyl)piperazin-1-yl)methyl)piperidin-1-yl)benzamide (Example 49)

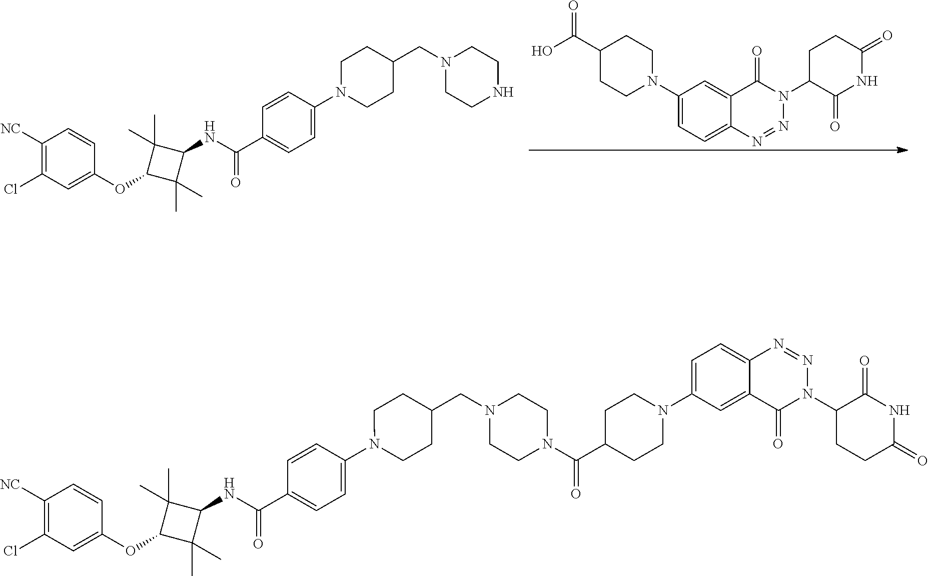 [SMILES: CC1(C)[C@H](NC(=O)c2ccc(N3CCC(CN4CCNCC4)CC3)cc2)C(C)(C)[C@H]1Oc1ccc(C#N)c(Cl)c1 ; O=C1CCC(n2nnc3ccc(N4CCC(C(=O)O)CC4)cc3c2=O)C(=O)N1 ; CC1(C)[C@H](NC(=O)c2ccc(N3CCC(CN4CCN(C(=O)C5CCN(c6ccc7nnn(C8CCC(=O)NC8=O)c(=O)c7c6)CC5)CC4)CC3)cc2)C(C)(C)[C@H]1Oc1ccc(C#N)c(Cl)c1]

1-(3-(2,6-dioxopiperidin-3-yl)-4-oxo-3,4-dihydrobenzo[d][1,2,3]triazin-6-yl)piperidine-4-carboxylic acid (14 mg, 0.035 mmol), N-((1r,3r)-3-(3-chloro-4-cyanophenoxy)-2,2,4,4-tetramethylcyclobutyl)-4-(4-(piperazin-1-ylmethyl)piperidin-1-yl)benzamide (20 mg, 0.035 mmol), EDCl HCl (8 mg, 0.043 mmol), HOBt H₂O (6 mg, 0.043 mmol), and DIPEA (0.027 mL, 0.158 mmol) were dissolved in DMF and stirred overnight at room temperature. The reaction product was extracted with water and ethyl acetate, and the organic layer was washed with brine, dried over anhydrous magnesium sulfate, filtered and concentrated. The mixture was subjected to column chromatography to obtain the desired compound (3 mg, 0.003 mmol, 9%) as an ivory solid.

¹H NMR (500 MHz, CDCl₃) δ 8.05 (s, 1H), 8.00 (d, J=9.2 Hz, 1H), 7.73-7.64 (m, 2H), 7.57 (d, J=8.7 Hz, 1H), 7.53 (d, J=2.9 Hz, 1H), 7.45 (dd, J=9.2, 2.9 Hz, 1H), 6.97 (d, J=2.4 Hz, 1H), 6.95-6.88 (m, 2H), 6.81 (dd, J=8.7, 2.4 Hz, 1H), 6.10 (d, J=8.1 Hz, 1H), 5.81-5.73 (m, 1H), 4.15 (d, J=8.1 Hz, 1H), 4.10-4.03 (m, 3H), 3.89-3.80 (m, 2H), 3.68-3.60 (m, 2H), 3.59-3.52 (m, 2H), 3.15-3.05 (m, 2H), 3.01-2.90 (m, 2H), 2.88-2.75 (m, 4H), 2.50-2.44 (m, 2H), 2.44-2.35 (m, 3H), 2.25 (d, J=7.2 Hz, 2H), 2.02-1.92 (m, 2H), 1.92-1.83 (m, 4H), 1.80-1.68 (m, 1H), 1.37-1.27 (m, 2H), 1.26 (s, 6H), 1.22 (s, 6H); LC/MS (ESI) m/z 931.1 [M+H]⁺, 929.0 [M−H]⁻

### Synthesis of N-((1r,3r)-3-(3-chloro-4-cyanophenoxy)-2,2,4,4-tetramethylcyclobutyl)-4-(1-(piperidin-4-ylmethyl)piperidin-4-yl)benzamide hydrochloride

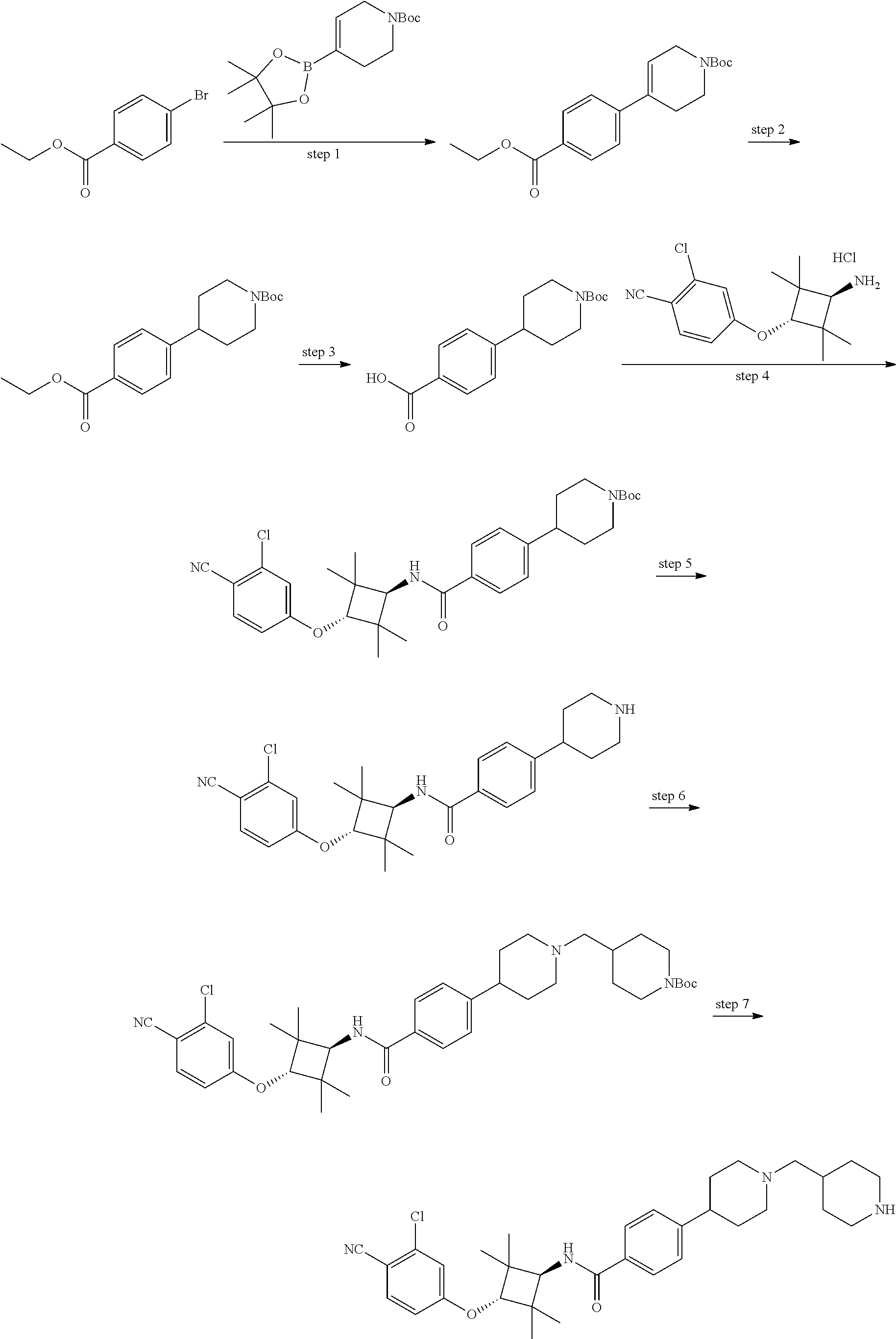

### Synthesis of tert-butyl 4-(4-(ethoxycarbonyl)phenyl)-3,6-dihydropyridine-1(2H)-carboxylate (Step 1)

Ethyl 4-bromobenzoate (100 mg, 0.436 mmol), tert-butyl 4-(4,4,5,5-tetramethyl-1,3,2-dioxaborolan-2-yl)-3,6-dihydropyridine-1(2H)-carboxylate (140 mg, 0.453 mmol), Potassium carbonate (80 mg, 0.580 mmol), and Pd(PPh₃)₄ (20 mg, 0.017 mmol) were dissolved in 1,4-dioxane (1 mL) and stirred at 100° C. for 12 hours under nitrogen. The reaction product was diluted with water and then extracted with ethyl acetate. The organic layer was washed with brine, the residue was removed with anhydrous magnesium sulfate, and concentrated. The mixture was subjected to column chromatography to obtain the desired compound (106 mg, 0.319 mmol, 73%) as a clear oil.

¹H NMR (300 MHz, CDCl₃) δ 8.06-7.95 (m, 2H), 7.51-7.38 (m, 2H), 6.16 (s, 1H), 4.37 (q, J=7.1 Hz, 2H), 4.19-4.04 (m, 2H), 3.65 (t, J=5.7 Hz, 2H), 2.61-2.48 (m, 2H), 1.50 (s, 9H), 1.39 (d, J=7.1 Hz, 3H).

### Synthesis of tert-butyl 4-(4-(ethoxycarbonyl)phenyl)piperidine-1-carboxylate (Step 2)

Tert-butyl 4-(4-(ethoxycarbonyl)phenyl)-3,6-dihydropyridine-1(2H)-carboxylate (106 mg, 0.319 mmol), and 10% Pd/C (25 mg) were dissolved in ethanol (5 ml) and stirred at room temperature for 2 hours under hydrogen. The reaction solution was filtered through cell pite and concentrated to obtain the desired compound (123 mg) as beige oil.

¹H NMR (300 MHz, CDCl₃) δ 7.98 (d, J=8.0 Hz, 2H), 7.36-7.19 (m, 2H), 4.37 (q, J=7.1 Hz, 2H), 4.33-4.16 (m, 2H), 2.93-2.74 (m, 2H), 2.75-2.64 (m, 1H), 1.91-1.75 (m, 2H), 1.69-1.57 (m, 2H), 1.49 (s, 9H), 1.38 (t, J=7.1 Hz, 3H).

### Synthesis of 4-(1-(tert-butoxycarbonyl)piperidin-4-yl)benzoic Acid (Step 3)

Tert-butyl 4-(4-(ethoxycarbonyl)phenyl)piperidine-1-carboxylate (106 mg, 0.319 mmol) was dissolved in the mixture of MeOH (1 ml), water (1 ml), and THF (3 ml), and LiOH·H₂O (33 mg, 0.797 mmol) was added and stirred overnight at 40° C. The reactant was neutralized to pH 6 with 1 N HCl aqueous solution and extracted with ethyl acetate. The organic layer was washed with brine, the residue was removed with anhydrous magnesium sulfate, and concentrated. The mixture was subjected to column chromatography to obtain the target compound as a beige solid.

¹H NMR (300 MHz, DMSO-d₆) δ 7.96-7.82 (m, 2H), 7.47-7.34 (m, 2H), 4.16-3.97 (m, 2H), 2.96-2.69 (m, 3H), 1.85-1.70 (m, 2H), 1.58-1.46 (m, 2H), 1.42 (s, 9H).

### Synthesis of tert-butyl 4-(4-(((1r,3r)-3-(3-chloro-4-cyanophenoxy)-2,2,4,4-tetramethylcyclobutyl)carbamoyl)phenyl)piperidine-1-carboxylate (Step 4)

4-((1r,3r)-3-amino-2,2,4,4-tetramethylcyclobutoxy)-2-chlorobenzonitrile hydrochloride (100 mg, 0.319 mmol) was dissolved in DMF, and 44-(1-(tert-butoxycarbonyl)piperidin-4-yl)benzoic acid (97 mg, 0.319 mmol), EDCl HCl (67 mg, 0.351 mmol), HOBt H₂O (54 mg, 0.351 mmol), and DIPEA (0.222 mL, 1.27 mmol) were added and stirred overnight at room temperature. The reactants are extracted with water and ethyl acetate. The organic layer was washed with brine, the residue was removed with anhydrous magnesium sulfate, and concentrated. The mixture was subjected to column chromatography to obtain the desired compound (113 mg, 0.199 mmol, 62%) as a beige solid.

¹H NMR (500 MHz, CDCl₃) δ 7.77-7.69 (m, 2H), 7.57 (d, J=8.7 Hz, 1H), 7.35-7.27 (m, 2H), 6.97 (d, J=2.4 Hz, 1H), 6.81 (dd, J=8.7, 2.4 Hz, 1H), 6.19 (d, J=8.1 Hz, 1H), 4.37-4.20 (m, 2H), 4.20-4.13 (m, 1H), 4.05 (s, 1H), 2.93-2.63 (m, 3H), 1.91-1.77 (m, 2H), 1.72-1.61 (m, 2H), 1.49 (s, 9H), 1.27 (s, 6H), 1.23 (s, 6H).

### Synthesis of N-((1r,3r)-3-(3-chloro-4-cyanophenoxy)-2,2,4,4-tetramethylcyclobutyl)-4-(piperidin-4-yl)benzamide (Step 5)

Tert-butyl 4-(4-(((1r,3r)-3-(3-chloro-4-cyanophenoxy)-2,2,4,4-tetramethylcyclobutyl)carbamoyl)phenyl)piperidine-1-carboxylate (113 mg, 0.199 mmol) was dissolved in DCM (4 mL), and 4.0 N HCl 1,4-dioxane (2 mL) was added and stirred at room temperature for 2 hours. The reactants were concentrated under reduced pressure, neutralized with saturated NaHCO₃ aqueous solution, and extracted with 10% MeOH/DCM solution. The organic layer was washed with brine, the residue was removed with anhydrous magnesium sulfate and concentrated to obtain the desired compound (95 mg) as a white solid.

¹H NMR (300 MHz, Methanol-d₄) δ 7.88-7.82 (m, 2H), 7.82-7.77 (m, 1H), 7.75 (d, J=8.7 Hz, 1H), 7.48-7.40 (m, 2H), 7.15 (d, J=2.4 Hz, 1H), 7.01 (dd, J=8.7, 2.4 Hz, 1H), 4.31 (s, 1H), 4.23-4.14 (m, 1H), 3.63-3.48 (m, 2H), 3.27-3.12 (m, 2H), 3.12-2.94 (m, 1H), 2.18-1.89 (m, 4H), 1.31 (s, 6H), 1.25 (s, 6H).

### Synthesis of tert-butyl 4-((4-(4-(((1r,3r)-3-(3-chloro-4-cyanophenoxy)-2,2,4,4-tetramethylcyclobutyl)carbamoyl)phenyl)piperidin-1-yl)methyl)piperidine-1-carboxylate (Step 6)

N-((1r,3r)-3-(3-chloro-4-cyanophenoxy)-2,2,4,4-tetramethylcyclobutyl)-4-(piperidin-4-yl)benzamide (100 mg, 0.453 mmol) was dissolved in MeOH (3 mL), and tert-butyl 4-formylpiperidine-1-carboxylate (116 mg, 0.545 mmol), and AcOH (0.3 mL) were added and stirred at room temperature for 1 hour. NaBH₃CN (113 mg, 1.81 mmol) was slowly added to the reactant and stirred at room temperature for 12 hours. The reactant was concentrated, neutralized with 5% K₂CO₃ aqueous solution, and then extracted with dichloromethane. The organic layer was washed with brine, the residue was removed with anhydrous magnesium sulfate, and concentrated. The mixture was subjected to column chromatography to obtain the desired compound (95 mg, 0.143 mmol, 71%) as a white solid.

¹H NMR (300 MHz, CDCl₃) δ 67.74-7.69 (m, 2H), 7.57 (d, J=8.7 Hz, 1H), 7.35-7.30 (m, 2H), 6.97 (d, J=2.4 Hz, 1H), 6.81 (dd, J=8.7, 2.4 Hz, 1H), 6.20 (d, J=8.1 Hz, 1H), 4.20-4.14 (m, 1H), 4.14-4.07 (m, 1H), 4.05 (s, 1H), 3.05-2.93 (m, 2H), 2.78-2.63 (m, 2H), 2.61-2.51 (m, 1H), 2.20 (d, J=7.0 Hz, 2H), 2.10-1.98 (m, 2H), 1.86-1.71 (m, 6H), 1.71-1.63 (m, 2H), 1.46 (s, 9H), 1.27 (s, 6H), 1.23 (s, 6H), 1.15-1.04 (m, 2H).

### Synthesis of N-((1r,3r)-3-(3-chloro-4-cyanophenoxy)-2,2,4,4-tetramethylcyclobutyl)-4-(1-(piperidin-4-ylmethyl)piperidin-4-yl)benzamide hydrochloride (Step 7)

N-((1r,3r)-3-(3-chloro-4-cyanophenoxy)-2,2,4,4-tetramethylcyclobutyl)-4-(piperidin-4-yl)benzamide (95 mg, 0.143 mmol) was dissolved in DCM (4 mL), and 4.0 N HCl 1,4-dioxane (2 mL) was added and stirred at room temperature for 2 hours. The reactant was concentrated under reduced pressure to obtain the desired compound (600 mg, 94%) as a white solid.

### Synthesis of N-((1r,3r)-3-(3-chloro-4-cyanophenoxy)-2,2,4,4-tetramethylcyclobutyl)-4-(1-((1-(3-(2,6-dioxopiperidin-3-yl)-4-oxo-3,4-dihydrobenzo[d][1,2,3]triazin-6-yl)piperidin-4-yl)methyl)piperidin-4-yl)benzamide (Example 50)

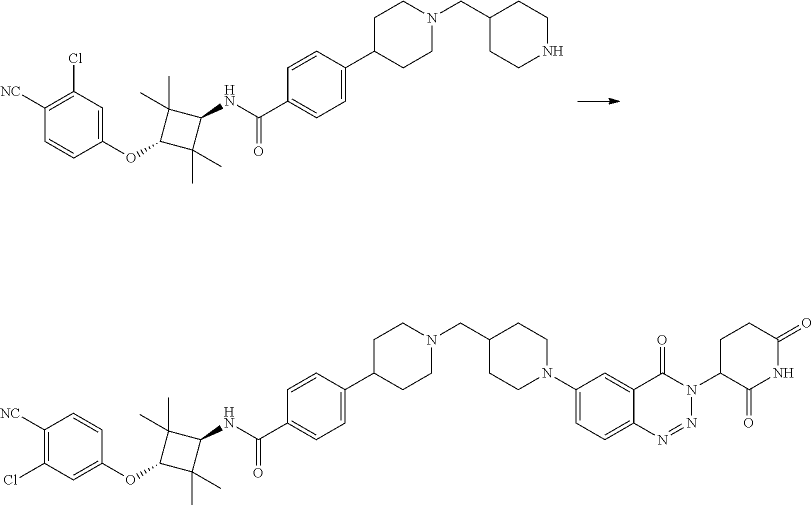 [SMILES: CC1(C)[C@H](NC(=O)c2ccc(C3CCN(CC4CCNCC4)CC3)cc2)C(C)(C)[C@H]1Oc1ccc(C#N)c(Cl)c1 ; CC1(C)[C@H](NC(=O)c2ccc(C3CCN(CC4CCN(c5ccc6nnn(C7CCC(=O)NC7=O)c(=O)c6c5)CC4)CC3)cc2)C(C)(C)[C@H]1Oc1ccc(C#N)c(Cl)c1]

N-((1r,3r)-3-(3-chloro-4-cyanophenoxy)-2,2,4,4-tetramethylcyclobutyl)-4-(1-(piperidin-4-ylmethyl)piperidin-4-yl)benzamide hydrochloride (9 mg, 0.033 mmol), and 3-(6-fluoro-4-oxobenzo[d][1,2,3]triazin-3(4H)-yl)piperidine-2,6-dione (9 mg, 0.033 mmol) were dissolved in DMF (1 mL), and DIPEA (0.017 mL, 0.099 mmol) was added and stirred at 90° C. for 12 hours. The reactant was diluted with water and extracted with ethyl acetate. The organic layer was washed with brine, the residue was removed with anhydrous magnesium sulfate, and concentrated. The mixture was subjected to column chromatography to obtain the desired compound (7 mg, 0.008 mmol, 25%) as a yellow solid.

¹H NMR (500 MHz, CDCl₃) δ 8.04 (s, 1H), 7.99 (d, J=9.2 Hz, 1H), 7.77-7.69 (m, 2H), 7.57 (d, J=8.7 Hz, 1H), 7.52 (d, J=2.9 Hz, 1H), 7.44 (dd, J=9.2, 2.9 Hz, 1H), 7.38-7.30 (m, 2H), 6.97 (d, J=2.4 Hz, 1H), 6.81 (dd, J=8.7, 2.4 Hz, 1H), 6.19 (d, J=8.1 Hz, 1H), 5.81-5.73 (m, 1H), 4.16 (d, J=8.1 Hz, 1H), 4.10-4.01 (m, 3H), 3.09-2.78 (m, 7H), 2.64-2.51 (m, 1H), 2.42-2.35 (m, 1H), 2.30-2.20 (m, 2H), 2.13-2.00 (m, 2H), 2.00-1.87 (m, 3H), 1.90-1.72 (m, 5H), 1.38-1.29 (m, 2H), 1.27 (s, 6H), 1.23 (s, 6H).

### Synthesis of N-((1r,3r)-3-(3-chloro-4-cyanophenoxy)-2,2,4,4-tetramethylcyclobutyl)-4-(4-((4-hydroxypiperidin-4-yl)methyl)piperazin-1-yl)benzamide hydrochloride

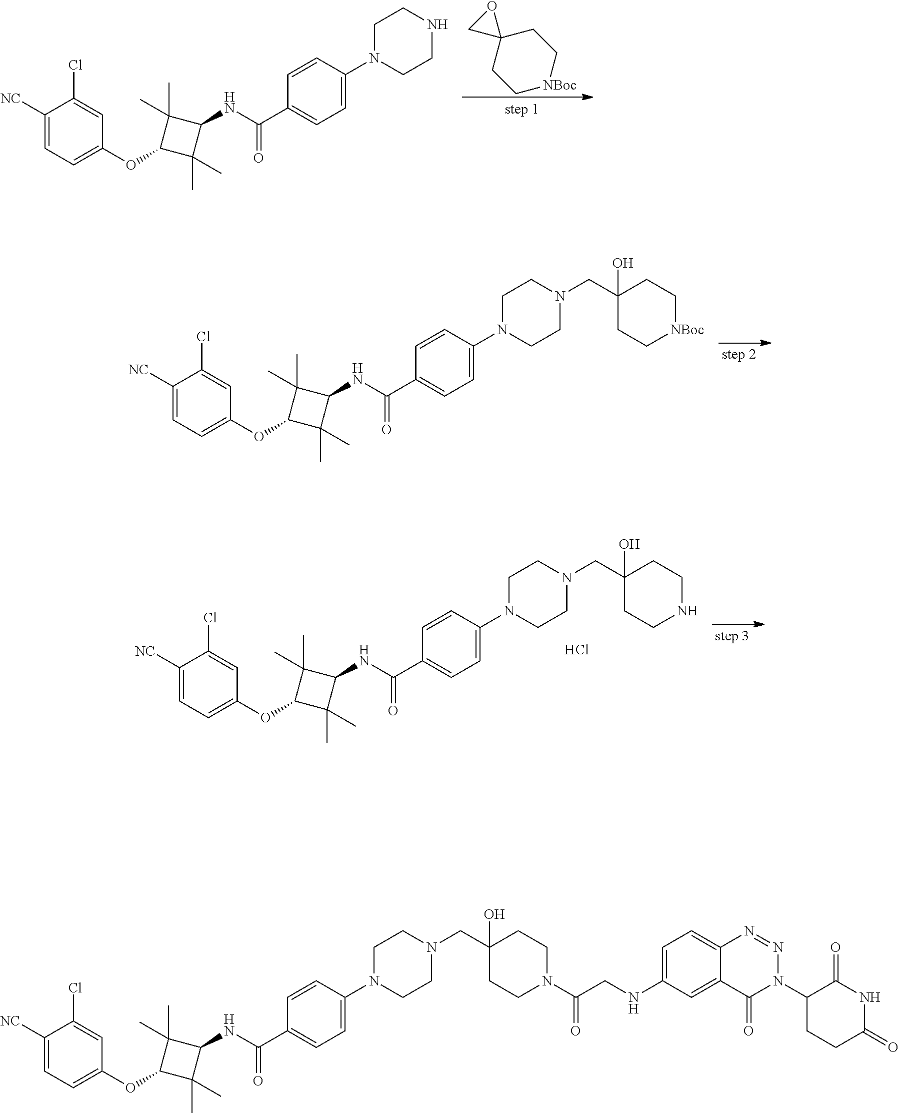

### Synthesis of tert-butyl 4-(4-(4-(((1r,3r)-3-(3-chloro-4-cyanophenoxy)-2,2,4,4-tetramethylcyclobutyl)carbamoyl)phenyl)piperazin-1-yl)methyl)-4-hydroxypiperidine-1-carboxylate (Step 1)

N-((1r,3r)-3-(3-chloro-4-cyanophenoxy)-2,2,4,4-tetramethylcyclobutyl)-4-(piperazin-1-yl)benzamide (200 mg, 0.428 mmol) was dissolved in MeOH, and tert-butyl 1-oxa-6-azaspiro[2.5]octane-6-carboxylate (137 mg, 0.642 mmol) was added and stirred overnight at room temperature. The reactant was concentrated and the desired compound (150 mg) was obtained as a white solid by column chromatography.

¹H NMR (300 MHz, DMSO-d₆) δ 7.91 (d, J=8.8 Hz, 1H), 7.74 (d, J=8.8 Hz, 2H), 7.51 (d, J=9.2 Hz, 1H), 7.21 (d, J=2.4 Hz, 1H), 7.05-6.91 (m, 3H), 4.32 (s, 1H), 4.25 (s, 1H), 4.05 (d, J=9.1 Hz, 1H), 3.68-3.55 (m, 2H), 3.29-3.17 (m, 3H), 3.17-3.00 (m, 2H), 2.75-2.59 (m, 4H), 2.31 (s, 2H), 1.50-1.41 (m, 4H), 1.39 (s, 9H), 1.22 (s, 6H), 1.12 (s, 6H).

### Synthesis of N-((1r,3r)-3-(3-chloro-4-cyanophenoxy)-2,2,4,4-tetramethylcyclobutyl-4-(4-((4-hydroxypiperidin-4-yl))methyl)piperazin-1-yl)benzamide hydrochloride (Step 2)

Tert-butyl 4-((4-(4-(((1r,3r)-3-(3-chloro-4-cyanophenoxy)-2,2,4,4-tetramethylcyclobutyl)carbamoyl)phenyl)piperazin-1-yl)methyl)-4-hydroxypiperidine-1-carboxylate (150 mg, 0.220 mmol) was dissolved in DCM (3 mL), and 4.0 N HCl 1,4-dioxane (1.5 mL) was added and stirred at room temperature for 2 hours. The reactant was concentrated under reduced pressure to obtain the desired compound (154 mg) as a white solid.

### Synthesis of N-((1r,3r)-3-(3-chloro-4-cyanophenoxy)-2,2,4,4-tetramethylcyclobutyl)-4-(4-((1-((3-(2,6-dioxopiperidin-3-yl)-4-oxo-3,4-dihydrobenzo[d][1,2,3]triazin-6-yl)glycyl-4-hydroxypiperidin-4-yl)methyl)piperazin-1-yl)benzamide (Example 53)

(3-(2,6-dioxopiperidin-3-yl)-4-oxo-3,4-dihydrobenzo[d][1,2,3]triazin-6-yl)glycine (11 mg, 0.032 mmol), N-((1r,3r)-3-(3-chloro-4-cyanophenoxy)-2,2,4,4-tetramethylcyclobutyl)-4-(4-((4-hydroxypiperidin-4-yl)methyl)piperazin-1-yl)benzamide hydrochloride (20 mg, 0.032 mmol), EDCl HCl (8 mg, 0.043 mmol), HOBt H₂O (6 mg, 0.043 mmol), and DIPEA (0.027 mL, 0.158 mmol) were dissolved in DMF and stirred overnight at room temperature. The reactant was extracted with water and ethyl acetate, and the organic layer was washed with brine, dried over anhydrous magnesium sulfate, filtered and concentrated. The mixture was subjected to column chromatography to obtain the desired compound (17 mg, 0.019 mmol, 59%) as an ivory solid.

¹H NMR (400 MHz, DMSO-d₆) δ 11.12 (s, 1H), 7.98-7.86 (m, 2H), 7.74 (d, J=8.6 Hz, 2H), 7.57-7.45 (m, 2H), 7.30-7.18 (m, 2H), 7.13 (s, 1H), 7.00 (dd, J=8.8, 2.5 Hz, 1H), 6.96 (d, J=8.6 Hz, 2H), 5.92-5.76 (m, 1H), 4.36 (s, 1H), 4.32 (s, 1H), 4.23-4.08 (m, 2H), 4.09-4.00 (m, 2H), 3.77-3.65 (m, 1H), 3.28-3.19 (m, 4H), 3.13-2.87 (m, 3H), 2.77-2.61 (m, 6H), 2.42-2.30 (m, 2H), 2.27-2.16 (m, 1H), 1.70-1.39 (m, 4H), 1.22 (s, 6H), 1.13 (s, 6H). ; LC/MS (ESI) m/z 893.0 [M+H]⁺, 890.9 [M−H]⁻

### Synthesis of N-((1r,3r)-3-(3-chloro-4-cyanophenoxy)-2,2,4,4-tetramethylcyclobutyl)-4-(4-((4-fluoropiperidin-4-yl)methyl)piperazin-1-yl)benzamide hydrochloride

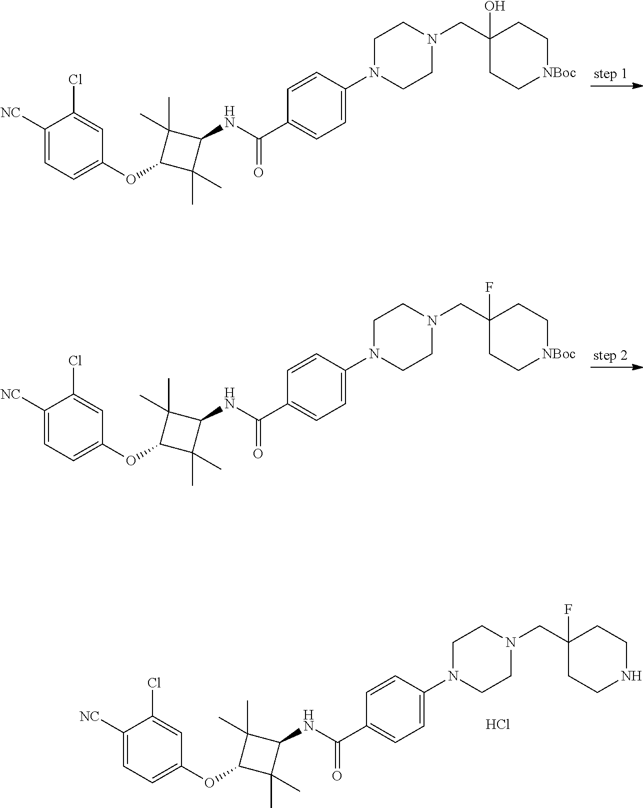 [SMILES: CC(C)(C)OC(=O)N1CCC(O)(CN2CCN(c3ccc(C(=O)N[C@H]4C(C)(C)[C@H](Oc5ccc(C#N)c(Cl)c5)C4(C)C)cc3)CC2)CC1 ; CC(C)(C)OC(=O)N1CCC(F)(CN2CCN(c3ccc(C(=O)N[C@H]4C(C)(C)[C@H](Oc5ccc(C#N)c(Cl)c5)C4(C)C)cc3)CC2)CC1 ; CC1(C)[C@H](NC(=O)c2ccc(N3CCN(CC4(F)CCNCC4)CC3)cc2)C(C)(C)[C@H]1Oc1ccc(C#N)c(Cl)c1 ; Cl]

### Synthesis of tert-butyl 4-((4-(4-(((1r,3r)-3-(3-chloro-4-cyanophenoxy)-2,2,4,4-tetramethylcyclobutyl)carbamoylphenyl)piperazin-1-yl)methyl)-4-fluoropiperidine-1-carboxylate (Step 1)

Tert-butyl 4-((4-(4-(((1r,3r)-3-(3-chloro-4-cyanophenoxy)-2,2,4,4-tetramethylcyclobutyl)carbamoyl)phenyl)piperazin-1-yl)methyl)-4-hydroxypiperidine-1-carboxylate (330 mg, 0.485 mmol) was dissolved in DCM (20 mL), and DAST (0.192 ml, 1.45 mmol) was slowly added at −78° C. and stirred at roam temperature for 2 hours. The reactants were neutralized with saturated NaHCO₃ aqueous solution and then extracted with dichloromethane. The organic layer was washed with brine, dried over anhydrous magnesium sulfate, filtered and concentrated. The mixture was subjected to column chromatography to obtain the desired compound (93 mg, 0.136 mmol, 28%) as a white solid.

¹H NMR (300 MHz, DMSO-d₆) δ 7.91 (d, J=8.8 Hz, 1H), 7.74 (d, J=8.8 Hz, 2H), 7.51 (d, J=9.2 Hz, 1H), 7.21 (d, J=2.4 Hz, 1H), 7.05-6.91 (m, 3H), 4.32 (s, 1H), 4.25 (s, 1H), 4.05 (d, J=9.1 Hz, 1H), 3.68-3.55 (m, 2H), 3.29-3.17 (m, 3H), 3.17-3.00 (m, 2H), 2.75-2.59 (m, 4H), 2.31 (s, 2H), 1.50-1.41 (m, 4H), 1.39 (s, 9H), 1.22 (s, 6H), 1.12 (s, 6H).

### Synthesis of N-((1r,3r)-3-(3-chloro-4-cyanophenoxy)-2,2,4,4-tetramethylcyclobutyl)-4-(4-((4-fluoropiperidin-4-yl)methyl)piperazin-1-yl)benzamide hydrochloride (Step 2)

Tert-butyl 4-((4-(4-(((1r,3r)-3-(3-chloro-4-cyanophenoxy)-2,2,4,4-tetramethylcyclobutyl)carbamoyl)phenyl)piperazin-1-yl)methyl)-4-fluoropiperidine-1-carboxylate (93 mg, 0.136 mmol) was dissolved in DCM (2 mL), and 4.0 N HCl 1,4-dioxane (0.3 mL) was added and stirred at room temperature for 2 hours. The reactant was concentrated under reduced pressure to obtain the desired compound (88 mg) as a white solid.

¹H NMR (300 MHz, DMSO-d₆) δ 11.22 (s, 1H), 9.29-9.02 (m, 2H), 7.91 (d, J=8.7 Hz, 1H), 7.88-7.78 (m, 2H), 7.67-7.53 (m, 1H), 7.21 (d, J=2.4 Hz, 1H), 7.15-6.96 (m, 3H), 4.34 (s, 1H), 4.07 (d, J=9.1 Hz, 1H), 3.77-3.59 (m, 5H), 3.56-3.38 (m, 4H), 3.38-3.19 (m, 4H), 3.13-2.95 (m, 2H), 2.34-2.20 (m, 2H), 1.22 (s, 6H), 1.13 (s, 6H).

### Synthesis of N-((1r,3r)-3-(3-chloro-4-cyanophenoxy)-2,2,4,4-tetramethylcyclobutyl)-4-(4-((1-(3-(2,6-dioxopiperidin-3-yl)-4-oxo-3,4-dihydrobenzo[d][1,2,3]triazin-6-yl)-4-fluoropiperidin-4-yl)methyl)piperazin-1-yl)benzamide (Example 61)

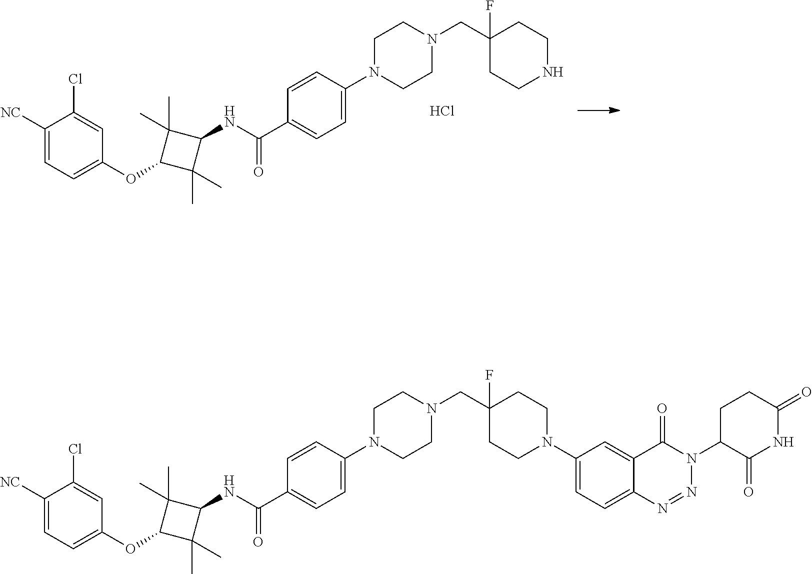 [SMILES: CC1(C)[C@H](NC(=O)c2ccc(N3CCN(CC4(F)CCNCC4)CC3)cc2)C(C)(C)[C@H]1Oc1ccc(C#N)c(Cl)c1 ; Cl ; CC1(C)[C@H](NC(=O)c2ccc(N3CCN(CC4(F)CCN(c5ccc6nnn(C7CCC(=O)NC7=O)c(=O)c6c5)CC4)CC3)cc2)C(C)(C)[C@H]1Oc1ccc(C#N)c(Cl)c1]

N-((1r,3r)-3-(3-chloro-4-cyanophenoxy)-2,2,4,4-tetramethylcyclobutyl)-4-(4-((4-fluoropiperidin-4-yl)methyl)piperazin-1-yl)benzamide hydrochloride (20 mg, 0.032 mmol), and 3-(6-fluoro-4-oxobenzo[d][1,2,3]triazin-3(4H)-yl)piperidine-2,6-dione (9 mg, 0.032 mmol) were dissolved in DMF (10 mL), and DIPEA (0.016 mL, 0.096 mmol) was added and stirred at 90° C. for 12 hours. The reactant was diluted with water and extracted with ethyl acetate. The organic layer was washed with brine, the residue was removed with anhydrous magnesium sulfate, and concentrated. The mixture was subjected to column chromatography to obtain the desired compound (11 mg, 0.013 mmol, 41%) as a yellow solid.

¹H NMR (300 MHz, CDCl₃) δ 8.12 (s, 1H), 8.01 (d, J=9.1 Hz, 1H), 7.75-7.66 (m, 2H), 7.61-7.53 (m, 2H), 7.46 (dd, J=9.2, 2.9 Hz, 1H), 6.97 (d, J=2.4 Hz, 1H), 6.91 (d, J=8.8 Hz, 2H), 6.81 (dd, J=8.8, 2.4 Hz, 1H), 6.12 (d, J=8.1 Hz, 1H), 5.84-5.72 (m, 1H), 4.15 (d, J=8.1 Hz, 1H), 4.05 (s, 1H), 3.97-3.82 (m, 2H), 3.48-3.35 (m, 2H), 3.35-3.20 (m, 4H), 3.04-2.78 (m, 3H), 2.78-2.67 (m, 4H), 2.62 (s, 1H), 2.54 (s, 1H), 2.45-2.32 (m, 1H), 2.22-2.07 (m, 2H), 1.91-1.52 (m, 2H), 1.26 (s, 6H), 1.22 (s, 6H). ; LC/MS (ESI) m/z 838.0 [M+H]⁺, 835.9 [M−H]⁻

### Synthesis of N-((1r,3r)-3-(3-chloro-4-cyanophenoxy)-2,2,4,4-tetramethylcyclobutyl)-4-(1-(2-((1-(1-(3-(2,6-dioxopiperidin-3-yl)-4-oxo-3,4-dihydrobenzo[d][1,2,3]triazin-6-yl)piperidine-4-carbonyl piperidin-4-yl)oxy)ethyl)piperidin-4-yl)benzamide (Example 74)

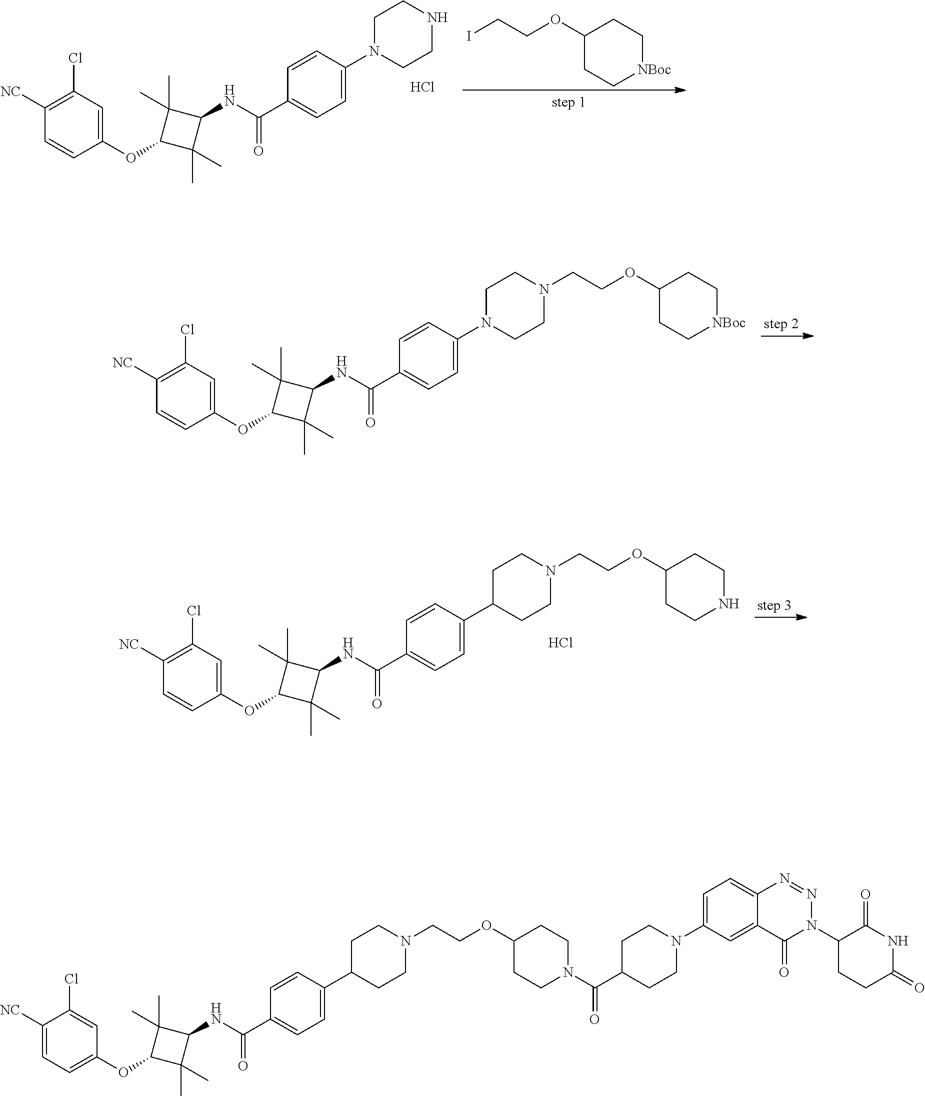

### Synthesis of tert-butyl 4-(2-(4-(4-(((1 r,3r)-3-(3-chloro-4-cyanophenoxy)-2,2,4,4-tetramethylcyclobutyl)carbamoyl)phenyl)piperidin-1-yl)ethoxy)piperidine-1-carboxylate (Step 1)

N-((1r,3r)-3-(3-chloro-4-cyanophenoxy)-2,2,4,4-tetramethylcyclobutyl)-4-(piperidin-4-yl)benzamide hydrochloride (160 mg, 0.318 mmol), tert-butyl 4-(2-iodoethoxy)piperidine-1-carboxylate (169 mg, 0.477 mmol), and K₂CO₃ (132 mg, 0.954 mmol) were added and stirred overnight at 60° C. The reactant was extracted with water and ethyl acetate, and the organic layer was washed with brine, dried over anhydrous magnesium sulfate, filtered and concentrated. The mixture was subjected to column chromatography to obtain the desired compound (191 mg, 0.275 mmol, 86%) as a white solid.

¹H NMR (300 MHz, CDCl₃) δ 7.77-7.69 (m, 2H), 7.57 (d, J=8.7 Hz, 1H), 7.39-7.30 (m, 2H), 6.97 (d, J=2.4 Hz, 1H), 6.81 (dd, J=8.7, 2.4 Hz, 1H), 6.21 (d, J=8.2 Hz, 1H), 4.20-4.13 (m, 1H), 4.09-4.02 (m, 1H), 3.91-3.69 (m, 4H), 3.56-3.45 (m, 1H), 3.42-3.27 (m, 2H), 3.16-3.01 (m, 2H), 2.94-2.79 (m, 2H), 2.75-2.59 (m, 1H), 2.56-2.37 (m, 2H), 2.23-2.01 (m, 2H), 1.98-1.78 (m, 4H), 1.60-1.48 (m, 2H), 1.46 (s, 9H), 1.27 (s, 6H), 1.23 (s, 6H).

### Synthesis of N-((1r,3r)-3-(3-chloro-4-cyanophenoxy)-2,2,4,4-tetramethylcyclobutyl)-4-(1-(2-(piperidin-4-yloxy)ethyl)piperidin-4-yl)benzamide hydrochloride (Step 2)

Tert-butyl 4-(2-(4-(4-(((1r,3r)-3-(3-chloro-4-cyanophenoxy)-2,2,4,4-tetramethylcyclobutyl)carbamoyl)phenyl)piperidin-1-yl)ethoxy)piperidine-1-carboxylate (205 mg, 0.295 mmol) was dissolved in DCM (4 mL), and 4.0 N HCl 1,4-dioxane (2 mL) was added and stirred at room temperature for 2 hours. The reactant was concentrated under reduced pressure to obtain the desired compound (190 mg) as a white solid.

### Synthesis of N-((1r,3r)-3-(3-chloro-4-cyanophenoxy)-2,2,4,4-tetramethylcyclobutyl-4-(1-(2-((1-(1-(3-(2,6-dioxopiperidin-3-yl)-4-oxo-3,4-dihydrobenzo[d][1,2,3]triazin-6-ylpiperidine-4-carbonyl)piperidin-4-yl)oxy)ethyl)piperidin-4-yl)benzamide (Example 74)

1-(3-(2,6-dioxopiperidin-3-yl)-4-oxo-3,4-dihydrobenzo[d][1,2,3]triazin-6-yl)piperidine-4-carboxylic acid (12 mg, 0.031 mmol), N-((1r,3r)-3-(3-chloro-4-cyanophenoxy)-2,2,4,4-tetramethylcyclobutyl)-4-(1-(piperidin-4-ylmethyl)piperidin-4-yl)benzamide hydrochloride (20 mg, 0.031 mmol), EDCl HCl (8 mg, 0.043 mmol), HOBt H₂O (6 mg, 0.043 mmol), and DIPEA (0.027 mL, 0.158 mmol) were dissolved in DMF and stirred overnight at room temperature. The reactant was extracted with water and ethyl acetate, and the organic layer was washed with brine, dried over anhydrous magnesium sulfate, filtered and concentrated. The mixture was subjected to column chromatography to obtain the desired compound (7 mg, 0.007 mmol, 23%) as an ivory solid.

¹H NMR (400 MHz, CDCl₃) δ 8.16 (s, 1H), 8.00 (d, J=9.1 Hz, 1H), 7.76-7.69 (m, 2H), 7.57 (d, J=8.7 Hz, 1H), 7.52 (d, J=2.8 Hz, 1H), 7.45 (dd, J=9.1, 2.8 Hz, 1H), 7.37-7.30 (m, 2H), 6.97 (d, J=2.4 Hz, 1H), 6.81 (dd, J=8.7, 2.4 Hz, 1H), 6.19 (d, J=8.1 Hz, 1H), 5.82-5.72 (m, 1H), 4.16 (d, J=8.1 Hz, 1H), 4.11-4.00 (m, 3H), 3.99-3.89 (m, 1H), 3.81-3.71 (m, 1H), 3.71-3.62 (m, 2H), 3.64-3.53 (m, 1H), 3.41-3.27 (m, 2H), 3.17-3.04 (m, 4H), 3.01-2.76 (m, 4H), 2.67 (t, J=6.1 Hz, 2H), 2.64-2.51 (m, 1H), 2.44-2.33 (m, 1H), 2.26-2.13 (m, 2H), 2.04-1.78 (m, 10H), 1.69-1.54 (m, 2H), 1.27 (s, 6H), 1.23 (s, 6H). ; LC/MS (ESI) m/z 960.0 [M+H]⁺, 957.9 [M−H]⁻

### Synthesis of N-((1r,3r)-3-(3-chloro-4-cyanophenoxy)-2,2,4,4-tetramethylcyclobutyl -4-(1-(1-((1-(1-(3-(2,6-dioxopiperidin-3-yl)-4-oxo-3,4-dihydrobenzo[d][1,2,3]triazin-6-yl)piperidine-4-carbonyl)piperidin-4-yl)methyl)piperidin-4-yl)-1H-pyrazol-4-yl)benzamide (Example 82)

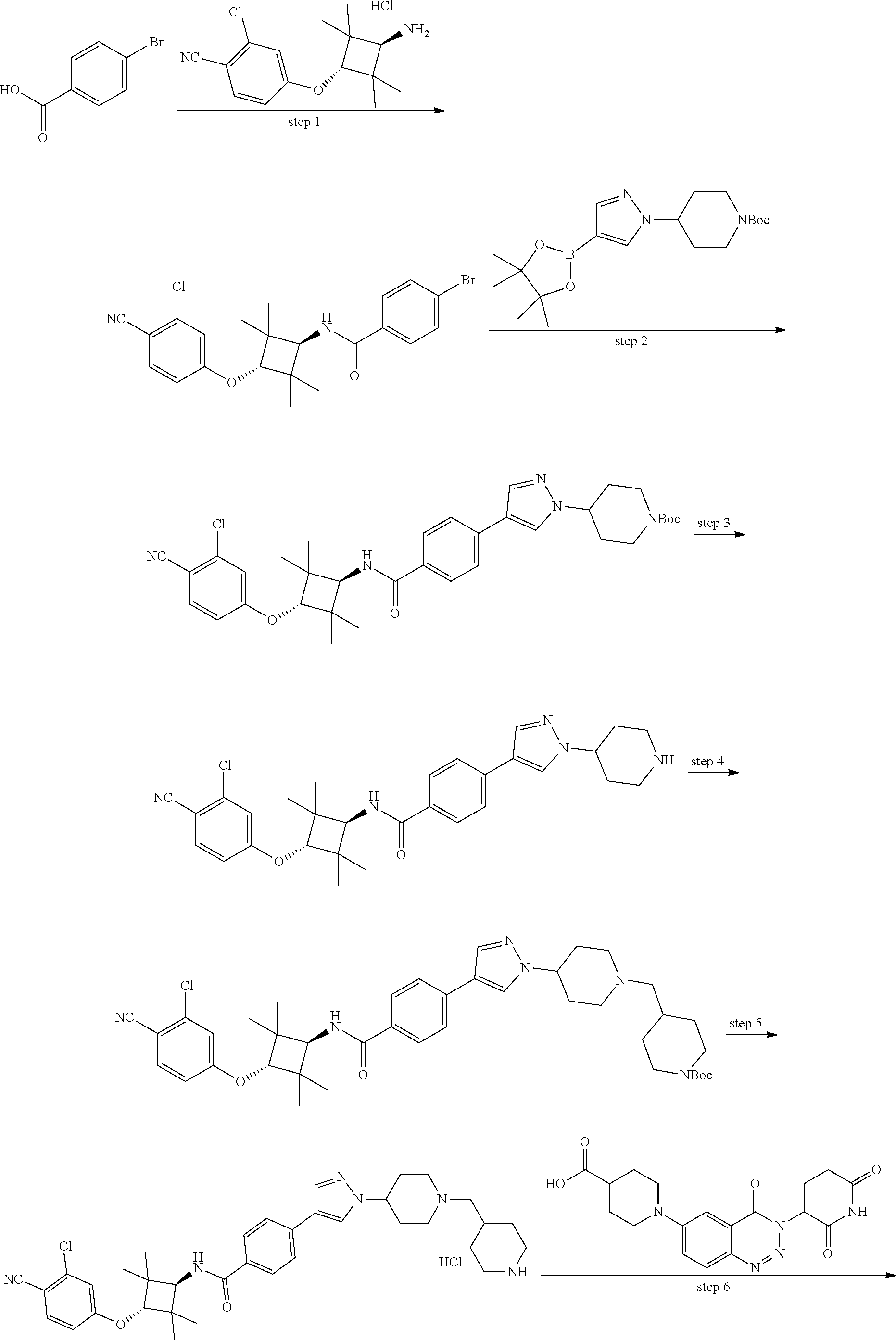

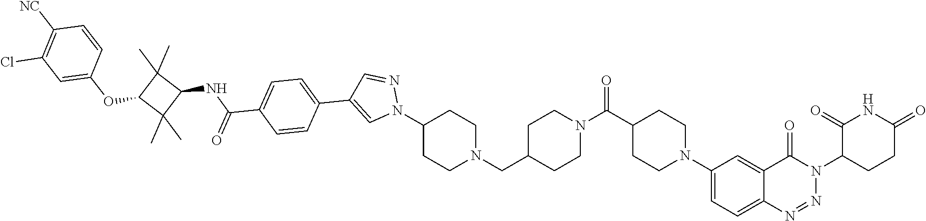 [SMILES: CC1(C)[C@H](NC(=O)c2ccc(-c3cnn(C4CCN(CC5CCN(C(=O)C6CCN(c7ccc8nnn(C9CCC(=O)NC9=O)c(=O)c8c7)CC6)CC5)CC4)c3)cc2)C(C)(C)[C@H]1Oc1ccc(C#N)c(Cl)c1]

### Synthesis of 4-bromo-N-((1r,3r)-3-(3-chloro-4-cyanophenoxy)-2,2,4,4-tetramethylcyclobutyl)benzamide (Step 1)

4-bromobenzoic acid (100 mg, 0.497 mmol), 4-((1r,3r)-3-amino-2,2,4,4-tetramethylcyclobutoxy)-2-chlorobenzonitrile hydrochloride (156 mg, 0.497 mmol), EDCl HCl (104 mg, 0.546 mmol), and HOBt H₂O (83 mg, 0.546 mmol) were dissolved in DMF, and DIPEA (0.346 mL, 1.98 mmol) was added and stirred overnight at room temperature. The reactant was extracted with water and ethyl acetate, and the organic layer was washed with brine, dried over anhydrous magnesium sulfate, filtered and concentrated. The mixture was subjected to column chromatography to obtain the desired compound (212 mg, 0.459 mmol, 92%) as a white solid.

¹H NMR (300 MHz, CDCl₃) δ 67.68-7.55 (m, 5H), 6.97 (d, J=2.4 Hz, 1H), 6.81 (dd, J=8.7, 2.4 Hz, 1H), 6.15 (d, J=8.1 Hz, 1H), 4.20-4.11 (m, 1H), 4.09-4.02 (m, 1H), 1.27 (s, 6H), 1.23 (s, 6H).

### tert-butyl 4-(4-(4-(((1r,3r)-3-(3-chloro-4-cyanophenoxy)-2,2,4,4-tetramethylcyclobutyl)carbamoyl)phenyl-1H-pyrazol-1-yl)piperidine-1-carboxylate (Step 2)

4-bromo-N-((1r,3r)-3-(3-chloro-4-cyanophenoxy)-2,2,4,4-tetramethylcyclobutyl)benzamide (150 mg, 0.324 mmol), tert-butyl 4-(4-(4,4,5,5-tetramethyl-1,3,2-dioxaborolan-2-yl)-1H-pyrazol-1-yl)piperidine-1-carboxylate (183 mg, 0.487 mmol), Potassium carbonate (179 mg, 1.29 mmol), and Pd(dppf)Cl₂ (23 mg, 0.032 mmol) were dissolved in DMF (3 mL) and stirred overnight at 80° C. under nitrogen. The reactant was extracted with water and ethyl acetate, and the organic layer was washed with brine, dried over anhydrous magnesium sulfate, filtered and concentrated. The mixture was subjected to column chromatography to obtain the desired compound (162 mg, 0.256 mmol, 79%) as a white solid.

¹H NMR (300 MHz, CDCl₃) δ 7.86-7.82 (m, 1H), 7.82-7.75 (m, 2H), 7.75-7.72 (m, 1H), 7.60-7.53 (m, 3H), 6.97 (d, J=2.4 Hz, 1H), 6.82 (dd, J=8.7, 2.4 Hz, 1H), 6.23 (d, J=8.1 Hz, 1H), 4.40-4.22 (m, 3H), 4.21-4.15 (m, 1H), 4.08-4.04 (m, 1H), 2.92 (t, J=12.9 Hz, 2H), 2.26-2.12 (m, 2H), 2.04-1.88 (m, 2H), 1.49 (s, 9H), 1.29 (s, 6H), 1.24 (s, 6H).

### Synthesis of N-((1r,3r)-3-(3-chloro-4-cyanophenoxy)-2,2,4,4-tetramethylcyclobutyl)-4-(1-(piperidin-4-yl)-1H-pyrazol-4-yl)benzamide (Step 3)

Tert-butyl 4-(4-(4-(((1r,3r)-3-(3-chloro-4-cyanophenoxy)-2,2,4,4-tetramethylcyclobutyl)carbamoyl)phenyl)-1H-pyrazol-1-yl)piperidine-1-carboxylate (162 mg, 0.256 mmol) was dissolved in DCM (4 mL), 4.0 N HCl 1,4-dioxane (2 mL) was added and stirred at room temperature for 2 hours. The reactant was concentrated under reduced pressure, neutralized with saturated NaHCO₃ aqueous solution, and extracted with 10% MeOH/DCM solution. The organic layer was washed with brine, the residue was removed with anhydrous magnesium sulfate and concentrated to obtain the desired compound (136 mg) as a white solid.

¹H NMR (300 MHz, DMSO-d₆) δ 8.34 (s, 1H), 7.97 (s, 1H), 7.91 (d, J=8.8 Hz, 1H), 7.88-7.82 (m, 2H), 7.80 (d, J=9.1 Hz, 1H), 7.74-7.67 (m, 2H), 7.66-7.54 (m, 1H), 7.22 (d, J=2.4 Hz, 1H), 7.01 (dd, J=8.8, 2.4 Hz, 1H), 4.34 (s, 1H), 4.28-4.13 (m, 1H), 4.09 (d, J=9.1 Hz, 1H), 3.13-2.99 (m, 2H), 2.68-2.54 (m, 2H), 2.06-1.91 (m, 2H), 1.91-1.71 (m, 2H), 1.25 (s, 6H), 1.14 (s, 6H).

### Synthesis of tert-butyl 4-((4-(4-(4-(((1r,3r)-3-(3-chloro-4-cyanophenoxy)-2,2,4,4-tetramethylcyclobutyl)carbamoylphenyl-1H-pyrazol-1-yl)piperidin-1-yl)methyl)piperidine-1-carboxylate (Step 4)

N-((1r,3r)-3-(3-chloro-4-cyanophenoxy)-2,2,4,4-tetramethylcyclobutyl)-4-(1-(piperidin-4-yl)-1H-pyrazol-4-yl)benzamide (136 mg, 0.256 mmol) was dissolved in MeOH (15 mL), and tert-butyl 4-formylpiperidine-1-carboxylate (218 mg, 1.02 mmol) and AcOH (0.3 mL) were added and stirred at room temperature for 1 hour. NaBH₃CN (64 mg, 1.02 mmol) was slowly added to the reaction mixture and stirred at room temperature for 12 hours. The reactants were concentrated, neutralized with 5% K₂CO₃ aqueous solution, and then extracted with dichloromethane. The organic layer was washed with brine, the residue was removed with anhydrous magnesium sulfate, and concentrated. The mixture was subjected to column chromatography to obtain the desired compound (147 mg, 0.201 mmol, 78%) as a white solid.

¹H NMR (300 MHz, CDCl₃) δ 7.85-7.81 (m, 1H), 7.81-7.74 (m, 3H), 7.60-7.52 (m, 3H), 6.97 (d, J=2.4 Hz, 1H), 6.81 (dd, J=8.7, 2.4 Hz, 1H), 6.22 (d, J=8.1 Hz, 1H), 4.22-4.03 (m, 5H), 3.07-2.93 (m, 2H), 2.70 (t, J=12.6 Hz, 2H), 2.28-2.01 (m, 8H), 1.80-1.70 (m, 2H), 1.67-1.57 (m, 1H), 1.46 (s, 9H), 1.29 (s, 6H), 1.24 (s, 6H), 1.17-0.98 (m, 2H).

### Synthesis of N-((1r,3r)-3-(3-chloro-4-cyanophenoxy)-2,2,4,4-tetramethylcyclobutyl-4-(1-(1-piperidin-4-ylmethyl)piperidin-4-yl)-1H-pyrazol-4-yl)benzamide (Step 5)

Tert-butyl 4-((4-(4-(4-(((1r,3r)-3-(3-chloro-4-cyanophenoxy)-2,2,4,4-tetramethylcyclobutyl)carbamoyl)phenyl)-1H-pyrazol-1-yl)piperidin-1-yl)methyl)piperidine-1-carboxylate (147 mg, 0.201 mmol) was dissolved in DCM (4 mL), and 4.0 N HCl 1,4-dioxane (2 mL) was added and stirred at room temperature for 2 hours. The reactant was concentrated under reduced pressure to obtain the desired compound (140 mg) as a white solid.

### Synthesis of N-((1r,3r)-3-(3-chloro-4-cyanophenoxy)-2,2,4,4-tetramethylcyclobutyl)-4-(1-(1-((1-(1-(3-(2,6-dioxopiperidin-3-yl)-4-oxo-3,4-dihydrobenzo[d][1,2,3]triazin-6-yl)piperidine-4-carbonyl)piperidin-4-yl)methyl)piperidin-4-yl)-1H-pyrazol-4-yl)benzamide (Example 82)

N-((1r,3r)-3-(3-chloro-4-cyanophenoxy)-2,2,4,4-tetramethylcyclobutyl)-4-(1-(1-(piperidin-4-ylmethyl)piperidin-4-yl)-1H-pyrazol-4-yl)benzamide (15 mg, 0.039 mmol), N-((1r,3r)-3-(3-chloro-4-cyanophenoxy)-2,2,4,4-tetramethylcyclobutyl)-4-(4-(2-(piperidin-4-yloxy)ethyl)piperazin-1-yl)benzamide hydrogen chloride (12 mg, 0.030 mmol), EDCl HCl (6 mg, 0.035 mmol), HOBt H₂O (5 mg, 0.035 mmol), and DIPEA (0.021 mL, 0.124 mmol) were dissolved in DMF and stirred overnight at room temperature. The reactant was extracted with water and ethyl acetate, and the organic layer was washed with brine, dried over anhydrous magnesium sulfate, filtered and concentrated. The mixture was subjected to column chromatography to obtain the desired compound (14 mg, 0.014 mmol, 46%) as an ivory solid.

¹H NMR (400 MHz, CDCl₃) δ 7.99 (d, J=9.2 Hz, 1H), 7.84 (s, 1H), 7.81-7.73 (m, 3H), 7.62-7.50 (m, 4H), 7.45 (dd, J=9.2, 2.9 Hz, 1H), 6.97 (d, J=2.4 Hz, 1H), 6.81 (dd, J=8.7, 2.4 Hz, 1H), 6.23 (d, J=8.1 Hz, 1H), 5.83-5.72 (m, 1H), 4.71-4.57 (m, 1H), 4.23-4.12 (m, 2H), 4.10-4.01 (m, 3H), 4.00-3.90 (m, 1H), 3.15-3.06 (m, 2H), 3.05-2.98 (m, 2H), 2.97-2.74 (m, 4H), 2.65-2.53 (m, 1H), 2.42-2.33 (m, 1H), 2.29-2.22 (m, 2H), 2.21-2.04 (m, 6H), 2.00-1.88 (m, 3H), 1.87-1.75 (m, 3H), 1.29 (s, 6H), 1.24 (s, 6H), 1.16-1.06 (m, 2H).

### Synthesis of N-((1r,3r)-3-(3-chloro-4-cyanophenoxy)-2,2,4,4-tetramethylcyclobutyl)-4-(3-(1-(3-(2,6-dioxopiperidin-3-yl)-4-oxo-3,4-dihydrobenzo[d][1,2,3]triazin-6-yl)piperidin-4-yl)prop-1-yn-1-yl)benzamide (Example 85)

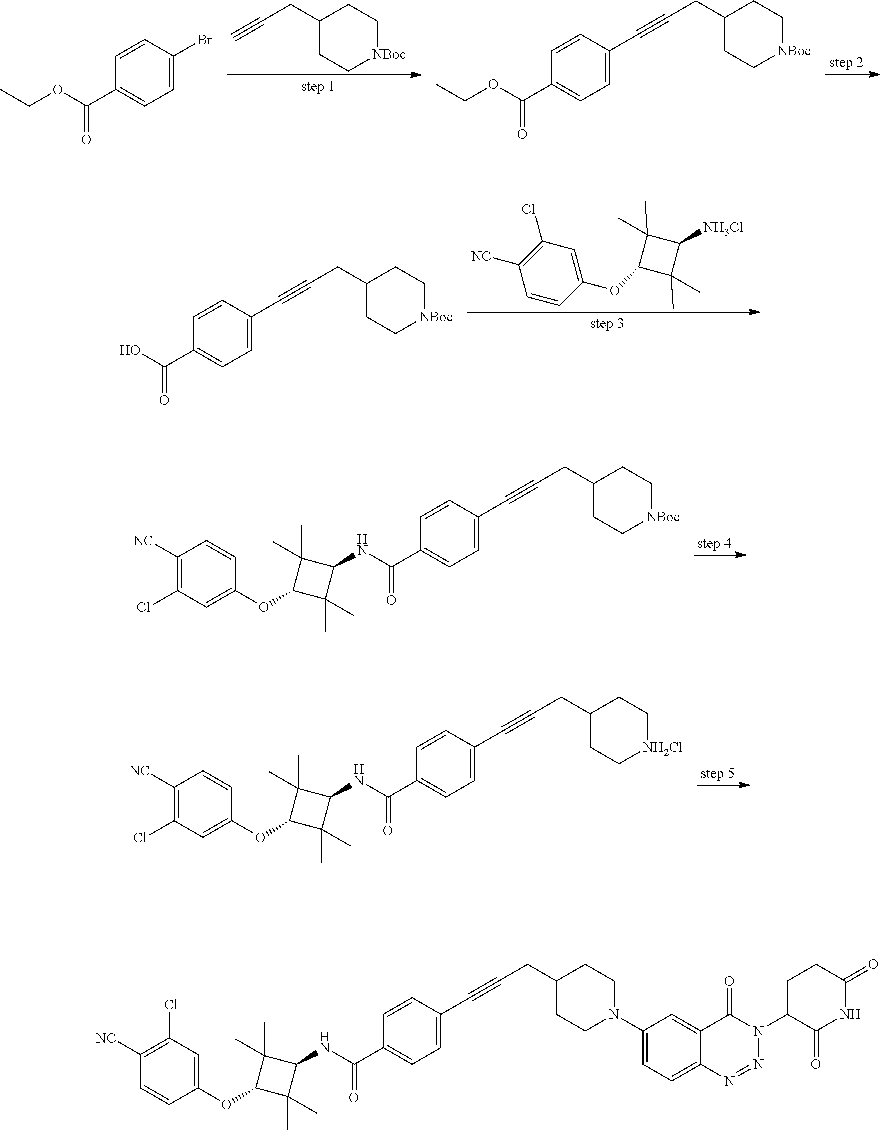

### Synthesis of tert-butyl 4-(3-(4-(ethoxycarbonyl)phenyl))prop-2-yn-1-yl)piperidine-1-carboxylate (Step 1)

Ethyl 4-bromobenzoate (246 mg, 1.07 mmol), tert-butyl 4-(prop-2-yn-1-yl)piperidine-1-carboxylate (200 mg, 0.895 mmol), TEA (0.149 ml, 1.07 mmol), copper iodide (17 mg, 0.089 mmol), and Pd(PPh₃)₄ (102 mg, 0.089 mmol) were dissolved in ACN (10 mL) and stirred at 90° C. for 3 hours under nitrogen. The reactant was extracted with water and ethyl acetate, and the organic layer was washed with brine, dried over anhydrous magnesium sulfate, filtered and concentrated. The mixture was subjected to column chromatography to obtain the desired compound (325 mg, 0.874 mmol, 97%) as a yellow oil.

¹H NMR (300 MHz, CDCl₃) δ 7.99-7.93 (m, 2H), 7.47-7.41 (m, 2H), 4.37 (q, J=7.1 Hz, 2H), 4.25-4.03 (m, 2H), 2.72 (t, J=12.5 Hz, 2H), 2.39 (d, J=6.4 Hz, 2H), 1.88-1.78 (m, 2H), 1.78-1.67 (m, 1H), 1.46 (s, 9H), 1.39 (t, J=7.1 Hz, 3H), 1.35-1.19 (m, 2H).

### Synthesis of 4-(3-(1-(tert-butoxycarbonyl)piperidin-4-yl)prop-1-yn-1-yl)benzoic Acid (Step 2)

Tert-butyl 4-(3-(4-(ethoxycarbonyl)phenyl)prop-2-yn-1-yl)piperidine-1-carboxylate (197 mg, 0.591 mmol) was dissolved in the mixture of MeOH (2 ml), water (2 ml), and THF (6 ml), and LiOH·H₂O (62 mg, 1.47 mmol) was added and stirred overnight at 40° C. The reactant was neutralized to pH 6 with 1 N HCl aqueous solution and then extracted with ethyl acetate. The organic layer was washed with brine, dried over anhydrous magnesium sulfate, filtered and concentrated. The mixture was subjected to column chromatography to obtain the desired compound (250 mg, 0.727 mmol, 83%) as a white solid.

¹H NMR (300 MHz, DMSO-d₆) δ 7.89 (d, J=7.8 Hz, 2H), 7.49 (d, J=7.8 Hz, 2H), 4.07-3.88 (m, 2H), 2.83-2.62 (m, 2H), 2.49-2.35 (m, 2H), 1.86-1.63 (m, 3H), 1.39 (s, 9H), 1.28-1.07 (m, 2H).

### Synthesis of tert-butyl 4-(3-(4-(((1r,3r)-3-(3-chloro-4-cyanophenoxy)-2,2,4,4-tetramethylcyclobutyl)carbamoyl)phenyl)prop-2-yn-1-yl)piperidine-1-carboxylate (Step 3)

Tert-butyl 4-(3-(4-(((1r,3r)-3-(3-chloro-4-cyanophenoxy)-2,2,4,4-tetramethylcyclobutyl)carbamoyl)phenyl)prop-2-yn-1-yl)piperidine-1-carboxylate (87 mg, 0.253 mmol), 4-((1r,3r)-3-amino-2,2,4,4-tetramethylcyclobutoxy)-2-chlorobenzonitrile hydrochloride (80 mg, 0.253 mmol), EDCl HCl (53 mg, 0.278 mmol), HOBt H₂O (42 mg, 0.278 mmol), and DIPEA (0.176 mL, 1.01 mmol) were dissolved in DMF and stirred overnight at room temperature. The reaction product was extracted with water and ethyl acetate, and the organic layer was washed with brine, dried over anhydrous magnesium sulfate, filtered and concentrated. The mixture was subjected to column chromatography to obtain the desired compound (132 mg, 0.218 mmol, 86%) as a white solid.

¹H NMR (300 MHz, CDCl₃) δ 7.76-7.64 (m, 2H), 7.57 (d, J=8.7 Hz, 1H), 7.51-7.41 (m, 2H), 6.97 (d, J=2.4 Hz, 1H), 6.81 (dd, J=8.7, 2.4 Hz, 1H), 6.18 (d, J=8.1 Hz, 1H), 4.26-4.08 (m, 3H), 4.06 (s, 1H), 2.73 (t, J=12.9 Hz, 2H), 2.40 (d, J=6.4 Hz, 2H), 1.88-1.78 (m, 2H), 1.78-1.65 (m, 1H), 1.46 (s, 9H), 1.37-1.30 (m, 2H), 1.27 (s, 6H), 1.23 (s, 6H).

### Synthesis of N-((1r,3r)-3-(3-chloro-4-cyanophenoxy)-2,2,4,4-tetramethylcyclobutyl)-4-(3-(piperidin-4-yl)prop-1-yn-1-yl)benzamide hydrochloride (Step 4)

Tert-butyl 4-(3-(4-(((1r,3r)-3-(3-chloro-4-cyanophenoxy)-2,2,4,4-tetramethylcyclobutyl)carbamoyl)phenyl)prop-2-yn-1-yl)piperidine-1-carboxylate (132 mg, 0.218 mmol) was dissolved in DCM (4 mL), and 4.0 N HCl 1,4-dioxane (2 mL) was added and stirred at room temperature for 2 hours. The reactant was concentrated under reduced pressure to obtain the desired compound (124 mg) as a white solid.

¹H NMR (300 MHz, DMSO-d₆) δ 8.72 (s, 2H), 7.91 (d, J=8.9 Hz, 2H), 7.89-7.80 (m, 2H), 7.60-7.47 (m, 2H), 7.21 (d, J=2.4 Hz, 1H), 7.01 (dd, J=8.9, 2.4 Hz, 1H), 4.33 (s, 1H), 4.07 (d, J=9.0 Hz, 1H), 3.78-3.65 (m, 1H), 3.54-3.44 (m, 1H), 3.31-3.24 (m, 2H), 2.95-2.83 (m, 2H), 2.01-1.79 (m, 3H), 1.62-1.44 (m, 2H), 1.23 (s, 6H), 1.14 (s, 6H).

### Synthesis of N-((1r,3r)-3-(3-chloro-4-cyanophenoxy)-2,2,4,4-tetramethylcyclobutyl)-4-(3-(1-(3-(2,6-dioxopiperidin-3-yl)-4-oxo-3,4-dihydrobenzo[d][1,2,3]triazin-6-yl)piperidin-4-yl)prop-1-yn-1-yl)benzamide (Example 85)

N-((1r,3r)-3-(3-chloro-4-cyanophenoxy)-2,2,4,4-tetramethylcyclobutyl)-4-(3-(piperidin-4-yl)prop-1-yn-1-yl)benzamide hydrochloride (16 mg, 0.030 mmol), and 3-(6-fluoro-4-oxobenzo[d][1,2,3]triazin-3(4H)-yl)piperidine-2,6-dione (8 mg, 0.030 mmol) were dissolved in DMF (1 mL), and DIPEA (0.016 mL, 0.090 mmol) was added and stirred at 90° C. for 12 hours. The reactant was diluted with water and extracted with ethyl acetate. The organic layer was washed with brine, the residue was removed with anhydrous magnesium sulfate, and concentrated. The mixture was subjected to column chromatography to obtain the desired compound (8 mg, 0.010 mmol, 35%) as a yellow solid.

¹H NMR (400 MHz, CDCl₃) δ 8.05 (s, 1H), 7.99 (d, J=9.2 Hz, 1H), 7.75-7.65 (m, 2H), 7.57 (d, J=8.8 Hz, 1H), 7.54 (d, J=2.9 Hz, 1H), 7.51-7.42 (m, 3H), 6.97 (d, J=2.4 Hz, 1H), 6.81 (dd, J=8.8, 2.4 Hz, 1H), 6.18 (d, J=8.1 Hz, 1H), 5.83-5.72 (m, 1H), 4.15 (d, J=8.0 Hz, 1H), 4.14-4.06 (m, 2H), 4.05 (s, 1H), 3.12-3.01 (m, 2H), 3.01-2.75 (m, 3H), 2.46 (d, J=6.3 Hz, 2H), 2.42-2.32 (m, 1H), 2.04-1.96 (m, 2H), 1.96-1.85 (m, 1H), 1.58-1.44 (m, 2H), 1.27 (s, 6H), 1.23 (s, 6H).

### Synthesis of N-((1r,3r)-3-(3-chloro-4-cyanophenoxy)-2,2,4,4-tetramethylcyclobutyl)-4-(4-((1-(3-(2,6-dioxopiperidin-3-yl)-4-oxo-3,4-dihydrobenzo[d][1,2,3]triazin-6-yl)piperidin-4-yl)methyl)-1H-1,2,3-triazol-1-yl)benzamide (Example 103)

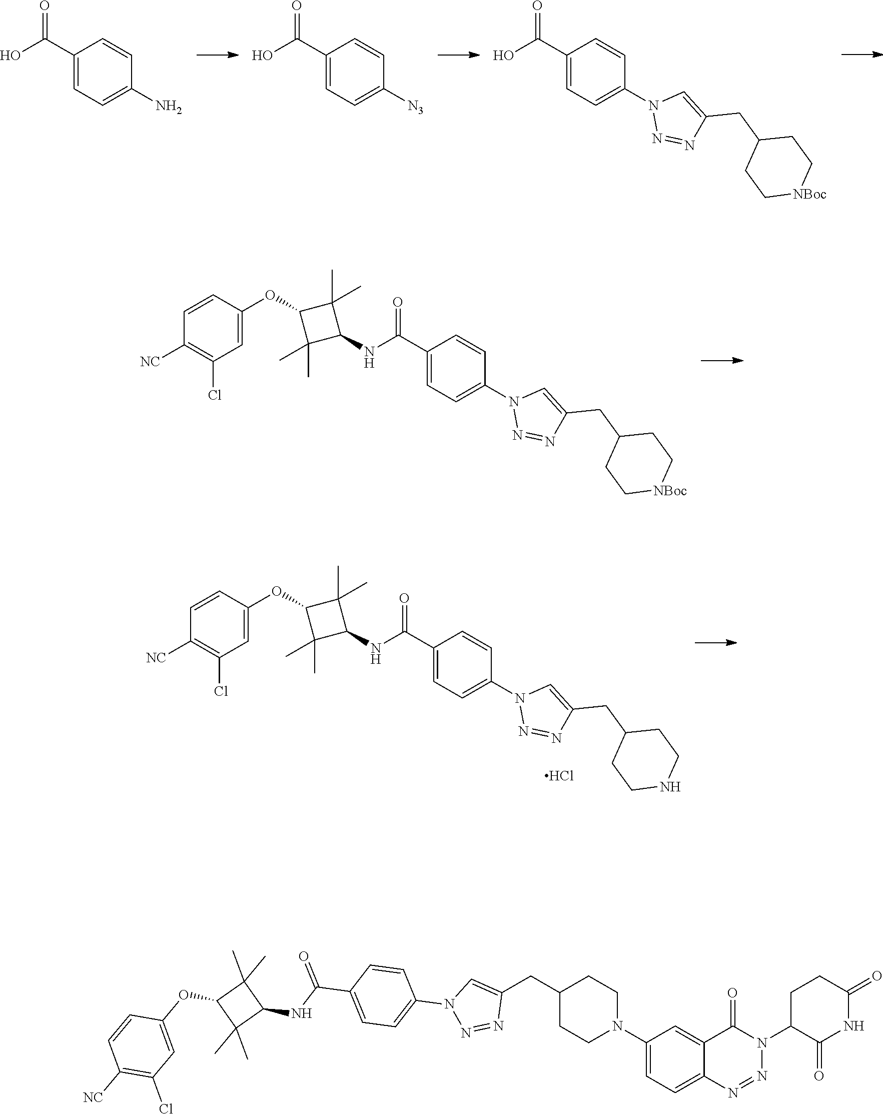

### Synthesis of 4-azidobenzoic Acid (Step 1)

4-amino benzoic acid (1 g, 7.2918 mmol, 1 eq) was dissolved in the mixture of water (200 mL) and conc. HCl (200 mL), and sodium nitrite (603 mg, 8.7501 mmol, 1.2 eq) was slowly added at 0° C. An aqueous solution (20 mL) in which sodium azide (711 mg, 10.9377 mmol, 1.5 eq) was dissolved was slowly added to the reaction solution at 0° C., followed by stirring at room temperature for 12 hours. The resulting precipitate was filtered and washed with water to obtain the target 4-azidobenzoic acid (49% yield) as a white solid.

¹H NMR (300 MHz, CDCl₃) δ 8.04-7.83 (m, 2H), 6.77-6.60 (m, 2H)

### Synthesis of 4-(4-((1-(tert-butoxycarbonyl)piperidin-4-yl)methyl)-1H-1,2,3-triazol-1-yl)benzoic Acid (Step 2)

4-azidobenzoic acid (30 mg, 0.1838 mmol, 1 eq), and tert-butyl 4-(prop-2-yn-1-yl)piperidine-1-carboxylate (41 mL, 0.1838 mmol, 1 eq) were dissolved in the mixture of H₂O (1 mL) and t-BuOH (1 mL), and sodium ascorbate (14 mg, 0.0735 mmol, 0.4 eq) and CuSO₄ (6 mg, 0.0367 mmol, 0.2 eq) were added and stirred at room temperature for 4 hours. The reactant was diluted with water and then extracted with ethyl acetate. The organic layer was washed with brine, the residue was removed with anhydrous magnesium sulfate, and concentrated. The mixture was subjected to silica gel column chromatography to obtain the desired compound (68% yield) as a white solid.

¹H NMR (300 MHz, Chloroform-6) 8.27 (d, J=8.7 Hz, 2H), 7.89 (d, J=8.8 Hz, 2H), 7.82 (s, 1H), 4.17-4.04 (m, 2H), 2.79-2.73 (m, 3H), 2.72-2.63 (m, 2H), 1.99-1.84 (m, 2H), 1.77-1.69 (m, 2H), 1.46 (s, 9H), 1.26-1.18 (m, 3H).

### Synthesis of tert-butyl 4-((1-(4-(((1r,3r)-3-(3-chloro-4-cyanophenoxy)-2,2,4,4-tetramethylcyclobutyl)carbamoyl)phenyl)-1H-1,2,3-triazol-4-yl)methyl)piperidine-1-carboxylate (Step 3)

4-(4-((1-(tert-butoxycarbonyl)piperidin-4-yl)methyl)-1H-1,2,3-triazol-1-yl)benzoic acid (100 mg, 0.2587 mmol, 1 eq), 4-((1r,3r)-3-amino-2,2,4,4-tetramethylcyclobutoxy)-2-chlorobenzonitrile hydrochloride (81 mg, 0.2587 mmol, 1 eq), EDCl·HCl (60 mg, 0.3104 mmol), and HOBt-H₂O (42 mg, 0.3104 mmol) were dissolved in DMF (1.5 mL), and DIPEA (0.18 mL, 1.0348 mmol) was added and stirred overnight at room temperature. The reactant was diluted with water and then extracted with ethyl acetate. The organic layer was washed with brine, the residue was removed with anhydrous magnesium sulfate, and concentrated. The mixture was subjected to silica gel column chromatography to obtain the desired compound (53% yield) as a white solid.

¹H NMR (300 MHz, DMSO-d₆) δ 8.69 (s, 1H), 8.09-7.90 (m, 8H), 7.23 (d, J=2.4 Hz, 1H), 7.03 (dd, J=8.8, 2.4 Hz, 1H), 4.35 (s, 1H), 4.12 (d, J=9.1 Hz, 1H), 3.94 (d, J=14.1 Hz, 2H), 2.67 (d, J=6.9 Hz, 3H), 1.68 (d, J=14.3 Hz, 2H), 1.39 (s, 9H), 1.27 (s, 6H), 1.16 (s, 6H), 1.10 (dd, J=12.5, 3.9 Hz, 2H).

### Synthesis of N-((1r,3r)-3-(3-chloro-4-cyanophenoxy)-2,2,4,4-tetramethylcyclobutyl)-4-(4-(piperidin-4-ylmethyl)-1H-1,2,3-triazol-1-yl)benzamide hydrochloride (Step 4)

Tert-butyl 4-((1-(4-(((1r,3r)-3-(3-chloro-4-cyanophenoxy)-2,2,4,4-tetramethylcyclobutyl)carbamoyl)phenyl)-1H-1,2,3-triazol-4-yl)methyl)piperidine-1-carboxylate (90 mg, 0.1390 mmol, 1 eq) was dissolved in DCM (10 mL), and 4N HCl 1,4-dioxane (0.1 mL, 0.4012 mmol, 3 eq) was added and stirred at room temperature for 1 hour. The reactant was concentrated to obtain the desired compound as a white solid.

¹H NMR (300 MHz, DMSO-d₆) δ 8.74 (s, 1H), 8.50 (s, 1H), 8.09-7.90 (m, 6H), 7.23 (d, J=2.4 Hz, 1H), 7.03 (dd, J=8.8, 2.4 Hz, 1H), 4.36 (s, 1H), 4.12 (d, J=9.2 Hz, 1H), 3.26 (d, J=12.5 Hz, 2H), 2.92-2.77 (m, 2H), 2.74-2.67 (m, 2H), 1.85 (d, J=17.3 Hz, 2H), 1.50-1.35 (m, 2H), 1.27 (s, 6H), 1.24 (s, 1H), 1.16 (s, 6H).

### N-((1r,3r)-3-(3-chloro-4-cyanophenoxy)-2,2,4,4-tetramethylcyclobutyl)-4-(4-((1-(3-(2,6-dioxopiperidin-3-yl)-4-oxo-3,4-dihydrobenzo[d][1,2,3]triazin-6-yl)piperidin-4-yl)methyl)-1H-1,2,3-triazol-1-yl)benzamide (Step 5, Example 103)

N-((1r,3r)-3-(3-chloro-4-cyanophenoxy)-2,2,4,4-tetramethylcyclobutyl)-4-(4-(piperidin-4-ylmethyl)-1H-1,2,3-triazol-1-yl)benzamide hydrochloride (20 mg, 0.0342 mmol, 1 eq) and 3-(6-fluoro-4-oxobenzo[d][1,2,3]triazin-3(4H)-yl)piperidine-2,6-dione (10 mg, 0.0342 mmol, 1.2 eq) were dissolved in DMF (0.5 mL), and DIPEA (23 μL, 0.1368 mmol, 4 eq) was added and stirred at 90° C. for 12 hours. The reactant was diluted with water and then extracted with ethyl acetate. The organic layer was washed with brine, the residue was removed with anhydrous magnesium sulfate, and concentrated. The mixture was subjected to silica gel column chromatography to obtain the desired compound (12% yield) as a white solid.

¹H NMR (300 MHz, DMSO-d₆) δ 11.13 (s, 1H), 8.73 (s, 1H), 8.12-7.96 (m, 6H), 7.92 (d, J=8.7 Hz, 1H), 7.73 (dd, J=9.2, 2.6 Hz, 1H), 7.38 (d, J=2.7 Hz, 1H), 7.23 (d, J=2.4 Hz, 1H), 7.03 (dd, J=8.8, 2.4 Hz, 1H), 5.88 (dd, J=12.1, 5.1 Hz, 1H), 4.35 (s, 1H), 4.12 (d, J=9.4 Hz, 2H), 3.08-2.87 (m, 3H), 2.71 (d, J=7.1 Hz, 3H), 2.31-2.17 (m, 2H), 1.84 (d, J=13.6 Hz, 2H), 1.34 (d, J=11.4 Hz, 2H), 1.27 (s, 6H), 1.16 (s, 6H).

### N-((1r,3r)-3-(3-chloro-4-cyanophenoxy)-2,2,4,4-tetramethylcyclobutyl)-4-(4-(3-(1-(3-(2,6-dioxopiperidin-3-yl)-4-oxo-3,4-dihydrobenzo[d][1,2,3]triazin-6-yl)-1H-1,2,3-triazol-4-yl)propyl)piperazin-1-yl)benzamide (Example 134)

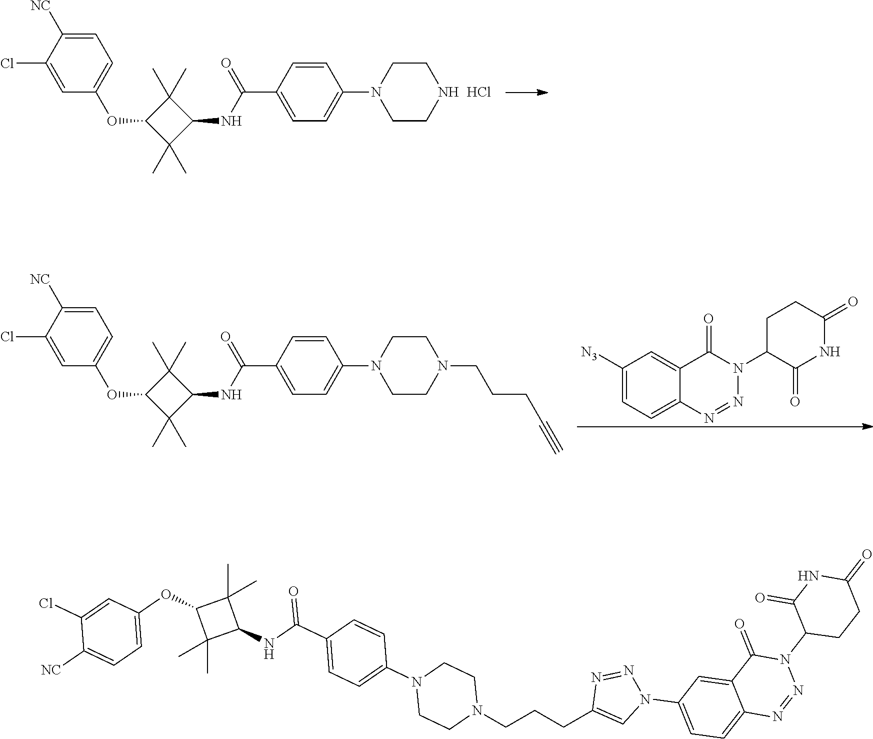 [SMILES: CC1(C)[C@H](NC(=O)c2ccc(N3CCNCC3)cc2)C(C)(C)[C@H]1Oc1ccc(C#N)c(Cl)c1 ; Cl ; C#CCCCN1CCN(c2ccc(C(=O)N[C@H]3C(C)(C)[C@H](Oc4ccc(C#N)c(Cl)c4)C3(C)C)cc2)CC1 ; [N-]=[N+]=Nc1ccc2nnn(C3CCC(=O)NC3=O)c(=O)c2c1 ; CC1(C)[C@H](NC(=O)c2ccc(N3CCN(CCCc4cn(-c5ccc6nnn(C7CCC(=O)NC7=O)c(=O)c6c5)nn4)CC3)cc2)C(C)(C)[C@H]1Oc1ccc(C#N)c(Cl)c1]

### N-((1r,3r)-3-(3-chloro-4-cyanophenoxy)-2,2,4,4-tetramethylcyclobutyl)-4-(4-(pent-4-yn-1-yl)piperazin-1-yl)benzamide (Step 1)

N-((1r,3r)-3-(3-chloro-4-cyanophenoxy)-2,2,4,4-tetramethylcyclobutyl)-4-(piperazin-1-yl)benzamide hydrochloride (20 mg, 00397 mmol, 1 eq) was dissolved in DMF (1 mL), and 5-iodopent-1-yne (7.7 mg, 0.0397 mmol, 1 eq), and potassium carbonate (7.7 mg, 0.1191 mmol, 3 eq) were added and stirred overnight at room temperature. The reactant was diluted with water and then extracted with ethyl acetate. The organic layer was washed with brine, the residue was removed with anhydrous magnesium sulfate, and concentrated. The mixture was subjected to silica gel column chromatography to obtain the desired compound (80% yield).

¹H NMR (400 MHz, Chloroform-6) 7.71 (d, J=8.7 Hz, 2H), 7.58 (d, J=8.7 Hz, 1H), 6.98 (d, J=2.2 Hz, 1H), 6.93 (d, J=8.8 Hz, 2H), 6.83 (dd, J=8.7, 2.2 Hz, 1H), 6.14 (d, J=8.1 Hz, 1H), 4.17 (d, J=8.1 Hz, 1H), 4.06 (s, 1H), 3.36-3.30 (m, 4H), 2.65-2.59 (m, 4H), 2.56-2.49 (m, 2H), 2.32-2.27 (m, 2H), 1.99 (d, J=2.5 Hz, 1H), 1.82-1.73 (m, 2H), 1.28 (s, 6H), 1.24 (s, 6H).

### Synthesis of N-((1r,3r)-3-(3-chloro-4-cyanophenoxy)-2,2,4,4-tetramethylcyclobutyl)-4-(4-(3-(1-(3-(2,6-dioxopiperidin-3-yl)-4-oxo-3,4-dihydrobenzo[d][1,2,3]triazin-6-yl)-1H-1,2,3-triazol-4-yl)propyl)piperazin-1-yl)benzamide (Step 2, Example 134)

N-((1r,3r)-3-(3-chloro-4-cyanophenoxy)-2,2,4,4-tetramethylcyclobutyl)-4-(4-(pent-4-yn-1-yl)piperazin-1-yl)benzamide (25 mg, 0.0468 mmol, 1 eq), and 3-(6-azido-4-oxobenzo[d][1,2,3]triazin-3(4H)-yl)piperidine-2,6-dione (17 mg, 0.0562 mmol, 1.2 eq) were dissolved in the mixture of H₂O (3 mL) and t-BuOH (3 mL), and sodium ascorbate (4 mg, 0.0187 mmol, 0.4 eq) and CuSO₄ (2 mg, 0.0093 mmol, 0.2 eq) were added and stirred overnight at 70° C. The reactant was diluted with water and then extracted with ethyl acetate. The organic layer was washed with brine, the residue was removed with anhydrous magnesium sulfate, and concentrated. The mixture was subjected to silica gel column chromatography to obtain the desired compound (10% yield) as a yellow solid.

¹H NMR (300 MHz, Methanol-d₄) δ 8.77 (d, J=2.2 Hz, 1H), 8.70-8.64 (m, 2H), 8.44 (d, J=8.8 Hz, 1H), 7.78 (s, 1H), 7.76-7.72 (m, 2H), 7.15 (d, J=2.4 Hz, 1H), 7.05-6.96 (m, 3H), 6.08-5.99 (m, 1H), 4.31 (s, 1H), 4.15 (s, 1H), 3.40-3.36 (m, 3H), 3.00-2.87 (m, 5H), 2.74-2.65 (m, 4H), 2.63-2.55 (m, 2H), 2.47-2.37 (m, 1H), 2.11-2.00 (m, 3H), 1.30 (s, 6H), 1.24 (s, 6H).

### N-((1r,4r)-4-(3-chloro-4-cyanophenoxy)cyclohexyl)-6-(4-((4-(3-(2,6-dioxopiperidin-3-yl)-4-oxo-3,4-dihydrobenzo[d][1,2,3]triazin-6-yl)piperazin-1-yl)methyl)piperidin-1-yl)nicotinamide (Example 160)

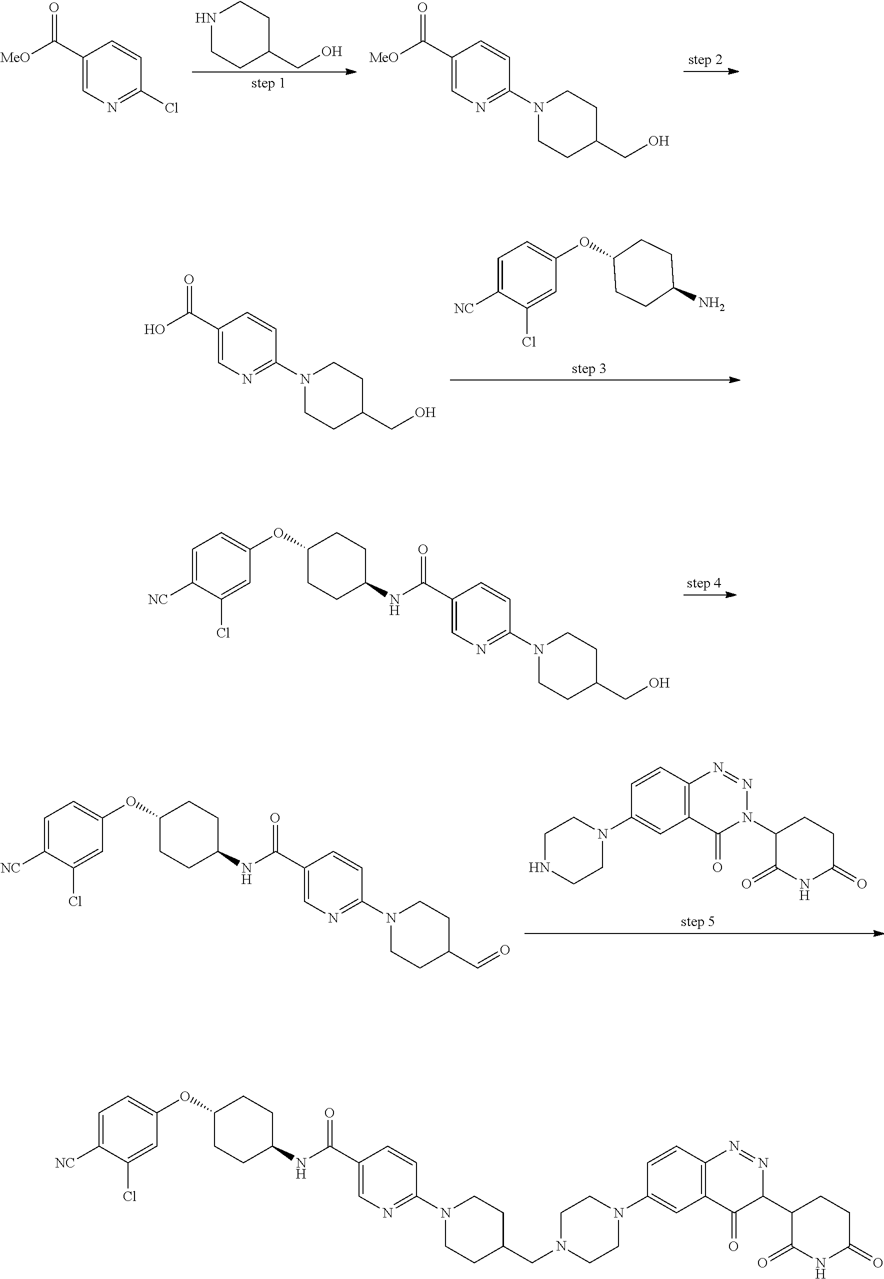

### Synthesis of methyl 6-(4-(hydroxymethyl)piperidin-1-yl)nicotinate (Step 1)

Methyl 6-chloronicotinate (4.1 g, 24.07 mmol), 4-piperidinemethanol (5.5 g, 48.14 mmol), and K₂CO₃ (5 g, 36.10 mmol) were suspended in DMF (30 ml) and stirred at 110° C. for 16 hours. After adding distilled water (30 ml) to the reaction solution, extraction was performed with EtOAc (30 ml×2), and the organic layer was dried over anhydrous sodium sulfate, filtered, and concentrated under reduced pressure. The resulting residue was subjected to MPLC (33% EtOAc/Hexane) to give 5.7 g (95%) of a white solid.

### Synthesis of 6-(4-(hydroxymethyl)piperidin-1-yl)nicotinic Acid (Step 2)

Methyl 6-(4-(hydroxymethyl)piperidin-1-yl)nicotinate (5.7 g, 23.05 mmol) was suspended in the mixture of THF (24 ml) and distilled water (8 mL), and KOH·H₂O (2.6 g, 46.10 mmol) was added and stirred at room temperature for 16 hours. After evaporating the solvent and adding distilled water, 1 N HCl was added and extracted with EtOAc (30 ml×2). The organic layer was dried over anhydrous sodium sulfate, filtered, and concentrated under reduced pressure to obtain 3.5 g (60%) of a white solid.

### Synthesis of N-((1r,4r)-4-(3-chloro-4-cyanophenoxy)cyclohexyl)-6-(4-(hydroxymethyl)piperidin-1-yl)nicotinamide (Step 3)

4-(((1r,4r)-4-aminocyclohexyl)oxy)-2-chlorobenzonitrile (143 mg, 0.57 mmol), 6-(4-(hydroxymethyl)piperidin-1-yl)nicotinic acid (134 mg, 0.57 mmol), HATU (323 mg, 0.85 mmol), and DIPEA (0.3 mL, 1.71 mmol) were suspended in DMF (6 ml) and stirred at room temperature for 16 hours. After adding distilled water (20 ml) to the reaction solution, extraction was performed with EtOAc (30 ml×2), and the organic layer was dried over anhydrous sodium sulfate, filtered, and concentrated under reduced pressure. The resulting residue was subjected to MPLC (5% MeOH/DCM) to give 120 mg (45%) of a white solid.

¹H NMR (600 MHz, MeOD) δ 8.55 (d, J=2.0 Hz, 1H), 7.91 (dd, J=9.1, 1.6 Hz, 1H), 7.69 (d, J=8.2 Hz, 1H), 7.19 (d, J=1.5 Hz, 1H), 7.03 (dd, J=8.8, 1.4 Hz, 1H), 6.80 (d, J=9.1 Hz, 1H), 4.45 (d, J=13.2 Hz, 3H), 3.97-3.87 (m, 1H), 3.42 (d, J=6.2 Hz, 2H), 2.93 (t, J=12.1 Hz, 2H), 2.20 (d, J=10.2 Hz, 2H), 2.06 (d, J=10.7 Hz, 2H), 1.82 (d, J=13.4 Hz, 2H), 1.78 (dd, J=10.8, 4.6 Hz, 1H), 1.66-1.52 (m, 4H), 1.22 (qd, J=12.3, 3.7 Hz, 2H).

### Synthesis of N-((1r,4r)-4-(3-chloro-4-cyanophenoxy)cyclohexyl)-6-(4-formylpiperidin-1-yl)nicotinamide (Step 4)

N-((1r,4r)-4-(3-chloro-4-cyanophenoxy)cyclohexyl)-6-(4-(hydroxymethyl)piperidin-1-yl)nicotinamide (50 mg, 0.11 mmol) was suspended in DCM (0.4 ml), and DMP (54 mg, 0.13 mmol) was added and stirred at room temperature for 3 hours. After adding Na₂S₂O₃ aqueous solution (15 ml), extraction was performed with DCM (25 ml×2), and the organic layer was dried over anhydrous sodium sulfate, filtered, and concentrated under reduced pressure. The obtained residue was subjected to MPLC (30% EtOAc/Hexane) to give 41 mg (83%) of a white solid.

¹H NMR (600 MHz, CDCl₃) δ 9.71 (s, 1H), 8.55 (d, J=2.3 Hz, 1H), 8.05 (dd, J=7.9, 1.0 Hz, 1H), 7.97 (dd, J=7.8, 1.7 Hz, 1H), 7.92 (dd, J=9.0, 2.5 Hz, 1H), 7.56 (d, J=8.7 Hz, 1H), 7.44 (td, J=7.6, 1.1 Hz, 1H), 7.19 (td, J=7.7, 1.7 Hz, 1H), 6.99 (d, J=2.4 Hz, 1H), 6.84 (dd, J=8.7, 2.4 Hz, 1H), 6.67 (d, J=9.0 Hz, 1H), 5.84 (d, J=7.6 Hz, 1H), 4.28 (ddt, J=13.8, 8.2, 3.7 Hz, 3H), 4.10-4.01 (m, 1H), 3.21 (ddd, J=13.6, 10.8, 3.0 Hz, 2H), 2.62-2.53 (m, 1H), 2.22 (dd, J=13.4, 2.6 Hz, 2H), 2.16 (s, 2H), 2.05-1.99 (m, 2H), 1.74-1.63 (m, 4H), 1.47-1.38 (m, 2H).

### Synthesis of N-((1r,4r)-4-(3-chloro-4-cyanophenoxy)cyclohexyl)-6-(4-((4-(3-(2,6-dioxopiperidin-3-yl)-4-oxo-3,4-dihydrobenzo[d][1,2,3]triazin-6-yl)piperazin-1-yl)methyl)piperidin-1-yl)nicotinamide (Step 5, Example 160)

N-((1r,4r)-4-(3-chloro-4-cyanophenoxy)cyclohexyl)-6-(4-formylpiperidin-1-yl)nicotinamide (250 mg, 0.54 mmol), and 3-(4-oxo-6-(piperazin-1-yl)benzo[d][1,2,3]triazin-3(4H)-yl)piperidine-2,6-dione (183 mg, 0.54 mmol) was suspended in DCM (25 ml), and sodium triacetoxyborohydride (340 mg, 1.61 mmol) was added and stirred at room temperature for 16 hours. NaHCO₃ aqueous solution (15 ml) was added to the reaction solution, followed by extraction with EtOAc (25 ml×2). The organic layer was dried over anhydrous sodium sulfate, filtered, and concentrated under reduced pressure. The resulting residue was subjected to MPLC (10% MeOH/DCM) to give 34 mg (8%) of a yellow solid.

¹H NMR (600 MHz, DMSO) δ 11.15 (s, 1H), 8.58 (d, J=2.3 Hz, 1H), 8.05-8.00 (m, 2H), 7.93 (dd, J=9.0, 2.4 Hz, 1H), 7.86 (d, J=8.8 Hz, 1H), 7.74 (dd, J=9.3, 2.6 Hz, 1H), 7.39 (d, J=2.2 Hz, 2H), 7.14 (dd, J=8.8, 2.4 Hz, 1H), 6.83 (d, J=9.2 Hz, 1H), 5.89 (dd, J=12.2, 5.2 Hz, 1H), 4.54 (s, 2H), 4.40 (d, J=13.0 Hz, 2H), 3.80 (s, 2H), 3.49 (s, 4H), 2.96 (dd, J=15.4, 10.1 Hz, 2H), 2.92-2.86 (m, 2H), 2.73-2.63 (m, 3H), 2.21 (d, J=7.1 Hz, 2H), 2.10 (d, J=13.8 Hz, 2H), 1.90 (d, J=9.9 Hz, 2H), 1.81 (d, J=11.7 Hz, 2H), 1.56-1.46 (m, 4H), 1.09 (dd, J=22.5, 10.2 Hz, 2H).

### N-((1r,4r)-4-(3-chloro-4-cyanophenoxy)cyclohexyl)-6-(4-(3-((3-(2,6-dioxopiperidin-3-yl)-4-oxo-3,4-dihydrobenzo[d][1,2,3]triazin-6-yl)oxy)propyl)piperazin-1-yl)nicotinamide (Example 161)

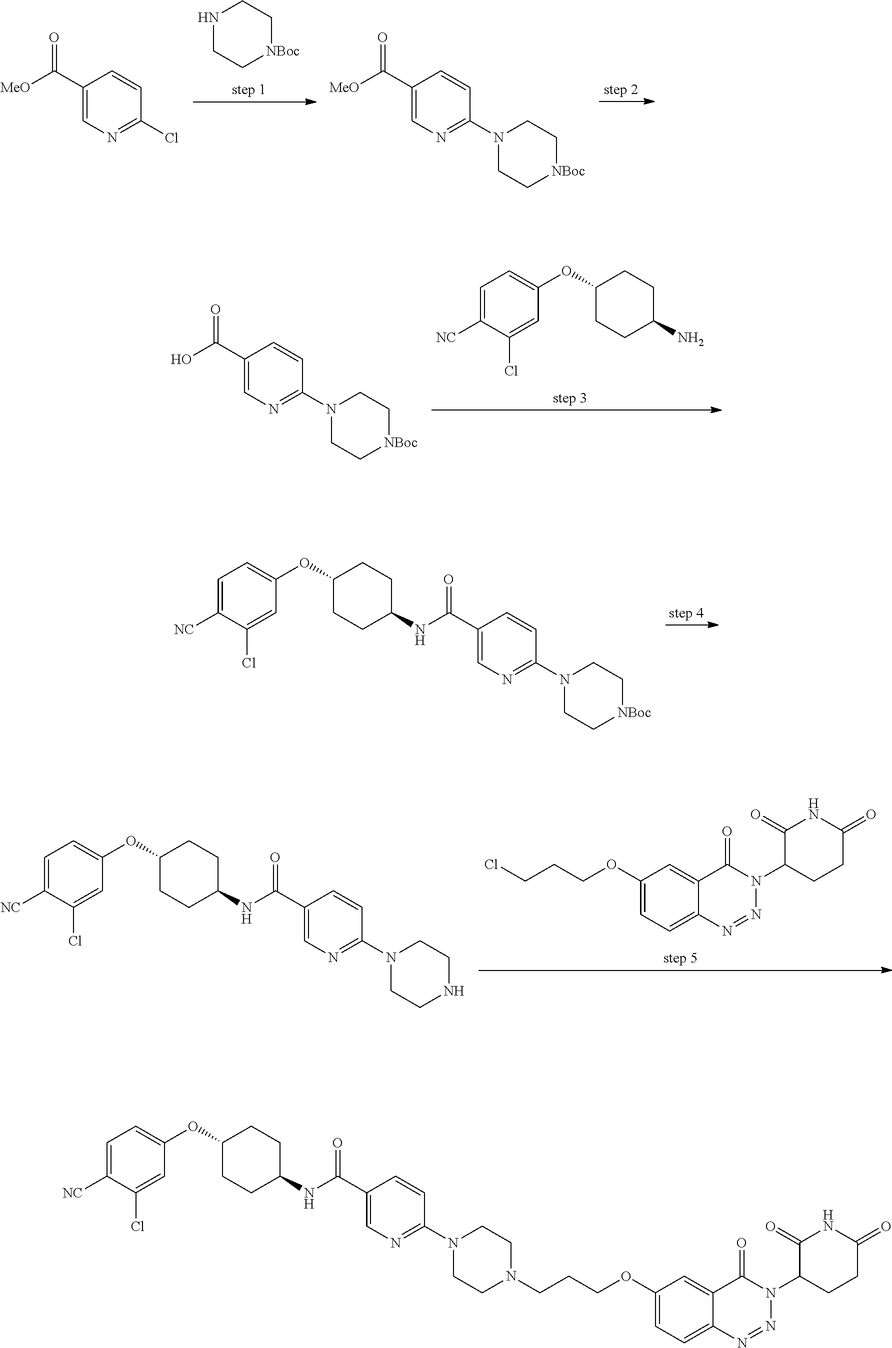

### Synthesis of tert-butyl 4-(5-(methoxycarbonyl)pyridin-2-yl)piperazine-1-carboxylate (Step 1)

Methyl 6-chloronicotinate (3 g, 17.48 mmol), 1-N-Boc-piperazine (3.9 g, 20.98 mmol), and K₂CO₃ (7.2 g, 52.45 mmol) were suspended in DMF (60 ml) and stirred at 110° C. for 16 hours. After adding distilled water (30 ml) to the reaction solution, extraction was performed with EtOAc (30 ml×2), and the organic layer was dried over anhydrous sodium sulfate, filtered, and concentrated under reduced pressure. 83%) was obtained. The resulting residue was subjected to MPLC (33% EtOAc/Hexane) to give 4.2 g (83%) of a white solid.

¹H NMR (600 MHz, DMSO) δ 8.65-8.61 (m, 1H), 7.95 (dd, J=9.1, 2.4 Hz, 1H), 6.86 (d, J=9.0 Hz, 1H), 3.77 (s, 3H), 3.64 (dd, J=6.2, 4.4 Hz, 4H), 3.44-3.38 (m, 4H), 1.41 (s, 9H).

### Synthesis of 6-(4-(tert-butoxycarbonyl)piperazin-1-yl)nicotinic Acid (Step 2)

Tert-butyl 4-(5-(methoxycarbonyl)pyridin-2-yl)piperazine-1-carboxylate (4.2 g, 14.46 mmol) was suspended in the mixture of THF (48 ml) and distilled water (16 mL), and LiOH·H₂O (1.8 g, 43.39 mmol) was added at room temperature for 16 hours. After evaporating the solvent and adding distilled water, 1 N HCl was added and extracted with EtOAc (30 ml×2). The organic layer was dried over anhydrous sodium sulfate, filtered, and concentrated under reduced pressure to obtain 3.7 g (84%) of a white solid.

¹H NMR (600 MHz, DMSO) δ 8.61 (d, J=2.1 Hz, 1H), 7.98 (dd, J=9.1, 2.3 Hz, 1H), 6.91 (d, J=9.1 Hz, 1H), 3.69-3.63 (m, 4H), 3.48-3.39 (m, 4H), 1.43 (s, 9H).

### Synthesis of tert-butyl 4-(5-(((1r,4r)-4-(3-chloro-4-cyanophenoxy)cyclohexyl)carbamoyl)pyridin-2-yl)piperazine-1-carboxylate (Step 3)

4-(((1r,4r)-4-aminocyclohexyl)oxy)-2-chlorobenzonitrile (769 mg, 3.25 mmol), 6-(4-(tert-butoxycarbonyl)piperazin-1-yl)nicotinic acid (1 g, 3.25 mmol), HATU (1.86 g, 4.88 mmol), and DIPEA (2 mL, 9.76 mmol) were suspended in DMF (10 ml) and stirred at room temperature for 16 hours. After adding distilled water (20 ml) to the reaction solution, extraction was performed with EtOAc (30 ml×2), and the organic layer was dried over anhydrous sodium sulfate, filtered, and concentrated under reduced pressure. The resulting residue was subjected to MPLC (5% MeOH/DCM) to give 920 mg (52%) of a white solid.

¹H NMR (600 MHz, DMSO) δ 8.75 (dd, J=4.4, 1.4 Hz, 1H), 8.52 (dd, J=8.4, 1.4 Hz, 1H), 8.50 (d, J=2.0 Hz, 2H), 8.25 (d, J=7.2 Hz, 2H), 8.15 (d, J=8.1 Hz, 3H), 7.85 (d, J=8.8 Hz, 2H), 7.51 (dd, J=8.4, 4.4 Hz, 1H), 7.37 (d, J=2.4 Hz, 2H), 7.13 (dd, J=8.8, 2.4 Hz, 2H), 7.10 (d, J=8.9 Hz, 2H), 4.54 (td, J=9.8, 4.5 Hz, 2H), 3.83-3.74 (m, 2H), 3.67-3.64 (m, 7H), 3.61 (dtd, J=10.5, 6.6, 4.0 Hz, 2H), 3.48-3.45 (m, 7H), 3.13 (qd, J=7.4, 4.3 Hz, 2H), 2.15-2.04 (m, 4H), 1.90 (d, J=10.5 Hz, 4H), 1.58-1.43 (m, 8H), 1.41 (s, 18H), 1.24 (dt, J=15.0, 7.6 Hz, 12H).

### Synthesis of N-((1r,4r)-4-(3-chloro-4-cyanophenoxy)cyclohexyl)-6-(piperazin-1-yl)nicotinamide hydrochloride (Step 4)

Tert-butyl 4-(5-(((1r,4r)-4-(3-chloro-4-cyanophenoxy)cyclohexyl)carbamoyl)pyridin-2-yl)piperazine-1-carboxylate (2.2 g, 4.07 mmol) was added to 4 M HCl in dioxane (40 mL) and stirred at room temperature for 1 hour. The reaction solution was concentrated to obtain 1.09 g (56%) of a white solid.

¹H NMR (600 MHz, DMSO) δ 9.77 (s, 2H), 8.62 (d, J=1.0 Hz, 1H), 8.56 (d, J=6.5 Hz, 1H), 8.31 (d, J=9.0 Hz, 1H), 7.84 (d, J=8.7 Hz, 1H), 7.37 (d, J=1.9 Hz, 1H), 7.27 (d, J=9.1 Hz, 1H), 7.13 (dd, J=8.8, 1.9 Hz, 1H), 4.58-4.49 (m, 1H), 4.02 (s, 4H), 3.85-3.76 (m, 1H), 3.23 (s, 4H), 2.10 (d, J=10.2 Hz, 2H), 1.90 (d, J=10.7 Hz, 2H), 1.57 (dd, J=24.1, 11.3 Hz, 2H), 1.48 (dd, J=22.9, 10.4 Hz, 2H).

### Synthesis of N-((1r,4r)-4-(3-chloro-4-cyanophenoxy)cyclohexyl)-6-(4-(3-((3-(2,6-dioxopiperidin-3-yl)-4-oxo-3,4-dihydrobenzo[d][1,2,3]triazin-6-yl)oxy)propyl)piperazin-1-yl)nicotinamide (Step 5, Example 161)

N-((1r,4r)-4-(3-chloro-4-cyanophenoxy)cyclohexyl)-6-(piperazin-1-yl)nicotinamide (272 mg, 0.57 mmol), 3-(6-(3-chloropropoxy)-4-oxobenzo[d][1,2,3]triazin-3(4H)-yl)piperidine-2,6-dione (200 mg, 0.57 mmol), K₂CO₃ (118 mg, 0.85 mmol), and KI (114 mg, 0.68 mmol) were suspended in DMF (2 ml) and stirred at 100° C. for 16 hours. After adding distilled water (15 ml) to the reaction solution, extraction was performed with EtOAc (25 ml×2), and the organic layer was dried over anhydrous sodium sulfate, filtered, and concentrated under reduced pressure. The obtained residue was subjected to MPLC (10% MeOH/DCM) to give 34 mg (8%) of a white solid.

¹H NMR (600 MHz, DMSO) δ 11.18 (s, 1H), 8.58 (d, J=2.3 Hz, 1H), 8.19 (d, J=9.0 Hz, 1H), 8.03 (d, J=7.5 Hz, 1H), 7.94 (dd, J=9.0, 2.4 Hz, 1H), 7.85 (d, J=8.8 Hz, 1H), 7.68 (dd, J=8.9, 2.8 Hz, 1H), 7.60 (d, J=2.8 Hz, 1H), 7.38 (d, J=2.4 Hz, 1H), 7.13 (dd, J=8.8, 2.4 Hz, 1H), 6.83 (d, J=9.1 Hz, 1H), 5.95 (dd, J=12.0, 5.4 Hz, 1H), 4.59-4.47 (m, 2H), 4.29 (t, J=6.4 Hz, 2H), 3.78 (d, J=7.0 Hz, 2H), 3.58 (s, 4H), 2.95 (ddd, J=16.7, 14.0, 5.4 Hz, 2H), 2.73-2.62 (m, 3H), 2.30-2.21 (m, 2H), 2.12-2.05 (m, 2H), 1.99 (dt, J=13.6, 6.6 Hz, 3H), 1.93-1.85 (m, 3H), 1.56-1.40 (m, 5H).

### N-((1r,3r)-3-(3-chloro-4-cyanophenoxy)-2,2,4,4-tetramethylcyclobutyl)-6-(4-((2-(3-(2,6-dioxopiperidin-3-yl)-4-oxo-3,4-dihydrobenzo[d][1,2,3]triazin-7-yl)-2,7-diazaspiro[3.5]nonan-7-yl)methyl)piperidin-1-yl)nicotinamide (Example 171)

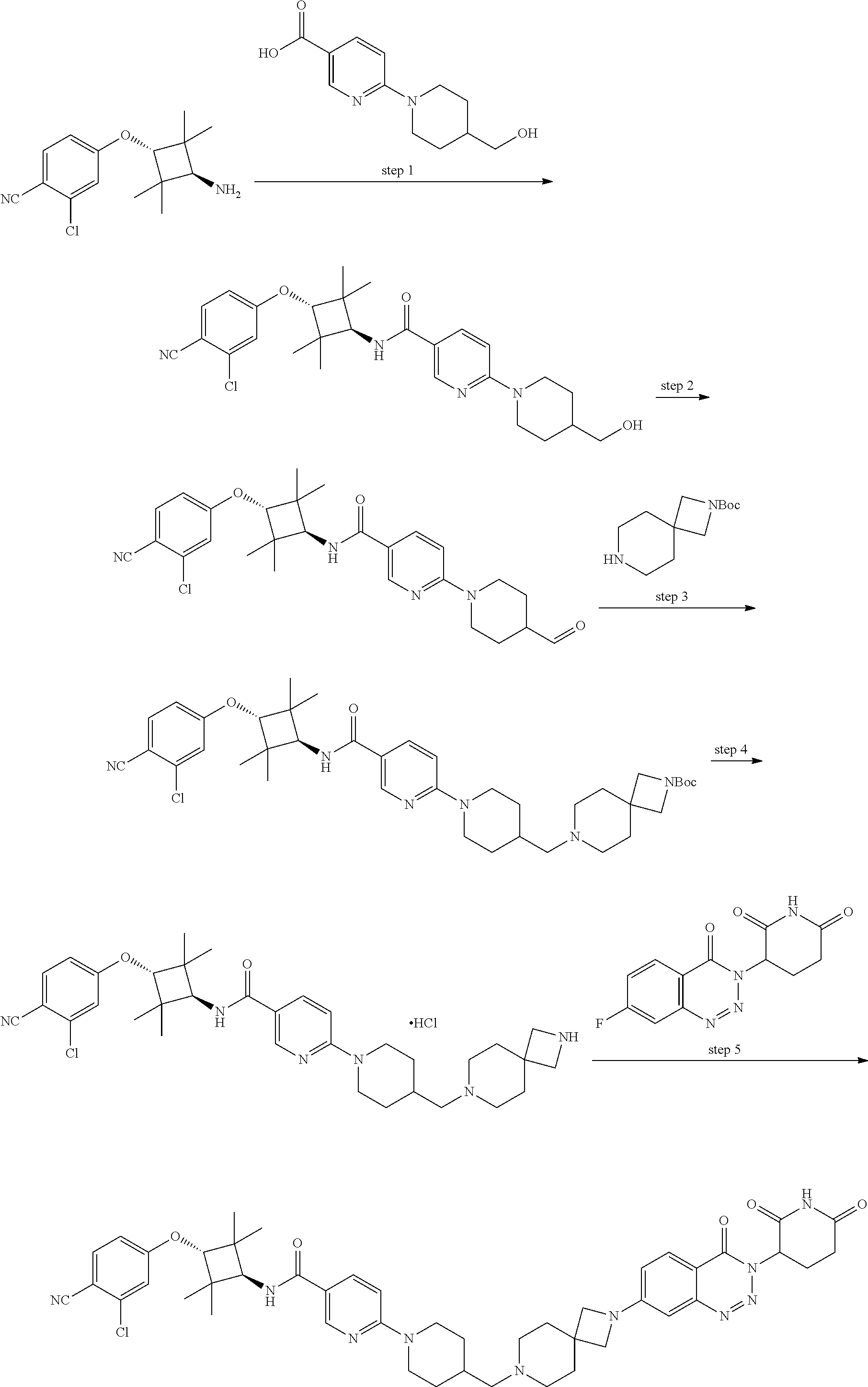

### Synthesis of N-((1r,3r)-3-(3-chloro-4-cyanophenoxy)-2,2,4,4-tetramethylcyclobutyl)-6-(4-(hydroxymethyl)piperidin-1-yl)nicotinamide (Step 1)

4-((1r,3r)-3-amino-2,2,4,4-tetramethylcyclobutoxy)-2-chlorobenzonitrile, (300 mg, 1.08 mmol), 6-(4-(hydroxymethyl)piperidin-1-yl)nicotinic acid (305 mg, 1.29 mmol), EDCl (247 mg, 1.29 mmol), HOBt (174 mg, 1.29 mmol), and DIPEA (0.15 mL, 0.86 mmol) were suspended in DMF (2.0 ml) and stirred at room temperature for 16 hours. After adding distilled water (15 ml) to the reaction solution, extraction was performed with EtOAc (25 ml×2), and the organic layer was dried over anhydrous sodium sulfate, filtered, and concentrated under reduced pressure. The resulting residue was subjected to MPLC (10% MeOH/DCM) to give 136 mg (25%) of a white solid.

¹H NMR (600 MHz, DMSO) δ 8.58 (d, J=2.2 Hz, 1H), 7.93-7.90 (m, 1H), 7.88 (d, J=8.8 Hz, 1H), 7.57 (d, J=9.3 Hz, 1H), 7.19 (d, J=2.4 Hz, 1H), 6.99 (dd, J=8.8, 2.4 Hz, 1H), 6.83 (d, J=9.0 Hz, 1H), 4.48 (t, J=5.2 Hz, 1H), 4.41 (d, J=13.2 Hz, 2H), 4.03 (d, J=9.1 Hz, 1H), 3.27-3.20 (m, 2H), 2.85 (td, J=13.0, 2.4 Hz, 2H), 1.70 (d, J=13.2 Hz, 2H), 1.68-1.59 (m, 1H), 1.19 (s, 6H), 1.09 (s, 6H), 1.09-1.03 (m, 2H).

### Synthesis of N-((1r,3r)-3-(3-chloro-4-cyanophenoxy)-2,2,4,4-tetramethylcyclobutyl)-6-(4-formylpiperidin-1-yl)nicotinamide (Step 2)

N-((1r,3r)-3-(3-chloro-4-cyanophenoxy)-2,2,4,4-tetramethylcyclobutyl)-6-(4-(hydroxymethyl)piperidin-1-yl)nicotinamide (130 mg, 0.27 mmol) was suspended in DCM (3.0 ml), and DMP (173 mg, 0.41 mmol) was added and stirred at room temperature for 3 hours. After adding Na₂S₂O₃ aqueous solution (15 ml) to the reaction solution, extraction was performed with DCM (25 ml×2), and the organic layer was dried over anhydrous sodium sulfate, filtered, and concentrated under reduced pressure. The resulting residue was subjected to MPLC (30% EtOAc/Hexane) to give 112 mg (84%) of a white solid.

¹H NMR (600 MHz, CDCl₃) δ 9.71 (d, J=0.7 Hz, 1H), 8.57 (d, J=2.0 Hz, 1H), 7.92 (dd, J=9.0, 2.5 Hz, 1H), 7.57 (d, J=8.7 Hz, 1H), 6.96 (d, J=2.4 Hz, 1H), 6.80 (dd, J=8.7, 2.4 Hz, 1H), 6.68 (d, J=9.0 Hz, 1H), 6.04 (d, J=8.2 Hz, 1H), 4.27 (dt, J=13.8, 4.0 Hz, 2H), 4.14 (d, J=8.2 Hz, 1H), 4.04 (s, 1H), 3.26-3.18 (m, 2H), 2.62-2.55 (m, 1H), 2.06-1.99 (m, 2H), 1.75-1.66 (m, 2H), 1.25 (s, 6H), 1.22 (s, 6H). m/z 495.24 [M+H]⁺

### Synthesis of tert-butyl 7-((1-(5-(((1r,3r)-3-(3-chloro-4-cyanophenoxy)-2,2,4,4-tetramethylcyclobutyl)carbamoyl)pyridin-2-yl)piperidin-4-yl)methyl)-2,7-diazaspiro[3.5]nonane-2-carboxylate (Step 3)

N-((1r,3r)-3-(3-chloro-4-cyanophenoxy)-2,2,4,4-tetramethylcyclobutyl)-6-(4-formylpiperidin-1-yl)nicotinamide (47 mg, 0.095 mmol), and tert-butyl 2,7-diazaspiro[3.5]nonane-2-carboxylate (30 mg, 0.11 mmol) were suspended in MeOH (3.0 ml), and sodium triacetoxyborohydride (40 mg, 0.19 mmol) was added and stirred at room temperature for 16 hours. NaHCO₃ aqueous solution (15 ml) was added to the reaction solution, followed by extraction with EtOAc (25 ml×2), and the organic layer was dried over anhydrous sodium sulfate, filtered, and concentrated under reduced pressure. The resulting residue was subjected to MPLC (10% MeOH/DCM) to give 38 mg (57%) of a white solid.

¹H NMR (600 MHz, CDCl₃) δ 8.55 (d, J=2.4 Hz, 1H), 7.89 (dd, J=9.0, 2.5 Hz, 1H), 7.57 (d, J=8.7 Hz, 1H), 6.96 (d, J=2.4 Hz, 1H), 6.80 (dd, J=8.7, 2.4 Hz, 1H), 6.65 (d, J=9.0 Hz, 1H), 6.03 (d, J=8.2 Hz, 1H), 4.40 (d, J=13.2 Hz, 2H), 4.14 (d, J=8.1 Hz, 1H), 4.03 (s, 1H), 3.40-3.29 (m, 4H), 3.02 (s, 4H), 2.89 (td, J=13.1, 2.5 Hz, 2H), 2.37 (d, J=6.9 Hz, 2H), 1.81 (d, J=10.9 Hz, 2H), 1.72-1.66 (m, 4H), 1.45 (s, 9H), 1.28-1.23 (m, 8H), 1.21 (s, 6H). m/z 706.47 [M+H]⁺

### Synthesis of 6-(4-((2,7-diazaspiro[3.5]nonan-7-yl)methyl)piperidin-1-yl)-N-((1r,3r)-3-(3-chloro-4-cyanophenoxy)-2,2,4,4-tetramethylcyclobutyl)nicotinamide hydrochloride (Step 4)

Tert-butyl 7-((1-(5-(((1r,3r)-3-(3-chloro-4-cyanophenoxy)-2,2,4,4-tetramethylcyclobutyl)carbamoyl)pyridin-2-yl)piperidin-4-yl)methyl)-2,7-diazaspiro[3.5]nonane-2-carboxylate (38 mg, 0.054 mmol) was suspended in DCM (1.0 ml), and 4 M HCl in dioxane (0.13 mL, 0.54 mmol) was added and stirred at room temperature for 1 hour. The reaction solution was concentrated to obtain 34 mg (98%) of a white solid.

¹H NMR (600 MHz, DMSO) δ 10.05 (s, 1H), 9.09 (s, 2H), 8.58 (s, 1H), 8.03 (s, 1H), 7.89 (d, J=8.7 Hz, 1H), 7.72 (s, 1H), 7.20 (d, J=2.4 Hz, 1H), 6.99 (dd, J=8.8, 2.4 Hz, 1H), 4.41 (d, J=12.9 Hz, 2H), 4.31 (s, 1H), 4.05 (d, J=9.1 Hz, 1H), 3.83-3.75 (m, 2H), 3.71 (dd, J=14.7, 8.5 Hz, 2H), 3.55 (s, 4H), 3.45-3.38 (m, 2H), 2.99 (s, 1H), 2.95-2.86 (m, 2H), 2.15 (d, J=13.8 Hz, 2H), 2.11-2.00 (m, 2H), 1.88 (d, J=14.8 Hz, 2H), 1.21 (s, 6H), 1.11 (s, 6H).

### Synthesis of N-((1r,3r)-3-(3-chloro-4-cyanophenoxy)-2,2,4,4-tetramethylcyclobutyl)-6-(4-((2-(3-(2,6-dioxopiperidin-3-yl)-4-oxo-3,4-dihydrobenzo[d][1,2,3]triazin-7-yl)-2,7-diazaspiro[3.5]nonan-7-yl)methyl)piperidin-1-yl)nicotinamide (Step 5, Example 171)

6-(4-((2,7-diazaspiro[3.5]nonan-7-yl)methyl)piperidin-1-yl)-N-((1r,3r)-3-(3-chloro-4-cyanophenoxy)-2,2,4,4-tetramethylcyclobutyl)nicotinamide hydrochloride (17 mg, 0.026 mmol), 3-(7-fluoro-4-oxobenzo[d][1,2,3]triazin-3(4H)-yl)piperidine-2,6-dione (11 mg, 0.040 mmol), and DIPEA (0.007 mL, 0.040 mmol) were suspended in DMSO (1.0 ml) and stirred at 90° C. for 16 hours. After adding distilled water (15 ml) to the reaction solution, extraction was performed with EtOAc (25 ml×2), and the organic layer was dried over anhydrous sodium sulfate, filtered, and concentrated under reduced pressure. The obtained residue was subjected to MPLC (10% MeOH/DCM) to give 6 mg (27%) of a yellow solid.

¹H NMR (600 MHz, CDCl₃) δ 68.57 (d, J=2.5 Hz, 1H), 8.11 (d, J=8.7 Hz, 1H), 7.98 (s, 1H), 7.90 (dd, J=9.0, 2.5 Hz, 1H), 7.57 (d, J=8.7 Hz, 1H), 6.96 (d, J=2.4 Hz, 1H), 6.86 (d, J=2.3 Hz, 1H), 6.79 (ddd, J=16.0, 8.8, 2.4 Hz, 2H), 6.67 (d, J=9.0 Hz, 1H), 6.04 (d, J=8.2 Hz, 1H), 5.78 (dd, J=11.9, 5.5 Hz, 1H), 4.42 (d, J=13.2 Hz, 2H), 4.14 (d, J=8.1 Hz, 1H), 4.04 (s, 1H), 3.79 (s, 4H), 2.99-2.89 (m, 3H), 2.87-2.77 (m, 2H), 2.49-2.32 (m, 5H), 2.19 (d, J=7.1 Hz, 2H), 1.93-1.85 (m, 6H), 1.84-1.78 (m, 1H), 1.25 (s, 6H), 1.24-1.12 (m, 8H). m/z 861.44 [M+H]⁺

### N-((1r,3r)-3-(3-chloro-4-cyanophenoxy)-2,2,4,4-tetramethylcyclobutyl)-6-(2-((1-(3-(2,6-dioxopiperidin-3-yl)-4-oxo-3,4-dihydrobenzo[d][1,2,3]triazin-7-yl)piperidin-4-yl)methyl)-2,7-diazaspiro[3.5]nonan-7-yl)nicotinamide (Example 173)

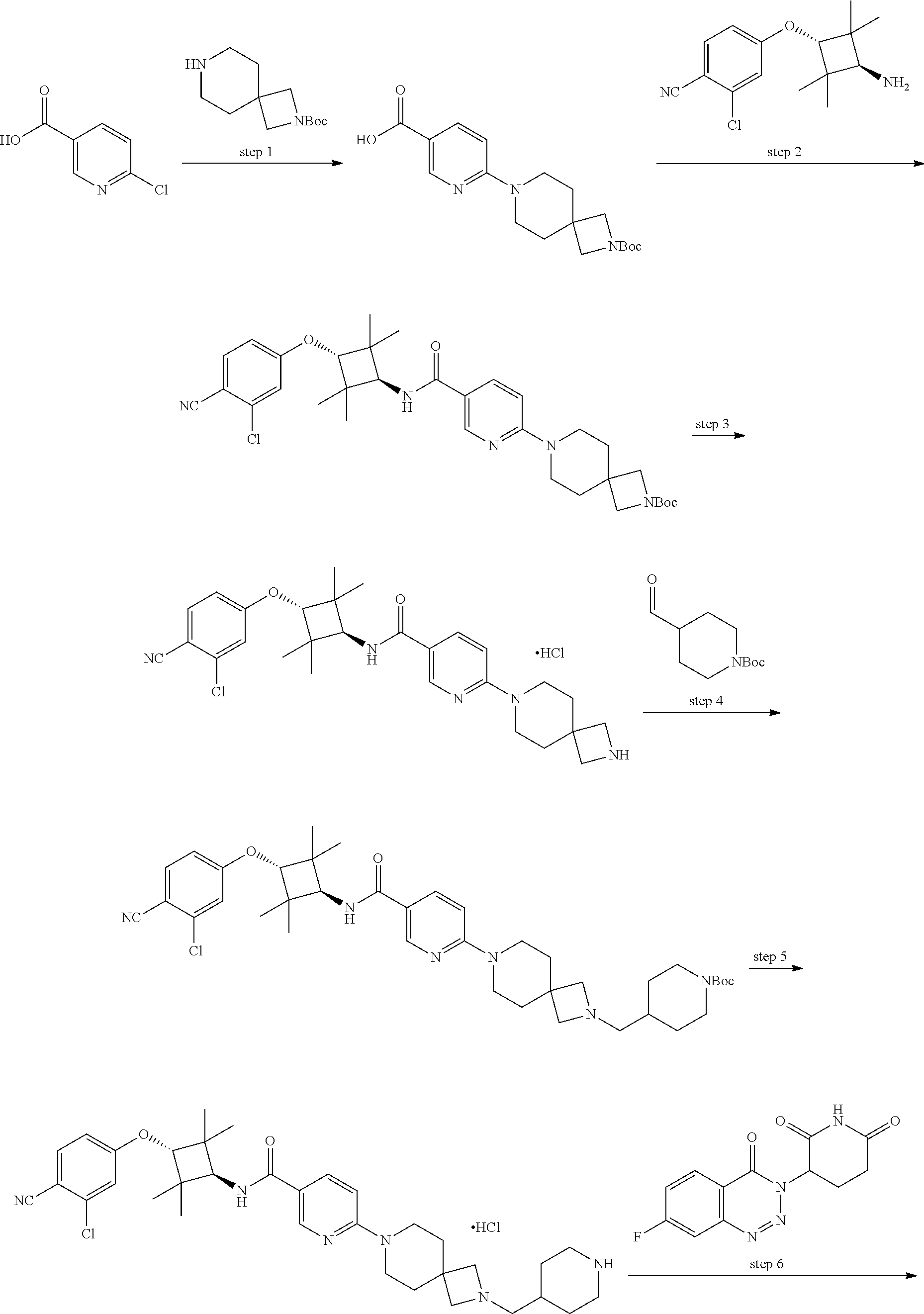

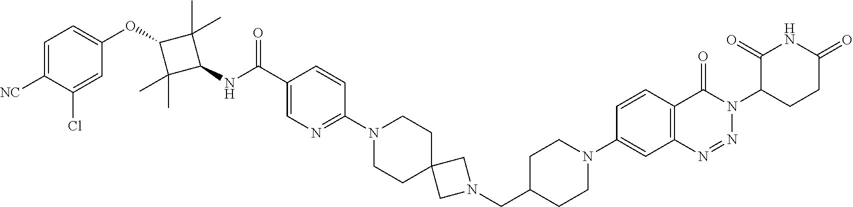 [SMILES: CC1(C)[C@H](NC(=O)c2ccc(N3CCC4(CC3)CN(CC3CCN(c5ccc6c(=O)n(C7CCC(=O)NC7=O)nnc6c5)CC3)C4)nc2)C(C)(C)[C@H]1Oc1ccc(C#N)c(Cl)c1]

### Synthesis of 6-(2-(tert-butoxycarbonyl)-2,7-diazaspiro[3.5]nonan-7-yl)nicotinic Acid (Step 1)

6-chloronicotinic acid (300 mg, 1.90 mmol), tert-butyl 2,7-diazaspiro[3.5]nonane-2-carboxylate (430 mg, 1.90 mmol), and DIPEA (0.66 mL, 3.80 mmol) were suspended in DMSO (2.0 ml) and stirred at 100° C. for 16 hours. After adding distilled water (15 ml) to the reaction solution, extraction was performed with EtOAc (25 ml×2), and the organic layer was dried over anhydrous sodium sulfate, filtered, and concentrated under reduced pressure. The resulting residue was subjected to MPLC (10% MeOH/DCM) to give 207 mg (32%) of a white solid.

¹H NMR (600 MHz, DMSO) δ 12.45 (s, 1H), 8.59 (dd, J=2.4, 0.5 Hz, 1H), 7.88 (dd, J=9.1, 2.4 Hz, 1H), 6.86 (d, J=9.1 Hz, 1H), 3.60 (s, 4H), 3.31 (s, 4H), 1.67 (t, J=5.5 Hz, 4H), 1.37 (s, 9H). m/z 348.14 [M+H]⁺

### Synthesis of tert-butyl 7-(5-(((1r,3r)-3-(3-chloro-4-cyanophenoxy)-2,2,4,4-tetramethylcyclobutyl)carbamoyl)pyridin-2-yl)-2,7-diazaspiro[3.5]nonane-2-carboxylate (Step 2)

4-((1r,3r)-3-amino-2,2,4,4-tetramethylcyclobutoxy)-2-chlorobenzonitrile, (138 mg 0.50 mmol), 6-(2-(tert-butoxycarbonyl)-2,7-diazaspiro[3.5]nonan-7-yl)nicotinic acid (207 mg, 0.60 mmol), EDCl (115 mg, 0.60 mmol), HOBt (81 mg, 0.60 mmol), and DIPEA (0.17 mL, 1.00 mmol) were suspended in DMF (2.0 ml) and stirred at room temperature for 16 hours. After adding distilled water (15 ml) to the reaction solution, extraction was performed with EtOAc (25 ml×2), and the organic layer was dried over anhydrous sodium sulfate, filtered, and concentrated under reduced pressure. 79%) was obtained. The resulting residue was subjected to MPLC (10% MeOH/DCM) to give 241 mg (79%) of a white solid.

¹H NMR (600 MHz, CDCl₃) δ 8.56 (d, J=2.4 Hz, 1H), 7.91 (dd, J=9.0, 2.5 Hz, 1H), 7.57 (d, J=8.7 Hz, 1H), 6.96 (d, J=2.4 Hz, 1H), 6.80 (dd, J=8.7, 2.4 Hz, 1H), 6.68 (d, J=9.0 Hz, 1H), 6.04 (d, J=8.1 Hz, 1H), 4.14 (d, J=8.1 Hz, 1H), 4.04 (s, 1H), 3.71 (s, 4H), 3.66-3.58 (m, 4H), 1.92-1.78 (m, 4H), 1.46 (d, J=5.4 Hz, 9H), 1.25 (s, 6H), 1.21 (s, 6H). m/z 608.34 [M+H]⁺

### Synthesis of N-((1r,3r)-3-(3-chloro-4-cyanophenoxy)-2,2,4,4-tetramethylcyclobutyl)-6-(2,7-diazaspiro[3.5]nonan-7-yl)nicotinamide hydrochloride (Step 3)

Tert-butyl 7-(5-(((1r,3r)-3-(3-chloro-4-cyanophenoxy)-2,2,4,4-tetramethylcyclobutyl)carbamoyl)pyridin-2-yl)-2,7-diazaspiro[3.5]nonane-2-carboxylate (241 mg, 0.40 mmol) was suspended in DCM (3.0 ml), and 4 M HCl in dioxane (1.0 mL, 4.0 mmol) was added and stirred at room temperature for 1 hour. The reaction solution was concentrated to obtain 217 mg (99%) of a pale yellow solid.

¹H NMR (600 MHz, DMSO) δ 9.15 (s, 2H), 8.58 (d, J=2.2 Hz, 1H), 8.20-8.12 (m, 1H), 7.91 (d, J=8.7 Hz, 1H), 7.88 (d, J=8.7 Hz, 1H), 7.21 (d, J=2.4 Hz, 1H), 7.15 (s, 1H), 7.01 (dd, J=8.8, 2.4 Hz, 1H), 4.33 (s, 1H), 4.07 (d, J=9.1 Hz, 1H), 3.79-3.72 (m, 4H), 3.69 (dd, J=5.2, 3.7 Hz, 4H), 1.87 (dd, J=21.6, 16.4 Hz, 4H), 1.23 (s, 6H), 1.12 (s, 6H).

### Synthesis of tert-butyl 4-((7-(5-(((1r,3r)-3-(3-chloro-4-cyanophenoxy)-2,2,4,4-tetramethylcyclobutyl)carbamoyl)pyridin-2-yl)-2,7-diazaspiro[3.5]nonan-2-yl)methyl)piperidine-1-carboxylate (Step 4)

N-((1r,3r)-3-(3-chloro-4-cyanophenoxy)-2,2,4,4-tetramethylcyclobutyl)-6-(2,7-diazaspiro[3.5]nonan-7-yl)nicotinamide hydrochloride (100 mg, 0.18 mmol), and tert-butyl 4-formylpiperidine-1-carboxylate (47 mg, 0.22 mmol) were suspended in MeOH (2.0 ml), and sodium triacetoxyborohydride (76 mg, 0.36 mmol) was added and stirred at room temperature for 16 hours. NaHCO₃ aqueous solution (15 ml) was added to the reaction solution, followed by extraction with EtOAc (25 ml×2), drying the organic layer over anhydrous sodium sulfate, filtration, and concentration under reduced pressure. The resulting residue was subjected to MPLC (10% MeOH/DCM) to give 60 mg (47%) of a white solid.

¹H NMR (600 MHz, CDCl₃) δ 8.55 (d, J=2.4 Hz, 1H), 7.90 (dd, J=9.0, 2.5 Hz, 1H), 7.57 (d, J=8.7 Hz, 1H), 6.96 (d, J=2.4 Hz, 1H), 6.80 (dd, J=8.7, 2.4 Hz, 1H), 6.66 (d, J=9.0 Hz, 1H), 6.03 (d, J=8.2 Hz, 1H), 4.14 (d, J=8.2 Hz, 1H), 4.04 (s, 3H), 3.64-3.56 (m, 4H), 3.08 (s, 4H), 2.67 (s, 2H), 2.38 (d, J=6.1 Hz, 2H), 1.88-1.78 (m, 4H), 1.68-1.65 (m, 2H), 1.49 (s, 1H), 1.46 (d, J=7.4 Hz, 9H), 1.25 (d, J=4.2 Hz, 6H), 1.22 (d, J=8.3 Hz, 6H), 1.09 (dt, J=12.1, 7.9 Hz, 2H). m/z 705.42 [M+H]⁺

### Synthesis of N-((1r,3r)-3-(3-chloro-4-cyanophenoxy)-2,2,4,4-tetramethylcyclobutyl)-6-(2-(piperidin-4-ylmethyl)-2,7-diazaspiro[3.5]nonan-7-yl)nicotinamide hydrochloride (Step 5)

Tert-butyl 4-((7-(5-(((1r,3r)-3-(3-chloro-4-cyanophenoxy)-2,2,4,4-tetramethylcyclobutyl)carbamoyl)pyridin-2-yl)-2,7-diazaspiro[3.5]nonan-2-yl)methyl)piperidine-1-carboxylate (60 mg, 0.085 mmol) was suspended in DCM (1.0 ml), and 4 M HCl in dioxane (0.2 mL, 0.85 mmol) was added and stirred at room temperature for 1 hour. The reaction solution was concentrated to obtain 53 mg (99%) of a white solid.

¹H NMR (600 MHz, DMSO) δ 11.03 (s, 1H), 8.79 (s, 1H), 8.61 (d, J=2.2 Hz, 1H), 8.03 (s, 1H), 7.91 (d, J=8.7 Hz, 1H), 7.71 (s, 1H), 7.21 (d, J=2.4 Hz, 1H), 7.01 (dd, J=8.8, 2.4 Hz, 1H), 4.32 (s, 1H), 4.06 (d, J=9.4 Hz, 1H), 4.03 (d, J=6.4 Hz, 1H), 3.88-3.82 (m, 1H), 3.69 (d, J=21.4 Hz, 2H), 3.26 (d, J=12.0 Hz, 2H), 3.19-3.14 (m, 4H), 2.83 (dd, J=22.7, 12.2 Hz, 2H), 1.99 (s, 2H), 1.92 (d, J=11.8 Hz, 2H), 1.81 (s, 2H), 1.44-1.34 (m, 2H), 1.22 (s, 6H), 1.12 (s, 6H).

### Synthesis of N-((1r,3r)-3-(3-chloro-4-cyanophenoxy)-2,2,4,4-tetramethylcyclobutyl)-6-(2-((1-(3-(2,6-dioxopiperidin-3-yl)-4-oxo-3,4-dihydrobenzo[d][1,2,3]triazin-7-yl)piperidin-4-yl)methyl)-2,7-diazaspiro[3.5]nonan-7-yl)nicotinamide (Step 6, Example 173)

N-((1r,3r)-3-(3-chloro-4-cyanophenoxy)-2,2,4,4-tetramethylcyclobutyl)-6-(2-(piperidin-4-ylmethyl)-2,7-diazaspiro[3.5]nonan-7-yl)nicotinamide hydrochloride (25 mg, 0.039 mmol), 3-(7-fluoro-4-oxobenzo[d][1,2,3]triazin-3(4H)-yl)piperidine-2,6-dione (16 mg, 0.058 mmol), and DIPEA (0.01 mL, 0.058 mmol) were suspended in DMSO (1.0 ml) and stirred at 90° C. for 16 hours. After adding distilled water (15 ml) to the reaction solution, extraction was performed with EtOAc (25 ml×2), and the organic layer was dried over anhydrous sodium sulfate, filtered, and concentrated under reduced pressure. The resulting residue was subjected to MPLC (10% MeOH/DCM) to give 4 mg (12%) of a yellow solid.

¹H NMR (600 MHz, DMSO) δ 11.13 (s, 1H), 8.59 (d, J=2.5 Hz, 1H), 7.97 (d, J=9.0 Hz, 1H), 7.91 (dd, J=8.9, 2.4 Hz, 1H), 7.89 (d, J=8.7 Hz, 1H), 7.58 (d, J=9.3 Hz, 1H), 7.52 (dd, J=9.2, 2.5 Hz, 1H), 7.40 (d, J=2.4 Hz, 1H), 7.20 (d, J=2.4 Hz, 1H), 6.99 (dd, J=8.8, 2.4 Hz, 1H), 6.85 (d, J=9.1 Hz, 1H), 5.88 (d, J=7.2 Hz, 1H), 4.29 (s, 1H), 4.07 (d, J=13.6 Hz, 2H), 4.03 (d, J=8.9 Hz, 1H), 3.57 (s, 4H), 2.95 (d, J=14.1 Hz, 6H), 2.68-2.59 (m, 4H), 2.30 (d, J=6.7 Hz, 2H), 2.24-2.19 (m, 1H), 1.77 (d, J=10.8 Hz, 2H), 1.71-1.63 (m, 4H), 1.58 (s, 2H), 1.27-1.15 (m, 6H), 1.10 (s, 6H). m/z 861.48 [M+H]⁺

### N-((1r,3r)-3-(3-chloro-4-cyanophenoxy)-2,2,4,4-tetramethylcyclobutyl)-6-(3-(4-(3-(2,6-dioxopiperidin-3-yl)-4-oxo-3,4-dihydrobenzo[d][1,2,3]triazin-7-yl)piperazin-1-yl)prop-1-yn-1-yl)nicotinamide (Example 177)

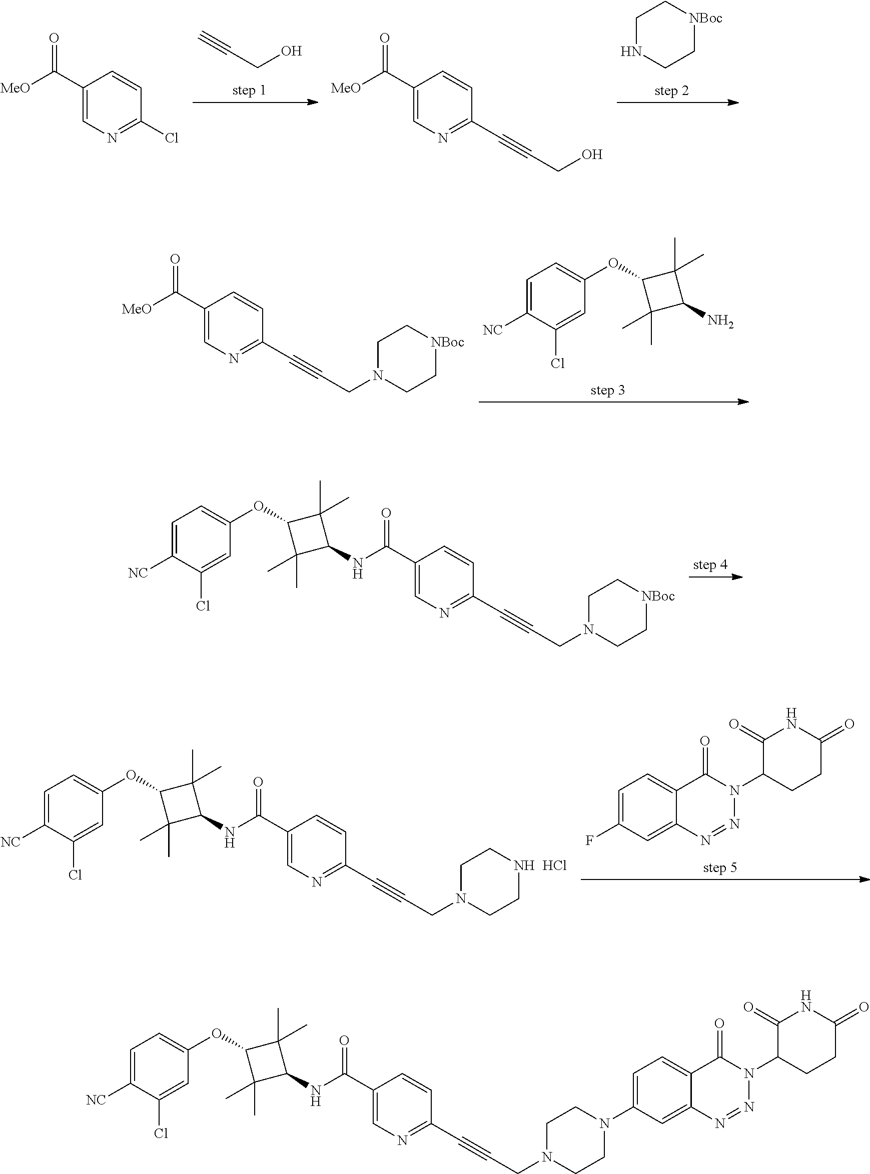

### Synthesis of methyl 6-(3-hydroxyprop-1-yn-1-yl)nicotinate (Step 1)

Methyl 6-chloronicotinate (949 mg, 5.53 mmol) was suspended in DMF (10.0 ml), and propargyl alcohol (0.5 mL, 8.3 mmol), triethylamine (3.0 mL, 22.1 mmol), Bis(triphenylphosphine)palladium(II) dichloride (194 mg, 0.28 mmol), and copper (1) iodide (53 mg, 0.28 mmol) were added and stirred at room temperature for 24 hours. After adding distilled water (10 mL) to the reaction solution, extraction was performed with EtOAc (10 ml×2), and the organic layer was dried over anhydrous magnesium sulfate, filtered, and concentrated under reduced pressure. The resulting residue was subjected to MPLC (20-80% EtOAc/Hexane, 0-10% MeOH/DCM) to give 755 mg (72%) of a brown oil.

¹H NMR (600 MHz, CDCl3) δ 9.16 (dd, J=2.1, 0.8 Hz, 1H), 8.27 (dd, J=8.1, 2.2 Hz, 1H), 7.50 (dd, J=8.1, 0.8 Hz, 1H), 4.56 (d, J=6.4 Hz, 2H), 3.96 (s, 3H), 2.53 (t, J=5.8 Hz, 1H), 1.69 (s, 1H).

### Synthesis of tert-butyl 4-(3-(5-(methoxycarbonyl)pyridin-2-yl)prop-2-yn-1-yl)piperazine-1-carboxylate (Step 2)

Methyl 6-(3-hydroxyprop-1-yn-1-yl)nicotinate (300 mg, 1.57 mmol) was suspended in DCM (3.0 ml), and triethylamine (0.4 mL, 3.1 mmol) and methanesulfonyl chloride (0.15 mL, 1.9 mmol) were added and stirred at room temperature for 30 minutes. After cooling the reaction solution to 0° C., tert-butyl piperazine-1-carboxylate (351 mg, 1.9 mmol) was added and stirred at room temperature for 16 hours. After adding distilled water (2 mL) to the reaction solution, extraction was performed with EtOAc (2 ml×2), and the organic layer was dried over anhydrous magnesium sulfate, filtered, and concentrated under reduced pressure. The resulting residue was subjected to MPLC (0-10% MeOH/DCM) to give 299 mg (53%) of a brown oil.

¹H NMR (600 MHz, CDCl3) δ 9.18 (dd, J=2.1, 0.8 Hz, 1H), 8.28 (dd, J=8.1, 2.2 Hz, 1H), 7.54 (dd, J=8.1, 0.8 Hz, 1H), 4.39 (s, 2H), 3.97 (s, 3H), 3.57-3.53 (t, J=5.0 Hz, 2H), 3.21-3.17 (t, J=5.0 Hz, 2H), 3.14 (s, 1H), 2.79 (s, 2H), 1.47 (s, 9H).

### Synthesis of tert-butyl 4-(3-(5-(((1r,3r)-3-(3-chloro-4-cyanophenoxy)-2,2,4,4-tetramethylcyclobutyl)carbam-oyl)pyridin-2-yl)prop-2-yn-1-yl)piperazine-1-carboxylate (Step 3)

Tert-butyl 4-(3-(5-(methoxycarbonyl)pyridin-2-yl)prop-2-yn-1-yl)piperazine-1-carboxylate (299 mg, 0.83 mmol) was suspended in the mixture of THF (1.5 mL) and distilled water (1.5 mL), and LiOH·H₂O (39 mg, 0.92 mmol) was added and stirred at room temperature for 30 minutes. After evaporating the solvent and adding distilled water, 1 N HCl was added and extracted with EtOAc (25 ml×2). The organic layer was dried over anhydrous magnesium sulfate, filtered, and concentrated under reduced pressure. The resulting residue was suspended in DMF (3.0 mL) and then 4-((1r,3r)-3-amino-2,2,4,4-tetramethylcyclobutoxy)-2-chlorobenzonitrile hydrochloride (262 mg, 0.83 mmol), HATU (476 mg, 1.25 mmol), and DIPEA (0.43 mL, 2.50 mmol) were added and stirred at room temperature for 16 hours. After adding distilled water (1 mL) and 1N HCl aqueous solution (1 mL) to the reaction solution, extraction was performed with EtOAc (2 ml×3), and the organic layer was washed with brine (2 mL×2), dried over anhydrous magnesium sulfate, filtered, and concentrated under reduced pressure. The resulting residue was subjected to MPLC (0-10% MeOH/DCM) to give 438 mg (87%) of a brown solid.

### Synthesis of N-((1r,3r)-3-(3-chloro-4-cyanophenoxy)-2,2,4,4-tetramethylcyclobutyl)-6-(3-(piperazin-1-yl)prop-1-yn-1-yl)nicotinamide hydrogen chloride (Step 4)

Tert-butyl 4-(3-(5-(((1r,3r)-3-(3-chloro-4-cyanophenoxy)-2,2,4,4-tetramethylcyclobutyl)carbam-oyl)-pyridin-2-yl)prop-2-yn-1-yl)piperazine-1-carboxylate (438 mg, 0.72 mmol) was suspended in DCM (2.0 ml), and 4 M HCl in dioxane (0.4 mL, 1.45 mmol) was added and stirred at room temperature for 6 hours. The reaction solution was concentrated under reduced pressure to obtain 392 mg (quant.) of a brown solid.

### Synthesis of N-((1r,3r)-3-(3-chloro-4-cyanophenoxy)-2,2,4,4-tetramethylcyclobutyl)-6-(3-(4-(3-(2,6-dioxopiperidin-3-yl)-4-oxo-3,4-dihydrobenzo[d][1,2,3]triazin-7-yl)piperazin-1-yl)prop-1-yn-1-yl)nicotinamide (Step 5, Example 177)

N-((1r,3r)-3-(3-chloro-4-cyanophenoxy)-2,2,4,4-tetramethylcyclobutyl)-6-(3-(piperazin-1-yl)prop-1-yn-1-yl)nicotinamide hydrogen chloride (100 mg, 0.19 mmol), 3-(7-fluoro-4-oxobenzo-[d][1,2,3]triazin-3(4H)-yl)piperidine-2,6-dione (51 mg, 0.19 mmol), and DIPEA (0.09 mL, 0.55 mmol) were suspended in DMSO (1.0 ml) and stirred at 90° C. for 16 hours. After adding distilled water (1 ml) to the reaction solution, extraction was performed with EtOAc (1 ml×2), and the organic layer was dried over anhydrous magnesium sulfate, filtered, and concentrated under reduced pressure. The resulting residue was subjected to MPLC (10% MeOH/DCM) to give 39 mg (28%) of a yellow solid.

¹H NMR (600 MHz, CDCl3) δ 9.01 (dd, J=2.2, 0.8 Hz, 1H), 8.21 (s, 1H), 8.17-8.10 (m, 2H), 7.58 (d, J=8.7 Hz, 1H), 7.39 (d, J=2.5 Hz, 1H), 7.32 (dd, J=9.1, 2.5 Hz, 1H), 7.09 (d, J=2.9 Hz, 1H), 6.97 (d, J=2.4 Hz, 1H), 6.81 (dd, J=8.7, 2.4 Hz, 1H), 6.23 (d, J=8.1 Hz, 1H), 5.79 (dd, J=11.9, 5.4 Hz, 1H), 4.18 (d, J=8.1 Hz, 2H), 4.07 (s, 1H), 3.58-3.51 (m, 4H), 3.45 (s, 2H), 3.00-2.89 (m, 2H), 2.87-2.80 (m, 4H), 2.80-2.76 (m, 1H), 2.42-2.34 (m, 1H), 1.29 (s, 6H), 1.25 (s, 6H).

### N-((1r,3r)-3-(3-chloro-4-cyanophenoxy)-2,2,4,4-tetramethylcyclobutyl)-6-((1-((1-(3-(2,6-dioxopiperidin-3-yl)-4-oxo-3,4-dihydrobenzo[d][1,2,3]triazin-7-yl)azetidin-3-yl)methyl)piperidin-4-yl)ethynyl)nicotinamide (Example 183)

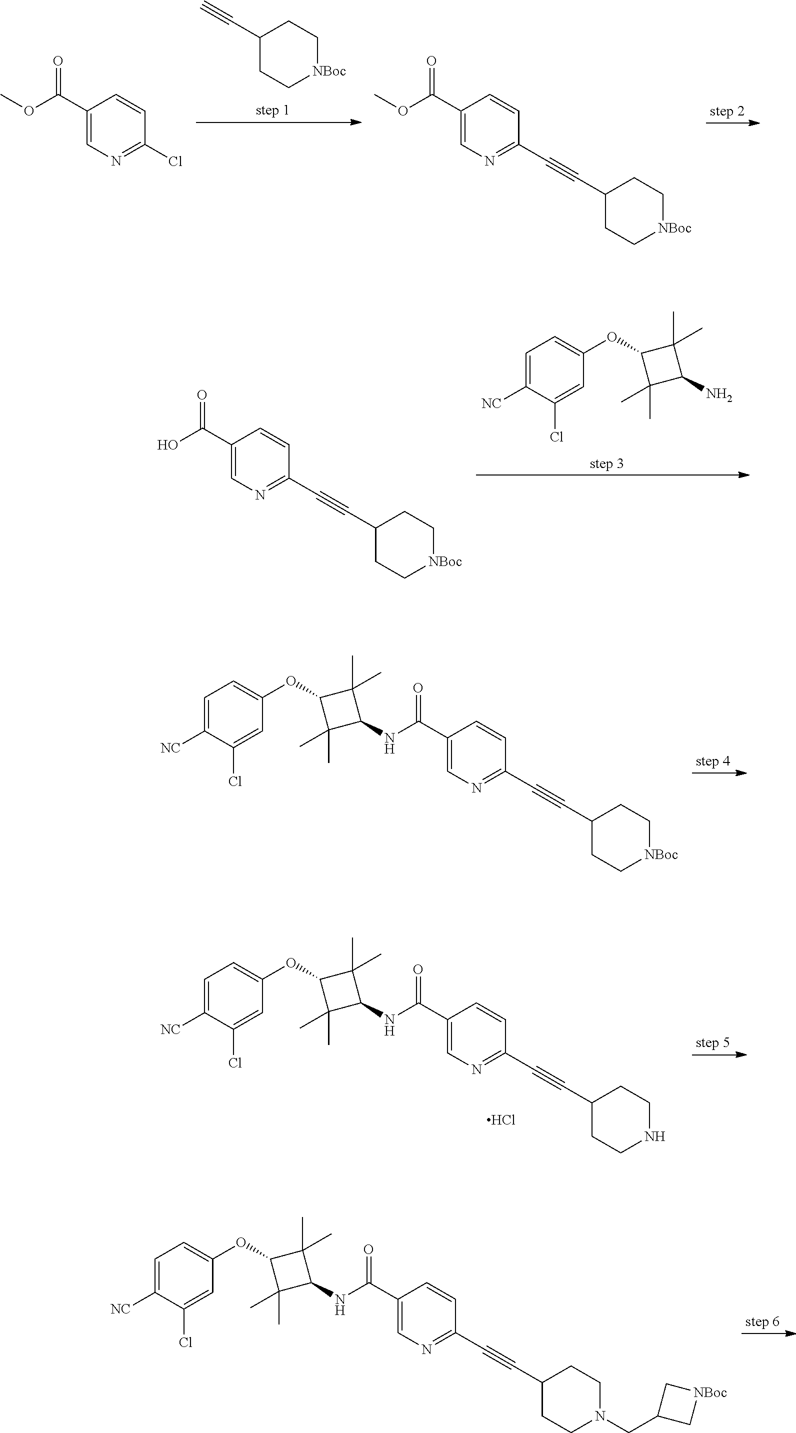

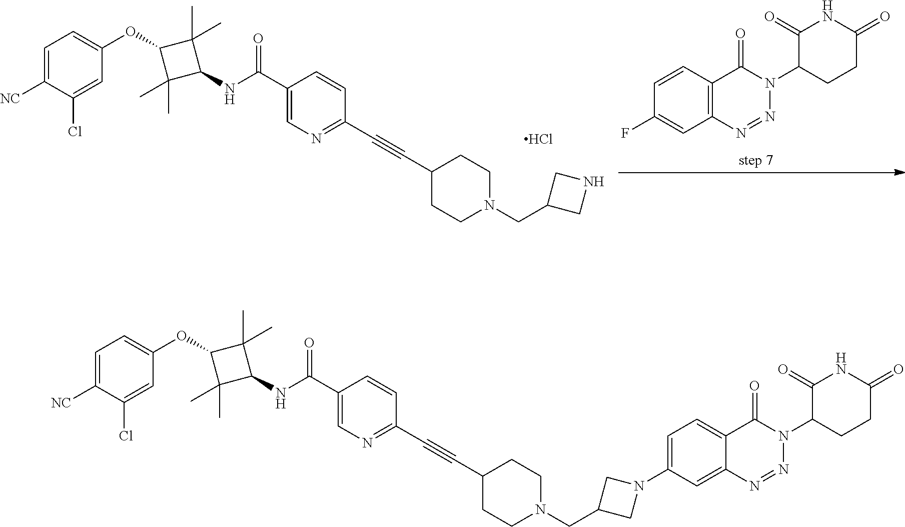 [SMILES: CC1(C)[C@H](NC(=O)c2ccc(C#CC3CCN(CC4CNC4)CC3)nc2)C(C)(C)[C@H]1Oc1ccc(C#N)c(Cl)c1 ; Cl ; O=C1CCC(n2nnc3cc(F)ccc3c2=O)C(=O)N1 ; CC1(C)[C@H](NC(=O)c2ccc(C#CC3CCN(CC4CN(c5ccc6c(=O)n(C7CCC(=O)NC7=O)nnc6c5)C4)CC3)nc2)C(C)(C)[C@H]1Oc1ccc(C#N)c(Cl)c1]

### Synthesis of methyl 6-((1-(tert-butoxycarbonyl)piperidin-4-yl)ethynyl)nicotinate (Step 1)

Methyl 6-chloronicotinate (500 mg, 2.91 mmol), and tert-butyl 4-ethynylpiperidine-1-carboxylate (914 mg, 4.37 mmol) were suspended in DMF (3.0 ml), and Pd(PPh₃)₂Cl₂ (102 mg, 0.146 mmol), CuI (28 mg, 0.146 mmol), and TEA (1.58 mL, 11.6 mmol) were added and stirred at 120° C. for 40 minutes in a microwave. After adding distilled water (15 ml) to the reaction solution, extraction was performed with EtOAc (25 ml×2), and the organic layer was dried over anhydrous sodium sulfate, filtered, and concentrated under reduced pressure. The resulting residue was subjected to MPLC (25% EtOAc/Hexane) to give 912 mg (91%) of a yellow solid.

¹H NMR (600 MHz, CDCl₃) δ 9.14 (d, J=1.2 Hz, 1H), 8.23 (dd, J=8.1, 2.2 Hz, 1H), 7.45 (d, J=8.2 Hz, 1H), 3.96 (d, J=5.0 Hz, 3H), 3.77 (s, 2H), 3.25-3.18 (m, 2H), 2.88-2.82 (m, 1H), 1.89 (d, J=2.8 Hz, 2H), 1.73 (ddd, J=17.8, 14.2, 10.1 Hz, 2H), 1.46 (d, J=5.0 Hz, 9H). m/z 345.13 [M+H]⁺

### Synthesis of 6-((1-(tert-butoxycarbonyl)piperidin-4-yl)ethynyl)nicotinic Acid (Step 2)

Tert-butyl 2-(3-(5-(methoxycarbonyl)pyridin-2-yl)prop-2-yn-1-yl)-2,7-diazaspiro[3.5]nonane-7-carboxylate (300 mg, 0.87 mmol) was suspended in the mixture of THF (3.0 ml) and distilled water (1.0 mL), and LiOH·H₂O (40 mg, 0.96 mmol) was added and stirred at room temperature for 1 hour. After evaporating the solvent and adding distilled water, 1 N HCl was added and extracted with EtOAc (25 ml×2). The organic layer was dried over anhydrous sodium sulfate, filtered, and concentrated under reduced pressure to obtain 313 mg (quant.) of a brown oil.

¹H NMR (600 MHz, DMSO) δ 8.85 (d, J=1.1 Hz, 1H), 8.03 (dd, J=7.9, 1.9 Hz, 1H), 7.34 (d, J=7.9 Hz, 1H), 3.64 (dd, J=8.9, 5.2 Hz, 2H), 3.10 (s, 2H), 2.86 (s, 1H), 1.82 (dd, J=7.2, 3.2 Hz, 2H), 1.54-1.46 (m, 2H), 1.39 (s, 9H).

### Synthesis of tert-butyl 4-((5-(((1r,3r)-3-(3-chloro-4-cyanophenoxy)-2,2,4,4-tetramethylcyclobutyl)carbamoyl)pyridin-2-yl)ethynyl)piperidine-1-carboxylate (Step 3)

4-((1r,3r)-3-amino-2,2,4,4-tetramethylcyclobutoxy)-2-chlorobenzonitrile, (249 mg 0.79 mmol), 6-((1-(tert-butoxycarbonyl)piperidin-4-yl)ethynyl)nicotinic acid (328 mg, 0.79 mmol), EDCl (182 mg, 0.95 mmol), HOBt (128 mg, 0.95 mmol), and DIPEA (0.55 mL, 3.16 mmol) were suspended in DMF (3.0 ml) and stirred at room temperature for 16 hours. After adding distilled water (15 ml) to the reaction solution, extraction was performed with EtOAc (25 ml×2), and the organic layer was dried over anhydrous sodium sulfate, filtered, and concentrated under reduced pressure. The obtained residue was subjected to MPLC (10% MeOH/DCM) to give 158 mg (34%) of a pale yellow solid.

¹H NMR (600 MHz, CDCl₃) δ 8.90 (dd, J=2.3, 0.7 Hz, 1H), 8.06 (dd, J=8.1, 2.3 Hz, 1H), 7.58 (d, J=8.7 Hz, 1H), 7.48 (dd, J=8.1, 0.8 Hz, 1H), 6.97 (d, J=2.4 Hz, 1H), 6.81 (dd, J=8.7, 2.4 Hz, 1H), 6.16 (d, J=8.0 Hz, 1H), 4.16 (d, J=8.1 Hz, 1H), 4.07 (s, 1H), 3.78 (s, 2H), 3.22 (ddd, J=13.3, 8.8, 3.3 Hz, 2H), 2.87 (td, J=8.3, 4.1 Hz, 1H), 1.90 (s, 2H), 1.74 (dd, J=11.0, 6.6 Hz, 2H), 1.47 (s, 9H), 1.28 (s, 6H), 1.24 (s, 6H). m/z 591.24 [M+H]⁺

### Synthesis of N-((1r,3r)-3-(3-chloro-4-cyanophenoxy)-2,2,4,4-tetramethylcyclobutyl)-6-(piperidin-4-ylethynyl)nicotinamide hydrochloride (Step 4)

Tert-butyl 4-((5-(((1r,3r)-3-(3-chloro-4-cyanophenoxy)-2,2,4,4-tetramethylcyclobutyl)carbamoyl)pyridin-2-yl)ethynyl)piperidine-1-carboxylate (158 mg, 0.267 mmol) was suspended in DCM (2.0 ml), and 4 M HCl in dioxane (0.67 mL, 2.67 mmol) was added and stirred at room temperature for 1 hour. The reaction solution was concentrated to obtain 132 mg (94%) of a white solid.

¹H NMR (600 MHz, DMSO) δ 8.92 (dd, J=2.3, 0.8 Hz, 1H), 8.56 (d, J=42.2 Hz, 3H), 8.18 (dd, J=8.1, 2.3 Hz, 1H), 8.12 (d, J=9.2 Hz, 1H), 7.89 (d, J=8.8 Hz, 1H), 7.63-7.59 (m, 1H), 7.21 (d, J=2.4 Hz, 1H), 7.00 (dd, J=8.8, 2.4 Hz, 1H), 4.31 (s, 1H), 4.07 (d, J=9.0 Hz, 1H), 3.22 (s, 2H), 3.09-2.97 (m, 3H), 2.07 (d, J=9.9 Hz, 2H), 1.85-1.76 (m, 2H), 1.22 (s, 6H), 1.12 (s, 6H). m/z 491.27 [M+H]⁺

### Synthesis of tert-butyl 3-((4-((5-(((1r,3r)-3-(3-chloro-4-cyanophenoxy)-2,2,4,4-tetramethylcyclobutyl)carbamoyl)pyridin-2-yl)ethynyl)piperidin-1-yl)methyl)azetidine-1-carboxylate (Step 5)

N-((1r,3r)-3-(3-chloro-4-cyanophenoxy)-2,2,4,4-tetramethylcyclobutyl)-6-(piperidin-4-ylethynyl)nicotinamide hydrochloride (132 mg, 0.251 mmol), and tert-butyl 3-formylazetidine-1-carboxylate (56 mg, 0.301 mmol) were suspended in MeOH (2.0 ml), and sodium triacetoxyborohydride (106 mg, 0.502 mmol) was added and stirred at room temperature for 16 hours. NaHCO₃ aqueous solution (15 ml) was added to the reaction solution, followed by extraction with EtOAc (25 ml×2), drying the organic layer over anhydrous sodium sulfate, filtration, and concentration under reduced pressure. The resulting residue was subjected to MPLC (10% MeOH/DCM) to give 62 mg (37%) of a white solid.

¹H NMR (600 MHz, CDCl₃) δ 8.90 (d, J=1.6 Hz, 1H), 8.05 (dd, J=8.1, 2.3 Hz, 1H), 7.58 (d, J=8.7 Hz, 1H), 7.48 (dd, J=8.1, 0.7 Hz, 1H), 6.97 (d, J=2.4 Hz, 1H), 6.81 (dd, J=8.7, 2.4 Hz, 1H), 6.16 (d, J=8.1 Hz, 1H), 4.16 (d, J=7.6 Hz, 1H), 4.07 (s, 1H), 4.03 (d, J=7.7 Hz, 2H), 3.63-3.57 (m, 2H), 2.81-2.68 (m, 4H), 2.61-2.56 (m, 1H), 2.28-2.17 (m, 2H), 2.01-1.93 (m, 2H), 1.87-1.78 (m, 2H), 1.44 (s, 9H), 1.28 (s, 6H), 1.26-1.20 (m, 6H). m/z 660.44 [M+H]⁺

### Synthesis of 6-((1-(azetidin-3-ylmethyl)piperidin-4-yl)ethynyl)-N-((1r,3r)-3-(3-chloro-4-cyanophenoxy)-2,2,4,4-tetramethylcyclobutyl)nicotinamide hydrochloride (Step 6)

Tert-butyl 3-((4-((5-(((1r,3r)-3-(3-chloro-4-cyanophenoxy)-2,2,4,4-tetramethylcyclobutyl)carbamoyl)pyridin-2-yl)ethynyl)piperidin-1-yl)methyl)azetidine-1-carboxylate (62 mg, 0.094 mmol) was suspended in DCM (1.0 ml), and 4 M HCl in dioxane (0.23 mL, 0.94 mmol) was added and stirred at room temperature for 1 hour. The reaction solution was concentrated to obtain 55 mg (98%) of a white solid.

¹H NMR (600 MHz, DMSO) δ 8.94 (d, J=2.2 Hz, 1H), 8.18 (dd, J=20.9, 8.1 Hz, 3H), 7.91 (d, J=8.7 Hz, 1H), 7.61 (d, J=8.0 Hz, 1H), 7.22 (d, J=2.3 Hz, 1H), 7.01 (dd, J=8.8, 2.1 Hz, 1H), 4.33 (d, J=6.5 Hz, 1H), 4.09 (d, J=9.1 Hz, 1H), 4.02 (s, 2H), 3.89 (d, J=7.2 Hz, 2H), 3.47-3.42 (m, 1H), 3.34 (dd, J=27.1, 22.4 Hz, 4H), 2.94 (d, J=12.0 Hz, 2H), 2.75 (s, 1H), 2.16 (d, J=27.0 Hz, 2H), 2.09-1.96 (m, 2H), 1.22 (t, J=16.9 Hz, 6H), 1.18-1.05 (m, 6H). m/z 560.30 [M+H]⁺

### Synthesis of N-((1r,3r)-3-(3-chloro-4-cyanophenoxy)-2,2,4,4-tetramethylcyclobutyl)-6-((1-((1-(3-(2,6-dioxopiperidin-3-yl)-4-oxo-3,4-dihydrobenzo[d][1,2,3]triazin-7-yl)azetidin-3-yl)methyl)piperidin-4-yl)ethynyl)nicotinamide (Step 7, Example 183)

6-((1-(azetidin-3-ylmethyl)piperidin-4-yl)ethynyl)-N-((1r,3r)-3-(3-chloro-4-cyanophenoxy)-2,2,4,4-tetramethylcyclobutyl)nicotinamide hydrochloride (20 mg, 0.034 mmol) 3-(7-fluoro-4-oxobenzo[d][1,2,3]triazin-3(4H)-yl)piperidine-2,6-dione (14 mg, 0.050 mmol), and DIPEA (0.01 mL, 0.050 mmol) were suspended in DMSO (1.0 ml) and stirred at 90° C. for 16 hours. After adding distilled water (15 ml) to the reaction solution, extraction was performed with EtOAc (25 ml×2), and the organic layer was dried over anhydrous sodium sulfate, filtered, and concentrated under reduced pressure. The resulting residue was subjected to MPLC (10% MeOH/DCM) to give 6 mg (22%) of a pale yellow solid.

¹H NMR (600 MHz, CDCl3) δ 8.91 (d, J=1.7 Hz, 1H), 8.11 (d, J=8.7 Hz, 1H), 8.08 (s, 1H), 8.06 (dd, J=8.1, 2.3 Hz, 1H), 7.58 (d, J=8.7 Hz, 1H), 7.49 (d, J=8.1 Hz, 1H), 6.97 (d, J=2.4 Hz, 1H), 6.86 (d, J=2.2 Hz, 1H), 6.81 (dd, J=8.7, 2.4 Hz, 1H), 6.77 (dd, J=8.7, 2.3 Hz, 1H), 6.17 (d, J=8.0 Hz, 1H), 5.78 (dd, J=11.8, 5.4 Hz, 1H), 4.21 (t, J=7.9 Hz, 2H), 4.16 (d, J=8.1 Hz, 1H), 4.07 (s, 1H), 3.82-3.73 (m, 2H), 3.08 (s, 1H), 2.99-2.89 (m, 2H), 2.87-2.82 (m, 1H), 2.82-2.77 (m, 2H), 2.73 (s, 4H), 2.40-2.34 (m, 1H), 2.33-2.22 (m, 2H), 2.03 (dd, J=23.4, 12.7 Hz, 3H), 1.86 (s, 2H), 1.28 (d, J=3.0 Hz, 6H), 1.25 (d, J=10.3 Hz, 6H). m/z 816.41 [M+H]⁺

The NMR and/or LC/MS results of other compounds synthesized are summarized in Table 3 below.

TABLE 3

| Example | NMR and/or LC/MS data |
|---|---|
| Example | (500 MHz, CDCl3) δ 8.57 (s, 1H), 8.36 (t, J = 5.1 Hz, 1H), 7.73 (d, J = 8.4 Hz, 2H), 7.68 (t, |
| 1 | J = 8.1 Hz, 1H), 7.57 (d, J = 8.7 Hz, 1H), 7.25 (s, 1H), 6.95 (m, 3H), 6.81 (d, J = 8.4 Hz, 2H), |
| | 6.19 (d, J = 8.2 Hz, 1H), 5.57 (m, 1H), 4.21-4.12 (m, 3H), 4.05 (s, 1H), 3.87 (t, J = 4.8 Hz, |
| | 2H), 3.74 (m,4H), 3.70-3.63 (m, 14H), 3.41 (q, J = 5.4 Hz, 2H), 2.95-2.73 (m, 3H), 2.29 (m, |
| | 1H), 1.27 (s, 6H), 1.22 (s, 6H). |
| Example | (500 MHz, CDCl3) δ 8.54 (s, 1H), 8.37 (t, J = 5.2 Hz, 1H), 7.75-7.71 (m, 2H), 7.68 (t, J = 8.1 |
| 2 | Hz, 1H), 7.57 (d, J = 8.7 Hz, 1H), 7.25 (s,1H), 6.99-6.91 (m, 3H), 6.81 (dd, J = 8.8, 2.5 Hz, |
| | 2H), 6.19 (d, J = 8.2 Hz, 1H), 5.48 (dd, J = 12.0, 5.4 Hz, 1H), 4.21-4.12 (m, 3H), 4.06 (s, |
| | 1H), 3.90-3.86 (m, 2H), 3.78-3.72 (m, 4H), 3.71-3.62 (m, 10H), 3.40 (q, J = 5.4 Hz, 2H), |
| | 2.95-2.83 (m, 2H), 2.76-2.65 (m, 1H), 2.29 (m, 1H), 1.28 (s, 3H), 1.26 (s, 3H), 1.23 (s, 6H). |
| Example | (500 MHz, CDCl3) δ 8.53 (s, 1H), 8.27 (t, J = 5.4 Hz, 1H), 7.73 (d, J = 8.6 Hz, 2H), 7.68 (t, |
| 3 | J = 8.2 Hz, 1H), 7.57 (d, J = 8.7 Hz, 1H), 7.24 (d, J = 7.7 Hz, 1H), 6.99-6.90 (m, 3H), 6.82 (m, |
| | 2H), 6.18 (d, J = 8.2 Hz, 1H), 5.61 (dd, J = 11.8, 5.4 Hz, 1H), 4.22-4.10 (m, 3H), 4.05 (s, |
| | 1H), 3.88 (t, J = 4.8 Hz, 2H), 3.77-3.55 (m, 20H), 3.34 (q, J = 6.5 Hz, 2H), 2.96-2.74 (m, 3H), |
| | 2.37-2.27 (m, 1H), 1.96 (p, J = 6.3 Hz, 2H), 1.27 (s, 6H), 1.22 (s, 6H). |
| Example | (500 MHz, CDCl3) δ 8.49 (s, 1H), 8.28 (t, J = 5.4 Hz, 1H), 7.76-7.72 (m, 2H), 7.69 (t, J = 8.1 |
| 4 | Hz, 1H), 7.57 (d, J = 8.7 Hz, 1H), 7.24 (d, J = 7.7 Hz, 1H), 6.97 (m,3H), 6.87-6.77 (m, 2H), |
| | 6.17 (d, J = 8.1 Hz, 1H), 5.62 (m, 1H), 4.18 (t, J = 4.8 Hz, 2H), 4.15 (d, J = 8.1 Hz, 1H), 4.05 |
| | (s, 1H), 3.88 (m, 2H), 3.73 (m, 2H), 3.70-3.66 (m, 8H), 3.65 (m, 8H), 3.60 (m, 4H), 3.34 (m, |
| | 2H), 2.97-2.78 (m, 3H), 2.37-2.28 (m, 1H), 1.96 (p, J = 6.3 Hz, 2H), 1.27 (s, 6H), 1.22 (s, |
| | 6H). |
| Example | (300 MHz, CDCl3) δ 8.30 (s, 1H), 8.17 (t, J = 5.0 Hz, 1H), 7.78-7.64 (m, 3H), 7.57 (d, J = 8.7 |
| 5 | Hz, 1H), 7.24 (d, J = 7.6 Hz, 1H), 7.01-6.90 (m, 3H), 6.85-6.75 (m, 2H), 6.16 (d, J = 8.1 Hz, |
| | 1H), 5.61 (dd, J = 11.3, 5.2 Hz, 1H), 4.23-4.12 (m, 3H), 4.05 (s, 1H), 3.88 (t, J = 4.8 Hz, 2H), |
| | 3.77-3.66 (m, 8H), 3.65-3.56 (m, 8H), 3.51 (t, J = 5.9 Hz, 2H), 3.24 (q, J = 6.0 Hz, 3H), 2.91 |
| | (s, 3H), 2.33 (dd, J = 12.8, 6.2 Hz, 1H), 1.75 (m, 4H), 1.27 (s, 6H), 1.22 (s, 6H). |
| Example | (500 MHz, CDCl3) δ 8.30 (s, 1H), 8.17 (t, J = 5.2 Hz, 1H), 7.76-7.71 (m, 2H), 7.68 (t, J = 8.1 |
| 6 | Hz, 1H), 7.57 (d, J = 8.7 Hz, 1H), 7.24 (d, J = 7.8 Hz, 1H), 6.99-6.93 (m, 3H), 6.83-6.77 (m, |
| | 2H), 6.16 (d, J = 8.2 Hz, 1H), 5.63 (m, 1H), 4.20-4.13 (m, 3H), 4.05 (s, 1H), 3.88 (t, J = 4.8 |
| | Hz, 2H), 3.73 (m, 2H), 3.70-3.63 (m, 16H), 3.59 (m, 2H), 3.52 (t, J = 6.1 Hz, 2H), 3.24 (q, J = |
| | 6.3 Hz, 2H), 2.98-2.78 (m, 3H), 2.37-2.30 (m, 1H), 1.82-1.71 (m, 4H), 1.27 (s, 6H), 1.22 (s, |
| | 6H). |
| Example | (300 MHz, MeOH-d4) δ 7.93-7.88 (m, 1H), 7.87 (s, 1H), 7.74 (t, J = 8.8 Hz, 3H), 7.65 (d, J = |
| 7 | 7.9 Hz, 1H), 7.49 (d, J = 9.0 Hz, 1H), 7.40 (d, J = 8.2 Hz, 1H), 7.14 (d, J = 2.4 Hz, 1H), 7.06- |
| | 6.94 (m, 3H), 5.88 (dd, J = 11.5, 5.3 Hz, 1H), 4.29 (s, 1H), 4.19-4.11 (m, 1H), 3.50 (d, J = |
| | 11.7 Hz, 2H), 3.35 (t, J = 4.9 Hz, 4H), 3.01-2.74 (m, 5H), 2.64 (t, J = 5.1 Hz, 4H), 2.37 (m, |
| | 3H), 1.96-1.52 (m, 6H), 1.29 (s, 6H), 1.23 (s, 6H). |
| Example | (500 MHz, CDCl3) δ 8.53 (s, 1H), 7.94 (d, J = 8.9 Hz, 1H), 7.73-7.69 (m, 2H), 7.57 (d, J = |
| 9 | 8.7 Hz, 1H), 7.25 (m, 1H), 7.13 (d, J = 2.6 Hz, 1H), 6.97 (d, J = 2.4 Hz, 1H), 6.94-6.89 (m, |
| | 2H), 6.81 (dd, J = 8.7, 2.4 Hz, 1H), 6.14 (d, J = 8.2 Hz, 1H), 6.07 (m, 1H), 5.79 (m, 1H), 4.66 |
| | (d, J = 13.5 Hz, 1H), 4.15 (d, J = 8.1 Hz, 1H), 4.05 (s, 1H), 4.00 (m, 2H), 3.82 (d, J = 13.5 |
| | Hz, 1H), 3.32 (m ,4H), 3.14-3.04 (m, 1H), 3.00-2.85 (m, 3H), 2.73 (td, J = 13.0, 2.7 Hz, 1H), |
| | 2.59 (m, 5H), 2.37 (m, 1H), 2.28 (d, J = 7.0 Hz, 2H), 2.01-1.75 (m, 6H), 1.27 (s, 6H), 1.22 (s, |
| | 6H). |
| Example | (500 MHz, CDCl3) δ 8.13 (s, 1H), 8.00 (d, J = 9.1 Hz, 1H), 7.74-7.67 (m, 2H), 7.57 (d, J = |
| 10 | 8.7 Hz, 1H), 7.52 (d, J = 2.9 Hz, 1H), 7.46 (dd, J = 9.3, 2.9 Hz, 1H), 6.97 (d, J = 2.4 Hz, 1H), |
| | 6.94-6.88 (m, 2H), 6.81 (dd, J = 8.8, 2.4 Hz, 1H), 6.13 (d, J = 8.1 Hz, 1H), 5.77 (dd, J = 11.6, |
| | 5.4 Hz, 1H), 4.65 (d, J = 13.2 Hz, 1H), 4.15 (d, J = 8.1 Hz, 1H), 4.11-4.03 (m, 3H), 3.96 (d, J |
| | = 13.5 Hz, 1H), 3.31 (m, 4H), 3.09 (t, J = 12.6 Hz, 3H), 3.01-2.78 (m, 4H), 2.58 (m, 5H), |
| | 2.43-2.34 (m, 1H), 2.29-2.23 (m, 2H), 2.07-1.91 (m, 4H), 1.89-1.79 (m, 4H), 1.64 (m, 4H), |
| | 1.27 (s, 6H), 1.22 (s, 6H). |
| Example | (500 MHz, CDCl3) δ 8.16 (s, 1H), 7.98 (d, J = 8.9 Hz, 1H), 7.70 (d, J = 8.6 Hz, 2H), 7.57 (d, |
| 11 | J = 8.7 Hz, 1H), 7.04 (d, J = 2.7 Hz, 1H), 6.98-6.94 (m, 2H), 6.92 (d, J = 8.8 Hz, 2H), 6.81 |
| | (dd, J = 8.7, 2.4 Hz, 1H), 6.13 (d, J = 8.1 Hz, 1H), 5.77 (m, 1H), 4.64 (d, J = 13.4 Hz, 1H), |
| | 4.31 (m, 5H), 4.15 (d, J = 8.0 Hz, 1H), 4.05 (s, 1H), 3.80 (p, J = 7.7 Hz, 1H), 3.60 (d, J = |
| | 13.5 Hz, 1H), 3.31 (t, J = 5.1 Hz, 4H), 3.07 (t, J = 12.6 Hz, 1H), 3.01-2.81 (m, 4H), 2.69- |
| | 2.58 (m, 5H), 2.38 (m, Hz, 1H), 2.27 (d, J = 6.8 Hz, 2H), 2.04 (d, J = 9.8 Hz, 1H), 1.90 (dd, |
| | J = 44.3, 13.5 Hz, 3H), 1.27 (s, 6H), 1.25 (m, 2H), 1.22 (s, 6H), 1.13 (m, 2H) |
| Example | (500 MHz, CDCl3) δ 8.13 (d, J = 9.1 Hz, 2H), 7.72-7.67 (m, 2H), 7.57 (d, J = 8.7 Hz, 1H), |
| 12 | 7.38 (d, J = 2.6 Hz, 1H), 7.32 (dd, J = 9.1, 2.6 Hz, 1H), 6.97 (d, J = 2.4 Hz, 1H), 6.94-6.90 |
| | (m, 2H), 6.81 (dd, J = 8.7, 2.4 Hz, 1H), 6.13 (d, J = 8.1 Hz, 1H), 5.79 (dd, J = 11.7, 5.3 Hz, |
| | 1H), 4.15 (d, J = 8.1 Hz, 1H), 4.04 (m, 3H), 3.32 (m, 4H), 3.07-2.86 (m, 5H), 2.60 (m, 4H), |
| | 2.41-2.35 (m, 1H), 2.30 (m, 2H), 1.96 (d, J = 13.3 Hz, 3H), 1.34 (m, 2H), 1.27 (s, 6H), 1.25 |
| | (m, 2H), 1.22 (s, 6H). |
| Example | (500 MHz, CDCl3) δ 8.24 (s, 1H), 7.90 (dd, J = 8.8, 2.8 Hz, 1H), 7.70 (dd, J = 8.8, 2.8 Hz, |
| 14 | 2H), 7.57 (dd, J = 8.8, 2.8 Hz, 1H), 7.31 (m, 1H), 7.23-7.12 (m, 2H), 6.99-6.88 (m, 3H), 6.81 |
| | (m, 1H), 6.23 (d, J = 8.3 Hz, 1H), 6.09 (s, 1H), 5.74 (dd, J = 12.1, 5.5 Hz, 1H), 4.21-4.11 (m, |
| | 3H), 4.06 (s, 1H), 3.92 (m, 2H), 3.83 (m, 2H), 3.74-3.63 (m, 12H), 3.61-3.56 (m, 2H), 3.44 |
| | (m, 2H), 3.41-3.37 (m, 2H), 3.33 (d, J = 6.5 Hz, 3H), 2.97-2.82 (m, 3H), 2.35 (m, 1H), 2.03 |
| | (d, J = 9.8 Hz, 1H), 1.74 (s, OH), 1.62 (m, 2H), 1.56 (m, 2H), 1.28 (m,8H), 1.23 (m, 6H). |
| Example | (500 MHz, CDCl3) δ 8.39 (s, 1H), 7.98 (d, J = 9.1 Hz, 1H), 7.73 (d, J = 8.4 Hz, 2H 7.57 (d, |
| 15 | J = 8.7 Hz, 1H), 7.50 (d, J = 2.8 Hz, 1H), 7.43 (dd, J = 9.2, 2.7 Hz, 1H), 7.00-6.92 (m, 3H), |
| | 6.81 (dd, J = 8.9, 2.3 Hz, 1H), 6.21 (m, 2H), 5.77 (dd, J = 11.5, 5.4 Hz, 1H), 4.20-4.15 (m, |
| | 3H), 4.05 (m, 3H), 3.88 (t, J = 4.7 Hz, 2H), 3.73 (m, 2H), 3.70-3.62 (m, 12H), 3.58 (m, 2H), |
| | 3.48 (t, J = 5.5 Hz, 2H), 3.27 (q, J = 6.2 Hz, 2H), 3.08-2.85 (m, 5H), 2.37 (m, 2H), 2.07-1.99 |
| | (m, 2H), 1.95 (m, 3H), 1.85 (m, Hz, 2H) 1.27 (s, 17H), 1.25 (m, 2H), 1.22 (s, 6H). |
| Example | (500 MHz, CDCl3) δ 8.32 (s, 1H), 7.94 (d, J = 8.8 Hz, 1H), 7.75-7.69 (m, 2H), 7.57 (d, J = |
| 16 | 8.7 Hz, 1H), 7.00 (d, J = 2.6 Hz, 1H), 6.97 (d, J = 2.4 Hz, 1H), 6.98-6.92 (m, 2H), 6.88 (dd, |
| | J = 8.9, 2.6 Hz, 1H), 6.81 (dd, J = 8.8, 2.4 Hz, 1H), 6.73 (m, 1H), 6.23 (d, J = 8.2 Hz, 1H), 5.76 |
| | (dd, J = 11.6, 5.4 Hz, 1H), 4.22 (dd, J = 7.9, 6.0 Hz, 2H), 4.20-4.11 (m, 4H), 4.06 (s, 1H), |
| | 3.87 (dd, J = 5.7, 3.8 Hz, 2H), 3.73 (m, 2H), 3.71-3.62 (m, 10H), 3.59 (m, 2H), 3.48 (m, 3H) |
| | 3.30 (q, J = 6.0 Hz, 2H), 2.99-2.81 (m, 3H), 2.41-2.33 (m, 1H), 2.04 (d, J = 9.9 Hz, 0H), 1.67- |
| | 1.60 (m, 4H), 1.27 (s, 6H), 1.25 (s, 2H), 1.22 (s, 2H). |
| Example | (500 MHz, CDCl3) δ 8.10 (s, 1H), 7.88 (d, J = 8.6 Hz, 1H), 7.72 (d, J = 8.1 Hz, 2H), 7.57 (d, |
| 17 | J = 8.6 Hz, 1H), 7.16 (d, J = 9.8 Hz, 2H), 6.98-6.92 (m, 3H), 6.81 (d, J = 8.8 Hz, 1H), 6.17 (d, |
| | J = 8.3 Hz, 1H), 5.72 (dd, J = 11.7, 5.5 Hz, 1H), 5.41 (m, 1H), 4.16 (q, J = 3.7, 2.4 Hz, 2H), |
| | 4.05 (s, 1H), 3.86 (t, J = 5.0 Hz, 2H), 3.72 (m, 2H), 3.69-3.63 (m, 12H), 3.61 (m, 2H), 3.51 (t, |
| | J = 5.8 Hz, 2H), 3.28 (q, J = 6.2 Hz, 2H), 2.97-2.88 (m, 2H), 2.81 (m, 1H), 2.36 (dd, J = 10.7, |
| | 6.7 Hz, 1H), 2.03 (d, J = 10.1 Hz, 1H), 1.78 (q, J = 6.9 Hz, 2H), 1.71 (p, J = 7.2, 6.8 Hz, 2H), |
| | 1.26 (s, 6H), 1.22 (s, 6H). |
| Example | (300 MHz, CDCl3) δ 8.34 (s, 1H), 8.14 (d, J = 9.8 Hz, 1H), 7.69 (d, J = 8.4 Hz, 2H), 7.64 (m, |
| 18 | 1H), 7.57 (d, J = 8.7 Hz, 1H), 6.96 (d, J = 2.4 Hz, 1H), 6.92 (d, J = 8.5 Hz, 2H), 6.81 (dd, J = |
| | 8.7, 2.4 Hz, 1H), 6.12 (d, J = 8.1 Hz, 1H), 5.79 (dd, J = 11.6, 5.1 Hz, 1H), 4.89 (d, J = 2.7 |
| | Hz, 2H), 4.15 (d, J = 8.1 Hz, 1H), 4.04 (s, 1H), 3.65-3.42 (m, 2H), 3.33 (m, 5H), 3.14 (d, J = |
| | 26.7 Hz, 1H), 3.03-2.85 (m, 2H), 2.78 (m, 1H), 2.49 (m, 4H), 2.09 (m, 2H), 1.65 (s, 8H), 1.26 |
| | (s, 6H), 1.22 (s, 6H). |
| Example | (300 MHz, CDCl3) δ 8.20 (d, J = 8.3 Hz, 1H), 7.95 (d, J = 8.8 Hz, 1H), 7.70 (d, J = 8.6 Hz, |
| 19 | 1H), 7.57 (d, J = 8.7 Hz, 1H), 7.25-7.20 (m, 1H), 7.13 (d, J = 2.6 Hz, 1H), 6.97 (d, J = 2.4 Hz, |
| | 1H), 6.91 (m, 1H), 6.81 (dd, J = 8.7, 2.4 Hz, 1H), 6.12 (d, J = 8.1 Hz, 1H), 6.01 (m, 1H), |
| | 5.84-5.74 (m, 1H), 5.40 (m, 1H), 4.15 (d, J = 8.1 Hz, 1H), 4.07-3.96 (m, 2H), 3.62 (d, J = |
| | 23.4 Hz, 2H), 3.36 (m, 4H), 3.03-2.79 (m, 4H), 2.53 (m, 3H), 2.28-1.95 (m, 4H), 1.63 (m, |
| | 5H), 1.26 (m, 10H), 1.22 (s, 6H), 1.15 (d, J = 7.7 Hz, 2H). |
| Example | (300 MHz, CDCl3) δ 8.33 (s, 1H), 8.01 (d, J = 9.1 Hz, 1H), 7.71 (d, J = 8.8 Hz, 2H), 7.58 (d, |
| 20 | J = 8.7 Hz, 1H), 7.54 (d, J = 2.8 Hz, 1H), 7.46 (dd, J = 9.3, 2.9 Hz, 1H), 6.98 (d, J = 2.4 Hz, |
| | 1H), 6.93 (d, J = 8.9 Hz, 2H), 6.82 (dd, J = 8.7, 2.4 Hz, 1H), 6.14 (d, J = 8.2 Hz, 1H), 5.79 |
| | (m, 1H), 4.17 (d, J = 8.1 Hz, 1H), 4.10 (m, 1H), 4.06 (m, 2H), 3.51 (m, 4H), 3.35 (m, 4H), |
| | 3.11 (t, J = 12.2 Hz, 2H), 3.03-2.86 (m, 3H), 2.81 (m, 2H), 2.53 (m, 4H), 2.43-2.34 (m, 1H), |
| | 2.16-2.01 (m, 3H), 2.00-1.87 (m, 4H), 1.79 (m, 3H), 1.27 (d, J = 2.3 Hz, 8H), 1.24 (s, 6H). |
| Example | (300 MHz, CDCl3) δ 8.12 (s, 1H), 7.98 (d, J = 8.8 Hz, 1H), 7.70 (d, J = 8.6 Hz, 2H), 7.57 (d, |
| 21 | J = 8.7 Hz, 1H), 7.04 (d, J = 2.6 Hz, 1H), 6.98-6.90 (m, 3H), 6.81 (dd, J = 8.7, 2.4 Hz, 1H), |
| | 6.11 (d, J = 8.1 Hz, 1H), 5.77 (m, 1H), 4.88 (s, 1H), 4.28 (m, 4H), 4.15 (d, J = 8.1 Hz, 1H), |
| | 4.04 (s, 1H), 3.78 (m, 1H), 3.58 (m, 2H), 3.34 (m, 4H), 3.22 (m, 1H), 3.02-2.82 (m, 3H), 2.46 |
| | (m, 4H), 2.12-1.95 (m, 4H), 1.66 (m, 4H), 1.26 (m, 8H), 1.22 (s, 6H). |
| Example | (300 MHz, CDCl3) δ 8.10 (s, 1H), 7.79 (t, J = 8.1 Hz, 1H), 7.73-7.64 (m, 3H), 7.57 (d, J = 8.7 |
| 22 | Hz, 1H), 7.23 (m, 1H), 6.97 (d, J = 2.4 Hz, 1H), 6.92 (d, J = 8.9 Hz, 2H), 6.81 (dd, J = 8.8, |
| | 2.4 Hz, 1H), 6.11 (d, J = 8.1 Hz, 1H), 5.81 (m, 1H), 4.15 (d, J = 8.1 Hz, 1H), 4.04 (s, 1H), |
| | 3.35 (m, 4H), 3.08 (m, 4H), 2.98-2.78 (m, 4H), 2.52 (m, 4H), 2.35 (m, 1H), 2.12 (t, J = 9.4 |
| | Hz, 2H), 2.03 (m, 1H), 1.92-1.82 (m, 4H), 1.26 (m, 8H), 1.22 (s, 6H). |
| Example | (300 MHz, CDCl3) δ 8.12 (s, 1H), 7.98 (d, J = 9.2 Hz, 1H), 7.70 (d, J = 8.7 Hz, 2H), 7.57 (d, |
| 23 | J = 8.7 Hz, 1H), 7.52 (d, J = 2.8 Hz, 1H), 7.49-7.41 (m, 1H), 7.34 (m, 1H), 6.97 (d, J = 2.4 |
| | Hz, 1H), 6.92 (m,, 2H), 6.81 (dd, J = 8.7, 2.4 Hz, 1H), 6.11 (d, J = 8.1 Hz, 1H), 5.76 (m, 1H), |
| | 4.15 (d, J = 8.0 Hz, 1H), 4.04 (s, 1H), 3.72 (d, J = 4.3 Hz, 1H), 3.50 (m, 2H), 3.43 (m, 2H), |
| | 3.34 (m, 4H), 3.04-2.78 (m, 4H), 2.51 (m, 4H), 2.42-2.30 (m, 1H), 2.12 (t, J = 9.7 Hz, 2H), |
| | 2.03 (m, 2H), 1.74 (m, 4H), 1.26 (s, 6H), 1.25 (m, 2H), 1.22 (s, 6H). |
| Example | (500 MHz, CDCl3) δ 8.14 (d, J = 8.7 Hz, 1H), 7.74-7.60 (m, 4H), 7.57 (d, J = 8.7 Hz, 1H), |
| 24 | 6.97 (d, J = 2.3 Hz, 1H), 6.92 (d, J = 8.5 Hz, 3H), 6.81 (dd, J = 8.7, 2.4 Hz, 1H), 6.12 (d, J = |
| | 8.1 Hz, 1H), 5.75 (m, 1H), 5.01-4.83 (m, 2H), 4.57 (d, J = 13.6 Hz, 1H), 4.15 (d, J = 8.1 Hz, |
| | 1H), 4.04 (s, 1H), 3.83 (m, 1H), 3.34 (m, 4H), 3.16-3.03 (m, 1H), 3.02-2.75 (m, 3H), 2.62 (m, |
| | 5H), 2.52-2.34 (m, 3H), 2.07-1.94 (m, 1H), 1.56-1.49 (m, 2H), 1.26 (m,10H), 1.22 (s, 6H). |
| Example | (500 MHz, CDCl3) 7.95 (d, J = 8.8 Hz, 1H), 7.71 (d, J = 8.4 Hz, 2H), 7.58 (d, J = 8.7 Hz, |
| 25 | 1H), 7.27 (dd, J = 8.9, 2.5 Hz, 1H), 7.15 (m, 1H), 6.98 (d, J = 2.3 Hz, 1H), 6.93 (d, J = 8.6 |
| | Hz, 2H), 6.83 (dd, J = 8.7, 2.3 Hz, 1H), 6.15 (d, J = 8.1 Hz, 1H), 6.10 (m, 1H), 5.80 (m 1H), |
| | 4.65 (d, J = 13.3 Hz, 1H), 4.17 (d, J = 8.1 Hz, 1H), 4.06 (s, 1H), 4.05-3.97 (m, 2H), 3.81 (d, |
| | J = 13.2 Hz, 1H), 3.35 (m, 4H), 3.10 (t, J = 12.5 Hz, 1H), 3.01-2.90 (m, 2H), 2.87 (m, 1H), 2.73 |
| | (t, J = 12.7 Hz, 1H), 2.64 (m, 4H), 2.48 (t, J = 7.8 Hz, 2H), 2.42-2.37 (m, 1H), 1.86 (m, 4H), |
| | 1.55 (q, J = 7.3 Hz, 2H), 1.28 (m, 10H), 1.24 (s, 6H). |
| Example | (300 MHz, CDCl3) δ 8.21 (s, 1H), 8.02 (d, J = 9.1 Hz, 1H), 7.72 (d, J = 8.8 Hz, 2H), 7.59 (d, |
| 26 | J = 8.7 Hz, 1H), 7.54 (d, J = 2.9 Hz, 1H), 7.47 (dd, J = 9.2, 2.9 Hz, 1H), 6.98 (d, J = 2.4 Hz, |
| | 1H), 6.94 (d, J = 8.8 Hz, 2H), 6.83 (dd, J = 8.7, 2.4 Hz, 1H), 6.14 (d, J = 8.1 Hz, 1H), 5.79 |
| | (dd, J = 11.4, 5.2 Hz, 1H), 4.64 (d, J = 13.1 Hz, 1H), 4.17 (d, J = 8.1 Hz, 1H), 4.08 (m, 3H), |
| | 3.95 (d, J = 13.3 Hz, 1H), 3.35 (t, J = 5.1 Hz, 4H), 3.11 (t, J = 12.2 Hz, 3H), 3.05-2.77 (m, |
| | 4H), 2.63 (m, 4H), 2.53-2.35 (m, 3H), 2.09-1.81 (m, 6H), 1.54 (q, J = 6.8, 6.3 Hz, 3H), 1.28 |
| | (m, f8H), 1.24 (s, 6H). |
| Example | (300 MHz, CDCl3) δ 8.39 (m, 1H), 7.97 (d, J = 8.8 Hz, 1H), 7.77-7.65 (m, 2H), 7.57 (d, J = |
| 27 | 8.7 Hz, 1H), 7.03 (dd, J = 4.4, 2.6 Hz, 1H), 6.97-6.88 (m, 4H), 6.81 (dd, J = 8.7, 2.4 Hz, 1H), |
| | 6.13 (d, J = 8.1 Hz, 1H), 5.82-5.71 (m, 1H), 4.61 (d, J = 13.2 Hz, 1H), 4.28 (m, 4H), 4.15 (d, |
| | J = 8.1 Hz, 1H), 4.07-4.01 (m, 1H), 3.78 (p, J = 7.3 Hz, 1H), 3.57 (d, J = 13.5 Hz, 1H), 3.33 |
| | (t, J = 5.0 Hz, 4H), 3.06 (t, J = 12.2 Hz, 1H), 3.01-2.75 (m, 3H), 2.61 (t, J = 5.1 Hz, 4H), 2.46 |
| | (t, J = 7.5 Hz, 2H), 2.41-2.31 (m, 1H), 1.82 (t, J = 13.8 Hz, 2H), 1.52 (q, J = 7.1 Hz, 2H), 1.26 |
| | (m, 8H), 1.22 (s, 6H). |
| Example | (300 MHz, CDCl3) δ 8.12 (s, 1H), 7.80 (t, J = 8.1 Hz, 1H), 7.73-7.64 (m, 3H), 7.57 (d, J = 8.7 |
| 28 | Hz, 1H), 7.24 (m, 1H), 6.97 (d, J = 2.4 Hz, 1H), 6.94-6.88 (m, 2H), 6.81 (dd, J = 8.7, 2.4 Hz, |
| | 1H), 6.12 (d, J = 8.1 Hz, 1H), 5.82 (m, 1H), 4.15 (d, J = 8.1 Hz, 1H), 4.04 (s, 1H), 3.48 (q, |
| | J = 7.0 Hz, 3H), 3.33 (m, 4H), 3.03-2.72 (m, 5H), 2.63 (m, 4H), 2.50 (t, J = 7.6 Hz, 2H), 2.35 |
| | (m, 1H), 1.83 (d, J = 12.2 Hz, 2H), 1.26 (m, 8H), 1.22 (m, 8H). |
| Example | (300 MHz, CDCl3) δ 8.18 (s, 1H), 7.98 (d, J = 9.2 Hz, 1H), 7.72-7.66 (m, 2H), 7.57 (d, J = |
| 29 | 8.7 Hz, 1H), 7.51 (d, J = 2.8 Hz, 1H), 7.43 (dd, J = 9.2, 2.9 Hz, 1H), 6.96 (d, J = 2.4 Hz, 1H), |
| | 6.94-6.88 (m, 2H), 6.81 (dd, J = 8.7, 2.4 Hz, 1H), 6.12 (d, J = 8.1 Hz, 1H), 5.76 (m, 1H), 4.15 |
| | (d, J = 8.1 Hz, 1H), 4.10-3.99 (m, 3H), 3.33 (t, J = 5.0 Hz, 4H), 3.10-2.80 (m, 5H), 2.62 (t, J = |
| | 5.0 Hz, 4H), 2.47 (t, J = 7.6 Hz, 2H), 2.40-2.25 (m, 1H), 2.03 (d, J = 5.6 Hz, 1H), 1.87 (d, J = |
| | 12.8 Hz, 2H), 1.26 (m, 10H), 1.22 (s, 6H). |
| Example | ¹H NMR (500 MHz, CDCl₃) δ 8.21 (s, 1H), 8.00 (d, J = 9.1 Hz, 1H), 7.70 (d, J = 8.4 Hz, 2H), |
| 35 | 7.57 (d, J = 8.7 Hz, 1H), 7.52 (d, J = 2.8 Hz, 1H), 7.45 (dd, J = 9.1, 2.8 Hz, 1H), 6.99-6.95 |
| | (m, 1H), 6.92 (d, J = 8.4 Hz, 2H), 6.84-6.78 (m, 1H), 6.12 (d, J = 8.1 Hz, 1H), 5.83-5.72 (m, |
| | 1H), 4.15 (d, J = 8.1 Hz, 1H), 4.10-4.01 (m, 3H), 3.95-3.85 (m, 1H), 3.79-3.69 (m, 1H), |
| | 3.61-3.49 (m, 3H), 3.43-3.28 (m, 6H), 3.16-3.05 (m, 2H), 3.00-2.88 (m, 2H), 2.88-2.76 (m, |
| | 2H), 2.67-2.57 (m, 4H), 2.55-2.46 (m, 2H), 2.43-2.33 (m, 1H), 2.07-1.77 (m, 8H), 1.64- |
| | 1.53 (m, 2H), 1.26 (s, 6H), 1.22 (s, 6H). |
| Example | ¹H NMR (500 MHz, CDCl₃) δ 8.33 (s, 1H), 7.97 (d, J = 8.8 Hz, 1H), 7.70 (d, J = 8.5 Hz, 2H), |
| 36 | 7.56 (d, J = 8.7 Hz, 1H), 7.04 (d, J = 2.4 Hz, 1H), 6.99-6.89 (m, 4H), 6.81 (dd, J = 8.7, 2.4 |
| | Hz, 1H), 6.12 (d, J = 8.1 Hz, 1H), 5.81-5.71 (m, 1H), 4.32 (t, J = 7.1 Hz, 2H), 4.27 (t, J = 8.2 |
| | Hz, 2H), 4.15 (d, J = 8.1 Hz, 1H), 4.04 (s, 1H), 3.89-3.83 (m, 1H), 3.83-3.75 (m, 1H), 3.61- |
| | 3.45 (m, 5H), 3.37-3.28 (m, 4H), 3.22-3.14 (m, 1H), 2.99-2.77 (m, 3H), 2.68-2.57 (m, 4H), |
| | 2.51 (t, J = 7.3 Hz, 2H), 2.42-2.33 (m, 1H), 1.89-1.77 (m, 4H), 1.69-1.58 (m, 2H), 1.26 (s, |
| | 6H), 1.22 (s, 6H). |
| Example | ¹H NMR (500 MHz, Methanol-d₄) δ 7.93 (d, J = 9.0 Hz, 1H), 7.77 (d, J = 8.5 Hz, 2H), 7.74 (d, |
| 37 | J = 8.8 Hz, 1H), 7.53 (d, J = 9.0 Hz, 1H), 7.41 (dd, J = 9.0, 2.5 Hz, 1H), 7.22-7.13 (m, 2H), |
| | 7.07-6.97 (m, 3H), 5.94-5.84 (m, 1H), 4.31 (s, 1H), 4.21 (s, 1H), 4.19-4.13 (m, 1H), 3.96- |
| | 3.85 (m, 1H), 3.85-3.72 (m, 1H), 3.70-3.56 (m, 4H), 3.49-3.40 (m, 1H), 3.40-3.35 (m, 4H), |
| | 3.02-2.80 (m, 3H), 2.77-2.64 (m, 4H), 2.64-2.54 (m, 2H), 2.40-2.30 (m, 1H), 1.96-1.75 (m, |
| | 4H), 1.66-1.49 (m, 2H), 1.30 (s, 6H), 1.24 (s, 6H).; LC/MS (ESI) m/z 921.4 |
| Example | ¹H NMR (500 MHz, CDCl₃) δ 8.33 (s, 0.5H), 8.25 (s, 0.5 H), 8.14 (d, J = 8.7 Hz, 1H), 7.69 (d, |
| 38 | J = 8.5 Hz, 2H), 7.67-7.59 (m, 2H), 7.57 (d, J = 8.7 Hz, 1H), 6.97 (d, J = 2.4 Hz, 1H), 6.92 |
| | (d, J = 8.6 Hz, 2H), 6.81 (dd, J = 8.7, 2.4 Hz, 1H), 6.11 (d, J = 8.1 Hz, 1H), 5.82-5.72 (m, |
| | 1H), 4.90 (s, 2H), 4.15 (d, J = 8.1 Hx, 1H), 4.04 (s, 1H), 3.87-3.77 (m, 1H), 3.73-3.64 (m, |
| | 1H), 3.64-3.45 (m, 4H), 3.41-3.25 (m, 5H), 3.02-2.79 (m, 3H), 2.74-2.57 (m, 4H), 2.57- |
| | 2.45 (m, 2H), 2.43-2.34 (m, 1H), 1.94-1.76 (m, 4H), 1.75-1.69 (m, 2H), 1.26 (s, 6H), 1.22 |
| | (s, 6H). |
| Example | ¹H NMR (500 MHz, CDCl₃) δ 8.20 (s, 1H), 7.99 (d, J = 9.1 Hz, 1H), 7.69 (d, J = 8.4 Hz, 2H), |
| 39 | 7.57 (d, J = 8.7 Hz, 1H), 7.53 (d, J = 2.8 Hz, 1H), 7.44 (dd, J = 9.1, 2.8 Hz, 1H), 6.97 (d, J = |
| | 2.4 Hz, 1H), 6.91 (d, J = 8.5 Hz, 2H), 6.81 (dd, J = 8.7, 2.4 Hz, 1H), 6.12 (d, J = 8.1 Hz, 1H), |
| | 5.81-5.72 (m, 1H), 4.15 (d, J = 8.1 Hz, 1H), 4.04 (s, 1H), 3.82-3.72 (m, 2H), 3.63-3.59 (m, |
| | 1H), 3.57 (t, J = 6.4 Hz, 2H), 3.40-3.34 (m, 2H), 3.34-3.26 (m, 4H), 3.01-2.78 (m, 3H), |
| | 2.68-2.56 (m, 4H), 2.51 (t, J = 7.3 Hz, 2H), 2.43-2.34 (m, 1H), 2.01-1.91 (m, 2H), 1.88- |
| | 1.79 (m, 2H), 1.79-1.70 (m, 2H), 1.26 (s, 6H), 1.22 (s, 6H). |
| Example | (500 MHz, CDCl3) δ 8.24 (s, 1H), 7.96 (d, J = 8.8 Hz, 1H), 7.74 (d, J = 8.4 Hz, 2H), 7.57 (d, |
| 40 | J = 8.7 Hz, 1H), 7.25 (m, 1H), 7.16 (d, J = 2.5 Hz, 1H), 6.95 (d, J = 9.1 Hz, 3H), 6.81 (m, |
| | 1H), 6.14 (d, J = 8.1 Hz, 1H), 5.96 (m, 1H), 5.80 (dd, J = 11.6, 5.4 Hz, 1H), 4.16 (d, J = 8.0 |
| | Hz, 1H), 4.06 (d, J = 4.6 Hz, 3H), 3.88 (t, J = 5.3 Hz, 2H), 3.67 (t, J = 4.9 Hz, 2H), 3.48 (q, |
| | J = 7.0 Hz, 1H), 3.39 (m, 2H), 3.35 (t, J = 5.3 Hz, 2H), 3.00-2.81 (m, 3H), 2.40 (m, 1H), 1.27 |
| | (s, 6H), 1.23 (s, 6H). |
| Example | (300 MHz, CDCl3) δ 7.89 (d+H102:H1033 (s, 2H), 2.56 (m, 4H), 2.23 (d, J = 7.0 Hz, 3H), |
| 41 | 2.03 (d, J = 5.9 Hz, 1H), 1.82 (d, J = 13.1 Hz, 2H), 1.26 (s, 6H), 1.22 (s, 6H).H85 |
| Example | (500 MHz, CDCl3) δ 8.18 (s, 1H), 8.01 (d, J = 9.4 Hz, 1H), 7.73 (d, J = 8.4 Hz, 2H), 7.57 (d, |
| 42 | J = 8.7 Hz, 1H), 7.53 (d, J = 2.8 Hz, 1H), 7.46 (dd, J = 9.2, 2.8 Hz, 1H), 6.97 (d, J = 2.3 Hz, |
| | 1H), 6.94 (d, J = 8.5 Hz, 2H), 6.81 (dd, J = 8.7, 2.3 Hz, 1H), 6.14 (d, J = 8.1 Hz, 1H), 5.78 |
| | (dd, J = 11.5, 5.4 Hz, 1H), 4.16 (d, J = 8.1 Hz, 1H), 4.10 (s, 1H), 4.07 (m, 1H), 3.82 (m, 2H), |
| | 3.74 (m, 2H), 3.36 (m, 2H), 3.31 (m, 2H), 3.15-3.07 (m, 2H), 3.00-2.92 (m, 3H), 2.89-2.84 |
| | (m, 2H), 2.38 (m, 1H), 2.05-1.96 (m, 2H), 1.91 (d, J = 3.8 Hz, 1H), 1.27 (s, 6H), 1.23 (s, 6H). |
| Example | (500 MHz, CDCl3) δ 8.21 (s, 1H), 7.98 (d, J = 8.8 Hz, 1H), 7.73 (d, J = 8.4 Hz, 2H), 7.57 (d, |
| 43 | J = 8.7 Hz, 1H), 7.06 (d, J = 2.5 Hz, 1H), 6.99-6.90 (m, 4H), 6.81 (dd, J = 8.8, 2.3 Hz, 1H), |
| | 6.15 (d, J = 8.2 Hz, 1H), 5.77 (dd, J = 11.4, 5.4 Hz, 1H), 4.36 (t, J = 7.0 Hz, 2H), 4.31 (t, J = |
| | 8.2 Hz, 2H), 4.16 (d, J = 8.1 Hz, 1H), 4.05 (s, 1H), 3.85 (m, 3H), 3.55 (t, J = 5.0 Hz, 2H), |
| | 3.33 (m, 4H), 2.98-2.83 (m, 3H), 2.37 (m, 1H), 1.27 (s, 6H), 1.23 (s, 6H). |
| Example | (400 MHz, CDCl3) δ 8.20 (m, 1H), 7.95 (d, J = 8.9 Hz, 1H), 7.69 (d, J = 8.8 Hz, 2H), 7.57 (d, |
| 44 | J = 8.7 Hz, 1H), 7.23 (m, 1H), 7.13 (d, J = 2.7 Hz, 1H), 6.96 (d, J = 2.4 Hz, 1H), 6.92 (d, J = |
| | 8.8 Hz, 2H), 6.81 (dd, J = 8.8, 2.4 Hz, 1H), 6.11 (d, J = 8.1 Hz, 1H), 6.02 (m, 1H), 5.77 (m, |
| | 1H), 4.64 (d, J = 13.0 Hz, 1H), 4.15 (d, J = 8.0 Hz, 1H), 4.04 (s, 1H), 3.98 (m, 2H), 3.79 (d, |
| | J = 13.6 Hz, 1H), 3.50 (d, J = 7.1 Hz, 1H), 3.46 (d, J = 7.0 Hz, 1H), 3.33 (m, 4H), 3.09 (t, J = |
| | 12.9 Hz, 1H), 3.01-2.82 (m, 3H), 2.70 (t, J = 12.7 Hz, 1H), 2.61 (m, 4H), 2.40 (m, 3H), 2.09- |
| | 1.99 (m, 1H), 1.84 (dd, J = 23.8, 13.5 Hz, 2H), 1.34 (m, 2H), 1.26 (s, 6H), 1.21 (m, 8H). |
| Example | (400 MHz, CDCl3) δ 8.14 (d, J = 8.8 Hz, 1H), 7.72-7.67 (m, 2H), 7.66-7.59 (m, 2H), 7.57 (d, |
| 45 | J = 8.7 Hz, 1H), 6.97 (d, J = 2.4 Hz, 1H), 6.94-6.89 (m, 2H), 6.81 (dd, J = 8.8, 2.4 Hz, 1H), |
| | 6.11 (d, J = 8.1 Hz, 1H), 5.75 (t, J = 5.4 Hz, 1H), 4.99-4.86 (m, 2H), 4.57 (d, J = 12.9 Hz, |
| | 1H), 4.15 (d, J = 8.0 Hz, 1H), 4.04 (s, 1H), 3.82 (t, J = 15.4 Hz, 1H), 3.50 (d, J = 7.0 Hz, 1H), |
| | 3.46 (d, J = 7.0 Hz, 1H), 3.32 (m, 6H), 3.16-2.97 (m, 2H), 2.96-2.84 (m, 2H), 2.61 (m, 5H), |
| | 2.44-2.34 (m, 3H), 2.03 (m, 1H), 1.80 (m, 2H), 1.32 (m, 3H), 1.26 (s, 6H), 1.22 (m, 8H). |
| Example | (500 MHz, CDCl3) δ 8.09 (s, 1H), 8.00 (d, J = 9.1 Hz, 1H), 7.70 (d, J = 8.8 Hz, 2H), 7.57 (d, |
| 46 | J = 8.7 Hz, 1H), 7.52 (d, J = 2.9 Hz, 1H), 7.45 (dd, J = 9.2, 2.9 Hz, 1H), 6.96 (d, J = 2.4 Hz, |
| | 1H), 6.94-6.87 (m, 2H), 6.81 (dd, J = 8.7, 2.4 Hz, 1H), 6.11 (d, J = 8.1 Hz, 1H), 5.77 (dd, J = |
| | 11.5, 5.4 Hz, 1H), 4.63 (d, J = 13.4 Hz, 1H), 4.15 (d, J = 8.1 Hz, 1H), 4.06 (m, 3H), 3.93 (d, |
| | J = 13.2 Hz, 1H), 3.48 (q, J = 7.0 Hz, 1H), 3.33 (m, 4H), 3.08 (q, J = 13.7 Hz, 3H), 2.99-2.77 |
| | (m, 4H), 2.58 (d, J = 26.6 Hz, 5H), 2.39 (m, 3H), 1.96 (m, 2H), 1.81 (m, 4H), 1.31 (m, 3H), |
| | 1.26 (m, 8H), 1.21 (m, 8H). |
| Example | (500 MHz, CDCl3) δ 8.11 (d, J = 3.4 Hz, 1H), 7.97 (d, J = 8.9 Hz, 1H), 7.72-7.67 (m, 2H), |
| 47 | 7.57 (d, J = 8.7 Hz, 1H), 7.04 (d, J = 2.6 Hz, 1H), 6.99-6.92 (m, 2H), 6.92 (d, J = 8.8 Hz, 2H), |
| | 6.81 (dd, J = 8.7, 2.4 Hz, 1H), 6.11 (d, J = 8.1 Hz, 1H), 5.77 (dd, J = 10.0, 5.4 Hz, 1H), 4.62 |
| | (d, J = 13.2 Hz, 1H), 4.30 (m, 4H), 4.15 (d, J = 8.1 Hz, 1H), 4.04 (s, 1H), 3.78 (p, J = 7.9 Hz, |
| | 1H), 3.57 (d, J = 13.6 Hz, 1H), 3.52-3.45 (m, 1H), 3.32 (m, 4H), 3.05 (t, J = 12.2 Hz, 1H), |
| | 2.99-2.88 (m, 2H), 2.87-2.78 (m, 1H), 2.60 (m, 5H), 2.38 (m, 3H), 1.81 (t, J = 16.3 Hz, 2H), |
| | 1.32 (q, J = 7.3 Hz, 2H), 1.26 (m, 8H), 1.21 (m, 8H). |
| Example | (500 MHz, CDCl3) δ 8.06 (s, 1H), 7.98 (d, J = 9.2 Hz, 1H), 7.69 (d, J = 8.4 Hz, 2H), 7.57 (d, |
| 48 | J = 8.7 Hz, 1H), 7.51 (d, J = 2.8 Hz, 1H), 7.43 (dd, J = 9.1, 2.9 Hz, 1H), 6.97 (d, J = 2.3 Hz, |
| | 1H), 6.92 (d, J = 8.5 Hz, 2H), 6.86-6.75 (m, 1H), 6.11 (d, J = 8.1 Hz, 1H), 5.76 (dd, J = 11.5, |
| | 5.3 Hz, 1H), 4.15 (d, J = 8.0 Hz, 1H), 4.05 (m, 3H), 3.33 (m, 4H), 3.05-2.89 (m, 4H), 2.88- |
| | 2.79 (m, 1H), 2.61 (m, 4H), 2.39 (m, 3H), 1.85 (d, J = 13.0 Hz, 2H), 1.32 (m, 5H), 1.26 (m, |
| | 8H), 1.22 (s, 6H). |
| Example | ¹H NMR (400 MHz, CDCl₃) δ 8.09 (s, 1H), 8.00 (d, J = 9.2 Hz, 1H), 7.72 (d, J = 8.2 Hz, 2H), |
| 51 | 7.57 (d, J = 8.7 Hz, 1H), 7.53 (d, J = 2.9 Hz, 1H), 7.45 (dd, J = 9.2, 2.9 Hz, 1H), 7.33 (d, J = |
| | 7.9 Hz, 2H), 6.97 (d, J = 2.4 Hz, 1H), 6.81 (dd, J = 8.7, 2.4 Hz, 1H), 6.19 (d, J = 8.1 Hz, 1H), |
| | 5.82-5.72 (m, 1H), 4.71-4.57 (m, 1H), 4.16 (d, J = 8.1 Hz, 1H), 4.12-4.01 (m, 3H), 4.01- |
| | 3.88 (m, 1H), 3.18-2.75 (m, 8H), 2.68-2.49 (m, 2H), 2.44-2.34 (m, 1H), 2.31-2.16 (m, 2H), |
| | 2.14-2.01 (m, 2H), 2.02-1.90 (m, 2H), 1.92-1.70 (m, 8H), 1.69-1.55 (m, 3H), 1.27 (s, 6H), |
| | 1.23 (s, 6H), 1.18-1.09 (m, 2H). |
| Example | ¹H NMR (400 MHz, CDCl₃) δ 8.12 (s, 1H), 7.97 (d, J = 8.7 Hz, 1H), 7.76-7.69 (m, 2H), 7.57 |
| 52 | (d, J = 8.7 Hz, 1H), 7.38-7.29 (m, 2H), 7.05 (d, J = 2.4 Hz, 1H), 6.99-6.92 (m, 2H), 6.81 (dd, |
| | J = 8.7, 2.4 Hz, 1H), 6.19 (d, J = 8.1 Hz, 1H), 5.81-5.72 (m, 1H), 4.70-4.57 (m, 1H), 4.41- |
| | 4.23 (m, 4H), 4.16 (d, J = 8.1 Hz, 1H), 4.05 (s, 1H), 3.86-3.74 (m, 1H), 3.68-3.53 (m, 1H), |
| | 3.13-2.97 (m, 3H), 2.97-2.76 (m, 3H), 2.72-2.61 (m, 1H), 2.61-2.49 (m, 1H), 2.42-2.33 (m, |
| | 1H), 2.32-2.19 (m, 2H), 2.15-1.99 (m, 2H), 1.99-1.91 (m, 1H), 1.91-1.70 (m, 6H), 1.27 (s, |
| | 6H), 1.23 (s, 6H), 1.19-1.04 (m, 2H). |
| Example | ¹H NMR (400 MHz, CDCl₃) δ 8.19-8.11 (m, 1H), 7.74-7.67 (m, 2H), 7.67-7.61 (m, 1H), 7.57 |
| 54 | (d, J = 8.8 Hz, 1H), 6.97 (d, J = 2.4 Hz, 1H), 6.94-6.87 (m, 2H), 6.81 (dd, J = 8.8, 2.4 Hz, |
| | 1H), 6.20-6.04 (m, 1H), 5.86-5.72 (m, 1H), 5.03-4.77 (m, 2H), 4.50-4.35 (m, 1H), 4.15 (d, |
| | J = 8.1 Hz, 1H), 4.05 (s, 1H), 3.70-3.41 (m, 2H), 3.41-3.23 (m, 4H), 3.16-2.86 (m, 3H), 2.86- |
| | 2.71 (m, 4H), 2.51-2.33 (m, 3H), 1.77-1.41 (m, 5H), 1.26 (s, 6H), 1.22 (s, 6H).; LC/MS |
| | (ESI) m/z 893.9 [M + H]⁺, 891.9 [M − H]⁻ |
| Example | ¹H NMR (400 MHz, CDCl₃) δ 8.15 (s, 1H), 8.00 (d, J = 9.2 Hz, 1H), 7.76-7.66 (m, 2H), 7.57 |
| 55 | (d, J = 8.7 Hz, 1H), 7.52 (d, J = 2.9 Hz, 1H), 7.45 (dd, J = 9.2, 2.9 Hz, 1H), 6.97 (d, J = 2.4 |
| | Hz, 1H), 6.91 (d, J = 8.9 Hz, 2H), 6.81 (dd, J = 8.9, 2.4 Hz, 1H), 6.12 (d, J = 8.1 Hz, 1H), |
| | 5.82-5.72 (m, 1H), 4.53-4.38 (m, 1H), 4.15 (d, J = 8.0 Hz, 1H), 4.13-3.97 (m, 3H), 3.83- |
| | 3.67 (m, 1H), 3.59-3.44 (m, 1H), 3.38-3.25 (m, 4H), 3.25-3.15 (m, 1H), 3.14-3.02 (m, 3H), |
| | 3.02-2.85 (m, 3H), 2.85-2.68 (m, 5H), 2.47-2.33 (m, 3H), 2.04-1.92 (m, 2H), 1.92-1.80 (m, |
| | 2H), 1.73-1.56 (m, 2H), 1.54-1.39 (m, 2H), 1.26 (s, 6H), 1.22 (s, 6H).; LC/MS (ESI) m/z |
| | 947.1 [M + H]⁺, 945.1 [M − H]⁻ |
| Example | ¹H NMR (400 MHz, CDCl₃) δ 8.11 (s, 1H), 7.98 (d, J = 8.8 Hz, 1H), 7.76-7.67 (m, 2H), 7.57 |
| 56 | (d, J = 8.7 Hz, 1H), 7.05 (d, J = 2.6 Hz, 1H), 6.99-6.93 (m, 2H), 6.93-6.87 (m, 2H), 6.81 (dd, |
| | J = 8.7, 2.6 Hz, 1H), 6.12 (d, J = 8.1 Hz, 1H), 5.82-5.71 (m, 1H), 4.51-4.42 (m, 1H), 4.39- |
| | 4.24 (m, 4H), 4.15 (d, J = 8.0 Hz, 1H), 4.04 (s, 1H), 3.80 (p, J = 7.4 Hz, 1H), 3.56-3.46 (m, |
| | 1H), 3.44-3.36 (m, 1H), 3.36-3.26 (m, 4H), 3.14-3.04 (m, 1H), 3.02-2.85 (m, 3H), 2.85- |
| | 2.75 (m, 4H), 2.44 (s, 2H), 2.42-2.32 (m, 1H), 1.74-1.54 (m, 2H), 1.52-1.40 (m, 2H), 1.26 |
| | (s, 6H), 1.22 (s, 6H).; LC/MS (ESI) m/z 919.1 [M + H]⁺, 917.0 [M − H]⁻ |
| Example | (300 MHz, CDCl3) δ 8.26 (s, 1H), 8.01 (d, J = 9.1 Hz, 1H), 7.76-7.68 (m, 2H), 7.57 (d, J = |
| 57 | 8.7 Hz, 1H), 7.53 (d, J = 2.8 Hz, 1H), 7.45 (dd, J = 9.2, 2.9 Hz, 1H), 6.98-6.89 (m, 3H), 6.81 |
| | (dd, J = 8.7, 2.4 Hz, 1H), 6.13 (d, J = 8.1 Hz, 1H), 5.78 (m, 1H), 4.15 (d, J = 8.0 Hz, 1H), |
| | 4.04 (s, 1H), 3.80 (m, 4H), 3.52 (t, J = 4.8 Hz, 4H), 3.34 (m, 6H), 3.02-2.81 (m, 3H), 2.74 (t, |
| | J = 5.1 Hz, 4H), 2.45-2.30 (m, 1H), 1.26 (s, 6H), 1.22 (s, 6H). |
| Example | (400 MHz, CDCl3) δ 8.21 (s, 1H), 8.01 (d, J = 9.0 Hz, 1H), 7.73-7.65 (m, 2H), 7.56 (d, J = |
| 58 | 8.7 Hz, 1H), 7.53 (d, J = 2.9 Hz, 1H), 7.45 (dd, J = 9.2, 2.9 Hz, 1H), 6.96 (d, J = 2.4 Hz, 1H), |
| | 6.94-6.88 (m, 2H), 6.81 (dd, J = 8.7, 2.4 Hz, 1H), 6.12 (d, J = 8.1 Hz, 1H), 5.77 (m, 1H), 4.61 |
| | (m, 1H), 4.15 (d, J = 8.1 Hz, 1H), 4.06 (m, 2H), 3.51 (t, J = 5.1 Hz, 3H), 3.31 (m, 4H), 3.21 |
| | (d, J = 13.7 Hz, 2H), 3.01 (m, 1H), 2.96 (s, 2H), 2.95 (s, 2H), 2.95-2.92 (m, 1H), 2.88 (s, 2H), |
| | 2.87-2.81 (m, 1H), 2.72 (q, J = 4.8 Hz, 3H), 2.59 (m, 4H), 2.42-2.34 (m, 1H), 2.26 (m, 1H), |
| | 1.87 (d, 3H), 1.26 (s, 6H), 1.22 (s, 6H), 1.11 (m, 2H). |
| Example | (400 MHz, CDCl3) δ 8.16 (d, J = 9.0 Hz, 1H), 7.69 (d, J = 9.1 Hz, 3H), 7.58 (s, 1H), 6.96 (d, |
| 59 | J = 2.4 Hz, 1H), 6.90 (d, J = 8.6 Hz, 2H), 6.81 (dd, J = 8.7, 2.4 Hz, 1H), 6.11 (d, J = 8.1 Hz, |
| | 1H), 5.77 (dd, J = 11.5, 5.6 Hz, 1H), 4.71 (s, 2H), 4.15 (d, J = 8.1 Hz, 1H), 4.04 (s, 1H), 3.70- |
| | 3.46 (m, 3H), 3.29 (m, 4H), 3.03-2.76 (m, 5H), 2.57 (m, 4H), 2.40 (m, 1H), 2.27 (d, J = 5.6 |
| | Hz, 2H), 2.03 (m, 2H), 1.88 (m, 2H), 1.26 (s, 6H), 1.22 (s, 6H), 0.86 (m, 4H). |
| Example | (300 MHz, CDCl3) δ 7.89 (d, J = 8.8 Hz, 1H), 7.69 (d, J = 8.5 Hz, 2H), 7.57 (d, J = 8.7 Hz, |
| 60 | 1H), 7.23 (dd, J = 8.9, 2.6 Hz, 1H), 7.14 (m, 1H), 6.97 (d, J = 2.4 Hz, 1H), 6.90 (d, J = 8.6 |
| | Hz, 2H), 6.81 (dd, J = 8.8, 2.4 Hz, 1H), 6.13 (d, J = 8.1 Hz, 1H), 5.85 (m, 1H), 5.72 (m, 1H), |
| | 4.15 (d, J = 8.1 Hz, 1H), 4.05 (s, 1H), 3.98 (m, 2H), 3.53 (m, 2H), 3.29 (m, 4H), 3.12 (m, 2H), |
| | 2.99-2.81 (m, 3H), 2.73 (s, 2H), 2.56 (m, 4H), 2.23 (d, J = 7.0 Hz, 3H), 2.03 (d, J = 5.9 Hz, |
| | 1H), 1.82 (d, J = 13.1 Hz, 2H), 1.26 (s, 6H), 1.22 (s, 6H). |
| Example | ¹H NMR (400 MHz, CDCl₃) δ 8.28 (s, 1H), 8.05-7.95 (m, 1H), 7.75-7.65 (m, 2H), 7.56 (d, |
| 62 | J = 8.8 Hz, 1H), 7.52 (d, J = 2.8 Hz, 1H), 7.44 (dd, J = 9.1, 2.8 Hz, 1H), 6.96 (d, J = 2.4 Hz, |
| | 1H), 6.95-6.87 (m, 2H), 6.81 (dd, J = 8.8, 2.4 Hz, 1H), 6.13 (d, J = 8.1 Hz, 1H), 5.83-5.72 |
| | (m, 1H), 4.54-4.39 (m, 1H), 4.15 (d, J = 8.1 Hz, 1H), 4.12-4.00 (m, 3H), 3.87-3.73 (m, 1H), |
| | 3.53-3.39 (m, 1H), 3.37-3.22 (m, 4H), 3.17-3.04 (m, 2H), 3.04-2.75 (m, 6H), 2.77-2.65 (m, |
| | 4H), 2.65-2.49 (m, 2H), 2.44-2.32 (m, 1H), 2.19-2.07 (m, 1H), 2.07-1.93 (m, 3H), 1.93- |
| | 1.79 (m, 2H), 1.72-1.47 (m, 2H), 1.26 (s, 6H), 1.22 (s, 6H).; LC/MS (ESI) m/z 949.0 [M + H]⁺, |
| | 946.9 [M − H]⁻ |
| Example | ¹H NMR (400 MHz, CDCl₃) δ 8.25 (s, 1H), 7.97 (d, J = 8.8 Hz, 1H), 7.74-7.66 (m, 2H), 7.56 |
| 63 | (d, J = 8.7 Hz, 1H), 7.04 (d, J = 2.6 Hz, 1H), 7.00-6.93 (m, 2H), 6.93-6.87 (m, 2H), 6.81 (dd, |
| | J = 8.8, 2.4 Hz, 1H), 6.13 (d, J = 8.1 Hz, 1H), 5.82-5.71 (m, 1H), 4.54-4.43 (m, 1H), 4.39- |
| | 4.25 (m, 4H), 4.15 (d, J = 8.1 Hz, 1H), 4.04 (s, 1H), 3.80 (p, J = 7.5 Hz, 1H), 3.51-3.39 (m, |
| | 2H), 3.36-3.23 (m, 4H), 3.09-2.78 (m, 4H), 2.78-2.67 (m, 4H), 2.64-2.51 (m, 2H), 2.42- |
| | 2.32 (m, 1H), 2.19-2.08 (m, 1H), 2.08-1.98 (m, 1H), 1.64-1.46 (m, 2H), 1.26 (s, 6H), 1.22 |
| | (s, 6H).; LC/MS (ESI) m/z 921.0 [M + H]⁺, 918.9 [M − H]⁻ |
| Example | ¹H NMR (400 MHz, CDCl₃) δ 8.07 (s, 1H), 8.02 (d, J = 9.1 Hz, 1H), 7.74-7.65 (m, 2H), 7.56 |
| 64 | (d, J = 8.7 Hz, 1H), 7.50 (d, J = 2.9 Hz, 1H), 7.43 (dd, J = 9.1, 2.9 Hz, 1H), 6.96 (d, J = 2.4 |
| | Hz, 1H), 6.94-6.87 (m, 2H), 6.80 (dd, J = 8.7, 2.4 Hz, 1H), 6.11 (d, J = 8.1 Hz, 1H), 5.83- |
| | 5.73 (m, 2H), 4.15 (d, J = 8.1 Hz, 1H), 4.07-4.01 (m, 1H), 4.01-3.92 (m, 2H), 3.67 (t, J = 5.7 |
| | Hz, 2H), 3.38-3.22 (m, 4H), 3.06-3.01 (m, 1H), 3.01-2.78 (m, 4H), 2.63-2.48 (m, 4H), 2.43- |
| | 2.30 (m, 3H), 1.26 (s, 6H), 1.22 (s, 6H).; LC/MS (ESI) m/z 818.0 [M + H]+, 815.9 [M − H]- |
| Example | ¹H NMR (400 MHz, CDCl₃) δ 8.23 (s, 1H), 7.99 (d, J = 9.1 Hz, 1H), 7.75-7.66 (m, 2H), 7.56 |
| 65 | (d, J = 8.8 Hz, 1H), 7.52 (d, J = 2.9 Hz, 1H), 7.45 (dd, J = 9.1, 2.9 Hz, 1H), 6.97 (d, J = 2.4 |
| | Hz, 1H), 6.95-6.87 (m, 2H), 6.81 (dd, J = 8.8, 2.4 Hz, 1H), 6.12 (d, J = 8.1 Hz, 1H), 5.82- |
| | 5.73 (m, 1H), 5.70-5.60 (m, 1H), 4.15 (d, J = 8.1 Hz, 1H), 4.12-4.02 (m, 5H), 3.72 (t, J = 5.7 |
| | Hz, 1H), 3.62 (t, J = 5.6 Hz, 1H), 3.36-3.25 (m, 4H), 3.17-3.05 (m, 2H), 3.01-2.90 (m, 4H), |
| | 2.90-2.75 (m, 2H), 2.59-2.48 (m, 4H), 2.42-2.34 (m, 1H), 2.30-2.23 (m, 1H), 2.23-2.15 (m, |
| | 1H), 2.05-1.92 (m, 2H), 1.92-1.83 (m, 2H), 1.26 (s, 6H), 1.22 (s, 6H).; LC/MS (ESI) m/z |
| | 929.0 [M + H]⁺, 926.9 [M − H]⁻ |
| Example | ¹H NMR (400 MHz, CDCl₃) δ 8.17 (s, 0.5H), 8.13 (s, 0.5H), 7.97 (dd, J = 8.9, 2.8 Hz, 1H), |
| 66 | 7.76-7.66 (m, 2H), 7.56 (d, J = 8.7 Hz, 1H), 7.09-7.03 (m, 1H), 7.00-6.88 (m, 4H), 6.81 (dd, |
| | J = 8.7, 2.4 Hz, 1H), 6.12 (d, J = 8.1 Hz, 1H), 5.81-5.72 (m, 1H), 5.68 (s, 0.5H), 5.62 (s, |
| | 0.5H), 4.40-4.25 (m, 4H), 4.20-4.10 (m, 2H), 4.04 (s, 1H), 3.92-3.86 (m, 1H), 3.85-3.78 (m, |
| | 1H), 3.78-3.72 (m, 1H), 3.49-3.41 (m, 1H), 3.36-3.25 (m, 4H), 3.02-2.95 (m, 2H), 2.95- |
| | 2.75 (m, 3H), 2.58-2.48 (m, 4H), 2.43-2.32 (m, 1H), 2.29-2.18 (m, 2H), 1.26 (s, 6H), 1.22 |
| | (s, 6H).; LC/MS (ESI) m/z 901.0 [M + H]⁺, 898.9 [M − H]⁻ |
| Example | (300 MHz, CDCl3) δ 8.04 (s, 1H), 7.97 (d, J = 8.9 Hz, 1H), 7.69 (d, J = 8.5 Hz, 2H), 7.57 (d, |
| 67 | J = 8.7 Hz, 1H), 6.99 (dd, J = 15.4, 2.5 Hz, 2H), 6.94-6.85 (m, 3H), 6.81 (dd, J = 8.8, 2.4 Hz, |
| | 1H), 6.11 (d, J = 8.1 Hx, 1H), 5.75 (m, 1H), 4.88 (s, 1H), 4.26 (m, 1H), 4.15 (d, J = 8.1 Hz, |
| | 1H), 4.04 (s, 1H), 3.80 (m, 2H), 3.30 (m, 4H), 3.04-2.78 (m, 4H), 2.57 (m, 4H), 2.31 (m, 5H), |
| | 2.03 (m, 5H), 1.25 (s, 6H), 1.22 (s, 6H), 0.87 (m, 4H). |
| Example | (400 MHz, CDCl3) δ 7.98 (d, J = 9.1 Hz, 1H), 7.69 (d, J = 8.5 Hz, 2H), 7.57 (d, J = 8.7 Hz, |
| 68 | 1H), 7.50 (d, J = 2.7 Hz, 1H), 7.43 (dd, J = 9.3, 2.8 Hz, 1H), 6.97 (d, J = 2.4 Hz, 1H), 6.90 (d, |
| | J = 8.6 Hz, 2H), 6.81 (dd, J = 8.7, 2.4 Hz, 1H), 6.12 (d, J = 8.2 Hz, 1H), 5.77 (dd, J = 11.3, |
| | 5.4 Hz, 1H), 4.15 (d, J = 8.1 Hz, 1H), 4.05 (m, 3H), 3.59 (m, 2H), 3.49 (s, 1H), 3.28 (t, J = |
| | 4.9 Hz, 4H), 3.14-3.03 (m, 2H), 3.01-2.74 (m, 5H), 2.56 (m, 4H), 2.40-2.26 (m, 4H), 2.01 (m, |
| | 5H), 1.86 (t, J = 11.3 Hz, 3H), 1.26 (m, 8H), 1.22 (s, 6H), 0.92-0.83 (m, 2H). |
| Example | (400 MHz, CDCl3) δ 7.95 (d, J = 8.9 Hz, 1H), 7.69 (d, J = 8.5 Hz, 2H), 7.57 (d, J = 8.6 Hz, |
| 69 | 1H), 7.03 (d, J = 2.6 Hz, 1H), 6.96 (d, J = 2.4 Hz, 1H), 6.94-6.87 (m, 3H), 6.81 (dd, J = 8.7, |
| | 2.4 Hz, 1H), 6.12 (d, J = 8.1 Hz, 1H), 5.76 (m, 1H), 4.24 (d, J = 7.2 Hz, 3H), 4.15 (d, J = 8.0 |
| | Hz, 2H), 4.04 (s, 1H), 3.56 (m, 2H), 3.29 (t, J = 5.0 Hz, 4H), 3.00-2.77 (m, 5H), 2.56 (t, J = |
| | 5.0 Hz, 4H), 2.41-2.32 (m, 2H), 2.28 (d, J = 6.9 Hz, 2H), 2.07-1.85 (m, 4H), 1.64 (m, 1H), |
| | 1.26 (m, 8H), 1.22 (s, 6H), 0.88-0.80 (m, 2H). |
| Example | (400 MHz, CDCl3) δ 8.18 (s, 1H), 7.98 (d, J = 9.1 Hz, 1H), 7.69 (d, J = 8.5 Hz, 2H), 7.57 (d, |
| 70 | J = 8.7 Hz, 1H), 7.50 (d, J = 2.8 Hz, 1H), 7.43 (dd, J = 9.2, 2.9 Hz, 1H), 6.97 (d, J = 2.4 Hz, |
| | 1H), 6.91 (d, J = 8.5 Hz, 2H), 6.81 (dd, J = 8.7, 2.4 Hz, 1H), 6.12 (d, J = 8.1 Hz, 1H), 5.76 |
| | (dd, J = 11.5, 5.3 Hz, 1H), 4.15 (d, J = 8.1 Hz, 1H), 4.09-3.98 (m, 3H), 3.30 (t, J = 4.9 Hz, |
| | 4H), 3.08-2.96 (m, 2H), 2.94-2.79 (m, 2H), 2.56 (t, J = 5.0 Hz, 4H), 2.42-2.32 (m, 1H), 2.28 |
| | (d, J = 6.5 Hz, 2H), 2.01 (m, 4H), 1.84 (m, 4H), 1.61 (m, 2H), 1.41-1.31 (m, 3H), 1.26 (m, |
| | 8H), 1.22 (s, 6H). |
| Example | (300 MHz, CDCl3) δ 8.43 (s, 1H), 7.98 (d, J = 9.1 Hz, 1H), 7.67 (d, J = 8.6 Hz, 2H), 7.56 (d, |
| 71 | J = 8.7 Hz, 1H), 7.50 (d, J = 2.8 Hz, 1H), 7.43 (dd, J = 9.3, 2.9 Hz, 1H), 6.97 (d, J = 2.4 Hz, |
| | 1H), 6.81 (dd, J = 8.7, 2.4 Hz, 1H), 6.55 (d, J = 8.7 Hz, 2H), 6.10 (d, J = 8.1 Hz, 1H), 5.84- |
| | 5.69 (m, 1H), 4.34 (m, 1H), 4.21-4.13 (m, 1H), 4.08-3.99 (m, 3H), 3.44 (m, 6H), 3.08 (t, J = |
| | 12.0 Hz, 2H), 2.98-2.69 (m, 5H), 2.43-2.28 (m, 1H), 2.10-1.88 (m, 6H), 1.55 (m, 4H), 1.26 (s, |
| | 6H), 1.22 (s, 6H). |
| Example | (300 MHz, CDCl3) δ 8.15 (s, 1H), 7.97 (d, J = 9.1 Hz, 1H), 7.67 (d, J = 8.6 Hz, 2H), 7.56 (d, |
| 72 | J = 8.7 Hz, 1H), 7.49 (d, J = 2.8 Hz, 1H), 7.41 (dd, J = 9.2, 2.9 Hz, 1H), 6.97 (d, J = 2.4 Hz, |
| | 1H), 6.81 (dd, J = 8.7, 2.4 Hz, 1H), 6.56 (d, J = 8.7 Hz, 2H), 6.08 (d, J = 8.1 Hz, 1H), 5.75 |
| | (m, 1H), 4.30 (s, 1H), 4.15 (d, J = 8.0 Hz, 1H), 4.02 (m, 3H), 3.57 (m, 1H), 3.39 (m, 2H), 2.92 |
| | (m, 6H), 2.59 (d, J = 9.2 Hz, 1H), 2.49-2.30 (m, 3H), 2.07-1.80 (m, 4H), 1.29-1.23 (m, 8H), |
| | 1.22 (s, 6H). |
| Example | (300 MHz, CDCl3) δ 8.30 (s, 1H), 7.96 (d, J = 8.9 Hz, 1H), 7.72-7.65 (m, 2H), 7.56 (d, J = |
| 73 | 8.7 Hz, 1H), 7.03 (d, J = 2.6 Hz, 1H), 6.96 (d, J = 2.4 Hz, 1H), 6.93 (dd, J = 8.8, 2.6 Hz, 1H), |
| | 6.81 (dd, J = 8.7, 2.4 Hz, 1H), 6.55 (d, J = 8.4 Hz, 2H), 6.09 (d, J = 8.1 Hz, 1H), 5.75 (m, |
| | 1H), 4.59 (d, J = 13.3 Hz, 2H), 4.35-4.21 (m, 5H), 4.15 (d, J = 8.1 Hz, 1H), 4.04 (s, 1H), |
| | 3.83-3.68 (m, 2H), 3.56 (m, 2H), 3.45-3.30 (m, 2H), 3.08-2.75 (m, 5H), 2.58 (m, 2H), 2.47- |
| | 2.30 (m, 3H), 2.02-1.52 (m, 6H), 1.26 (s, 6H), 1.22 (s, 6H), 1.07 (q, J = 12.3 Hz, 2H). |
| Example | ¹H NMR (400 MHz, CDCl₃) δ 8.15 (s, 1H), 7.97 (d, J = 8.8 Hz, 1H), 7.76-7.69 (m, 2H), 7.57 |
| 75 | (d, J = 8.7 Hz, 1H), 7.36-7.30 (m, 2H), 7.05 (d, J = 2.6 Hz, 1H), 6.97 (d, J = 2.4 Hz, 1H), |
| | 6.94 (dd, J = 8.8, 2.6 Hz, 1H), 6.81 (dd, J = 8.7, 2.4 Hz, 1H), 6.19 (d, J = 8.1 Hz, 1H), 5.83- |
| | 5.72 (m, 1H), 4.38-4.23 (m, 4H), 4.16 (d, J = 8.1 Hz, 1H), 4.08-4.01 (m, 1H), 3.94-3.85 (m, |
| | 1H), 3.85-3.74 (m, 1H), 3.73-3.63 (m, 2H), 3.63-3.57 (m, 1H), 3.57-3.41 (m, 2H), 3.23- |
| | 3.14 (m, 1H), 3.14-3.05 (m, 2H), 3.01-2.76 (m, 3H), 2.67 (t, J = 6.1 Hz, 2H), 2.63-2.52 (m, |
| | 1H), 2.43-2.33 (m, 1H), 2.27-2.12 (m, 2H), 1.93-1.75 (m, 6H), 1.72-1.65 (m, 2H), 1.27 (s, |
| | 6H), 1.23 (s, 6H).; LC/MS (ESI) m/z 932.0 [M + H]⁺, 929.9 [M − H]⁻ |
| Example | ¹H NMR (400 MHz, CDCl₃) δ 8.14 (s, 1H), 7.99 (d, J = 9.2 Hz, 1H), 7.77-7.68 (m, 2H), 7.57 |
| 76 | (d, J = 8.7 Hz, 1H), 7.53 (d, J = 2.9 Hz, 1H), 7.44 (dd, J = 9.2, 2.9 Hz, 1H), 7.33 (d, J = 8.2 |
| | Hz, 2H), 6.97 (d, J = 2.4 Hz, 1H), 6.81 (dd, J = 8.7, 2.4 Hz, 1H), 6.19 (d, J = 8.1 Hz, 1H), |
| | 5.81-5.73 (m, 1H), 4.21-4.12 (m, 1H), 4.09-4.02 (m, 1H), 3.89-3.71 (m, 4H), 3.70-3.59 (m, |
| | 1H), 3.42-3.25 (m, 3H), 3.03-2.77 (m, 5H), 2.75-2.53 (m, 2H), 2.45-2.32 (m, 2H), 2.08- |
| | 1.94 (m, 3H), 1.94-1.82 (m, 3H), 1.81-1.67 (m, 3H), 1.27 (s, 6H), 1.23 (s, 6H).; LC/MS |
| | (ESI) m/z 849.0 [M + H]⁺, 846.9 [M − H]⁻ |
| Example | ¹H NMR (400 MHz, DMSO-d₆) δ 11.23 (s, 1H), 9.03 (s, H), 8.71-8.68 (m, 2H), 8.5 (d, J = 9.5 |
| 77 | Hz, 1H), 7.90 (d, J = 8.8 Hz, 1H), 7.76 (d, J = 8.8 Hz, 2H), 7.51 (d, J = 9.1 Hz, 1H), 7.21 (d, |
| | J = 2.4 Hz, 1H), 7.02-6.98 (m, 3H), 6.07-6.03 (m, 1H), 4.32 (s, 1H), 4.06 (m, J = 9.0 Hz, 1H), |
| | 3.29 (s, 3H), 3.00-2.96 (m, 3H), 2.77-2.73 (m, 3H), 2.77-2.31 (m, 3H), 2.76-2.73 (m, 3H), |
| | 2.67 (s, 1H), 2.67-2.63 (m, 3H), 2.33 (s, 2H), 1.22 (s, 6H), 1.13 (s, 6H); LC/MS (ESI) m/z |
| | 817.9 [M + H]⁺ |
| Example | ¹H NMR (300 MHz, Methanol-d₄) δ 8.62 (d, J = 2.5 Hz, 1H), 8.03-7.96 (m, 2H), 7.74 (d, J = |
| 78 | 8.7 Hz, 1H), 7.68 (m, J = 9.2, 3.2 Hz, 1H), 7.53 (d, J = 2.8 Hz, 1H), 7.15 (d, J = 2.4 Hz, 1H), |
| | 7.00 (m, J = 8.8, 2.4 Hz, 1H), 6.86 (d, J = 9.1 Hz, 1H), 5.90 (m, J = 11.6, 5.2 Hz, 1H), 4.30 |
| | (s, 1H), 4.21-4.11 (m, 3H), 3.72 (t, J = 5.0 Hz, 4H), 3.09 (t, J = 12.4 Hz, 2H), 2.91-2.80 (m, |
| | 2H), 2.61 (t, J = 5.0 Hz, 4H), 2.35 (d, J = 6.4 Hz, 3H), 2.02 (m, J = 14.8, 9.0 Hz, 5H), 1.30 (s, |
| | 6H), 1.24 (s, 6H); LC/MS (ESI) m/z, [M + H]⁺: 822.0. [M − H]⁻: 820.0. |
| Example | ¹H NMR (300 MHz, Methanol-d₄) δ 8.70 (d, J = 1.3 Hz, 1H), 8.25 (d, J = 1.4 Hz, 1H), 8.11 (d, |
| 79 | J = 8.9 Hz, 1H), 8.00 (d, J = 9.2 Hz, 1H), 7.74 (d, J = 8.7 Hz, 1H), 7.69 (dd, J = 9.3, 2.8 Hz, |
| | 1H), 7.53 (d, J = 2.9 Hz, 1H), 7.17 (d, J = 2.4 Hz, 1H), 7.01 (dd, J = 8.8, 2.5 Hz, 1H), 5.90 |
| | (dd, J = 11.7, 5.2 Hz, 1H), 4.34 (s, 1H), 4.22-4.06 (m, 4H), 3.82 (d, J = 5.3 Hz, 4H), 3.11 (d, |
| | J = 12.2 Hz, 2H), 2.92-2.81 (m, 2H), 2.65 (s, 4H), 2.38 (d, J = 7.2 Hz, 3H), 2.02 (dd, J = |
| | 14.1, 9.2 Hz, 6H), 1.30 (s, 6H), 1.23 (s, 6H); LC/MS (ESI) m/z [M + H]⁺: 821.9; [M − H]⁻: |
| | 819.9 |
| Example | ¹H NMR (300 MHz, Methanol-d₄) δ 8.63 (d, J = 2.5 Hz, 1H), 8.05-7.90 (m, 2H), 7.78-7.72 (m, |
| 80 | 1H), 7.37 (t, J = 6.8 Hz, 1H), 7.20-7.10 (m, 2H), 7.05-6.97 (m, 2H), 6.87 (d, J = 9.1 Hz, 1H), |
| | 5.93-5.86 (m, 1H), 4.57 (d, J = 12.9 Hz, 1H), 4.41-4.31 (m, 2H), 4.30-4.24 (m, 2H), 4.19-4.13 |
| | (m, 1H), 4.03 (d, J = 7.0 Hz, 1H), 3.80 (d, J = 13.5 Hz, 1H), 3.73 (s, 4H), 3.17 (t, J = 12.7 Hz, |
| | 2H), 2.99-2.71 (m, 4H), 2.65 (s, 4H), 2.38 (t, J = 6.0 Hz, 2H), 2.09-1.84 (m, 4H), 1.30 (s, 6H), |
| | 1.24 (s, 6H). |
| Example | ¹H NMR (400 MHz, Methanol-d₄) δ 8.62 (d, J = 2.5 Hz, 1H), 8.03-7.95 (m, 2H), 7.74 (d, J = |
| 81 | 8.7 Hz, 1H), 7.69 (d, J = 9.3, 2.9 Hz, 1H), 7.55 (d, J = 2.9 Hz, 1H), 7.15 (d, J = 2.4 Hz, 1H), |
| | 7.00 (d, J = 8.8, 2.4 Hz, 1H), 6.85 (d, J = 9.1 Hz, 1H), 5.94-5.87 (m, 1H), 4.56 (d, J = 13.3 |
| | Hz, 2H), 4.30 (s, 1H), 4.22-4.12 (m, 4H), 3.70 (t, J = 5.0 Hz, 4H), 3.55-3.46 (m, 2H), 3.24- |
| | 3.12 (m, 4H), 3.11-3.03 (m, 1H), 3.00-2.79 (m, 4H), 2.69 (t, J = 12.6 Hz, 2H), 2.57 (t, J = 5.2 |
| | Hz, 4H), 2.41-2.33 (m, 2H), 2.30 (d, J = 6.7 Hz, 2H), 2.02-1.79 (m, 8H), 1.30 (s, 6H), 1.24 (s, |
| | 6H). |
| Example | ¹H NMR (400 MHz, CDCl₃) δ 8.24 (s, 1H), 8.00-7.95 (m, 1H), 7.86-7.82 (m, 1H), 7.81-7.73 |
| 83 | (m, 3H), 7.61-7.53 (m, 3H), 7.04 (d, J = 2.6 Hz, 1H), 6.97 (d, J = 2.4 Hz, 1H), 6.95 (dd, J = |
| | 8.9, 2.6 Hz, 1H), 6.81 (dd, J = 8.7, 2.4 Hz, 1H), 6.23 (d, J = 8.1 Hz, 1H), 5.85-5.71 (m, 1H), |
| | 4.71-4.57 (m, 1H), 4.39-4.23 (m, 4H), 4.23-4.12 (m, 2H), 4.07 (s, 1H), 3.86-3.74 (m, 1H), |
| | 3.65-3.54 (m, 1H), 3.13-2.76 (m, 6H), 2.71-2.61 (m, 1H), 2.44-2.33 (m, 1H), 2.26 (d, J = |
| | 6.8 Hz, 2H), 2.23-2.02 (m, 6H), 1.97-1.89 (m, 1H), 1.89-1.83 (m, 1H), 1.80-1.73 (m, 1H), |
| | 1.29 (s, 6H), 1.24 (s, 6H), 1.18-1.04 (m, 2H). |
| Example | ¹H NMR (400 MHz, CDCl₃) δ 8.21 (s, 1H), 7.98 (d, J = 9.2 Hz, 1H), 7.84 (s, 1H), 7.81-7.75 |
| 84 | (m, 3H), 7.61-7.54 (m, 3H), 7.52 (d, J = 2.8 Hz, 1H), 7.44 (dd, J = 9.3, 2.8 Hz, 1H), 6.97 (d, |
| | J = 2.4 Hz, 1H), 6.82 (dd, J = 8.7, 2.4 Hz, 1H), 6.22 (d, J = 8.1 Hz, 1H), 5.81-5.73 (m, 1H), |
| | 4.25-4.13 (m, 2H), 4.11-4.00 (m, 3H), 3.09-2.76 (m, 7H), 2.43-2.34 (m, 1H), 2.27 (d, J = |
| | 7.0 Hz, 2H), 2.24-2.01 (m, 6H), 1.99-1.89 (m, 2H), 1.88-1.77 (m, 1H), 1.39-1.27 (m, 8H), |
| | 1.24 (s, 6H). |
| Example | ¹H NMR (400 MHz, CDCl₃) δ 8.16 (s, 1H), 8.01 (d, J = 9.1 Hz, 1H), 7.79-7.69 (m, 2H), 7.59 |
| 86 | (d, J = 8.7 Hz, 1H), 7.54 (d, J = 2.8 Hz, 1H), 7.52-7.43 (m, 3H), 6.99 (d, J = 2.4 Hz, 1H), |
| | 6.83 (dd, J = 8.7, 2.4 Hz, 1H), 6.21 (d, J = 8.1 Hz, 1H), 5.85-5.74 (m, 1H), 4.82-4.64 (m, |
| | 1H), 4.18 (d, J = 8.1 Hz, 1H), 4.15-4.04 (m, 3H), 4.04-3.95 (m, 1H), 3.23-3.04 (m, 3H), |
| | 3.05-2.77 (m, 4H), 2.70-2.57 (m, 1H), 2.53-2.44 (m, 2H), 2.44-2.34 (m, 1H), 2.11-1.83 (m, |
| | 7H), 1.42-1.31 (m, 2H), 1.29 (s, 6H), 1.25 (s, 6H). |
| Example | ¹H NMR (400 MHz, CDCl₃) δ 8.08 (s, 1H), 8.01 (s, 1H), 7.97 (d, J = 8.9 Hz, 1H), 7.74-7.67 |
| 87 | (m, 2H), 7.57 (d, J = 8.7 Hz, 1H), 7.51-7.44 (m, 2H), 7.05 (d, J = 2.6 Hz, 1H), 7.00-6.92 (m, |
| | 2H), 6.81 (dd, J = 8.7, 2.4 Hz, 1H), 6.18 (d, J = 8.1 Hz, 1H), 5.81-5.71 (m, 1H), 4.75-4.63 |
| | (m, 1H), 4.42-4.22 (m, 4H), 4.19-4.13 (m, 1H), 4.05 (s, 1H), 3.86-3.75 (m, 1H), 3.70-3.57 |
| | (m, 1H), 3.17-3.05 (m, 1H), 3.02-2.76 (m, 4H), 2.73-2.61 (m, 1H), 2.52-2.42 (m, 2H), 2.42- |
| | 2.32 (m, 1H), 2.03-1.82 (m, 2H), 1.40-1.30 (m, 2H), 1.27 (s, 6H), 1.23 (s, 6H). |
| Example | ¹H NMR (400 MHz, CDCl₃) δ 8.21 (s, 1H), 7.97 (d, J = 9.2 Hz, 1H), 7.75-7.67 (m, 2H), 7.57 |
| 88 | (d, J = 8.7 Hz, 1H), 7.51 (d, J = 2.9 Hz, 1H), 7.50-7.45 (m, 2H), 7.43 (dd, J = 9.2, 2.9 Hz, |
| | 1H), 6.97 (d, J = 2.4 Hz, 1H), 6.81 (dd, J = 8.7, 2.4 Hz, 1H), 6.19 (d, J = 8.1 Hz, 1H), 5.84- |
| | 5.69 (m, 1H), 4.16 (d, J = 8.1 Hz, 1H), 4.13-3.95 (m, 3H), 3.10-2.79 (m, 7H), 2.44-2.38 (m, |
| | 2H), 2.38-2.32 (m, 1H), 2.25-2.15 (m, 2H), 2.02-1.87 (m, 4H), 1.87-1.79 (m, 3H), 1.65- |
| | 1.53 (m, 1H), 1.49-1.36 (m, 2H), 1.36-1.29 (m, 2H), 1.27 (s, 6H), 1.23 (s, 6H). |
| Example | ¹H NMR (400 MHz, CDCl₃) δ 8.05-7.97 (m, 2H), 7.76-7.68 (m, 2H), 7.59 (d, J = 8.7 Hz, 1H), |
| 89 | 7.58-7.53 (m, 1H), 7.52-7.43 (m, 2H), 6.99 (d, J = 2.4 Hz, 1H), 6.83 (dd, J = 8.7, 2.4 Hz, |
| | 1H), 6.19 (d, J = 8.2 Hz, 1H), 5.83-5.75 (m, 1H), 4.18 (d, J = 8.1 Hz, 1H), 4.15-4.06 (m, 2H), |
| | 3.11-2.79 (m, 6H), 2.66-2.56 (m, 1H), 2.47-2.37 (m, 2H), 2.31-2.19 (m, 2H), 2.07-1.96 (m, |
| | 2H), 1.96-1.85 (m, 2H), 1.76-1.64 (m, 2H), 1.46-1.39 (m, 2H), 1.30 (s, 6H), 1.25 (s, 6H), |
| | 0.95-0.84 (m, 4H). |
| Example | ¹H NMR (300 MHz, Chloroform-d) δ 8.19 (s, 1H), 7.96 (dd, J = 8.8, 4.5 Hz, 3H), 7.91-7.80 |
| 90 | (m, 3H), 7.58 (d, J = 8.7 Hz, 1H), 7.03 (d, J = 2.6 Hz, 1H), 6.98 (d, J = 2.4 Hz, 1H), 6.94 (dd, |
| | J = 8.9, 2.6 Hz, 1H), 6.82 (dd, J = 8.7, 2.4 Hz, 1H), 6.27 (d, J = 8.1 Hz, 1H), 5.84-5.71 (m, |
| | 1H), 4.65 (d, J = 13.5 Hz, 1H), 4.38-4.22 (m, 4H), 4.08 (s, 1H), 3.86-3.72 (m, 1H), 3.65-3.53 |
| | (m, 1H), 3.14-2.84 (m, 5H), 2.84-2.74 (m, 2H), 2.72-2.57 (m, 1H), 2.45-2.33 (m, 1H), 2.09 (s, |
| | 1H), 1.87 (t, J = 15.3 Hz, 2H), 1.31 (s, 7H), 1.25 (s, 9H). |
| Example | ¹H NMR (300 MHz, Chloroform-d) δ 8.72-8.65 (m, 2H), 8.43 (d, J = 8.7 Hz, 1H), 8.27 (s, 1H), |
| 91 | 7.72 (d, J = 8.9 Hz, 2H), 7.59 (d, J = 8.7 Hz, 1H), 6.99 (d, J = 2.4 Hz, 1H), 6.94 (d, J = 8.9 |
| | Hz, 2H), 6.83 (dd, J = 8.8, 2.4 Hz, 1H), 6.13 (d, J = 8.0 Hz, 1H), 5.92-5.78 (m, 1H), 4.17 (d, |
| | J = 8.1 Hz, 1H), 4.06 (s, 1H), 3.97-3.81 (m, 2H), 3.42-3.34 (m, 3H), 3.07-2.85 (m, 3H), 2.84- |
| | 2.74 (m, 4H), 2.58-2.40 (m, 1H), 1.61 (s, 3H), 1.28 (s, 6H), 1.24 (s, 6H), 0.96-0.80 (m, 2H). |
| Example | ¹H NMR (400 MHz, CDCl₃) δ 8.25 (s, 1H), 8.01 (d, J = 9.0 Hz, 1H), 7.75-7.67 (m, 2H), 7.59 |
| 92 | (d, J = 8.7 Hz, 1H), 7.34-7.29 (m, 2H), 7.24 (d, J = 2.7 Hz, 1H), 7.16 (dd, J = 9.0, 2.7 Hz, |
| | 1H), 6.99 (d, J = 2.4 Hz, 1H), 6.83 (dd, J = 8.7, 2.4 Hz, 1H), 6.20 (d, J = 8.2 Hz, 1H), 5.82- |
| | 5.73 (m, 1H), 4.22-4.14 (m, 1H), 4.11-4.04 (m, 1H), 3.81-3.67 (m, 2H), 3.49-3.37 (m, 2H), |
| | 3.18-3.05 (m, 2H), 3.02-2.92 (m, 2H), 2.92-2.85 (m, 3H), 2.85-2.72 (m, 4H), 2.72-2.65 (m, |
| | 2H), 2.46-2.35 (m, 1H), 1.27 (s, 6H), 1.24 (s, 6H). |
| Example | ¹H NMR (400 MHz, CDCl₃) δ 8.35-8.25 (m, 1H), 8.01 (d, J = 9.1 Hz, 1H), 7.76-7.69 (m, 2H), |
| 93 | 7.59 (d, J = 8.7 Hz, 1H), 7.54 (d, J = 2.8 Hz, 1H), 7.48 (dd, J = 9.1, 2.8 Hz, 1H), 7.36-7.30 |
| | (m, 2H), 6.99 (d, J = 2.4 Hz, 1H), 6.83 (dd, J = 8.7, 2.4 Hz, 1H), 6.26 (d, J = 8.1 Hz, 1H), |
| | 5.83-5.75 (m, 1H), 4.18 (d, J = 8.1 Hz, 1H), 4.15-4.04 (m, 3H), 3.82-3.70 (m, 2H), 3.53- |
| | 3.39 (m, 2H), 3.16-3.04 (m, 2H), 3.04-2.92 (m, 3H), 2.92-2.81 (m, 4H), 2.81-2.70 (m, 4H), |
| | 2.69-2.62 (m, 1H), 2.62-2.52 (m, 2H), 2.44-2.35 (m, 1H), 1.97-1.84 (m, 4H), 1.29 (s, 6H), |
| | 1.24 (s, 6H). |
| Example | ¹H NMR (400 MHz, CDCl₃) δ 8.48 (s, 0.5H), 8.38 (s, 0.5H), 7.99 (d, J = 8.8 Hz, 1H), 7.76- |
| 94 | 7.68 (m, 2H), 7.59 (d, J = 8.7 Hz, 1H), 7.35-7.30 (m, 2H), 7.08-7.04 (m, 1H), 6.99 (d, J = |
| | 2.4 Hz, 1H), 6.96 (dd, J = 8.8, 2.6 Hz, 1H), 6.82 (dd, J = 8.7, 2.4 Hz, 1H), 6.26 (d, J = 8.1 |
| | Hz, 1H), 5.82-5.71 (m, 1H), 4.38-4.30 (m, 2H), 4.30-4.23 (m, 2H), 4.18 (d, J = 8.1 Hz, 1H), |
| | 4.07 (s, 1H), 3.81-3.66 (m, 2H), 3.65-3.50 (m, 2H), 3.33-3.22 (m, 1H), 3.03-2.80 (m, 7H), |
| | 2.80-2.65 (m, 4H), 2.65-2.51 (m, 2H), 2.43-2.34 (m, 1H), 1.29 (s, 6H), 1.25 (s, 6H). |
| Example | ¹H NMR (400 MHz, CDCl₃) δ 8.32 (s, 1H), 8.00-7.93 (m, 1H), 7.76-7.67 (m, 2H), 7.57 (d, |
| 95 | J = 8.7 Hz, 1H), 7.52-7.44 (m, 2H), 7.09-7.03 (m, 1H), 6.99-6.91 (m, 2H), 6.81 (dd, J = 8.7, |
| | 2.4 Hz, 1H), 6.23 (d, J = 8.1 Hz, 1H), 5.82-5.72 (m, 1H), 4.38-4.25 (m, 4H), 4.16 (d, J = 8.1 |
| | Hz, 1H), 4.06 (s, 1H), 4.02-3.92 (m, 1H), 3.86-3.75 (m, 1H), 3.66-3.49 (m, 2H), 3.34-3.23 |
| | (m, 1H), 3.03-2.75 (m, 5H), 2.42-2.32 (m, 1H), 2.02-1.90 (m, 2H), 1.84-1.74 (m, 2H), 1.28 |
| | (s, 6H), 1.23 (s, 6H). |
| Example | ¹H NMR (400 MHz, CDCl₃) δ 8.38 (s, 1H), 7.98 (d, J = 8.8 Hz, 1H), 7.75-7.68 (m, 2H), 7.58 |
| 96 | (d, J = 8.7 Hz, 1H), 7.52-7.45 (m, 2H), 7.05 (d, J = 2.6 Hz, 1H), 6.98 (d, J = 2.4 Hz, 1H), |
| | 6.95 (dd, J = 8.8, 2.6 Hz, 1H), 6.82 (dd, J = 8.7, 2.4 Hz, 1H), 6.22 (d, J = 8.1 Hz, 1H), 5.84- |
| | 5.73 (m, 1H), 4.69-4.57 (m, 1H), 4.38-4.25 (m, 4H), 4.17 (d, J = 8.0 Hz, 1H), 4.07 (s, 1H), |
| | 3.86-3.75 (m, 1H), 3.66-3.54 (m, 1H), 3.14-3.02 (m, 1H), 3.01-2.81 (m, 3H), 2.81-2.71 (m, |
| | 2H), 2.71-2.58 (m, 2H), 2.44-2.34 (m, 1H), 2.32-2.13 (m, 4H), 2.01-1.90 (m, 2H), 1.88- |
| | 1.71 (m, 5H), 1.29 (s, 6H), 1.25 (s, 6H), 1.18-1.05 (m, 2H). |
| Example | ¹H NMR (400 MHz, CDCl₃) δ 8.35 (s, 1H), 8.00 (d, J = 9.1 Hz, 1H), 7.75-7.68 (m, 2H), 7.58 |
| 97 | (d, J = 8.8 Hz, 1H), 7.53 (d, J = 2.9 Hz, 1H), 7.52-7.43 (m, 3H), 6.98 (d, J = 2.4 Hz, 1H), |
| | 6.82 (dd, J = 8.8, 2.4 Hz, 1H), 6.22 (d, J = 8.1 Hz, 1H), 5.83-5.75 (m, 1H), 4.69-4.57 (m, |
| | 1H), 4.17 (d, J = 8.1 Hz, 1H), 4.13-4.02 (m, 3H), 4.00-3.90 (m, 1H), 3.17-3.09 (m, 2H), |
| | 3.09-3.02 (m, 1H), 3.01-2.84 (m, 3H), 2.84-2.70 (m, 3H), 2.68-2.55 (m, 2H), 2.44-2.35 (m, |
| | 1H), 2.31-2.12 (m, 4H), 2.04-1.89 (m, 6H), 1.85-1.72 (m, 5H), 1.29 (s, 6H), 1.25 (s, 6H), |
| | 1.18-1.02 (m, 2H). |
| Example | ¹H NMR (400 MHz, CDCl₃) δ 8.53 (s, 1H), 7.95 (d, J = 8.9 Hz, 1H), 7.75-7.68 (m, 2H), 7.59 |
| 98 | (d, J = 8.7 Hz, 1H), 7.52-7.46 (m, 2H), 7.26 (dd, J = 8.9, 2.6 Hz, 2H), 7.15 (d, J = 2.6 Hz, |
| | 1H), 6.99 (d, J = 2.4 Hz, 1H), 6.83 (dd, J = 8.7, 2.4 Hz, 1H), 6.22 (d, J = 8.1 Hz, 1H), 6.08 (t, |
| | J = 3.9 Hz, 1H), 5.87-5.75 (m, 1H), 4.73-4.59 (m, 1H), 4.17 (d, J = 8.1 Hz, 1H), 4.08 (s, 1H), |
| | 4.05-3.97 (m, 2H), 3.89-3.76 (m, 1H), 3.16-3.03 (m, 1H), 3.03-2.82 (m, 3H), 2.83-2.58 (m, |
| | 4H), 2.45-2.35 (m, 1H), 2.33-2.12 (m, 4H), 2.05-1.91 (m, 3H), 1.91-1.74 (m, 4H), 1.29 (s, |
| | 6H), 1.25 (s, 6H), 1.22-1.06 (m, 2H). |
| Example | ¹H NMR (400 MHz, CDCl₃) δ 8.36 (s, 1H), 7.99 (d, J = 9.1 Hz, 1H), 7.75-7.68 (m, 2H), 7.59 |
| 99 | (d, J = 8.7 Hz, 1H), 7.53 (d, J = 2.8 Hz, 1H), 7.51-7.47 (m, 2H), 7.45 (dd, J = 9.1, 2.8 Hz, |
| | 1H), 6.99 (d, J = 2.4 Hz, 1H), 6.83 (dd, J = 8.7, 2.4 Hz, 1H), 6.22 (d, J = 8.1 Hz, 1H), 5.83- |
| | 5.73 (m, 1H), 4.17 (d, J = 8.1 Hz, 1H), 4.12-4.01 (m, 3H), 3.09-2.97 (m, 3H), 2.97-2.79 (m, |
| | 3H), 2.78-2.59 (m, 2H), 2.43-2.35 (m, 1H), 2.30-2.16 (m, 3H), 2.03-1.88 (m, 5H), 1.88- |
| | 1.74 (m, 3H), 1.38-1.31 (m, 2H), 1.29 (s, 6H), 1.25 (s, 6H). |
| Example | ¹H NMR (300 MHz, Methanol-d₄) δ 8.76 (s, 2H), 7.99 (d, J = 9.2 Hz, 1H), 7.74 (d, J = 8.7 Hz, |
| 100 | 1H), 7.72-7.65 (m, 1H), 7.53 (d, J = 2.8 Hz, 1H), 7.15 (d, J = 2.4 Hz, 1H), 7.04-6.97 (m, 1H), |
| | 5.94-5.86 (m, 1H), 4.29 (s, 1H), 4.21-4.09 (m, 3H), 3.96 (t, J = 5.0 Hz, 4H), 3.08 (t, J = 12.2 |
| | Hz, 2H), 3.00-2.79 (m, 3H), 2.54 (t, J = 5.0 Hz, 4H), 2.41-2.34 (m, 1H), 2.32 (d, J = 6.6 Hz, |
| | 2H), 2.00 (d, J = 12.5 Hz, 3H), 1.30 (s, 6H), 1.23 (s, 6H); LC/MS (ESI) m/z [M + H]+: 823.1 |
| Example | ¹H NMR (400 MHz, Chloroform-d) δ 8.34-8.26 (m, 1H), 7.96 (d, J = 7.1 Hz, 2H), 7.90-7.83 |
| 101 | (m, 3H), 7.59 (d, J = 8.7 Hz, 1H), 7.04 (d, J = 2.1 Hz, 1H), 6.99 (d, J = 2.3 Hz, 1H), 6.96 (dd, |
| | J = 8.9, 2.6 Hz, 1H), 6.84 (dd, J = 8.7, 2.3 Hz, 1H), 6.32 (d, J = 8.1 Hz, 1H), 5.83-5.74 (m, |
| | 1H), 4.71 (d, J = 13.5 Hz, 1H), 4.34-4.31 (m, 2H), 4.20 (d, J = 8.1 Hz, 1H), 4.10 (s, 1H), |
| | 3.90-3.81 (m, 1H), 3.78-3.70 (m, 1H), 3.36-3.14 (m, 2H), 3.02-2.78 (m, 4H), 2.44-2.34 (m, |
| | 1H), 2.28 (d, J = 12.7 Hz, 1H), 2.15 (s, 1H), 1.81-1.70 (m, 2H), 1.32 (s, 6H), 1.27 (s, 6H). |
| Example | ¹H NMR (400 MHz, Chloroform-d) δ 7.97 (dd, J = 22.8, 8.9 Hz, 5H), 7.86 (d, J = 8.6 Hz, 2H), |
| 102 | 7.80 (s, 1H), 7.58 (d, J = 8.7 Hz, 1H), 7.53 (d, J = 2.7 Hz, 1H), 7.46 (dd, J = 9.2, 2.8 Hz, 1H), |
| | 6.98 (d, J = 2.3 Hz, 1H), 6.82 (dd, J = 8.7, 2.4 Hz, 1H), 6.23 (d, J = 8.1 Hz, 1H), 5.80-5.74 |
| | (m, 1H), 4.72 (d, J = 12.1 Hz, 1H), 4.19 (d, J = 8.0 Hz, 1H), 4.07-4.05 (m, 1H), 3.52-3.45 (m, |
| | 1H), 3.35-3.27 (m, 1H), 3.21-3.07 (m, 4H), 2.87-2.80 (m, 3H), 2.42-2.27 (m, 3H), 2.03-1.89 |
| | (m, 4H), 1.81-1.72 (m, 2H), 1.31 (s, 6H), 1.25 (s, 6H), 0.91-0.81 (m, 2H). |
| Example | ¹H NMR (300 MHz, CDCl₃) δ 8.09-7.82 (m, 6H), 7.60 (d, J = 8.6 Hz, 1H), 7.56-7.43 (m, 2H), |
| 104 | 7.00 (d, J = 2.3 Hz, 1H), 6.89-6.80 (m, 1H), 6.26 (d, J = 7.8 Hz, 1H), 5.79 (dd, J = 11.4, 5.3 |
| | Hz, 1H), 4.66 (s, 1H), 4.21 (d, J = 8.2 Hz, 1H), 4.12-3.82 (m, 4H), 3.50 (q, J = 6.9 Hz, 2H), |
| | 3.20-2.73 (m, 8H), 2.62 (d, J = 12.1 Hz, 1H), 2.40 (d, J = 21.0 Hz, 1H), 2.11-1.78 (m, 6H), |
| | 1.33 (s, 6H), 1.27 (s, 6H), 1.25 (s, 1H). |
| Example | ¹H NMR (300 MHz, CDCl₃) δ 8.20 (s, 1H), 8.07 (d, J = 9.1 Hz, 1H), 7.70 (d, J = 8.8 Hz, 2H), |
| 105 | 7.64-7.56 (m, 3H), 7.53 (dd, J = 9.2, 2.9 Hz, 1H), 6.98 (d, J = 2.3 Hz, 1H), 6.92 (d, J = 8.9 |
| | Hz, 2H), 6.83 (dd, J = 8.7, 2.4 Hz, 1H), 6.13 (d, J = 8.1 Hz, 1H), 5.81 (dd, J = 11.4, 5.0 Hz, |
| | 1H), 4.79 (t, J = 11.2 Hz, 1H), 4.06 (s, 1H), 3.78 (s, 2H), 3.50 (q, J = 7.0 Hz, 1H), 3.27 (d, J = |
| | 11.6 Hz, 2H), 3.03-2.80 (m, 4H), 2.72 (s, 3H), 2.41 (d, J = 4.6 Hz, 3H), 2.29-2.14 (m, 3H), |
| | 2.07 (s, 1H), 1.28 (s, 6H), 1.24 (s, 6H), 0.94-0.79 (m, 2H). |
| Example | ¹H NMR (300 MHz, Chloroform-d) δ 8.08 (s, 1H), 7.99 (s, 1H), 7.96 (s, 2H), 7.90 (d, J = 8.7 |
| 106 | Hz, 2H), 7.85 (s, 1H), 7.60 (d, J = 8.7 Hz, 1H), 7.25 (d, J = 2.6 Hz, 1H), 7.15 (d, J = 2.6 Hz, |
| | 1H), 7.00 (d, J = 2.4 Hz, 1H), 6.84 (dd, J = 8.7, 2.4 Hz, 1H), 6.26 (d, J = 8.1 Hz, 1H), 6.02 (s, |
| | 1H), 5.84-5.75 (m, 1H), 4.70 (d, J = 14.1 Hz, 1H), 4.21 (d, J = 8.2 Hz, 1H), 4.10 (s, 1H), 4.01 |
| | (d, J = 3.4 Hz, 2H), 3.83 (d, J = 13.1 Hz, 1H), 3.20-3.03 (m, 2H), 2.96 (d, J = 5.2 Hz, 2H), |
| | 2.91 (d, J = 4.4 Hz, 1H), 2.85-2.70 (m, 4H), 2.41 (s, 1H), 2.01-1.85 (m, 3H), 1.33 (s, 6H), |
| | 1.27 (s, 6H). |
| Example | ¹H NMR (300 MHz, Chloroform-d) δ 8.15 (s, 1H), 8.01-7.94 (m, 3H), 7.91-7.83 (m, 3H), 7.60 |
| 107 | (d, J = 8.7 Hz, 1H), 7.17 (d, J = 2.5 Hz, 1H), 7.00 (d, J = 2.4 Hz, 1H), 6.84 (dd, J = 8.7, 2.4 |
| | Hz, 1H), 6.27 (d, J = 8.3 Hz, 1H), 6.03 (s, 1H), 5.84-5.75 (m, 1H), 4.74 (d, J = 12.6 Hz, 1H), |
| | 4.20 (d, J = 7.8 Hz, 1H), 4.10 (s, 1H), 4.08-4.05 (m, 2H), 4.00-3.91 (m, 1H), 3.41-3.17 (m, |
| | 3H), 3.05-2.86 (m, 4H), 2.43-2.27 (m, 2H), 1.86-1.75 (m, 2H), 1.33 (s, 6H), 1.27 (s, 6H). |
| Example | ¹H NMR (400 MHz, CDCl₃) δ 8.36 (s, 1H), 7.99 (d, J = 9.1 Hz, 1H), 7.74-7.66 (m, 2H), 7.57 |
| 108 | (d, J = 8.7 Hz, 1H), 7.52 (d, J = 2.9 Hz, 1H), 7.45 (dd, J = 9.1, 2.9 Hz, 1H), 7.34-7.28 (m, |
| | 2H), 6.97 (d, J = 2.4 Hz, 1H), 6.81 (dd, J = 8.7, 2.4 Hz, 1H), 6.21 (d, J = 8.1 Hz, 1H), 5.82- |
| | 5.72 (m, 1H), 4.68-4.54 (m, 1H), 4.16 (d, J = 8.1 Hz, 1H), 4.11-4.01 (m, 3H), 3.99-3.87 (m, |
| | 1H), 3.16-3.01 (m, 3H), 3.01-2.75 (m, 6H), 2.70-2.42 (m, 10H), 2.42-2.33 (m, 1H), 2.23 (d, |
| | J = 6.9 Hz, 2H), 2.03-1.69 (m, 8H), 1.27 (s, 6H), 1.23 (s, 6H), 1.19-1.02 (m, 2H). |
| Example | ¹H NMR (400 MHz, CDCl₃) δ 8.47 (s, 1H), 7.98 (d, J = 8.9 Hz, 1H), 7.77-7.69 (m, 2H), 7.59 |
| 109 | (d, J = 8.7 Hz, 1H), 7.37-7.30 (m, 2H), 7.06 (d, J = 2.6 Hz, 1H), 7.02-6.93 (m, 2H), 6.83 (dd, |
| | J = 8.7, 2.4 Hz, 1H), 6.23 (d, J = 8.2 Hz, 1H), 5.83-5.73 (m, 1H), 4.68-4.57 (m, 1H), 4.40- |
| | 4.25 (m, 4H), 4.18 (d, J = 8.2 Hz, 1H), 4.07 (s, 1H), 3.86-3.76 (m, 1H), 3.65-3.53 (m, 1H), |
| | 3.13-2.97 (m, 2H), 2.97-2.81 (m, 4H), 2.75-2.44 (m, 10H), 2.43-2.34 (m, 1H), 2.25 (d, J = |
| | 6.9 Hz, 2H), 1.98-1.74 (m, 4H), 1.29 (s, 6H), 1.25 (s, 6H), 1.19-1.05 (m, 2H). |
| Example | ¹H NMR (400 MHz, CDCl₃) δ 8.63 (s, 1H), 7.95 (d, J = 8.9 Hz, 1H), 7.77-7.68 (m, 2H), 7.59 |
| 110 | (d, J = 8.7 Hz, 1H), 7.36-7.30 (m, 2H), 7.26 (dd, J = 8.9, 2.6 Hz, 1H), 7.15 (d, J = 2.6 Hz, |
| | 1H), 6.98 (d, J = 2.4 Hz, 1H), 6.83 (dd, J = 8.7, 2.4 Hz, 1H), 6.23 (d, J = 8.2 Hz, 1H), 6.13- |
| | 6.01 (m, 1H), 5.86-5.75 (m, 1H), 4.72-4.58 (m, 1H), 4.18 (d, J = 8.1 Hz, 1H), 4.07 (s, 1H), |
| | 4.05-3.94 (m, 2H), 3.87-3.76 (m, 1H), 3.15-3.02 (m, 1H), 3.02-2.79 (m, 5H), 2.79-2.43 (m, |
| | 10H), 2.43-2.35 (m, 1H), 2.25 (d, J = 7.0 Hz, 2H), 1.99-1.76 (m, 4H), 1.29 (s, 6H), 1.25 (s, |
| | 6H), 1.21-1.07 (m, 2H). |
| Example | ¹H NMR (400 MHz, CDCl₃) δ 8.47 (s, 1H), 7.99 (d, J = 9.1 Hz, 1H), 7.77-7.69 (m, 2H), 7.59 |
| 111 | (d, J = 8.7 Hz, 1H), 7.53 (d, J = 2.8 Hz, 1H), 7.45 (dd, J = 9.1, 2.8 Hz, 1H), 7.37-7.30 (m, |
| | 2H), 6.99 (d, J = 2.4 Hz, 1H), 6.83 (dd, J = 8.7, 2.4 Hz, 1H), 6.23 (d, J = 8.1 Hz, 1H), 5.83- |
| | 5.73 (m, 1H), 4.18 (d, J = 8.1 Hz, 1H), 4.10-4.00 (m, 3H), 3.08-2.81 (m, 7H), 2.75-2.44 (m, |
| | 9H), 2.43-2.34 (m, 1H), 2.26 (d, J = 7.0 Hz, 2H), 1.99-1.77 (m, 4H), 1.39-1.30 (m, 2H), 1.29 |
| | (s, 6H), 1.25 (s, 6H). |
| Example | ¹H NMR (400 MHz, CDCl₃) δ 8.08 (s, 1H), 7.98 (d, J = 8.9 Hz, 1H), 7.86 (s, 1H), 7.83-7.76 |
| 112 | (m, 3H), 7.63-7.55 (m, 3H), 7.28-7.24 (m, 1H), 7.17 (d, J = 2.6 Hz, 1H), 7.00 (d, J = 2.4 Hz, |
| | 1H), 6.84 (dd, J = 8.7, 2.4 Hz, 1H), 6.23 (d, J = 8.1 Hz, 1H), 6.07-6.01 (m, 1H), 5.84-5.75 |
| | (m, 1H), 4.73-4.62 (m, 1H), 4.25-4.15 (m, 2H), 4.09 (s, 1H), 4.05-3.97 (m, 2H), 3.88-3.77 |
| | (m, 1H), 3.18-3.09 (m, 1H), 3.08-2.72 (m, 6H), 2.48-2.37 (m, 1H), 2.29 (d, J = 7.0 Hz, 2H), |
| | 2.26-2.04 (m, 6H), 2.04-1.88 (m, 2H), 1.88-1.77 (m, 1H), 1.32 (s, 6H), 1.27 (s, 6H), 0.94- |
| | 0.80 (m, 2H). |
| Example | ¹H NMR (400 MHz, CDCl₃) δ 8.24-8.14 (m, 2H), 7.85 (s, 1H), 7.83-7.75 (m, 3H), 7.71-7.64 |
| 113 | (m, 2H), 7.62-7.54 (m, 3H), 7.00 (d, J = 2.4 Hz, 1H), 6.84 (dd, J = 8.7, 2.4 Hz, 1H), 6.24 (d, |
| | J = 8.1 Hz, 1H), 5.86-5.77 (m, 1H), 4.92 (s, 2H), 4.66-4.56 (m, 1H), 4.26-4.15 (m, 2H), 4.09 |
| | (s, 1H), 3.90-3.80 (m, 1H), 3.20-3.08 (m, 1H), 3.08-2.79 (m, 5H), 2.75-2.65 (m, 1H), 2.47- |
| | 2.38 (m, 1H), 2.33-2.24 (m, 2H), 2.23-2.08 (m, 7H), 2.01-1.92 (m, 1H), 1.92-1.76 (m, 3H), |
| | 1.31 (s, 6H), 1.26 (s, 6H). |
| Example | ¹H NMR (400 MHz, CDCl₃) δ 8.95 (d, J = 2.3 Hz, 1H), 8.40 (s, 1H), 8.16-8.07 (m, 2H), 8.04 |
| 114 | (s, 1H), 7.98 (d, J = 8.8 Hz, 1H), 7.59 (d, J = 8.7 Hz, 1H), 7.56 (d, J = 8.2 Hz, 1H), 7.06 (d, |
| | J = 2.6 Hz, 1H), 6.99 (d, J = 2.4 Hz, 1H), 6.96 (dd, J = 8.8, 2.6 Hz, 1H), 6.83 (dd, J = 8.7, 2.4 |
| | Hz, 1H), 6.26 (d, J = 8.1 Hz, 1H), 5.84-5.74 (m, 1H), 4.70-4.59 (m, 1H), 4.41-4.25 (m, 4H), |
| | 4.25-4.17 (m, 2H), 4.08 (s, 1H), 3.81 (p, J = 7.4 Hz, 1H), 3.67-3.56 (m, 1H), 3.16-2.78 (m, |
| | 6H), 2.75-2.62 (m, 1H), 2.46-2.35 (m, 1H), 2.34-2.03 (m, 8H), 2.00-1.91 (m, 1H), 1.90- |
| | 1.76 (m, 2H), 1.30 (s, 6H), 1.26 (s, 6H), 1.20-1.05 (m, 2H). |
| Example | ¹H NMR (400 MHz, CDCl₃) δ 8.96 (d, J = 2.3 Hz, 1H), 8.56 (s, 1H), 8.14 (s, 1H), 8.11 (dd, |
| 115 | J = 8.3, 2.3 Hz, 1H), 8.04 (s, 1H), 7.99 (d, J = 9.1 Hz, 1H), 7.63-7.50 (m, 3H), 7.46 (dd, J = |
| | 9.1, 2.9 Hz, 1H), 6.98 (d, J = 2.4 Hz, 1H), 6.82 (dd, J = 8.8, 2.4 Hz, 1H), 6.27 (d, J = 8.2 Hz, |
| | 1H), 5.86-5.74 (m, 1H), 4.27-4.15 (m, 2H), 4.11-4.01 (m, 3H), 3.12-2.99 (m, 4H), 2.99- |
| | 2.79 (m, 3H), 2.45-2.35 (m, 1H), 2.34-2.26 (m, 2H), 2.26-2.08 (m, 6H), 2.00-1.90 (m, 2H), |
| | 1.89-1.80 (m, 1H), 1.39-1.28 (m, 8H), 1.25 (s, 6H). |
| Example | ¹H NMR (300 MHz, CDCl₃) δ 8.22 (s, 1H), 7.99 (d, J = 9.1 Hz, 1H), 7.76-7.67 (m, 2H), 7.57 |
| 116 | (d, J = 8.7 Hz, 1H), 7.54-7.40 (m, 4H), 6.97 (d, J = 2.4 Hz, 1H), 6.81 (dd, J = 8.7, 2.4 Hz, |
| | 1H), 6.20 (d, J = 8.1 Hz, 1H), 5.82-5.71 (m, 1H), 4.72-4.55 (m, 1H), 4.15 (d, J = 8.1 Hz, |
| | 1H), 4.12-4.00 (m, 3H), 4.00-3.88 (m, 1H), 3.19-2.73 (m, 9H), 2.69-2.50 (m, 2H), 2.50- |
| | 2.30 (m, 4H), 2.14-1.73 (m, 12H), 1.27 (s, 6H), 1.23 (s, 6H), 1.16-0.99 (m, 2H), 0.95-0.78 |
| | (m, 2H). |
| Example | ¹H NMR (400 MHz, CDCl₃) δ 8.29-8.18 (m, 1H), 7.99 (d, J = 8.8 Hz, 1H), 7.77-7.68 (m, 2H), |
| 117 | 7.59 (d, J = 8.7 Hz, 1H), 7.54-7.45 (m, 2H), 7.06 (d, J = 2.6 Hz, 1H), 6.99 (d, J = 2.4 Hz, |
| | 1H), 6.96 (dd, J = 8.8, 2.6 Hz, 1H), 6.83 (dd, J = 8.7, 2.4 Hz, 1H), 6.22 (d, J = 8.1 Hz, 1H), |
| | 5.82-5.73 (m, 1H), 4.68-4.58 (m, 1H), 4.40-4.24 (m, 4H), 4.18 (d, J = 8.0 Hz, 1H), 4.08 (s, |
| | 1H), 3.86-3.74 (m, 1H), 3.66-3.54 (m, 1H), 3.14-3.03 (m, 1H), 3.03-2.78 (m, 5H), 2.74- |
| | 2.61 (m, 1H), 2.48-2.34 (m, 3H), 2.29-2.15 (m, 2H), 2.03-1.91 (m, 2H), 1.90-1.74 (m, 4H), |
| | 1.52-1.36 (m, 2H), 1.29 (s, 6H), 1.25 (s, 6H), 1.20-1.03 (m, 2H). |
| Example | ¹H NMR (400 MHz, CDCl₃) δ 8.48-8.30 (m, 1H), 7.93 (d, J = 8.9 Hz, 1H), 7.75-7.66 (m, 2H), |
| 118 | 7.57 (d, J = 8.7 Hz, 1H), 7.51-7.42 (m, 2H), 7.24 (dd, J = 8.9, 2.7 Hz, 1H), 7.17-7.10 (m, |
| | 1H), 6.97 (d, J = 2.4 Hz, 1H), 6.81 (dd, J = 8.7, 2.4 Hz, 1H), 6.20 (d, J = 8.2 Hz, 1H), 6.09- |
| | 6.00 (m, 1H), 5.81-5.69 (m, 1H), 4.69-4.57 (m, 1H), 4.16 (d, J = 8.1 Hz, 1H), 4.06 (s, 1H), |
| | 4.02-3.91 (m, 2H), 3.85-3.72 (m, 1H), 3.16-3.01 (m, 1H), 3.01-2.79 (m, 5H), 2.77-2.65 (m, |
| | 1H), 2.46-2.32 (m, 3H), 2.19 (d, J = 6.9 Hz, 2H), 2.01-1.86 (m, 4H), 1.86-1.75 (m, 4H), |
| | 1.47-1.35 (m, 2H), 1.27 (s, 6H), 1.23 (s, 6H), 1.17-1.09 (m, 2H). |
| Example | ¹H NMR (400 MHz, CDCl₃) δ 8.29 (s, 1H), 8.17-8.10 (m, 1H), 7.73-7.66 (m, 2H), 7.66-7.60 |
| 119 | (m, 2H), 7.57 (d, J = 8.7 Hz, 1H), 7.50-7.43 (m, 2H), 6.97 (d, J = 2.4 Hz, 1H), 6.81 (dd, J = |
| | 8.7, 2.4 Hz, 1H), 6.19 (d, J = 8.1 Hz, 1H), 5.83-5.73 (m, 1H), 4.90 (s, 2H), 4.62-4.50 (m, |
| | 1H), 4.16 (d, J = 8.1 Hz, 1H), 4.06 (s, 1H), 3.86-3.74 (m, 1H), 3.18-3.04 (m, 1H), 3.02-2.80 |
| | (m, 5H), 2.73-2.60 (m, 1H), 2.46-2.33 (m, 3H), 2.20 (d, J = 6.7 Hz, 2H), 2.06-1.88 (m, 4H), |
| | 1.88-1.71 (m, 4H), 1.50-1.34 (m, 2H), 1.27 (s, 6H), 1.23 (s, 6H), 1.16-0.99 (m, 2H). |
| Example | ¹H NMR (300 MHz, Methanol-d₄) δ 8.65 (s, 1H), 8.06 (s, 4H), 8.02 (d, J = 9.2 Hz, 1H), 7.75 |
| 120 | (d, J = 8.7 Hz, 1H), 7.70 (dd, J = 9.2, 2.8 Hz, 1H), 7.55 (d, J = 2.8 Hz, 1H), 7.17 (d, J = 2.4 |
| | Hz, 1H), 7.05-6.98 (m, 1H), 5.95-5.86 (m, 1H), 4.33 (s, 1H), 4.22 (s, 1H), 3.92-3.85 (m, 2H), |
| | 3.62-3.52 (m, 4H), 2.94-2.83 (m, 3H), 2.83-2.74 (m, 4H), 2.40-2.31 (m, 1H), 1.34 (s, 6H), |
| | 1.27 (s, 6H). |
| Example | ¹H NMR (300 MHz, Chloroform-d) δ 8.13 (s, 1H), 8.05-7.87 (m, 7H), 7.60 (d, J = 8.7 Hz, 1H), |
| 121 | 7.54 (d, J = 2.8 Hz, 1H), 7.47 (dd, J = 9.2, 2.8 Hz, 1H), 7.00 (d, J = 2.4 Hz, 1H), 6.84 (dd, J = |
| | 8.7, 2.4 Hz, 1H), 6.27 (d, J = 8.1 Hz, 1H), 5.84-5.73 (m, 1H), 4.21 (d, J = 8.1 Hz, 1H), 4.10 |
| | (s, 1H), 3.85 (s, 2H), 3.65 (d, J = 28.6 Hz, 4H), 3.17-3.04 (m, 2H), 3.03-2.93 (m, 2H), 2.92- |
| | 2.73 (m, 3H), 2.63 (d, J = 23.3 Hz, 4H), 2.44-2.34 (m, 1H), 2.02-1.85 (m, 4H), 1.33 (s, 6H), |
| | 1.27 (s, 6H). |
| Example | ¹H NMR (300 MHz, Chloroform-d) δ 8.13 (s, 1H), 8.05-7.88 (m, 7H), 7.60 (d, J = 8.7 Hz, 1H), |
| 122 | 7.06 (d, J = 2.6 Hz, 1H), 7.00 (d, J = 2.4 Hz, 1H), 6.96 (dd, J = 8.9, 2.6 Hz, 1H), 6.84 (dd, J = |
| | 8.7, 2.4 Hz, 1H), 6.27 (d, J = 7.9 Hz, 1H), 5.82-5.74 (m, 1H), 4.24-4.19 (m, 1H), 4.10 (s, 1H), |
| | 3.85 (s, 2H), 3.84-3.70 (m, 4H), 3.54-3.46 (m, 1H), 3.44-3.39 (m, 2H), 3.04-2.81 (m, 4H), |
| | 2.68-2.57 (m, 4H), 2.45-2.35 (m, 1H), 1.33 (s, 6H), 1.28 (s, 6H). |
| Example | ¹H NMR (300 MHz, Chloroform-d) δ 8.11 (s, 1H), 8.05-7.87 (m, 5H), 7.60 (d, J = 8.7 Hz, 1H), |
| 123 | 7.28-7.23 (m, 4H), 7.15 (d, J = 2.6 Hz, 1H), 7.00 (d, J = 2.4 Hz, 1H), 6.84 (dd, J = 8.8, 2.4 |
| | Hz, 1H), 6.27 (d, J = 8.2 Hz, 1H), 6.00-5.95 (m, 1H), 5.84-5.75 (m, 1H), 4.21 (d, J = 8.1 Hz, |
| | 1H), 4.10 (s, 1H), 4.02 (d, J = 3.5 Hz, 1H), 3.86 (s, 1H), 3.83-3.75 (m, 2H), 3.71-3.65 (m, |
| | 1H), 3.58-3.45 (m, 3H), 2.97-2.93 (m, 1H), 2.90-2.80 (m, 1H), 2.75-2.52 (m, 4H), 2.46-2.35 |
| | (m, 1H), 1.33 (s, 6H), 1.28 (s, 6H). |
| Example | ¹H NMR (500 MHz, CDCl₃) δ 0 8.92 (dd, J = 2.4, 0.8 Hz, 1H), 8.18 (s, 1H), 8.12-8.07 (m, |
| 124 | 2H), 8.02 (d, J = 0.8 Hz, 1H), 8.00 (d, J = 9.1 Hz, 1H), 7.58 (d, J = 8.7 Hz, 1H), 7.55 (dd, J = |
| | 8.3, 0.8 Hz, 1H), 7.52 (d, J = 2.9 Hz, 1H), 7.45 (dd, J = 9.1, 2.9 Hz, 1H), 6.97 (d, J = 2.4 Hz, |
| | 1H), 6.81 (dd, J = 8.7, 2.4 Hz, 1H), 6.21 (d, J = 8.1 Hz, 1H), 5.81-5.74 (m, 1H), 4.68-4.59 |
| | (m, 1H), 4.23-4.14 (m, 2H), 4.11-4.01 (m, 3H), 4.00-3.91 (m, 1H), 3.15-3.05 (m, 3H), 3.05- |
| | 2.90 (m, 4H), 2.88-2.77 (m, 2H), 2.63-2.54 (m, 1H), 2.43-2.35 (m, 1H), 2.25 (d, J = 6.9 Hz, |
| | 2H), 2.23-2.16 (m, 3H), 2.16-2.04 (m, 3H), 2.02-1.90 (m, 3H), 1.90-1.84 (m, 2H), 1.84- |
| | 1.74 (m, 2H), 1.29 (s, 6H), 1.24 (s, 6H), 1.18-1.05 (m, 2H). |
| Example | ¹H NMR (400 MHz, CDCl₃) δ 8.94 (d, J = 2.4 Hz, 1H), 8.31 (s, 1H), 8.14-8.09 (m, 2H), 8.05 |
| 125 | (s, 1H), 7.97 (d, J = 8.9 Hz, 1H), 7.60 (d, J = 8.7 Hz, 1H), 7.59-7.56 (m, 1H), 7.27 (dd, J = |
| | 8.9, 2.7 Hz, 1H), 7.16 (d, J = 2.7 Hz, 1H), 7.00 (d, J = 2.4 Hz, 1H), 6.84 (dd, J = 8.7, 2.4 Hz, |
| | 1H), 6.24 (d, J = 8.2 Hz, 1H), 6.06 (t, J = 3.8 Hz, 1H), 5.85-5.76 (m, 1H), 4.72-4.63 (m, 1H), |
| | 4.25-4.16 (m, 2H), 4.09 (s, 1H), 4.02 (d, J = 3.7 Hz, 2H), 3.88-3.79 (m, 1H), 3.17-3.08 (m, |
| | 1H), 3.07-2.95 (m, 4H), 2.95-2.82 (m, 1H), 2.80-2.71 (m, 1H), 2.45-2.38 (m, 1H), 2.28 (d, |
| | J = 7.1 Hz, 2H), 2.26-2.07 (m, 6H), 2.02-1.95 (m, 1H), 1.94-1.87 (m, 1H), 1.86-1.80 (m, 1H), |
| | 1.31 (s, 6H), 1.26 (s, 6H), 1.23-1.12 (m, 2H). |
| Example | ¹H NMR (400 MHz, CDCl₃) δ 8.90 (d, J = 2.3 Hz, 1H), 8.05 (dd, J = 8.1, 2.3 Hz, 1H), 7.99 (d, |
| 126 | J = 9.1 Hz, 1H), 7.57 (d, J = 8.7 Hz, 1H), 7.52 (d, J = 2.9 Hz, 1H), 7.50-7.41 (m, 2H), 6.97 |
| | (d, J = 2.4 Hz, 1H), 6.81 (dd, J = 8.7, 2.4 Hz, 1H), 6.18 (d, J = 8.1 Hz, 1H), 5.82-5.70 (m, |
| | 1H), 4.68-4.54 (m, 1H), 4.16 (d, J = 8.0 Hz, 1H), 4.11-4.01 (m, 3H), 3.97-3.87 (m, 1H), |
| | 3.15-3.04 (m, 2H), 3.01-2.91 (m, 2H), 2.91-2.75 (m, 4H), 2.64-2.53 (m, 1H), 2.48-2.34 (m, |
| | 3H), 2.18 (d, J = 6.8 Hz, 2H), 2.02-1.91 (m, 4H), 1.91-1.80 (m, 5H), 1.80-1.69 (m, 3H), |
| | 1.69-1.58 (m, 1H), 1.49-1.34 (m, 3H), 1.28 (s, 6H), 1.23 (s, 6H), 1.17-1.01 (m, 2H). |
| Example | ¹H NMR (400 MHz, CDCl₃) δ 8.90 (d, J = 2.3 Hz, 1H), 8.13 (s, 1H), 8.05 (dd, J = 8.1, 2.3 Hz, |
| 127 | 1H), 7.97 (d, J = 8.8 Hz, 1H), 7.57 (d, J = 8.7 Hz, 1H), 7.47 (d, J = 8.1 Hz, 1H), 7.04 (d, J = |
| | 2.6 Hz, 1H), 6.97 (d, J = 2.4 Hz, 1H), 6.94 (dd, J = 8.8, 2.6 Hz, 1H), 6.81 (dd, J = 8.7, 2.4 |
| | Hz, 1H), 6.18 (d, J = 8.1 Hz, 1H), 5.81-5.71 (m, 1H), 4.66-4.54 (m, 1H), 4.39-4.22 (m, 4H), |
| | 4.16 (d, J = 8.0 Hz, 1H), 4.06 (s, 1H), 3.78 (p, J = 7.3 Hz, 1H), 3.62-3.52 (m, 1H), 3.11-3.00 |
| | (m, 1H), 3.00-2.79 (m, 4H), 2.70-2.59 (m, 1H), 2.43 (d, J = 6.6 Hz, 2H), 2.41-2.33 (m, 1H), |
| | 2.18 (d, J = 6.9 Hz, 2H), 2.05-1.88 (m, 3H), 1.87-1.72 (m, 5H), 1.69-1.58 (m, 1H), 1.50- |
| | 1.35 (m, 2H), 1.28 (s, 6H), 1.24 (s, 6H), 1.17-1.03 (m, 2H). |
| Example | ¹H NMR (400 MHz, CDCl₃) δ 8.90 (d, J = 2.3 Hz, 1H), 8.15 (s, 1H), 8.05 (dd, J = 8.1, 2.3 Hz, |
| 128 | 1H), 7.94 (d, J = 8.8 Hz, 1H), 7.57 (d, J = 8.7 Hz, 1H), 7.47 (d, J = 8.1 Hz, 1H), 7.24 (dd, J = |
| | 8.8, 2.6 Hz, 1H), 7.13 (d, J = 2.6 Hz, 1H), 6.97 (d, J = 2.4 Hz, 1H), 6.81 (dd, J = 8.7, 2.4 Hz, |
| | 1H), 6.17 (d, J = 8.1 Hz, 1H), 6.02 (d, J = 3.9 Hz, 1H), 5.82-5.73 (m, 1H), 4.68-4.57 (m, |
| | 1H), 4.16 (d, J = 8.0 Hz, 1H), 4.07 (s, 1H), 4.01-3.93 (m, 2H), 3.83-3.71 (m, 1H), 3.15-3.03 |
| | (m, 1H), 3.02-2.91 (m, 2H), 2.91-2.80 (m, 3H), 2.76-2.67 (m, 1H), 2.49-2.34 (m, 3H), 2.18 |
| | (d, J = 7.0 Hz, 2H), 2.01-1.90 (m, 3H), 1.89-1.73 (m, 4H), 1.49-1.36 (m, 2H), 1.28 (s, 6H), |
| | 1.24 (s, 1H), 1.18-1.07 (m, 2H). |
| Example | ¹H NMR (400 MHz, CDCl₃) δ 8.90 (d, J = 2.3 Hz, 1H), 8.16-8.02 (m, 2H), 7.97 (d, J = 9.2 |
| 129 | Hz, 1H), 7.58 (d, J = 8.7 Hz, 1H), 7.51 (d, J = 2.9 Hz, 1H), 7.47 (d, J = 8.1 Hz, 1H), 7.43 (dd, |
| | J = 9.2, 2.9 Hz, 1H), 6.97 (d, J = 2.4 Hz, 1H), 6.81 (dd, J = 8.7, 2.4 Hz, 1H), 6.18 (d, J = 8.1 |
| | Hz, 1H), 5.81-5.71 (m, 1H), 4.16 (d, J = 8.0 Hz, 1H), 4.10-3.98 (m, 3H), 3.09-2.77 (m, 7H), |
| | 2.44 (d, J = 6.6 Hz, 2H), 2.41-2.34 (m, 1H), 2.20 (d, J = 7.0 Hz, 2H), 2.02-1.76 (m, 7H), |
| | 1.64-1.60 (m, 1H), 1.50-1.37 (m, 2H), 1.35-1.29 (m, 2H), 1.28 (s, 6H), 1.24 (s, 6H). |
| Example | ¹H NMR (500 MHz, CDCl₃) δ 8.91 (s, 1H), 8.28 (s, 1H), 8.05 (dd, J = 8.1, 2.3 Hz, 1H), 7.99 |
| 130 | (d, J = 9.1 Hz, 1H), 7.58 (d, J = 8.7 Hz, 1H), 7.52 (d, J = 2.8 Hz, 1H), 7.47 (d, J = 8.1 Hz, |
| | 1H), 7.45 (dd, J = 9.1, 2.8 Hz, 1H), 6.97 (d, J = 2.4 Hz, 1H), 6.81 (dd, J = 8.7, 2.4 Hz, 1H), |
| | 6.21 (d, J = 8.2 Hz, 1H), 5.82-5.73 (m, 1H), 4.68-4.56 (m, 1H), 4.16 (d, J = 8.0 Hz, 1H), |
| | 4.11-4.00 (m, 3H), 4.00-3.88 (m, 1H), 3.16-3.02 (m, 3H), 3.02-2.88 (m, 2H), 2.88-2.76 (m, |
| | 3H), 2.76-2.63 (m, 2H), 2.63-2.52 (m, 1H), 2.44-2.34 (m, 1H), 2.27-2.11 (m, 3H), 2.04- |
| | 1.89 (m, 5H), 1.89-1.72 (m, 7H), 1.28 (s, 6H), 1.23 (s, 6H), 1.17-1.02 (m, 2H). |
| Example | ¹H NMR (400 MHz, CDCl₃) δ 8.92 (d, J = 2.2 Hz, 1H), 8.13 (s, 1H), 8.07 (dd, J = 8.1, 2.2 Hz, |
| 131 | 1H), 8.03-7.96 (m, 1H), 7.63-7.57 (m, 1H), 7.50 (d, J = 8.1 Hz, 1H), 7.09-7.05 (m, 1H), |
| | 7.00-6.98 (m, 1H), 6.98-6.95 (m, 1H), 6.86-6.80 (m, 1H), 6.20 (d, J = 8.1 Hz, 1H), 5.83- |
| | 5.76 (m, 1H), 4.68-4.59 (m, 1H), 4.40-4.26 (m, 4H), 4.18 (d, J = 8.0 Hz, 1H), 4.09 (s, 1H), |
| | 3.82 (q, J = 7.3 Hz, 1H), 3.65-3.56 (m, 1H), 3.14-3.03 (m, 1H), 3.03-2.84 (m, 3H), 2.83- |
| | 2.72 (m, 2H), 2.72-2.62 (m, 2H), 2.47-2.37 (m, 1H), 2.31-2.12 (m, 4H), 2.04-1.90 (m, 3H), |
| | 1.90-1.73 (m, 4H), 1.30 (s, 6H), 1.26 (s, 6H), 1.19-1.05 (m, 2H). |
| Example | ¹H NMR (500 MHz, CDCl₃) δ 8.90 (d, J = 2.3 Hz, 1H), 8.13 (s, 1H), 8.05 (dd, J = 8.2, 2.3 Hz, |
| 132 | 1H), 7.95 (d, J = 8.9 Hz, 1H), 7.58 (d, J = 8.7 Hz, 1H), 7.48 (d, J = 8.2 Hz, 1H), 7.24 (dd, J = |
| | 8.9, 2.6 Hz, 1H), 7.14 (d, J = 2.6 Hz, 1H), 6.97 (d, J = 2.3 Hz, 1H), 6.81 (dd, J = 8.8, 2.3 Hz, |
| | 1H), 6.18 (d, J = 8.1 Hz, 1H), 6.06-5.99 (m, 1H), 5.82-5.75 (m, 1H), 4.68-4.59 (m, 1H), 4.16 |
| | (d, J = 8.0 Hz, 1H), 4.07 (s, 1H), 4.03-3.96 (m, 2H), 3.83-3.75 (m, 1H), 3.14-3.05 (m, 1H), |
| | 3.03-2.80 (m, 3H), 2.79-2.62 (m, 4H), 2.44-2.35 (m, 1H), 2.27-2.13 (m, 4H), 2.02-1.91 (m, |
| | 3H), 1.91-1.75 (m, 4H), 1.28 (s, 6H), 1.24 (s, 6H), 1.19-1.08 (m, 2H). |
| Example | ¹H NMR (500 MHz, CDCl₃) δ 8.90 (d, J = 2.3 Hz, 1H), 8.18-8.08 (m, 2H), 8.05 (dd, J= 8.1, |
| 133 | 2.3 Hz, 1H), 7.67-7.61 (m, 2H), 7.58 (d, J = 8.7 Hz, 1H), 7.48 (d, J = 8.1 Hz, 1H), 6.97 (d, |
| | J = 2.4 Hz, 1H), 6.81 (dd, J = 8.7, 2.4 Hz, 1H), 6.18 (d, J = 8.1 Hz, 1H), 5.84-5.75 (m, 1H), |
| | 4.95-4.85 (m, 2H), 4.61-4.52 (m, 1H), 4.16 (d, J = 8.0 Hz, 1H), 4.07 (s, 1H), 3.84-3.76 (m, |
| | 1H), 3.17-3.07 (m, 1H), 3.07-2.79 (m, 3H), 2.79-2.70 (m, 2H), 2.70-2.61 (m, 2H), 2.43- |
| | 2.36 (m, 1H), 2.28-2.12 (m, 4H), 2.01-1.87 (m, 3H), 1.87-1.73 (m, 4H), 1.28 (s, 6H), 1.24 |
| | (s, 6H), 1.20-1.04 (m, 2H). |
| Example | (400 MHz, CDCl3) δ 8.23 (d, J = 3.8 Hz, 1H), 8.14 (dd, J = 9.2, 4.0 Hz, 1H), 7.76-7.70 (m, |
| 135 | 2H), 7.64 (m, 2H), 7.59 (d, J = 8.7 Hz, 1H), 6.99 (d, J = 2.4 Hz, 1H), 6.83 (dd, J = 8.7, 2.4 |
| | Hz, 1H), 6.57 (dd, J = 8.8, 3.1 Hz, 2H), 6.16 (dd, J = 8.1, 5.7 Hz, 1H), 5.80 (m, 1H), 4.85 (s, |
| | 2H), 4.18 (d, J = 8.1 Hz, 1H), 4.07 (s, 1H), 3.88 (m, 2H), 3.72-3.61 (m, 3H), 3.53 (m, 1H), |
| | 3.44-3.19 (m, 4H), 3.12 (m, 1H), 3.04-2.85 (m, 3H), 2.40 (m, 1H), 2.07 (m, 1H), 1.29 (s, 6H), |
| | 1.24 (s, 6H). |
| Example | (300 MHz, CDCl3) δ 8.30 (d, J = 3.1 Hz, 1H), 7.95 (d, J = 8.9 Hz, 1H), 7.73 (d, J = 1.9 Hz, |
| 136 | 2H), 7.58 (d, J = 8.7 Hz, 1H), 7.26 (dd, J = 8.9, 2.7 Hz, 1H), 7.15 (d, J = 2.6 Hz, 1H), 6.98 (d, |
| | J = 2.4 Hz, 1H), 6.82 (dd, J = 8.7, 2.4 Hz, 1H), 6.58 (d, J = 8.8 Hz, 2H), 6.14 (d, J = 8.1 Hz, |
| | 1H), 5.90 (t, J = 3.9 Hz, 1H), 5.79 (m, 1H), 4.17 (d, J = 8.0 Hz, 1H), 4.06 (s, 1H), 3.97-3.80 |
| | (m, 4H), 3.74-3.61 (m, 3H), 3.53-3.44 (m, 2H), 3.40-3.16 (m, 3H), 3.01-2.81 (m, 3H), 2.44- |
| | 2.34 (m, 1H), 1.28 (s, 6H), 1.23 (s, 6H). |
| Example | (400 MHz, CDCl3) δ 8.19 (s, 1H), 8.13 (d, J = 9.1 Hz, 1H), 7.68 (d, J = 8.4 Hz, 2H), 7.62 (m, |
| 137 | 2H), 7.56 (d, J = 8.7 Hz, 1H), 6.96 (d, J=2.4 Hz, 1H), 6.81 (dd, J = 8.7, 2.4 Hz, 1H), 6.61 (d, |
| | J = 8.4 Hz, 2H), 6.10 (d, J = 8.1 Hz, 1H), 5.74 (m, 1H), 4.88 (s, 2H), 4.54 (d, J = 13.9 Hz, |
| | 1H), 4.15 (d, J = 8.1 Hz, 1H), 4.04 (s, 1H), 3.79 (d, J = 13.4 Hz, 1H), 3.56 (m, 2H), 3.21 (m, |
| | 2H), 3.14-2.91 (m, 5H), 2.88-2.60 (m, 5H), 2.57-2.30 (m, 5H), 1.83 (m, 3H), 1.26 (s, 6H), |
| | 1.22 (s, 6H). |
| Example | (400 MHz, CDCl3) δ 8.51 (m, 1H), 7.93 (d, J = 8.9 Hz, 1H), 7.69 (d, J = 8.4 Hz, 2H), 7.56 (d, |
| 138 | J = 8.7 Hz, 1H), 7.22 (m, 1H), 7.19-7.04 (m, 1H), 6.97 (d, J = 2.3 Hz, 1H), 6.81 (dd, J = 8.7, |
| | 2.3 Hz, 1H), 6.61 (m, 2H), 6.15 (dd, J = 8.2, 4.1 Hz, 1H), 6.06 (d, J = 16.5 Hz, 1H), 5.75-5.56 |
| | (m, 1H), 4.61 (d, J = 13.3 Hz, 1H), 4.16 (d, J = 8.1 Hz, 1H), 4.06 (d, J = 2.8 Hz, 1H), 3.97 (d, |
| | J = 3.8 Hz, 2H), 3.77 (d, J = 13.6 Hz, 1H), 3.53 (m, 2H), 3.25 (m, 2H), 3.14-2.48 (m, 12H), |
| | 2.34 (m, 3H), 1.89 (m, 2H), 1.27 (s, 6H), 1.22 (s, 6H), 1.12 (m, 3H). |
| Example | (500 MHz, CDCl3) δ 8.31 (d, J = 15.4 Hz, 1H), 7.98 (d, J = 9.2 Hz, 1H), 7.69 (d, J = 8.3 Hz, |
| 139 | 2H), 7.56 (d, J = 8.7 Hz, 1H), 7.51 (m, 1H), 7.43 (dd, J = 9.2, 2.8 Hz, 1H), 6.96 (m, 1H), 6.80 |
| | (m, 1H), 6.61 (d, J = 8.3 Hz, 2H), 6.13 (d, J = 8.2 Hz, 1H), 5.69 (m, 1H), 4.60 (d, J = 13.3 Hz, |
| | 1H), 4.15 (d, J = 8.1 Hz, 1H), 4.08-4.01 (m, 3H), 3.91 (d, J = 13.3 Hz, 1H), 3.70 (s, 1H), 3.56 |
| | (m, 2H), 3.22 (m, 2H), 3.08 (m, 3H), 3.02-2.85 (m, 4H), 2.80 (m, 2H), 2.67 (m, 2H), 2.57 (m, |
| | 3H), 2.34 (m, 3H), 1.85 (m, 7H), 1.26 (s, 6H), 1.22 (s, 6H), 1.09 (m, 2H). |
| Example | (500 MHz, CDCl3) δ 8.48 (d, J = 39.8 Hz, 1H), 7.95 (d, J = 8.8 Hz, 1H), 7.69 (d, J = 8.3 Hz, |
| 140 | 2H), 7.56 (d, J = 8.7 Hz, 1H), 7.02 (m, 1H), 6.96 (m, 1H), 6.93 (dd, J = 8.7, 2.5 Hz, 1H), 6.80 |
| | (dd, J = 8.8, 2.3 Hz, 1H), 6.61 (d, J = 8.4 Hz, 2H), 6.16 (t, J = 7.2 Hz, 1H), 5.73-5.59 (m, 1H), |
| | 4.58 (d, J = 13.3 Hz, 1H), 4.27 (m, 4H), 4.16 (d, J = 8.1 Hz, 1H), 4.05 (s, 1H), 3.77 (m, 1H), |
| | 3.55 (m, 3H), 3.23 (m, 2H), 3.10-2.83 (m, 5H), 2.82-2.54 (m, 6H), 2.32 (m, 3H), 1.95-1.69 |
| | (m, 3H), 1.29-1.24 (s, 6H), 1.22 (s, 6H), 1.09 (m, 2H). |
| Example | ¹H NMR (500 MHz, CDCl₃) δ 8.23 (s, 1H), 8.03-7.97 (m, 1H), 7.75-7.68 (m, 2H), 7.60-7.55 |
| 141 | (m, 1H), 7.53 (d, J = 2.7 Hz, 1H), 7.50-7.47 (m, 2H), 7.45 (dd, J = 9.2, 2.8 Hz, 1H), 6.99- |
| | 6.94 (m, 1H), 6.84-6.78 (m, 1H), 6.21 (d, J = 8.1 Hz, 1H), 5.81-5.74 (m, 1H), 4.16 (d, J = |
| | 8.1 Hz, 1H), 4.12-4.03 (m, 3H), 4.03-3.94 (m, 1H), 3.86-3.77 (m, 1H), 3.52-3.39 (m, 2H), |
| | 3.16-3.06 (m, 2H), 3.01-2.88 (m, 3H), 2.88-2.77 (m, 2H), 2.42-2.32 (m, 1H), 2.04-1.84 (m, |
| | 6H), 1.84-1.71 (m, 2H), 1.28 (s, 6H), 1.23 (s, 6H). |
| Example | ¹H NMR (500 MHz, CDCl₃) δ 8.20 (s, 1H), 8.04-7.99 (m, 1H), 7.74-7.68 (m, 2H), 7.60-7.54 |
| 142 | (m, 2H), 7.51-7.43 (m, 3H), 6.99-6.94 (m, 1H), 6.83-6.78 (m, 1H), 6.20 (d, J = 8.1 Hz, 1H), |
| | 5.83-5.75 (m, 1H), 4.15 (d, J = 8.2 Hz, 1H), 4.06 (s, 1H), 3.87-3.77 (m, 2H), 3.48-3.40 (m, |
| | 2H), 3.03-2.77 (m, 5H), 2.43-2.33 (m, 1H), 2.11-2.05 (m, 2H), 1.95-1.85 (m, 2H), 1.27 (s, |
| | 6H), 1.23 (s, 6H). |
| Example | ¹H NMR (500 MHz, CDCl₃) δ 8.18 (s, 1H), 7.96 (dd, J = 9.0, 1.6 Hz, 1H), 7.75-7.69 (m, 2H), |
| 143 | 7.57 (dd, J = 8.8, 1.6 Hz, 1H), 7.52-7.46 (m, 2H), 7.26-7.23 (m, 1H), 7.17-7.12 (m, 1H), |
| | 6.99-6.94 (m, 1H), 6.84-6.78 (m, 1H), 6.20 (d, J = 8.1 Hz, 1H), 6.04-5.97 (m, 1H), 5.83- |
| | 5.75 (m, 1H), 4.16 (d, J = 8.1 Hz, 1H), 4.06 (s, 1H), 4.04-4.00 (m, 2H), 4.00-3.95 (m, 1H), |
| | 3.77-3.69 (m, 1H), 3.67-3.59 (m, 1H), 3.45-3.36 (m, 1H), 3.05-2.79 (m, 4H), 2.44-2.35 (m, |
| | 1H), 2.05-1.92 (m, 2H), 1.89-1.77 (m, 2H), 1.28 (s, 6H), 1.23 (s, 6H). |
| Example | ¹H NMR (500 MHz, CDCl₃) δ 8.20-8.15 (m, 1H), 8.12 (s, 1H), 7.77-7.70 (m, 2H), 7.70-7.65 |
| 144 | (m, 2H), 7.60 (dd, J = 8.8, 1.6 Hz, 1H), 7.54-7.46 (m, 2H), 7.02-6.97 (m, 1H), 6.87-6.80 (m, |
| | 1H), 6.27-6.17 (m, 1H), 5.86-5.77 (m, 1H), 4.94 (s, 2H), 4.18 (d, J = 8.1 Hz, 1H), 4.08 (s, |
| | 1H), 3.99-3.88 (m, 1H), 3.83-3.73 (m, 1H), 3.65-3.54 (m, 1H), 3.51-3.42 (m, 1H), 3.06- |
| | 2.82 (m, 4H), 2.46-2.37 (m, 1H), 2.06-1.92 (m, 2H), 1.92-1.76 (m, 2H), 1.30 (s, 6H), 1.26 |
| | (s, 6H). |
| Example | ¹H NMR (500 MHz, CDCl₃) δ 8.71 (s, 2H), 8.04 (s, 1H), 8.00 (d, J = 9.1 Hz, 1H), 7.57 (d, J = |
| 145 | 8.7 Hz, 1H), 7.53 (d, J = 2.8 Hz, 1H), 7.45 (dd, J = 9.1, 2.8 Hz, 1H), 6.96 (d, J = 2.3 Hz, 1H), |
| | 6.80 (dd, J = 8.7, 2.3 Hz, 1H), 5.92 (d, J = 8.1 Hz, 1H), 5.81-5.73 (m, 1H), 4.69-4.59 (m, |
| | 1H), 4.13 (d, J = 8.1 Hz, 1H), 4.10-4.02 (m, 3H), 4.00-3.87 (m, 5H), 3.15-3.05 (m, 3H), |
| | 3.01-2.89 (m, 2H), 2.89-2.75 (m, 2H), 2.65-2.55 (m, 1H), 2.54-2.43 (m, 4H), 2.42-2.34 (m, |
| | 1H), 2.28-2.20 (m, 2H), 2.03-1.91 (m, 3H), 1.91-1.77 (m, 4H), 1.24 (s, 6H), 1.22 (s, 6H), |
| | 1.18-1.06 (m, 2H). |
| Example | ¹H NMR (500 MHz, CDCl₃) δ 8.71 (s, 2H), 8.06 (s, 1H), 7.98 (d, J = 8.8 Hz, 1H), 7.57 (d, J = |
| 146 | 8.7 Hz, 1H), 7.05 (d, J = 2.4 Hz, 1H), 6.98-6.92 (m, 2H), 6.80 (dd, J = 8.7, 2.4 Hz, 1H), 5.93 |
| | (d, J = 8.1 Hz, 1H), 5.81-5.73 (m, 1H), 4.69-4.59 (m, 1H), 4.38-4.24 (m, 4H), 4.13 (d, J = |
| | 8.1 Hz, 1H), 4.03 (s, 1H), 3.98-3.86 (m, 4H), 3.79 (p, J = 7.8 Hz, 1H), 3.64-3.55 (m, 1H), |
| | 3.13-3.03 (m, 1H), 3.01-2.88 (m, 2H), 2.88-2.76 (m, 1H), 2.72-2.61 (m, 1H), 2.56-2.42 (m, |
| | 4H), 2.43-2.34 (m, 1H), 2.25 (d, J = 7.1 Hz, 2H), 1.99-1.92 (m, 1H), 1.90-1.76 (m, 3H), 1.24 |
| | (s, 6H), 1.21 (s, 6H), 1.17-1.07 (m, 2H). |
| Example | ¹H NMR (500 MHz, CDCl₃) δ 8.71 (s, 2H), 8.12 (s, 1H), 7.95 (d, J = 8.9 Hz, 1H), 7.57 (d, J = |
| 147 | 8.7 Hz, 1H), 7.26-7.23 (m, 1H), 7.14 (d, J = 2.7 Hz, 1H), 6.96 (d, J = 2.4 Hz, 1H), 6.80 (dd, |
| | J = 8.9, 2.4 Hz, 1H), 6.06-5.99 (m, 1H), 5.93 (d, J = 8.1 Hz, 1H), 5.82-5.73 (m, 1H), 4.70- |
| | 4.61 (m, 1H), 4.13 (d, J = 8.1 Hz, 1H), 4.04 (s, 1H), 4.02-3.97 (m, 2H), 3.97-3.89 (m, 4H), |
| | 3.85-3.77 (m, 1H), 3.17-3.07 (m, 1H), 3.02-2.79 (m, 3H), 2.79-2.70 (m, 1H), 2.56-2.44 (m, |
| | 4H), 2.43-2.36 (m, 1H), 2.26 (d, J = 7.0 Hz, 2H), 2.03-1.95 (m, 1H), 1.95-1.88 (m, 1H), |
| | 1.89-1.79 (m, 1H), 1.25 (s, 6H), 1.22 (s, 6H), 1.20-1.14 (m, 2H). |
| Example | ¹H NMR (500 MHz, CDCl₃) δ 8.70 (s, 2H), 8.15 (d, J = 8.6 Hz, 1H), 8.07 (s, 1H), 7.67-7.61 |
| 148 | (m, 2H), 7.57 (d, J = 8.7 Hz, 1H), 6.96 (d, J = 2.4 Hz, 1H), 6.80 (dd, J = 8.6, 2.4 Hz, 1H), |
| | 5.92 (d, J = 8.1 Hz, 1H), 5.82-5.75 (m, 1H), 4.96-4.86 (m, 2H), 4.63-4.54 (m, 1H), 4.13 (d, |
| | J = 8.1 Hz, 1H), 4.04 (s, 1H), 3.97-3.88 (m, 4H), 3.87-3.79 (m, 1H), 3.19-3.10 (m, 1H), |
| | 3.03-2.79 (m, 3H), 2.73-2.64 (m, 1H), 2.54-2.44 (m, 4H), 2.44-2.35 (m, 1H), 2.25 (d, J = |
| | 7.0 Hz, 2H), 1.99-1.92 (m, 1H), 1.91-1.78 (m, 2H), 1.25 (s, 6H), 1.22 (s, 6H), 1.20-1.10 (m, |
| | 2H). |
| Example | ¹H NMR (300 MHz, CDCl₃) δ 8.20 (d, J = 12.5 Hz, 1H), 7.98-7.81 (m, 6H), 7.57 (d, J = 8.7 |
| 149 | Hz, 2H), 7.27-7.21 (m, 1H), 6.97 (s, 1H), 6.81 (d, J = 8.7 Hz, 1H), 6.49 (d, J = 8.7 Hz, 1H), |
| | 5.73 (d, J = 11.4 Hz, 1H), 4.66 (s, 1H), 4.08 (d, J = 4.5 Hz, 2H), 3.38 (s, 1H), 2.98-2.80 (m, |
| | 2H), 2.40-2.28 (m, 2H), 1.28 (s, 6H), 1.23 (s, 6H). |
| Example | ¹H NMR (300 MHz, Chloroform-d) δ 8.64 (s, 1H), 8.09 (s, 1H), 8.02-7.84 (m, 6H), 7.60 (d, |
| 150 | J = 8.7 Hz, 1H), 7.49 (d, J = 2.8 Hz, 1H), 7.42 (dd, J = 9.2, 2.8 Hz, 1H), 7.00 (d, J = 2.4 Hz, |
| | 1H), 6.84 (dd, J = 8.7, 2.4 Hz, 1H), 6.69 (t, J = 5.6 Hz, 1H), 6.29 (d, J = 8.1 Hz, 1H), 5.86- |
| | 5.77 (m, 1H), 4.62 (d, J = 5.6 Hz, 2H), 4.20 (d, J = 8.0 Hz, 1H), 4.10 (s, 1H), 4.03 (d, J = |
| | 13.2 Hz, 2H), 3.06-2.91 (m, 5H), 2.45-2.37 (m, 2H), 2.01-1.86 (m, 3H), 1.33 (s, 6H), 1.27 (s, |
| | 6H). |
| Example | ¹H NMR (300 MHz, Chloroform-d) δ 8.16 (s, 1H), 7.86 (s, 5H), 7.66-7.62 (m, 1H), 7.56 (d, |
| 151 | J = 8.7 Hz, 1H), 7.01-6.98 (m, 1H), 6.95 (d, J = 1.9 Hz, 1H), 6.93-6.86 (m, 2H), 6.79 (dd, J = |
| | 7.6, 1.6 Hz, 2H), 6.42-6.35 (m, 1H), 5.75-5.69 (m, 1H), 4.54 (s, 2H), 4.22-4.14 (m, 4H), 4.06 |
| | (s, 1H), 3.40-3.37 (m, 2H), 2.79-2.77 (m, 1H), 2.31-2.27 (m, 2H), 1.27 (s, 6H), 1.22 (s, 6H). |
| Example | ¹H NMR (300 MHz, Chloroform-d) δ 8.69 (s, 1H), 8.15 (s, 1H), 7.96-7.80 (m, 5H), 7.60 (d, |
| 152 | J = 8.7 Hz, 1H), 7.52 (s, 1H), 7.15 (d, J = 9.1 Hz, 1H), 7.08 (s, 1H), 7.00 (d, J = 2.4 Hz, 1H), |
| | 6.87-6.81 (m, 1H), 6.47 (d, J = 7.3 Hz, 1H), 5.75-5.66 (m, 1H), 5.58-5.50 (m, 1H), 4.72-4.64 |
| | (m, 2H), 4.24-4.20 (m, 1H), 4.12 (s, 1H), 4.02-3.98 (m, 1H), 2.91-2.83 (m, 2H), 2.38-2.29 (m, |
| | 1H), 2.04 (s, 1H), 1.34 (s, 6H), 1.28 (s, 6H). |
| Example | ¹H NMR (300 MHz, DMSO-d₆) δ 11.14 (s, 1H), 8.79 (s, 1H), 8.04 (q, J = 8.9 Hz, 5H), 7.93 |
| 153 | (dd, J = 8.8, 4.3 Hz, 2H), 7.46-7.40 (m, 1H), 7.34 (d, J = 9.3 Hz, 1H), 7.23 (d, J = 2.3 Hz, |
| | 1H), 7.15-7.12 (m, 1H), 7.05-7.00 (m, 1H), 5.92-5.83 (m, 1H), 4.36-4.33 (m, 1H), 4.14-4.09 |
| | (m, 1H), 3.67-3.56 (m, 2H), 3.12-3.05 (m, 2H), 2.75-2.72 (m, 1H), 2.70-2.65 (m, 2H), 1.26 (s, |
| | 6H), 1.16 (s, 6H). |
| Example | ¹H NMR (300 MHz, DMSO-d₆) δ 11.15 (s, 1H), 8.71 (s, 1H), 8.11-7.97 (m, 7H), 7.92 (d, J = |
| 154 | 8.7 Hz, 1H), 7.75-7.68 (m, 1H), 7.38 (d, J = 2.7 Hz, 1H), 7.23 (d, J = 2.4 Hz, 1H), 7.07-7.00 |
| | (m, 1H), 5.94-5.85 (m, 1H), 4.35 (s, 1H), 4.15-4.06 (m, 3H), 3.12-2.85 (m, 6H), 2.77-2.60 (m, |
| | 3H), 2.31-2.19 (m, 1H), 2.00-1.92 (m, 1H), 1.85-1.76 (m, 2H), 1.69-1.57 (m, 2H), 1.26 (s, |
| | 6H), 1.16 (s, 6H). |
| Example | ¹H NMR (300 MHz, DMSO-d₆) δ 8.77 (s, 1H), 8.42 (s, 1H), 8.09-7.98 (m, 6H), 7.92 (d, J = |
| 155 | 8.7 Hz, 1H), 7.25-7.23 (m, 1H), 7.14-7.08 (m, 1H), 7.05-7.01 (m, 1H), 6.90-6.85 (m, 1H), |
| | 5.89-5.80 (m, 1H), 4.36 (s, 1H), 4.27-4.18 (m, 3H), 4.14-4.05 (m, 3H), 2.95-2.88 (m, 2H), |
| | 2.76-2.70 (m, 2H), 2.30-2.25 (m, 1H), 2.00-1.91 (m, 1H), 1.83-1.76 (m, 1H), 1.26 (s, 6H), |
| | 1.16 (s, 6H). |
| Example | ¹H NMR (300 MHz, DMSO-d₆) δ 11.14 (s, 1H), 8.69 (s, 1H), 8.32 (s, 1H), 8.06-8.01 (m, 2H), |
| 156 | 7.98 (s, 1H), 7.96-7.85 (m, 3H), 7.56-7.49 (m, 1H), 7.39-7.33 (m, 1H), 7.25-7.22 (m, 1H), |
| | 7.06-6.98 (m, 2H), 5.91-5.79 (m, 1H), 4.39-4.32 (m, 1H), 4.14-4.08 (m, 1H), 3.92-3.85 (m, |
| | 2H), 3.51-3.41 (m, 3H), 2.93-2.85 (m, 2H), 2.76-2.60 (m, 3H), 1.27 (s, 6H), 1.16 (s, 6H). |
| Example | ¹H NMR (300 MHz, CDCl₃) δ 8.27 (s, 1H), 7.98-7.89 (m, 4H), 7.83 (d, J = 8.8 Hz, 2H), 7.57 |
| 157 | (d, J = 8.7 Hz, 1H), 6.98-6.95 (m, 2H), 6.87 (dd, J = 8.9, 2.6 Hz, 1H), 6.81 (dd, J = 8.7, 2.4 |
| | Hz, 2H), 6.33 (d, J = 8.1 Hz, 1H), 5.74 (dd, J = 11.5, 5.2 Hz, 1H), 4.18 (ddd, J = 15.8, 13.1, |
| | 8.0 Hz, 5H), 4.07 (s, 1H), 3.90 (t, J = 6.3 Hz, 2H), 3.70-3.62 (m, 4H), 3.58 (d, J = 5.0 Hz, |
| | 2H), 3.53-3.47 (m, 2H), 3.12 (t, J = 6.3 Hz, 2H), 3.01-2.77 (m, 4H), 2.42-2.32 (m, 1H), 2.04 |
| | (s, 1H), 1.29 (s, 6H), 1.24 (s, 6H). |
| Example | ¹H NMR (300 MHz, CDCl₃) δ 8.24 (s, 1H), 8.00-7.92 (m, 4H), 7.85 (d, J = 8.6 Hz, 2H), 7.60 |
| 158 | (d, J = 8.7 Hz, 1H), 7.51-7.40 (m, 2H), 7.00 (d, J = 2.3 Hz, 1H), 6.84 (dd, J = 8.7, 2.4 Hz, |
| | 1H), 6.39 (d, J = 7.9 Hz, 2H), 5.81-5.73 (m, 1H), 4.20 (d, J = 8.0 Hz, 1H), 4.04 (d, J = 13.2 |
| | Hz, 2H), 3.90 (t, J = 6.4 Hz, 2H), 3.68 (d, J = 5.2 Hz, 4H), 3.62-3.56 (m, 2H), 3.51-3.44 (m, |
| | 2H), 3.16-2.86 (m, 8H), 2.45-2.35 (m, 2H), 1.88 (dd, J = 23.4, 12.7 Hz, 4H), 1.32 (s, 6H), |
| | 1.27 (s, 6H). |
| Example | ¹H NMR (300 MHz, CDCl₃) δ 8.61 (s, 1H), 7.99 (s, 1H), 7.95-7.84 (m, 5H), 7.60 (d, J = 8.7 |
| 159 | Hz, 1H), 7.43 (s, 1H), 7.25-7.16 (m, 2H), 7.00 (d, J = 2.4 Hz, 1H), 6.84 (dd, J = 8.7, 2.4 Hz, |
| | 1H), 6.41 (d, J = 7.9 Hz, 1H), 5.91 (d, J = 4.9 Hz, 1H), 5.76-5.62 (m, 1H), 4.21 (d, J = 8.1 Hz, |
| | 1H), 4.10 (s, 1H), 4.05-4.00 (m, 2H), 3.93 (d, J = 3.1 Hz, 2H), 3.70-3.52 (m, 8H), 3.14 (t, J = |
| | 6.3 Hz, 2H), 2.99-2.78 (m, 3H), 2.38 (d, J = 8.3 Hz, 1H), 2.07 (s, 1H), 1.32 (s, 6H), 1.27 (s, |
| | 6H). |
| Example | 1H NMR (600 MHz, DMSO) δ 11.15 (s, 1H), 8.62 (s, 1H), 8.03 (d, J = 9.0 Hz, 1H), 7.95-7.88 |
| 162 | (m, 2H), 7.76-7.71 (m, 1H), 7.58 (d, J = 9.2 Hz, 1H), 7.39 (s, 1H), 7.21 (d, J = 1.4 Hz, 1H), |
| | 7.00 (dd, J = 8.8, 1.4 Hz, 1H), 6.86 (d, J = 9.1 Hz, 1H), 5.89 (dd, J = 11.7, 4.8 Hz, 1H), 4.42 |
| | (d, J = 12.8 Hz, 2H), 4.30 (s, 1H), 4.05 (d, J = 9.2 Hz, 1H), 3.49 (s, 3H), 3.00-2.86 (m, 3H), |
| | 2.76-2.59 (m, 3H), 2.53 (s, 4H), 2.40 (d, J = 21.6 Hz, 1H), 2.22 (dd, J = 14.8, 6.4 Hz, 3H), |
| | 1.86 (dd, J = 50.1, 12.1 Hz, 4H), 1.22 (d, J = 9.8 Hz, 6H), 1.15-1.07 (m, 6H). |
| Example | 1H NMR (600 MHz, DMSO) δ 11.18 (s, 1H), 8.61 (d, J = 2.4 Hz, 1H), 8.18 (d, J = 9.0 Hz, |
| 163 | 1H), 7.94 (dd, J = 8.9, 2.4 Hz, 1H), 7.89 (d, J = 8.7 Hz, 1H), 7.67 (dd, J = 8.9, 2.8 Hz, 1H), |
| | 7.61 (s, 1H), 7.59 (d, J = 2.8 Hz, 1H), 7.20 (d, J = 2.4 Hz, 1H), 6.99 (dd, J = 8.8, 2.4 Hz, 1H), |
| | 6.84 (d, J = 9.1 Hz, 1H), 5.95 (dd, J = 12.0, 5.2 Hz, 1H), 4.29 (s, 1H), 4.23 (t, J = 6.5 Hz, |
| | 2H), 4.08 (dd, J = 10.5, 5.2 Hz, 1H), 4.04 (d, J = 9.2 Hz, 1H), 3.58 (s, 3H), 3.16 (d, J = 5.2 |
| | Hz, 2H), 3.00-2.91 (m, 1H), 2.71-2.63 (m, 2H), 2.46-2.38 (m, 3H), 2.35-2.29 (m, 2H), 2.29- |
| | 2.22 (m, 1H), 2.03-1.94 (m, 1H), 1.85-1.76 (m, 2H), 1.53 (dd, J = 14.0, 7.2 Hz, 2H), 1.48 (dd, |
| | J = 14.5, 7.7 Hz, 3H), 1.22 (s, 3H), 1.20 (s, 5H), 1.10 (s, 5H). |
| Example | 1H NMR (600 MHz, DMSO) δ 11.15 (s, 1H), 8.57 (d, J = 2.3 Hz, 1H), 8.02 (d, J = 9.0 Hz, |
| 164 | 1H), 7.97 (d, J = 7.5 Hz, 1H), 7.92 (dd, J = 8.9, 2.4 Hz, 1H), 7.86 (d, J = 8.8 Hz, 1H), 7.58 |
| | (dd, J = 9.1, 2.3 Hz, 1H), 7.47 (d, J = 2.3 Hz, 1H), 7.39 (d, J = 2.4 Hz, 1H), 7.14 (dd, J = 8.8, |
| | 2.4 Hz, 1H), 6.44 (d, J = 8.9 Hz, 1H), 5.90 (dd, J = 12.2, 5.5 Hz, 1H), 4.58-4.46 (m, 1H), 3.80 |
| | (m, 1H), 3.70 (s, 1H), 3.57 (s, 1H), 3.50 (s, 4H), 3.03 (t, J = 9.5 Hz, 1H), 2.95 (m, 1H), 2.74- |
| | 2.61 (m, 2H), 2.60-2.53 (m, 4H), 2.46-2.41 (m, 2H), 2.30 (m, 1H), 2.24 (m, 1H), 2.15 (m, |
| | 1H), 2.12-2.03 (m, 2H), 1.90 (m, 2H), 1.73-1.58 (m, 3H), 1.58-1.39 (m, 5H). |
| Example | 1H NMR (600 MHz, DMSO) δ 11.17 (s, 1H), 8.10 (d, J = 8.8 Hz, 1H), 7.91 (d, J = 8.7 Hz, |
| 165 | 1H), 7.76 (d, J = 8.8 Hz, 2H), 7.65 (d, J = 9.4 Hz, 2H), 7.41 (d, J = 9.2 Hz, 1H), 7.21 (d, J = |
| | 2.4 Hz, 1H), 7.01 (dd, J = 8.8, 2.4 Hz, 1H), 6.55 (d, J = 8.9 Hz, 1H), 5.93 (dd, J = 12.4, 5.2 |
| | Hz, 1H), 4.33 (s, 1H), 4.06 (d, J = 9.1 Hz, 1H), 3.65 (s, 2H), 3.55-3.49 (m, 2H), 3.31-3.24 |
| | (m, 4H), 3.02-2.90 (m, 2H), 2.74-2.63 (m, 2H), 2.38-2.30(m, 1H), 2.30-2.24 (m, 1H), 2.21- |
| | 2.13 (m, 1H), 1.93-1.85 (m, 2H), 1.72-1.66 (m, 1H), 1.22 (s, 6H), 1.13 (s, 6H). |
| Example | 1H NMR (600 MHz, MeOD) δ 8.10 (d, J = 9.1 Hz, 1H), 7.78 (dd, J = 9.3, 2.7 Hz, 1H), 7.73 (t, |
| 166 | J = 8.6 Hz, 2H), 7.67 (d, J = 2.7 Hz, 1H), 7.13 (d, J = 2.4 Hz, 1H), 6.99 (dd, J = 8.8, 2.4 Hz, |
| | 1H), 6.62 (d, J = 8.8 Hz, 1H), 5.92 (dd, J = 11.8, 5.5 Hz, 1H), 4.29 (s, 1H), 4.14 (s, 1H), 3.63- |
| | 3.59 (m, 2H), 3.55-3.49 (m, 2H), 3.43-3.35 (m, 4H), 3.12-3.06 (m, 1H), 3.00-2.92 (m, 2H), |
| | 2.91-2.80 (m, 3H), 2.47-2.39 (m, 1H), 2.39-2.34 (m, 1H), 2.33-2.26 (m, 2H), 2.05-1.99 (m, |
| | 2H), 1.86-1.78 (m, 2H), 1.28 (s, 6H), 1.22 (s, 6H). |
| Example | 1H NMR (600 MHz, CDCl3) δ 8.51 (d, J = 2.4 Hz, 1H), 8.17 (s, 1H), 8.13 (d, J = 9.0 Hz, 1H), |
| 167 | 7.86 (dd, J = 9.0, 2.4 Hz, 1H), 7.56 (d, J = 8.7 Hz, 1H), 7.38 (d, J = 2.4 Hz, 1H), 7.31 (dd, J = |
| | 9.1, 2.4 Hz, 1H), 6.99 (d, J = 2.4 Hz, 1H), 6.84 (dd, J = 8.7, 2.3 Hz, 1H), 6.62 (d, J = 9.1 Hz, |
| | 1H), 5.78 (dd, J = 11.8, 5.4 Hz, 1H), 5.74 (d, J = 7.7 Hz, 1H), 4.40 (d, J = 13.2 Hz, 2H), 4.32- |
| | 4.26 (m, 1H), 4.08-4.01 (m, 1H), 3.50-3.40 (m, 4H), 3.09 (s, 4H), 2.99-2.79 (m, 5H), 2.43- |
| | 2.35 (m, 3H), 2.22 (d, J = 10.9 Hz, 2H), 2.16 (d, J = 11.5 Hz, 2H), 1.97-1.86 (m, 4H), 1.82 (d, |
| | J = 11.6 Hz, 2H), 1.71-1.63 (m, 3H), 1.44-1.37 (m, 2H), 1.25-1.18 (m, J = 12.2, 8.6 Hz, 2H); |
| | LC/MS (ESI) m/z [M + H]+: 544.0. |
| Example | 1H NMR (600 MHz, CDCl3) δ 8.51 (d, J = 2.2 Hz, 1H), 8.04 (s, 1H), 7.98 (d, J = 9.2 Hz, 1H), |
| 168 | 7.86 (dd, J = 9.0, 2.5 Hz, 1H), 7.56 (d, J = 8.7 Hz, 1H), 7.52 (d, J = 2.9 Hz, 1H), 7.45-7.42 |
| | (m, 1H), 6.99 (d, J = 2.4 Hz, 1H), 6.84 (dd, J = 8.7, 2.4 Hz, 1H), 6.62 (d, J = 9.0 Hz, 1H), |
| | 5.76 (dd, J = 11.7, 5.4 Hz, 1H), 5.73 (d, J = 7.7 Hz, 1H), 4.40 (d, J = 13.2 Hz, 2H), 4.29 (ddd, |
| | J = 14.1, 10.3, 4.0 Hz, 1H), 4.07-4.01 (m, 1H), 3.52-3.41 (m, 4H), 3.07 (s, 4H), 3.00-2.81 (m, |
| | 6H), 2.39 (d, J = 5.7 Hz, 2H), 2.22 (d, J = 10.7 Hz, 2H), 2.16 (d, J = 11.3 Hz, 2H), 1.94-1.85 |
| | (m, 4H), 1.82 (d, J = 11.8 Hz, 2H), 1.71-1.65 (m, 2H), 1.40 (dd, J = 24.4, 10.7 Hz, 3H), 1.24- |
| | 1.17 (m, 2H); LC/MS (ESI) m/z [M + H]+: 833.43. |
| Example | 1H NMR (600 MHz, CDCl3) δ 8.56 (d, J = 2.4 Hz, 1H), 8.13 (d, J = 9.0 Hz, 1H), 8.10 (s, 1H), |
| 169 | 7.90 (dd, J = 9.0, 2.5 Hz, 1H), 7.57 (d, J = 8.7 Hz, 1H), 7.37 (d, J = 2.5 Hz, 1H), 7.31 (dd, J = |
| | 9.1, 2.6 Hz, 1H), 6.96 (d, J = 2.4 Hz, 1H), 6.80 (dd, J = 8.7, 2.4 Hz, 1H), 6.66 (d, J = 9.0 Hz, |
| | 1H), 6.04 (d, J = 8.2 Hz, 1H), 5.78 (dd, J = 11.9, 5.5 Hz, 1H), 4.41 (d J = 13.4 Hz, 2H), 4.14 |
| | (d, J = 8.2 Hz, 1H), 4.04 (s, 1H), 3.51-3.40 (m, 4H), 3.09 (s, 4H), 3.00-2.87 (m, 4H), 2.83 |
| | (ddd, J = 12.7, 8.5, 5.3 Hz, 2H), 2.40 (d, J = 7.3 Hz, 2H), 2.38-2.35 (m, 1H), 1.97-1.87 (m, |
| | 4H), 1.83 (d, J = 12.2 Hz, 2H), 1.31-1.23 (m, 8H), 1.23-1.17 (m, 6H); LC/MS (ESI) m/z |
| | [M + H]+: 861.48. |
| Example | 1H NMR (600 MHz, CDCl3) δ 8.56 (d, J = 2.4 Hz, 1H), 8.04 (s, 1H), 7.98 (d, J = 9.2 Hz, 1H), |
| 170 | 7.90 (dd, J = 9.0, 2.5 Hz, 1H), 7.57 (d, J = 8.7 Hz, 1H), 7.52 (d, J = 2.9 Hz, 1H), 7.46-7.42 |
| | (m, 1H), 6.96 (d, J = 2.4 Hz, 1H), 6.80 (dd, J = 8.7, 2.4 Hz, 1H), 6.65 (d, J = 9.0 Hz, 1H), |
| | 6.03 (d, J = 8.2 Hz, 1H), 5.76 (dd, J = 11.8, 5.4 Hz, 1H), 4.41 (d, J = 13.2 Hz, 2H), 4.14 (d, |
| | J = 8.1 Hz, 1H), 4.04 (s, 1H), 3.54-3.41 (m, 4H), 3.08 (s, 4H), 3.01-2.87 (m, 4H), 2.86-2.79 (m, |
| | 1H), 2.44-2.34 (m, 3H), 1.99-1.86 (m, 4H), 1.83 (d, J = 12.2 Hz, 2H), 1.40-1.23 (m, 8H), 1.22 |
| | (d, J = 9.2 Hz, 6H); LC/MS (ESI) m/z [M + H]+: 861.48. |
| Example | 1H NMR (600 MHz, CDCl3) δ 8.56 (d, J = 2.4 Hz, 1H), 8.03 (s, 1H), 7.96 (d, J = 8.9 Hz, 1H), |
| 172 | 7.90 (dd, J = 9.0, 2.5 Hz, 1H), 7.57 (d, J = 8.7 Hz, 1H), 7.03 (d, J = 2.6 Hz, 1H), 6.96 (d, J = |
| | 2.4 Hz, 1H), 6.90 (dd, J = 8.9, 2.6 Hz, 1H), 6.80 (dd, J = 8.7, 2.4 Hz, 1H), 6.66 (d, J = 9.0 |
| | Hz, 1H), 6.04 (d, J = 8.1 Hz, 1H), 5.76 (dd, J = 11.6, 5.4 Hz, 1H), 4.42 (d, J = 13.1 Hz, 2H), |
| | 4.14 (d, J = 8.0 Hz, 1H), 4.04 (s, 1H), 3.79 (s, 4H), 3.00-2.90 (m, 3H), 2.87-2.77 (m, 2H), |
| | 2.38 (dd, J = 9.0, 4.4 Hz, 4H), 2.21-2.17 (m, 2H), 1.87 (dd, J = 10.8, 5.6 Hz, 4H), 1.81 (s, |
| | 1H), 1.25 (s, 6H), 1.20 (d, J = 13.3 Hz, 8H); LC/MS (ESI) m/z [M + H]+: 861.44. |
| Example | 1H NMR (600 MHz, CDCl3) δ 8.56 (d, J = 2.4 Hz, 1H), 8.06 (s, 1H), 7.98 (d, J = 9.2 Hz, 1H), |
| 174 | 7.90 (dd, J = 9.0, 2.5 Hz, 1H), 7.57 (d, J = 8.7 Hz, 1H), 7.51 (d, J = 2.9 Hz, 1H), 7.45-7.40 |
| | (m, 1H), 6.96 (d, J = 2.4 Hz, 1H), 6.80 (dd, J = 8.7, 2.4 Hz, 1H), 6.67 (d, J = 9.1 Hz, 1H), |
| | 6.03 (d, J = 8.1 Hz, 1H), 5.76 (dd, J = 11.7, 5.4 Hz, 1H), 4.14 (d, J = 8.1 Hz, 1H), 4.03 (d, J = |
| | 10.3 Hz, 3H), 3.70-3.57 (m, 4H), 3.08 (s, 4H), 3.01-2.89 (m, 4H), 2.86-2.79 (m, 1H), 2.44- |
| | 2.35 (m, 3H), 1.87 (d, J = 12.3 Hz, 2H), 1.82 (s, 4H), 1.63 (s, 2H), 1.38-1.23 (m, 8H), 1.22 |
| | (d, J = 7.2 Hz, 6H); LC/MS (ESI) m/z [M + H]+: 861.43. |
| Example | 1H NMR (600 MHz, CDCl3) δ 8.56 (s, 1H), 8.10 (d, J = 8.8 Hz, 2H), 7.90 (d, J = 9.0 Hz, 1H), |
| 175 | 7.57 (d, J = 8.7 Hz, 1H), 6.96 (s, 1H), 6.86 (s, 1H), 6.80 (d, J = 8.7 Hz, 1H), 6.76 (d, J = 8.7 |
| | Hz, 1H), 6.67 (d, J = 8.7 Hz, 1H), 6.03 (d, J = 7.8 Hz, 1H), 5.77 (dd, J = 11.4, 4.5 Hz, 1H), |
| | 4.15 (dd, J = 14.5, 7.4 Hz, 2H), 4.04 (s, 1H), 3.78-3.75 (m, 2H), 3.62 (s, 4H), 3.12 (s, 4H), |
| | 3.00-2.88 (m, 3H), 2.81 (t, J = 14.6 Hz, 3H), 2.37 (d, J = 9.9 Hz, 1H), 1.83 (s, 4H), 1.25 (s, |
| | 6H), 1.21 (s, 6H); LC/MS (ESI) m/z [M + H]+: 861.44. |
| Example | 1H NMR (600 MHz, CDCl3) δ 8.56 (d, J = 2.5 Hz, 1H), 8.02 (s, 1H), 7.95 (d, J = 8.9 Hz, 1H), |
| 176 | 7.90 (dd, J = 9.0, 2.5 Hz, 1H), 7.57 (d, J = 8.7 Hz, 1H), 7.01 (d, J = 2.6 Hz, 1H), 6.96 (d, J = |
| | 2.4 Hz, 1H), 6.88 (dd, J = 8.9, 2.6 Hz, H), 6.80 (dd, J = 8.7, 2.4 Hz, 1H), 6.67 (d, J = 9.0 Hz, |
| | 1H), 6.03 (d, J = 8.2 Hz, 1H), 5.76 (dd, J = 11.7, 5.4 Hz, 1H), 4.15 (dd, J = 15.3, 7.9 Hz, 2H), |
| | 4.04 (s, 1H), 3.79-3.75 (m, 2H), 3.68-3.57 (m, 4H), 3.11 (s, 4H), 2.95 (ddd, J = 15.0, 9.8, 3.8 |
| | Hz, 3H), 2.83 (dd, J = 11.2, 7.1 Hz, 2H), 2.79 (t, J = 7.5 Hz, 2H), 2.40-2.35 (m, 1H), 1.88- |
| | 1.76 (m, 4H), 1.33-1.23 (m, 6H), 1.22 (d, J = 7.2 Hz, 6H); LC/MS (ESI) m/z [M + H]+: 861.43. |
| Example | 1H NMR (600 MHz, CDCl3) δ 9.00 (dd, J = 2.2, 0.8 Hz, 1H), 8.18-8.09 (m, 2H), 8.02 (d, J = |
| 178 | 9.1 Hz, 1H), 7.58 (d, J = 8.7 Hz, 1H), 7.54 (d, J = 2.9 Hz, 1H), 7.45 (dd, J = 9.2, 2.9 Hz, 1H), |
| | 7.08 (s, 1H), 6.97 (d, J = 2.4 Hz, 1H), 6.81 (dd, J = 8.7, 2.4 Hz, 1H), 6.22 (d, J = 8.1 Hz, 1H), |
| | 5.78 (dd, J = 11.8, 5.4 Hz, 1H), 4.18 (d, J = 8.1 Hz, 2H), 4.07 (s, 2H), 3.62-3.51 (m, 4H), |
| | 3.42 (d, J = 22.3 Hz, 2H), 3.05-2.90 (m, 2H), 2.89-2.81 (m, 1H), 2.80-2.71 (m, 4H), 2.46-2.32 |
| | (m, 1H), 1.32 (s, 6H), 1.24 (s, 6H). |
| Example | 1H NMR (600 MHz, CDCl3) δ 9.01 (s, 1H), 8.23 (s, 1H), 8.15-8.10 (m, 1H), 8.07 (d, J = 8.3 |
| 179 | Hz, 1H), 7.58 (dd, J = 8.7, 2.2 Hz, 1H), 7.37 (s, 1H), 7.31 (d, J = 9.0 Hz, 1H), 6.97 (s, 1H), |
| | 6.81 (d, J = 8.7 Hz, 1H), 6.22 (d, J = 8.1 Hz, 1H), 5.81-5.75 (m, 1H), 4.17 (d, J = 8.0 Hz, 1H), |
| | 4.07 (s, 1H), 3.50 (s, 2H), 3.45 (s, 4H), 3.26 (s, 4H), 2.94 (dd, J = 30.6, 16.1 Hz, 2H), 2.84 |
| | (d, J = 16.4 Hz, 1H), 2.38 (d, J = 10.2 Hz, 1H), 1.94 (s, 4H), 1.28 (s, 6H), 1.25 (d, J = 9.1 Hz, |
| | 5H). |
| Example | 1H NMR (600 MHz, CDCl3) δ 8.99 (d, J = 1.7 Hz, 1H), 8.12 (dd, J = 8.3, 2.3 Hz, 1H), 8.09 |
| 180 | (s, 1H), 8.07 (d, J = 8.3 Hz, 1H), 7.99 (d, J = 9.2 Hz, 1H), 7.58 (d, J = 8.7 Hz, 1H), 7.53 (d, |
| | J = 2.9 Hz, 1H), 7.48-7.42 (m, 1H), 6.98-6.95 (m, 1H), 6.81 (dd, J = 8.7, 2.4 Hz, 1H), 6.20 (d, |
| | J = 8.1 Hz, 1H), 5.77 (dd, J = 11.7, 5.4 Hz, 1H), 4.17 (d, J = 8.1 Hz, 1H), 4.07 (s, 1H), 3.50 (s, |
| | 2H), 3.49-3.43 (m, 4H), 3.25 (s, 4H), 3.01-2.91 (m, 2H), 2.86-2.79 (m, 1H), 2.41-2.35 (m, |
| | 1H), 2.00-1.90 (m, 4H), 1.28 (s, 6H), 1.25 (d, J = 8.3 Hz, 6H). |
| Example | 1H NMR (600 MHz, CDCl3) δ 8.95 (d, J = 2.0 Hz, 1H), 8.29 (s, J = 12.8 Hz, 1H), 8.10 (dd, |
| 181 | J = 8.1, 2.3 Hz, 1H), 7.95 (d, J = 8.9 Hz, 1H), 7.58 (d, J = 8.7 Hz, 1H), 7.54 (d, J = 8.1 Hz, 1H), |
| | 7.02 (d, J = 2.6 Hz, 1H), 6.97 (d, J = 2.4 Hz, 1H), 6.90 (dd, J = 8.9, 2.6 Hz, 1H), 6.81 (dd, J = |
| | 8.7, 2.4 Hz, 1H), 6.24 (d, J = 8.0 Hz, 1H), 5.76 (dd, J = 11.6, 5.4 Hz, 1H), 4.17 (d, J = 8.1 |
| | Hz, 1H), 4.07 (s, J = 4.0 Hz, 1H), 3.82 (s, J = 10.3 Hz, 3H), 3.62 (s, 2H), 3.02-2.88 (m, 2H), |
| | 2.87-2.76 (m, 1H), 2.64 (d, J = 16.8 Hz, 2H), 2.62 (s, 1H), 2.41-2.31 (m, 1H), 1.95 (t, J = 5.3 |
| | Hz, 4H), 1.29 (s, 6H), 1.24 (s, 6H). |
| Example | 1H NMR (600 MHz, CDCl3) δ 9.02-8.97 (m, 1H), 8.18-8.07 (m, 2H), 7.96 (d, J = 8.9 Hz, 1H), |
| 182 | 7.58 (d, J = 8.7 Hz, 1H), 7.05 (s, 1H), 7.03 (d, J = 2.6 Hz, 1H), 6.97 (d, J = 2.4 Hz, 1H), 6.91 |
| | (dd, J = 8.9, 2.6 Hz, 1H), 6.81 (dd, J = 8.7, 2.4 Hz, 1H), 6.22 (d, J = 8.1 Hz, 1H), 5.77 (dd, |
| | J = 11.6, 5.4 Hz, 1H), 4.18 (d, J = 8.1 Hz, 1H), 4.07 (s, 1H), 3.81 (s, 3H), 3.36 (s, 2H), 2.94 |
| | (ddd, J = 16.9, 10.0, 3.7 Hz, 2H), 2.88-2.75 (m, 1H), 2.56 (s, J = 35.7 Hz, 2H), 2.38 (dt, J = |
| | 10.6, 5.8 Hz, 1H), 1.94 (t, J = 5.2 Hz, 4H), 1.28 (s, 6H), 1.24 (s, 6H). |
| Example | 1H NMR (600 MHz, CDCl3) δ 8.90 (d, J = 1.6 Hz, 1H), 8.06 (dd, J = 8.1, 2.3 Hz, 1H), 8.03 |
| 184 | (s, 1H), 7.96 (d, J = 8.9 Hz, 1H), 7.58 (d, J = 8.7 Hz, 1H), 7.49 (d, J = 7.5 Hz, 1H), 7.02 (d, |
| | J = 2.6 Hz, 1H), 6.97 (d, J = 2.4 Hz, 1H), 6.90 (dd, J = 8.9, 2.6 Hz, 1H), 6.81 (dd, J = 8.7, 2.4 |
| | Hz, 1H), 6.17 (d, J = 8.2 Hz, 1H), 5.77 (dd, J = 11.6, 5.4 Hz, 1H), 4.25-4.18 (m, 2H), 4.16 (d, |
| | J = 8.0 Hz, 1H), 4.07 (s, 1H), 3.80-3.74 (m, 2H), 3.06 (s, 2H), 2.99-2.91 (m, 2H), 2.83 (dd, |
| | J = 12.8, 8.0 Hz, 2H), 2.79 (s, 4H), 2.70 (s, 2H), 2.40-2.35 (m, 2H), 2.32-2.25 (m, 2H), 2.03- |
| | 1.95 (m, 2H), 1.88-1.82 (m, 2H), 1.34-1.27 (m, 6H), 1.27-1.20 (m, 6H); LC/MS (ESI) m/z |
| | [M + H]+: 842.47. |
| Example | 1H NMR (600 MHz, CDCl3) δ 8.58 (d, J = 2.3 Hz, 1H), 8.23 (s, 1H), 8.13 (d, J = 9.0 Hz, 1H), |
| 185 | 7.90 (dd, J = 8.9, 2.4 Hz, 1H), 7.57 (d, J = 8.7 Hz, 1H), 7.42-7.36 (m, 1H), 7.32 (dd, J = 9.1, |
| | 2.5 Hz, 1H), 6.96 (d, J = 2.4 Hz, 1H), 6.80 (dd, J = 8.7, 2.4 Hz, 1H), 6.39 (d, J = 8.9 Hz, 1H), |
| | 6.04 (d, J = 8.2 Hz, 1H), 5.79 (dd, J = 11.9, 5.4 Hz, 1H), 4.14 (d, J = 8.1 Hz, 1H), 4.04 (s, |
| | 1H), 3.66 (d, J = 42.0 Hz, 2H), 3.53-3.40 (m, 4H), 3.21 (s, 2H), 3.13 (s, 4H), 3.03-2.89 (m, |
| | 2H), 2.84 (dd, J = 10.4, 7.5 Hz, 1H), 2.66-2.53 (m, 2H), 2.38 (dd, J = 8.6, 4.3 Hz, 2H), 2.18 |
| | (s, 1H), 1.91 (s, 4H), 1.83-1.73 (m, 1H), 1.25 (s, 6H), 1.22 (s, 6H); LC/MS (ESI) m/z [M + H]+: |
| | 847.42. |
| Example | 1H NMR (600 MHz, CDCl3) δ 8.58 (d, J = 2.3 Hz, 1H), 8.08 (s, 1H), 7.99 (d, J = 9.1 Hz, 1H), |
| 186 | 7.90 (dd, J = 8.9, 2.4 Hz, 1H), 7.57 (d, J = 8.7 Hz, 1H), 7.52 (d, J = 2.9 Hz, 1H), 7.44 (dd, J = |
| | 9.2, 2.9 Hz, 1H), 6.96 (d, J = 2.4 Hz, 1H), 6.80 (dd, J = 8.7, 2.4 Hz, 1H), 6.39 (d, J = 8.9 Hz, |
| | 1H), 6.03 (d, J = 8.1 Hz, 1H), 5.76 (dd, J = 11.7, 5.4 Hz, 1H), 4.14 (d, J = 8.1 Hz, 1H), 4.04 |
| | (s, 1H), 3.71 (s, 1H), 3.63 (d, J = 17.4 Hz, 1H), 3.50-3.41 (m, 4H), 3.20 (s, 2H), 3.11 (s, 4H), |
| | 3.00-2.89 (m, 2H), 2.86-2.79 (m, 1H), 2.58 (d, J = 6.3 Hz, 2H), 2.42-2.33 (m, 2H), 2.17 (s, |
| | 1H), 1.89 (s, 4H), 1.81-1.74 (m, 1H), 1.25 (s, 6H), 1.21 (s, 6H); LC/MS (ESI) m/z [M + H]+: |
| | 847.42. |
| Example | 1H NMR (600 MHz, CDCl3) δ 8.58 (d, J = 2.3 Hz, 1H), 8.13 (d, J = 9.0 Hz, 1H), 8.08 (s, 1H), |
| 187 | 7.90 (dd, J = 8.9, 2.5 Hz, 1H), 7.57 (d, J = 8.7 Hz, 1H), 7.38 (d, J = 2.3 Hz, 1H), 7.32 (dd, J = |
| | 9.1, 2.6 Hz, 1H), 6.97 (d, J = 2.4 Hz, 1H), 6.81 (dd, J = 8.7, 2.4 Hz, 1H), 6.39 (d, J = 8.9 Hz, |
| | 1H), 6.03 (d, J = 8.2 Hz, 1H), 5.79 (dd, J = 11.9, 5.5 Hz, 1H), 4.14 (d, J = 8.1 Hz, 1H), 4.04 |
| | (s, 1H), 3.70 (m, 1H), 3.64 (m, 1H), 3.56-3.41 (m, 4H), 3.21 (s, 2H), 3.13 (s, 4H), 2.99-2.89 |
| | (m, 2H), 2.86-2.79 (m, 1H), 2.65-2.56 (m, 2H), 2.42-2.34 (m, 2H), 2.18 (s, 1H), 1.92 (s, 4H), |
| | 1.79 (d, J = 7.9 Hz, 1H), 1.25 (s, 6H), 1.22 (s, 6H); LC/MS (ESI) m/z [M + H]+: 847.35. |
| Example | 1H NMR (600 MHz, CDCl3) δ 8.58 (s, 1H), 7.99 (d, J = 9.1 Hz, 1H), 7.90 (d, J = 8.8 Hz, 1H), |
| 188 | 7.57 (d, J = 8.6 Hz, 1H), 7.53 (s, 1H), 7.45 (d, J = 9.2 Hz, 1H), 6.97 (s, 1H), 6.81 (d, J = 8.7 |
| | Hz, 1H), 6.39 (d, J = 8.8 Hz, 1H), 6.03 (d, J = 8.0 Hz, 1H), 5.77 (dd, J = 11.6, 5.2 Hz, 1H), |
| | 4.14 (d, J = 7.9 Hz, 1H), 4.04 (s, 1H), 3.70 (s, 1H), 3.63 (s, 1H), 3.46 (d, J = 5.4 Hz, 4H), |
| | 3.21 (s, 2H), 3.11 (s, 4H), 2.95 (dd, J = 27.8, 15.5 Hz, 2H), 2.85 (d, J = 16.8 Hz, 1H), 2.58 |
| | (d, J = 6.9 Hz, 2H), 2.41-2.35 (m, 2H), 2.20-2.15 (m, 1H), 1.90 (s, 4H), 1.80-1.75 (m, 1H), |
| | 1.25 (s, 4H), 1.22 (s, 4H); LC/MS (ESI) m/z [M + H]+: 847.35. |
| Example | 1H NMR (600 MHz, CDCl3) δ 8.59 (d, J = 2.3 Hz, 1H), 8.11 (d, J = 8.7 Hz, 1H), 8.07 (s, 1H), |
| 189 | 7.91 (dd, J = 8.9, 2.4 Hz, 1H), 7.57 (d, J = 8.7 Hz, 1H), 6.96 (d, J = 2.4 Hz, 1H), 6.86 (d, J = |
| | 2.3 Hz, 1H), 6.80 (dd, J = 8.7, 2.4 Hz, 1H), 6.78 (dd, J = 8.7, 2.3 Hz, 1H), 6.40 (d, J = 8.9 |
| | Hz, 1H), 6.04 (d, J = 8.2 Hz, 1H), 5.78 (dd, J = 11.9, 5.4 Hz, 1H), 4.14 (d, J = 8.1 Hz, 1H), |
| | 4.04 (s, 1H), 3.79 (s, 4H), 3.72 (s, 1H), 3.68-3.58 (m, 1H), 3.49 (s, 1H), 3.26 (s, 1H), 2.99- |
| | 2.88 (m, 2H), 2.85-2.80 (m, 1H), 2.60 (m, 1H), 2.45 (s, 2H), 2.39 (m, 4H), 2.18 (dd, J = 11.5, |
| | 4.8 Hz, 1H), 1.89 (t, J = 5.2 Hz, 4H), 1.84-1.76 (m, 1H), 1.25 (s, 6H), 1.22 (s, 6H); LC/MS |
| | (ESI) m/z [M + H]+: 847.40. |
| Example | 1H NMR (600 MHz, CDCl3) δ 8.61-8.57 (m, 1H), 8.12 (d, J = 8.7 Hz, 1H), 7.92-7.88 (m, 1H), |
| 190 | 7.57 (d, J = 8.7 Hz, 1H), 7.03 (d, J = 11.6 Hz, 1H), 6.98-6.94 (m, 1H), 6.81 (dd, J = 8.7, 2.4 |
| | Hz, 1H), 6.78 (dd, J = 8.7, 2.3 Hz, 1H), 6.38 (d, J = 8.8 Hz, 1H), 6.08-6.03 (m, 1H), 5.81 (dd, |
| | J = 9.7, 5.4 Hz, 1H), 4.17 (s, 2H), 4.16-4.13 (m, 1H), 4.05 (d, J = 3.4 Hz, 1H), 3.73 (d, J = |
| | 19.5 Hz, 1H), 3.64 (d, J = 13.0 Hz, 1H), 3.51 (d, J = 3.6 Hz, 4H), 3.21 (d, J = 8.2 Hz, 1H), |
| | 2.94 (d, J = 16.4 Hz, 1H), 2.90-2.77 (m, 2H), 2.64-2.55 (m, 2H), 2.45-2.36 (m, 2H), 2.20 (s, |
| | 1H), 1.80-1.72 (m, 1H), 1.33-1.24 (m, 6H), 1.22 (s, 6H); LC/MS (ESI) m/z [M + H]+: 819.40. |
| Example | 1H NMR (600 MHz, CDCl3) δ 8.58 (s, 1H), 8.13 (d, J = 9.0 Hz, 1H), 8.09 (d, J = 22.0 Hz, |
| 191 | 1H), 7.90 (d, J = 8.8 Hz, 1H), 7.57 (d, J = 8.7 Hz, 1H), 7.38 (s, 1H), 7.32 (d, J = 9.0 Hz, 1H), |
| | 6.96 (s, 1H), 6.80 (d, J = 8.7 Hz, 1H), 6.39 (d, J = 8.8 Hz, 1H), 6.04 (s, 1H), 5.79 (d, J = 11.2 |
| | Hz, 1H), 4.14 (d, J = 7.9 Hz, 1H), 4.06 (d, J = 12.8 Hz, 2H), 4.04 (s, 1H), 3.71 (s, 1H), 3.62 |
| | (s, 1H), 3.48 (s, 1H), 3.24 (s, 1H), 3.04 (t, J = 12.5 Hz, 2H), 2.98-2.88 (m, 2H), 2.83 (d, J = |
| | 15.9 Hz, 1H), 2.61 (d, J = 23.8 Hz, 4H), 2.53 (s, 2H), 2.45-2.35 (m, 3H), 2.17 (s, 1H), 2.07- |
| | 1.97 (m, 2H), 1.80 (d, J = 8.4 Hz, 1H), 1.65 (d, J = 11.5 Hz, 2H), 1.59 (s, 4H), 1.25 (s, 6H), |
| | 1.21 (s, 6H); LC/MS (ESI) m/z [M + H]+: 890.39. |
| Example | 1H NMR (600 MHz, CDCl3) δ 8.58 (d, J = 2.4 Hz, 1H), 8.10 (s, 1H), 7.99 (d, J = 9.1 Hz, 1H), |
| 192 | 7.90 (dd, J = 8.9, 2.5 Hz, 1H), 7.57 (d, J = 8.7 Hz, 1H), 7.53 (d, J = 2.9 Hz, 1H), 7.45-7.42 |
| | (m, 1H), 6.96 (d, J = 2.4 Hz, 1H), 6.80 (dd, J = 8.7, 2.4 Hz, 1H), 6.39 (d, J = 8.9 Hz, 1H), |
| | 6.03 (d, J = 8.2 Hz, 1H), 5.77 (dd, J = 11.7, 5.4 Hz, 1H), 4.14 (d, J = 8.1 Hz, 1H), 4.08 (d, J = |
| | 13.0 Hz, 2H), 4.04 (s, 1H), 3.71 (s, 1H), 3.62 (s, 1H), 3.48 (s, 1H), 3.23 (s, 1H), 3.04 (t, J = |
| | 11.6 Hz, 2H), 3.00-2.90 (m, 2H), 2.86-2.79 (m, 1H), 2.59 (dd, J = 17.8, 10.0 Hz, 4H), 2.52 (s, |
| | 2H), 2.40 (ddd, J = 13.5, 11.6, 5.6 Hz, 3H), 2.17 (s, 1H), 2.00 (d, J = 11.5 Hz, 2H), 1.78 (td, |
| | J = 16.3, 8.0 Hz, 1H), 1.63 (d, J = 12.0 Hz, 2H), 1.59 (s, 4H), 1.25 (s, 6H), 1.21 (s, 6H); |
| | LC/MS (ESI) m/z [M + H]+: 890.42. |
| Example | 1H NMR (600 MHz, CDCl3) δ 8.56 (d, J = 2.1 Hz, 1H), 8.13 (d, J = 9.0 Hz, 1H), 8.03 (s, 1H), |
| 193 | 7.90 (dd, J = 9.0, 2.2 Hz, 1H), 7.57 (d, J = 8.7 Hz, 1H), 7.38 (d, J = 2.1 Hz, 1H), 7.32 (dd, J = |
| | 9.0, 2.3 Hz, 1H), 6.96 (d, J = 2.1 Hz, 1H), 6.81 (dd, J = 8.7, 2.1 Hz, 1H), 6.66 (d, J = 9.1 Hz, |
| | 1H), 6.04 (d, J = 8.1 Hz, 1H), 5.79 (dd, J = 12.0, 5.4 Hz, 1H), 4.41 (d, J = 12.8 Hz, 2H), 4.14 |
| | (d, J = 8.1 Hz, 1H), 4.06 (d, J = 13.1 Hz, 2H), 4.04 (s, 1H), 3.04 (t, J = 11.8 Hz, 2H), 2.98- |
| | 2.94 (m, 2H), 2.91 (d, J = 12.1 Hz, 2H), 2.86-2.79 (m, 1H), 2.63 (s, 4H), 2.49 (s, 4H), 2.41- |
| | 2.35 (m, 1H), 2.22 (d, J = 5.7 Hz, 2H), 2.08-1.96 (m, 3H), 1.87 (d, J = 12.8 Hz, 2H), 1.81 (s, |
| | 1H), 1.66 (dd, J = 21.7, 10.9 Hz, 2H), 1.25 (s, 6H), 1.22 (s, 6H), 1.19 (d, J = 11.9 Hz, 2H); |
| | LC/MS (ESI) m/z [M + H]+: 904.52. |
| Example | 1H NMR (600 MHz, CDCl3) δ 8.56 (d, J = 2.1 Hz, 1H), 8.07 (s, 1H), 7.99 (d, J = 9.1 Hz, 1H), |
| 194 | 7.90 (dd, J = 9.0, 2.3 Hz, 1H), 7.57 (d, J = 8.7 Hz, 1H), 7.52 (d, J = 2.6 Hz, 1H), 7.44 (dd, J = |
| | 9.2, 2.7 Hz, 1H), 6.96 (d, J = 2.2 Hz, 1H), 6.80 (dd, J = 8.7, 2.2 Hz, 1H), 6.66 (d, J = 9.1 Hz, |
| | 1H), 6.03 (d, J = 8.1 Hz, 1H), 5.77 (dd, J = 11.6, 5.3 Hz, 1H), 4.41 (d, J = 13.3 Hz, 2H), 4.14 |
| | (d, J = 8.1 Hz, 1H), 4.08 (d, J = 13.0 Hz, 2H), 4.03 (s, 1H), 3.03 (t, J = 12.1 Hz, 2H), 2.99- |
| | 2.94 (m, 2H), 2.91 (t, J = 9.7 Hz, 2H), 2.84 (d, J = 16.2 Hz, 1H), 2.61 (s, 4H), 2.50 (d, J = |
| | 12.7 Hz, 4H), 2.38 (dd, J = 9.1, 4.7 Hz, 1H), 2.21 (d, J = 6.8 Hz, 2H), 2.00 (d, J = 11.5 Hz, |
| | 2H), 1.87 (d, J = 12.9 Hz, 2H), 1.81 (s, 1H), 1.63 (d, J = 12.3 Hz, 2H), 1.25 (s, 6H), 1.21 (s, |
| | 6H), 1.19 (s, 2H); LC/MS (ESI) m/z [M + H]+: 903.45. |
| Example | ¹H NMR (600 MHz, CDCl₃) δ 8.90 (d, J = 1.8 Hz, 1H), 8.12 (d, J = 9.0 Hz, 1H), 8.05 (dd, J = |
| 195 | 8.1, 2.3 Hz, 1H), 8.05-8.01 (m, 1H), 7.58 (d, J = 8.7 Hz, 1H), 7.49-7.46 (m, 1H), 7.37 (d, J = |
| | 2.5 Hz, 1H), 7.31 (dd, J = 9.1, 2.5 Hz, 1H), 6.97 (d, J = 2.4 Hz, 1H), 6.81 (dd, J = 8.7, 2.4 |
| | Hz, 1H), 6.17 (d, J = 8.0 Hz, 1H), 5.78 (dd, J = 11.9, 5.4 Hz, 1H), 4.16 (d, J = 8.1 Hz, 1H), |
| | 4.07 (s, 1H), 4.02 (d, J = 12.9 Hz, 2H), 3.05-2.99 (m, 2H), 2.97-2.89 (m, 2H), 2.87-2.82 (m, |
| | 1H), 2.81-2.74 (m, 2H), 2.71-2.66 (m, 1H), 2.41-2.35 (m, 1H), 2.28-2.16 (m, 4H), 2.02 (s, |
| | 1H), 2.00-1.95 (m, 2H), 1.93 (d, J = 12.4 Hz, 2H), 1.87-1.79 (m, 2H), 1.36-1.30 (m, 2H), |
| | 1.30-1.26 (m, J = 11.8 Hz, 6H), 1.26-1.21 (m, 6H); LC/MS (ESI) m/z [M + H]+: 844.39. |
| Example | ¹H NMR (600 MHz, CDCl₃) δ 8.90 (s, 1H), 8.05 (d, J = 8.0 Hz, 1H), 8.02 (s, 1H), 7.98 (d, J = |
| 196 | 9.2 Hz, 1H), 7.57 (d, J = 8.7 Hz, 1H), 7.51 (s, 1H), 7.48 (d, J = 8.1 Hz, 1H), 7.43 (d, J = 9.2 |
| | Hz, 1H), 6.97 (s, 1H), 6.81 (d, J = 8.7 Hz, 1H), 6.16 (d, J = 7.8 Hz, 1H), 5.76 (dd, J = 11.0, |
| | 4.6 Hz, 1H), 4.16 (d, J = 8.0 Hz, 1H), 4.06 (s, 1H), 4.04 (d, J = 13.5 Hz, 2H), 3.05-2.98 (m, |
| | 2H), 2.98-2.91 (m, 2H), 2.88-2.80 (m, 1H), 2.75 (s, 2H), 2.67 (s, 1H), 2.37 (d, J = 9.4 Hz, |
| | 1H), 2.26-2.14 (m, 4H), 2.06-2.00 (m, 1H), 1.96 (s, 2H), 1.91 (d, J = 12.9 Hz, 2H), 1.86-1.79 |
| | (m, 2H), 1.31-1.26 (m, 8H), 1.23 (s, 6H); LC/MS (ESI) m/z [M + H]+: 844.40. |
| Example | ¹H NMR (600 MHz, CDCl₃) δ 8.57 (d, J = 2.4 Hz, 1H), 8.15 (d, J = 8.9 Hz, 1H), 8.00 (s, 1H), |
| 197 | 7.91 (dd, J = 9.0, 2.2 Hz, 1H), 7.57 (d, J = 8.6 Hz, 1H), 7.38 (d, J = 2.4 Hz, 1H), 7.32 (dd, J = |
| | 9.0, 2.4 Hz, 1H), 6.96 (d, J = 2.2 Hz, 1H), 6.80 (dd, J = 8.7, 2.1 Hz, 1H), 6.66 (d, J = 9.1 Hz, |
| | 1H), 6.04 (d, J = 8.1 Hz, 1H), 5.79 (dd, J = 11.9, 5.3 Hz, 1H), 4.42 (d, J = 13.3 Hz, 2H), 4.14 |
| | (d, J = 8.1 Hz, 1H), 4.04 (s, 1H), 3.56-3.45 (m, 4H), 3.00-2.89 (m, 4H), 2.87-2.80 (m, 1H), |
| | 2.66-2.59 (m, 4H), 2.52-2.45 (m, 2H), 2.41-2.35 (m, J = 11.4, 6.5 Hz, 1H), 1.83 (d, J = 12.3 |
| | Hz, 2H), 1.70-1.64 (m, 1H), 1.55-1.50 (m, J = 8.1 Hz, 2H), 1.31-1.26 (m, J = 10.3, 3.1 Hz, |
| | 2H), 1.25 (s, 6H), 1.21 (s, 6H); LC/MS (ESI) m/z [M + H]+: 835.44. |
| Example | ¹H NMR (600 MHz, CDCl₃) δ 8.56 (d, J = 2.4 Hz, 1H), 8.02 (s, 1H), 8.01 (s, 1H), 7.90 (dd, |
| 198 | J = 9.0, 2.5 Hz, 1H), 7.57 (d, J = 8.7 Hz, 1H), 7.53 (d, J = 2.9 Hz, 1H), 7.45 (dd, J = 9.2, 2.9 |
| | Hz, 1H), 6.96 (d, J = 2.4 Hz, 1H), 6.80 (dd, J = 8.7, 2.4 Hz, 1H), 6.66 (d, J = 9.0 Hz, 1H), |
| | 6.04 (d, J = 8.2 Hz, 1H), 5.77 (dd, J = 11.8, 5.4 Hz, 1H), 4.42 (d, J = 13.4 Hz, 2H), 4.14 (d, |
| | J = 8.1 Hz, 1H), 4.04 (s, 1H), 3.56-3.47 (m, 4H), 3.00-2.97 (m, 1H), 2.96-2.92 (m, 2H), 2.92- |
| | 2.90 (m, 1H), 2.86-2.82 (m, 1H), 2.65-2.59 (m, 4H), 2.50-2.44 (m, 2H), 2.41-2.36 (m, 1H), |
| | 1.83 (d, J = 10.9 Hz, 2H), 1.69-1.64 (m, 1H), 1.55-1.50 (m, 2H), 1.31-1.26 (m, 2H), 1.25 (s, |
| | 6H), 1.21 (s, 6H); LC/MS (ESI) m/z [M + H]+: 835.43. |

### Evaluation of the Compounds of the Present Invention

Assessment of AR Proteolytic Activity

Androgen receptor (AR) proteolytic activity of the compound according to the present invention was evaluated as follows: Specifically, 5.5×10⁵ LNCap cells were injected into each well of a 6-well plate. The next day, each well was treated with the compound to a final concentration of 100 nM, 1 μM, and 10 μM. One well was treated with DMSO at the same percentage. After 24 hours of treatment, cells were collected and lysed using RIPA Lysis buffer (50 mM Tris-HCl, pH7.5, 150 mM NaCl, 1% Triton X-100, 2 mM EDTA, 0.1% SDS, 0.5% sodium deoxycholate and protease inhibitor cocktail) to make cell lysates. Western blotting was performed, and the results were quantified using Image J program.

Cytotoxicity Assessment

In order to evaluate the androgen receptor (AR) cytotoxicity of the compounds according to the present invention, the following experiment was conducted. Specifically, 1.5×10³ (LNCaP) or 4×10³ (VCaP, 22RV1), respectively, of LNCaP, VCaP, and 22RV1 cells were injected into each well of a 96-well plate. The next day, each well was treated with the compound at a final concentration of 3 nM, 10 nM, 30 nM, 100 nM, 300 nM, 1 μM, 3 μM, and 10 μM. One well was treated with DMSO at the same percentage, and these experiments were replicated twice at the same concentration to confirm the reproducibility of the study. After 6 days of treatment, Cell-titer-glo (promega) was treated and reacted for 10 minutes, and cell viability was measured with a multimode plate reader (PerkinElmer, Inc.), and the change was quantified compared to when DMSO was treated.

The evaluation results are summarized and shown in Table 4 below.

TABLE 4

| | | | | Degradation |
|---|---|---|---|---|
| | | | | (1 μM |
| | | | | treated in |
| | | | | LNCaP, |
| | LNCAP | VCaP | 22RV1 | % AR |
| Example | (CC50, μM) | (CC50, μM) | (CC50, μM) | remaining) |
| Example 1 | 5.689 | | | 72 |
| Example 2 | 4.929 | | | 100 |
| Example 3 | 2.98 | | 0.4305 | 47 |
| Example 4 | 5.322 | | 4.462 | 58 |
| Example 5 | 1.601 | | | 6 |
| Example 6 | 5.931 | | 7.099 | 66 |
| Example 7 | 0.9018 | | | 24.3 |
| Example 8 | 0.163 | 0.6854 | | 8.1 |
| Example 9 | 0.1166 | | | 39.9 |
| Example 10 | 0.1898 | 0.2565 | | 12.5 |
| Example 11 | 0.0914 | 0.07469 | | 7 |
| Example 12 | 1.306 | | | 7 |
| Example 13 | 0.8035 | | | 8.3 |
| Example 14 | 4.021 | | | 35.3 |
| Example 15 | 1.311 | | | 13.6 |
| Example 16 | 1.952 | | | 12.6 |
| Example 17 | 0.8352 | | | 6.7 |
| Example 18 | 0.1959 | | | 23 |
| Example 19 | 0.2084 | 0.07632 | | 23 |
| Example 20 | 0.1784 | 0.0542 | | 26 |
| Example 21 | 0.3118 | | | 21 |
| Example 22 | 13.37 | 0.1352 | | 74 |
| Example 23 | 0.302 | 0.1909 | | 5 |
| Example 24 | 1.479 | 0.1072 | | 13 |
| Example 25 | 2.132 | 0.0457 | | 50 |
| Example 26 | 0.3609 | 0.05161 | | 7 |
| Example 27 | 0.5525 | 0.03711 | | 14 |
| Example 28 | 1.283 | 1.579 | | 69 |
| Example 29 | 0.4249 | 0.1199 | | 6 |
| Example 30 | 0.8469 | 0.07998 | | 4 |
| Example 31 | 0.8518 | 0.05709 | | 5 |
| Example 32 | 1.297 | 0.1411 | | 2 |
| Example 33 | 0.7616 | 0.05696 | | 14 |
| Example 34 | 0.5234 | 0.07023 | | 11 |
| Example 35 | | 0.05246 | | 6.4 |
| Example 36 | | 0.03654 | | 9.8 |
| Example 37 | | 0.02905 | | 14.9 |
| Example 38 | | 0.04321 | | 27.3 |
| Example 39 | | 0.0866 | | 2.1 |
| Example 40 | | 0.02429 | | 19.7 |
| Example 41 | | 0.02005 | | 12.3 |
| Example 42 | | 0.04943 | | 10 |
| Example 43 | | 0.03733 | | 33.6 |
| Example 44 | | 0.02593 | | 3.7 |
| Example 45 | | 0.04659 | | 21.1 |
| Example 46 | | 0.05222 | | 15.6 |
| Example 47 | 0.1681 | 0.02367 | | 43.6 |
| Example 48 | | 0.07697 | | 35.2 |
| Example 49 | | 0.0678 | | 8.6 |
| Example 50 | | 0.2228 | | 6.8 |
| Example 51 | | 0.2279 | | 10.4 |
| Example 52 | | 0.0594 | | 32.2 |
| Example 53 | | 0.0197 | | 5.8 |
| Example 54 | | 0.5534 | | 7.7 |
| Example 55 | | 0.03654 | | 8.6 |
| Example 56 | | 0.03609 | | 21.3 |
| Example 57 | | 0.2253 | | 13.3 |
| Example 58 | | 0.33 | | 11.4 |
| Example 59 | | ~1 | | |
| Example 60 | | 3.284 | | 20.3 |
| Example 61 | | 0.2897 | | 25 |
| Example 62 | | 0.03452 | | 15 |
| Example 63 | 0.05321 | 0.01485 | | 6 |
| Example 64 | | 0.07618 | | 56 |
| Example 65 | | 0.04399 | | 25 |
| Example 66 | | 0.01825 | | 35 |
| Example 67 | | 0.1064 | | 88 |
| Example 68 | | 0.1622 | | 26 |
| Example 69 | | 0.3449 | | |
| Example 70 | | 0.2052 | | 99 |
| Example 71 | | 0.07047 | | 37 |
| Example 72 | | 0.2627 | | 92 |
| Example 73 | | 0.07952 | | 40 |
| Example 74 | | 0.07324 | | 20 |
| Example 75 | | 0.03007 | | 9 |
| Example 76 | | 0.035 | | 30 |
| Example 77 | | 0.04664 | | 74 |
| Example 78 | | 0.007684 | | 24 |
| Example 79 | | 0.2003 | | 45 |
| Example 80 | | 0.010 | | 56 |
| Example 81 | | 0.02383 | | 18 |
| Example 82 | | 0.01439 | | 27 |
| Example 83 | 0.0137 | 0.006 | | 43 |
| Example 84 | | 0.01544 | | 18 |
| Example 85 | | 0.1709 | | 21 |
| Example 86 | | 0.03492 | | 20 |
| Example 87 | | 0.03191 | | 9 |
| Example 88 | | 0.01453 | | 14 |
| Example 89 | | 0.1118 | | |
| Example 90 | | 0.01957 | | 5 |
| Example 91 | | 0.2624 | | |
| Example 92 | | >10 | | |
| Example 93 | 0.0515 | 0.02243 | | |
| Example 94 | 0.1076 | 0.00784 | | |
| Example 95 | | 0.004497 | | |
| Example 96 | | 0.008029 | | |
| Example 97 | 0.07673 | 0.002079 | | |
| Example 98 | | 0.006718 | | |
| Example 99 | 0.03152 | 0.002 | | 20 |
| Example 100 | | 0.002 | | |
| Example 101 | 0.007056 | 0.01553 | | 40 |
| Example 102 | 0.01585 | 0.00588 | | |
| Example 103 | 0.1435 | 0.0123 | | |
| Example 104 | | 0.01685 | | |
| Example 105 | 0.02595 | 0.08016 | | |
| Example 106 | 0.06906 | 0.01126 | | |
| Example 107 | 0.08987 | 0.003592 | | |
| Example 108 | | 0.01885 | | |
| Example 109 | | 0.01301 | | |
| Example 110 | 0.4563 | 0.003647 | | |
| Example 111 | 0.03724 | 0.002288 | | |
| Example 112 | | 0.016 | | |
| Example 113 | | 0.030 | | |
| Example 114 | | 0.014 | | |
| Example 115 | 0.1821 | 0.038 | | |
| Example 116 | | 0.01607 | | |
| Example 117 | 0.1655 | 0.013 | | |
| Example 118 | | 0.017 | | |
| Example 119 | | 0.036 | | |
| Example 120 | | 0.0297 | | |
| Example 121 | | 0.018 | | |
| Example 122 | | 0.018 | | |
| Example 123 | | 0.055 | | |
| Example 124 | | 0.013 | | |
| Example 125 | | 0.0155 | | |
| Example 126 | | 0.040 | | |
| Example 127 | | 0.027 | | |
| Example 128 | | 0.011 | | |
| Example 129 | | 0.0194 | | |
| Example 130 | | 0.017 | | |
| Example 131 | | 0.0185 | | |
| Example 132 | | 0.015 | | |
| Example 133 | | 0.015 | | |
| Example 134 | | 0.0645 | | |
| Example 135 | | ~0.01 | | |
| Example 136 | | 0.0677 | | |
| Example 137 | | 0.094 | | |
| Example 138 | | 0.06374 | | |
| Example 139 | | 0.0695 | | |
| Example 140 | | 0.030 | | |
| Example 141 | | 0.0115 | | |
| Example 142 | | 0.0071 | | |
| Example 143 | 0.0176 | 0.0069 | | 18 |
| Example 144 | 0.1436 | 0.021 | | 14.7 |
| Example 145 | | ~0.01 | | |
| Example 146 | | ~0.01 | | |
| Example 147 | | ~0.01 | | |
| Example 148 | | ~0.010 | | |
| Example 149 | | >10 | | |
| Example 150 | | 0.02439 | | |
| Example 151 | | 0.02562 | | |
| Example 152 | | 0.2329 | | |
| Example 153 | | 0.04537 | | |
| Example 154 | | 0.0416 | | |
| Example 155 | | 0.177 | | |
| Example 156 | | 0.9363 | | |
| Example 157 | | 0.03214 | | |
| Example 158 | | 0.03875 | | |
| Example 159 | | 0.1088 | | |
| Example 160 | | | | 31 |
| Example 161 | | | | 36 |
| Example 162 | | | | 30 |
| Example 163 | | | | 10 |
| Example 164 | | | | 13 |
| Example 165 | | | | 8 |
| Example 166 | | | | 13 |
| Example 167 | | | | 19 |
| Example 168 | | | | 10 |
| Example 169 | | | | 16 |
| Example 170 | | | | 16 |
| Example 171 | | | | 11 |
| Example 172 | | | | 7 |
| Example 173 | | | | 9 |
| Example 174 | | | | 15 |
| Example 175 | | | | 10 |
| Example 176 | | | | 6 |
| Example 177 | | | | 56 |
| Example 178 | | | | 23 |
| Example 179 | | | | 8 |
| Example 180 | | | | 18 |
| Example 181 | | | | 18 |
| Example 182 | | | | 16 |
| Example 183 | | | | 7.5 |
| Example 184 | | | | 11 |
| Example 185 | | | | 10 |
| Example 186 | | | | 11 |
| Example 187 | | | | 27 |
| Example 188 | | | | 21 |
| Example 189 | | | | 14 |
| Example 190 | | | | 11 |
| Example 191 | | | | 33 |
| Example 192 | | | | 22 |
| Example 193 | | | | 12 |
| Example 194 | | | | 9 |
| Example 195 | | | | 3 |
| Example 196 | | | | 4 |
| Example 197 | | | | 16 |
| Example 198 | | | | 10 |

As shown in Table 4, the compounds according to the present invention showed goad activity in inhibiting or degrading the androgen receptor, and showed excellent cytotoxicity in the evaluation of cancer cell lines such as LNCaP, VCaP, and 22RV1, which are AR-dependent prostate cancer cell lines.

In addition, the compounds of Example 11, 20, 33, 34, 44, 47, 63, 75, 76, 78, 82-84, 88, 90, 94-104, 106-108, 110-112, 114, 116-118, 121, 122, 124, 125, 128-133, 142, 143, 150, 165, 166, 169-176, 183-186, and 193-196 were preferred in various aspects such as cytotoxicity (inhibition of cancer cell growth) to LNCaP, VCaP, 22RV1, etc., androgen receptor proteolytic activity, and (metabolic) stability.

All documents mentioned herein are incorporated herein by reference as if their contents were set forth herein. When incorporating elements of the present invention or preferred embodiment(s) thereof, the articles “a,” “an,” “the,” and “said” are intended to mean that there is more than one element.

As used herein, the singular “a” and “an” may include the plural form unless the context clearly dictates otherwise.

The terms “comprising”, “including” and “having” are intended to be inclusive and mean that there may be additional elements other than those listed. Although the invention has been described in terms of a particular embodiment or embodiments, it should not be construed as limiting the details of these embodiments.

1. A compound of Chemical Formula 1: 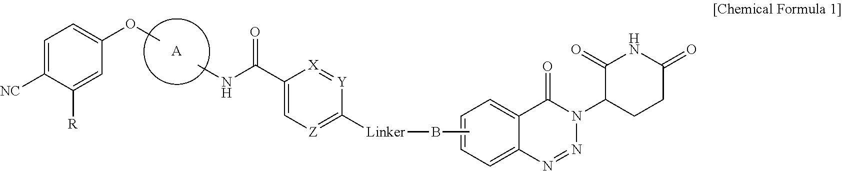
or a pharmaceutically acceptable salt thereof,
in the Chemical Formula 1
R is H, C₁₋₆alkyl, halogen, or haloC₁₋₆alkyl,
A is C₃₋₁₀cycloalkyl or heterocycle, wherein these have optionally one or more hydrogens replaced by C₁₋₆alkyl, C₁₋₆alkoxy, halogen, haloC₁₋₆alkyl, ═O, or ═S,
X, Y, and Z are each independently CH or N,
Linker-B is a linker that connects the moieties on both sides of Linker-B.

2. The compound of claim 1 or a pharmaceutically acceptable salt thereof, wherein the Chemical Formula 1 is the Chemical Formula 1a: 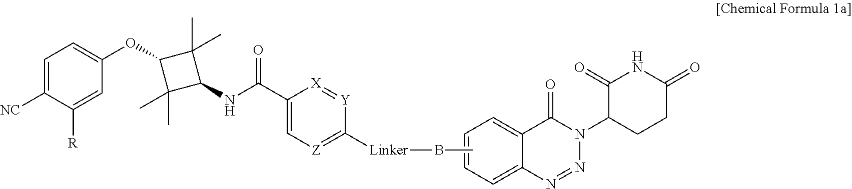
in the Chemical Formula 1a,
R is H, C₁₋₆alkyl, halogen, or haloC₁₋₆alkyl,
X, Y, and Z are each independently CH or N,
Linker-B is a linker that connects the moieties on both sides of Linker-B.

3. The compound of claim 2 or a pharmaceutically acceptable salt thereof, wherein R is halogen or CF₃ in the Chemical Formula 1a.

4. The compound of claim 1 or a pharmaceutically acceptable salt thereof, wherein the Linker has the structure of Chemical Formula 2: 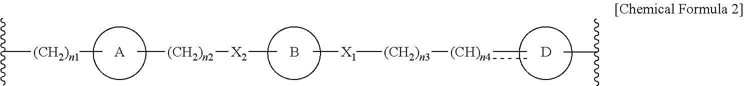
in the Chemical Formula 2,
A is any one of the following structures: 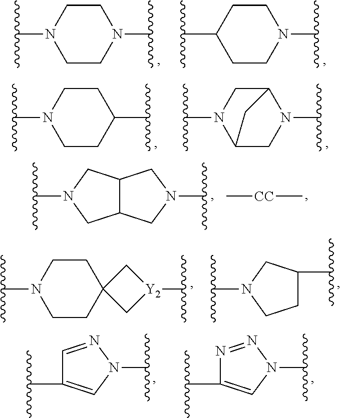
B is direct bond, 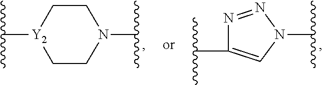
X₁, and X₂ are each independently direct bond, O, or C(O),
D is direct bond, NH, or any one of the following structures: 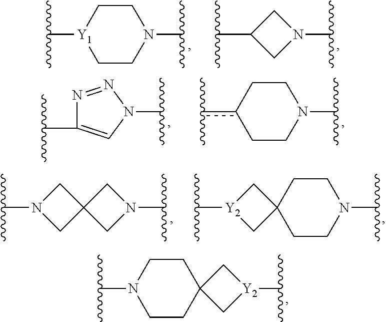
Y₁ and Y₂ are each independently N, CH, C(OH), or CF,
n₁, n₂, and n₃ are each independently an integer of from 0 to 5,
n4 is 0 or 1, [symbol]
is single bond or double bond.

5. The compound of claim 4 or a pharmaceutically acceptable salt thereof, wherein the Linker is any one of the following structures:

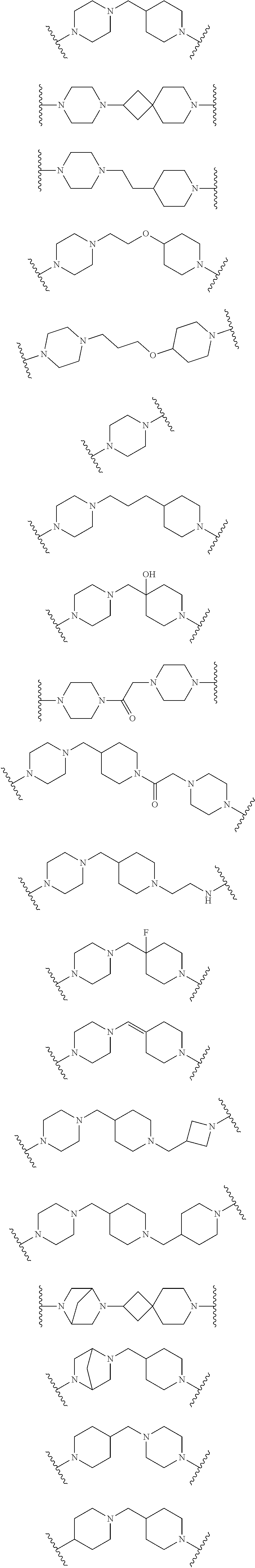

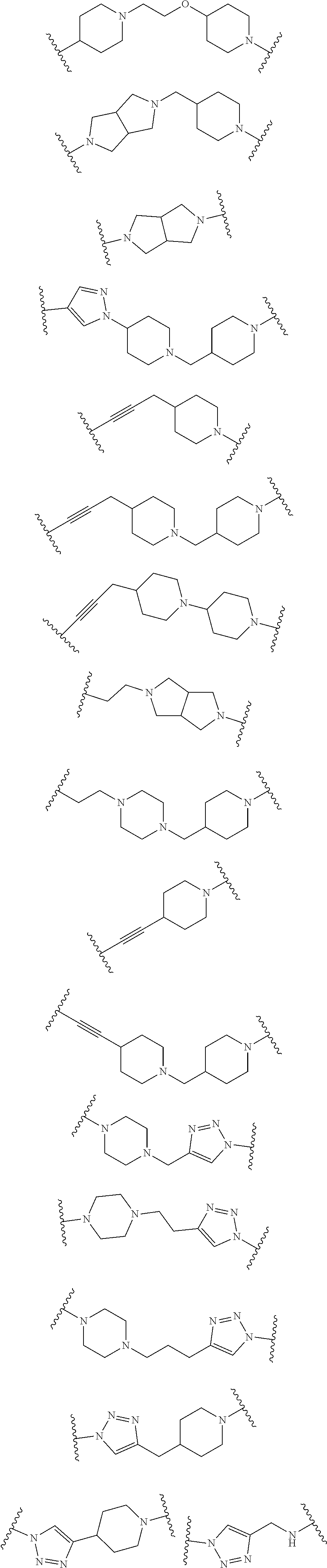

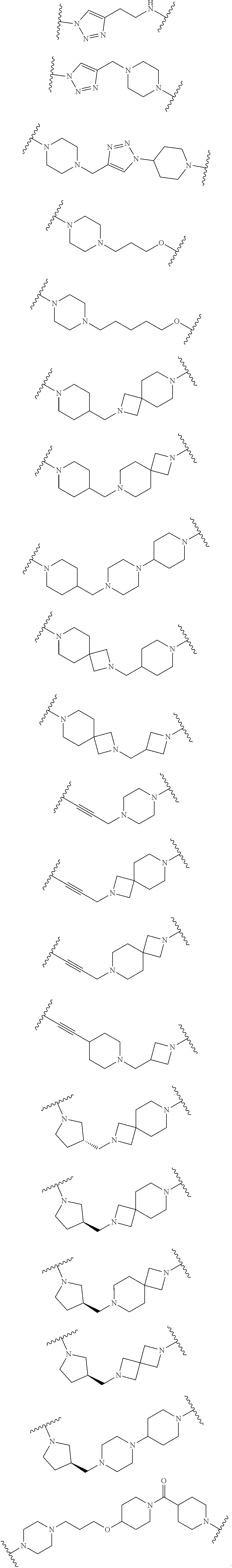

6. The compound of claim 5 or a pharmaceutically acceptable salt thereof, wherein the Linker is any one of the following structures:

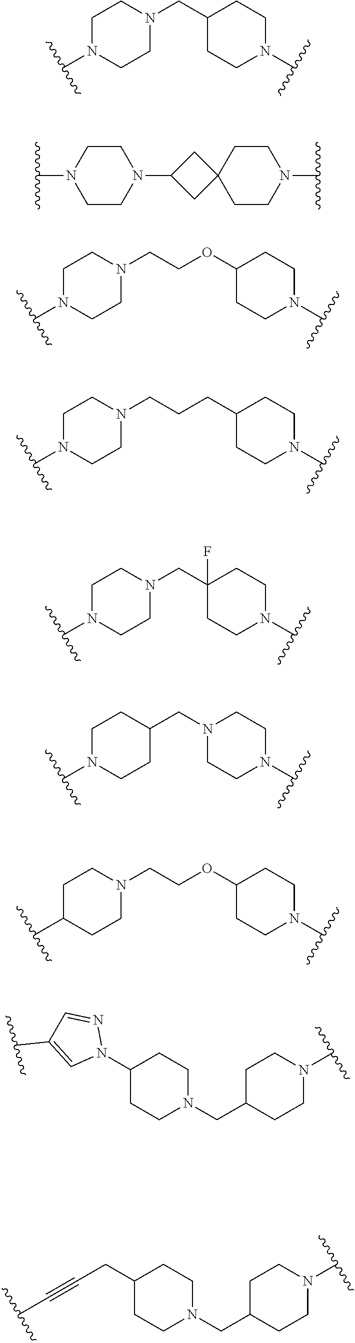

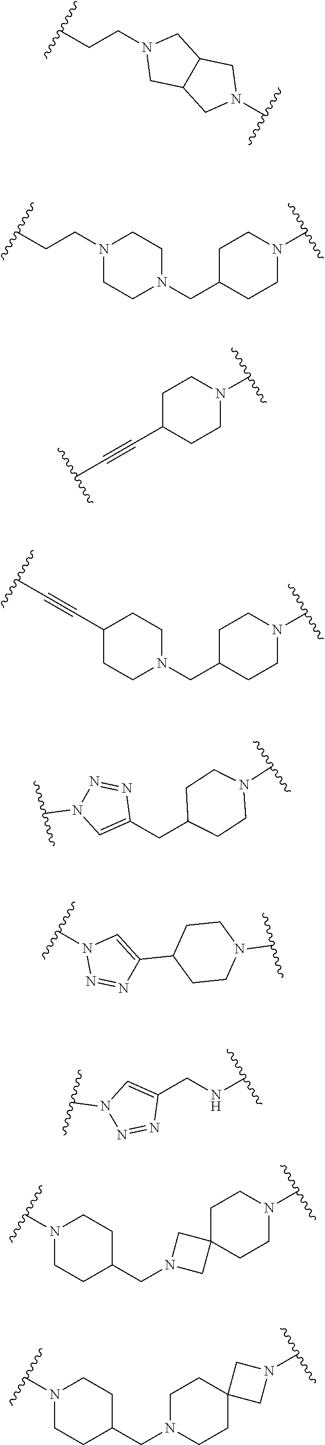

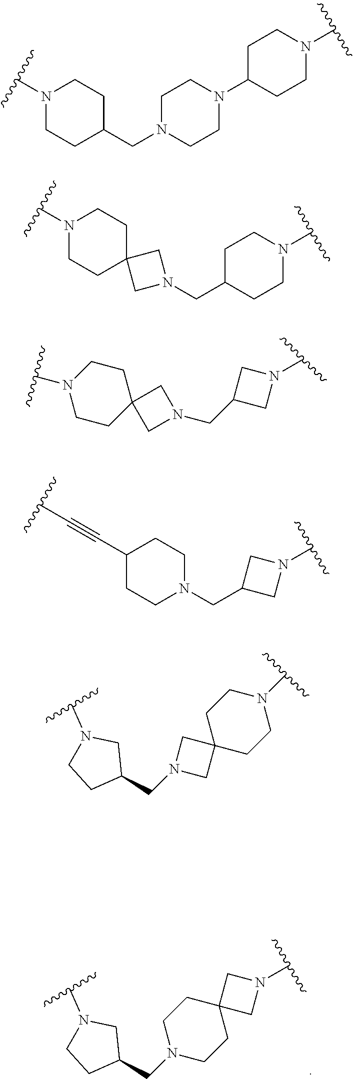 [SMILES: *N1CCC(CN2CCN(C3CCN(*)CC3)CC2)CC1 ; *N1CCC(CN2CC3(CCN(*)CC3)C2)CC1 ; *N1CCC2(CC1)CN(CC1CN(*)C1)C2 ; *C#CC1CCN(CC2CN(*)C2)CC1 ; *N1CCC2(CC1)CN(C[C@H]1CCN(*)C1)C2 ; *N1CC[C@H](CN2CCC3(CC2)CN(*)C3)C1]

7. The compound of claim 1 or a pharmaceutically acceptable salt thereof, wherein B is direct bond, *—C(O)—CH₂—O—*, *—C(O)—CH₂—NH—*, 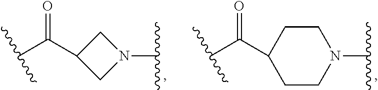 [SMILES: *C(=O)C1CN(*)C1 ; *C(=O)C1CCN(*)CC1]
O, or NH.

8. The compound of claim 7 or a pharmaceutically acceptable salt thereof, wherein B is direct bond, *—C(O)—CH₂—O—*, *—C(O)—CH₂—NH—*, 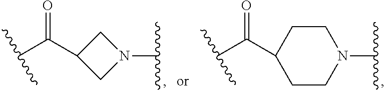 [SMILES: *C(=O)C1CN(*)C1 ; *C(=O)C1CCN(*)CC1]
and B is connected like

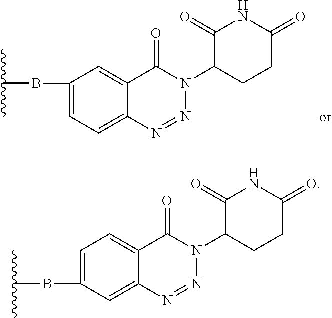

9. The compound of claim 1 or a pharmaceutically acceptable salt thereof, wherein the compound is any one of the following compounds:

| Compound | |
|---|---|
| No. | IUPAC name |
| 1 | N-((1r,3r)-3-(3-chloro-4-cyanophenoxy)-2,2,4,4-tetramethylcyclobutyl)-4-((17-((3-(2,6- |
| | dioxopiperidin-3-yl)-4-oxo-3,4-dihydrobenzo[d][1,2,3]triazin-5-yl)amino)-3,6,9,12,15- |
| | pentaoxaheptadecyl)oxy)benzamide |
| 2 | N-((1r,3r)-3-(3-chloro-4-cyanophenoxy)-2,2,4,4-tetramethylcyclobutyl)-4-((14-((3-(2,6- |
| | dioxopiperidin-3-yl)-4-oxo-3,4-dihydrobenzo[d][1,2,3]triazin-5-yl)amino)-3,6,9,12- |
| | tetraoxatetradecyl)oxy)benzamide |
| 3 | N-((1r,3r)-3-(3-chloro-4-cyanophenoxy)-2,2,4,4-tetramethylcyclobutyl)-4-((18-((3-(2,6- |
| | dioxopiperidin-3-yl)-4-oxo-3,4-dihydrobenzo[d][1,2,3]triazin-5-yl)amino)-3,6,9,12,15- |
| | pentaoxaoctadecyl)oxy)benzamide |
| 4 | N-((1r,3r)-3-(3-chloro-4-cyanophenoxy)-2,2,4,4-tetramethylcyclobutyl)-4-((18-((3-(2,6- |
| | dioxopiperidin-3-yl)-4-oxo-3,4-dihydrobenzo[d][1,2,3]triazin-5-yl)amino)-3,6,9,12,15- |
| | pentaoxaoctadecyl)oxy)benzamide |
| 5 | N-((1r,3r)-3-(3-chloro-4-cyanophenoxy)-2,2,4,4-tetramethylcyclobutyl)-4-((19-((3-(2,6- |
| | dioxopiperidin-3-yl)-4-oxo-3,4-dihydrobenzo[d][1,2,3]triazin-5-yl)amino)-3,6,9,12,15- |
| | pentaoxanonadecyl)oxy)benzamide |
| 6 | N-((1r,3r)-3-(3-chloro-4-cyanophenoxy)-2,2,4,4-tetramethylcyclobutyl)-4-((22-((3-(2,6- |
| | dioxopiperidin-3-yl)-4-oxo-3,4-dihydrobenzo[d][1,2,3]triazin-5-yl)amino)-3,6,9,12,15,18- |
| | hexaoxadocosyl)oxy)benzamide |
| 7 | N-((1r,3r)-3-(3-chloro-4-cyanophenoxy)-2,2,4,4-tetramethylcyclobutyl)-4-(4-((1-(3-(2,6- |
| | dioxopiperidin-3-yl)-4-oxo-3,4-dihydrobenzo[d][1,2,3]triazin-5-yl)piperidin-4- |
| | yl)methyl)piperazin-1-yl)benzamide |
| 8 | N-((1r,3r)-3-(3-chloro-4-cyanophenoxy)-2,2,4,4-tetramethylcyclobutyl)-4-(4-((1-(3-(2,6- |
| | dioxopiperidin-3-yl)-4-oxo-3,4-dihydrobenzo[d][1,2,3]triazin-6-yl)piperidin-4- |
| | yl)methyl)piperazin-1-yl)benzamide |
| 9 | N-((1r,3r)-3-(3-chloro-4-cyanophenoxy)-2,2,4,4-tetramethylcyclobutyl)-4-(4-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)piperazin-1-yl)benzamide |
| 10 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)benzamide |
| 11 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)benzamide |
| 12 | N-((1r,3r)-3-(3-chloro-4-cyanophenoxy)-2,2,4,4-tetramethylcyclobutyl)-4-(4-((1-(3-(2,6- |
| | dioxopiperidin-3-yl)-4-oxo-3,4-dihydrobenzo[d][1,2,3]triazin-7-yl)piperidin-4- |
| | yl)methyl)piperazin-1-yl)benzamide |
| 13 | N-((1r,3r)-3-(3-chloro-4-cyanophenoxy)-2,2,4,4-tetramethylcyclobutyl)-4-(4-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)methyl)piperazin-1-yl)benzamide |
| 14 | N-((1r,3r)-3-(3-chloro-4-cyanophenoxy)-2,2,4,4-tetramethylcyclobutyl)-4-((22-((3-(2,6- |
| | dioxopiperidin-3-yl)-4-oxo-3,4-dihydrobenzo[d][1,2,3]triazin-5-yl)amino)-21-oxo- |
| | 3,6,9,12,15-pentaoxa-20-azadocosyl)oxy)benzamide |
| 15 | N-(1-(4-(((1r,3r)-3-(3-chloro-4-cyanophenoxy)-2,2,4,4- |
| | tetramethylcyclobutyl)carbamoyl)phenoxy)-3,6,9,12,15-pentaoxanonadecan-19-yl)-1-(3- |
| | (2,6-dioxopiperidin-3-yl)-4-oxo-3,4-dihydrobenzo[d][1,2,3]triazin-6-yl)piperidine-4- |
| | carboxamide |
| 16 | N-(1-(4-(((1r,3r)-3-(3-chloro-4-cyanophenoxy)-2,2,4,4- |
| | tetramethylcyclobutyl)carbamoyl)phenoxy)-3,6,9,12,15-pentaoxanonadecan-19-yl)-1-(3- |
| | (2,6-dioxopiperidin-3-yl)-4-oxo-3,4-dihydrobenzo[d][1,2,3]triazin-6-yl)azetidine-3- |
| | carboxamide |
| 17 | N-((1r,3r)-3-(3-chloro-4-cyanophenoxy)-2,2,4,4-tetramethylcyclobutyl)-4-((19-((3-(2,6- |
| | dioxopiperidin-3-yl)-4-oxo-3,4-dihydrobenzo[d][1,2,3]triazin-6-yl)amino)-3,6,9,12,15- |
| | pentaoxanonadecyl)oxy)benzamide |
| 18 | N-((1r,3r)-3-(3-chloro-4-cyanophenoxy)-2,2,4,4-tetramethylcyclobutyl)-4-(4-(7-(2-((3-(2,6- |
| | dioxopiperidin-3-yl)-4-oxo-3,4-dihydrobenzo[d][1,2,3]triazin-6-yl)oxy)acetyl)-7- |
| | azaspiro[3.5]nonan-2-yl)piperazin-1-yl)benzamide |
| 19 | N-((1r,3r)-3-(3-chloro-4-cyanophenoxy)-2,2,4,4-tetramethylcyclobutyl)-4-(4-(7-((3-(2,6- |
| | dioxopiperidin-3-yl)-4-oxo-3,4-dihydrobenzo[d][1,2,3]triazin-6-yl)glycyl)-7- |
| | azaspiro[3.5]nonan-2-yl)piperazin-1-yl)benzamide |
| 20 | N-((1r,3r)-3-(3-chloro-4-cyanophenoxy)-2,2,4,4-tetramethylcyclobutyl)-4-(4-(7-(1-(3-(2,6- |
| | dioxopiperidin-3-yl)-4-oxo-3,4-dihydrobenzo[d][1,2,3]triazin-6-yl)piperidine-4-carbonyl)-7- |
| | azaspiro[3.5]nonan-2-yl)piperazin-1-yl)benzamide |
| 21 | N-((1r,3r)-3-(3-chloro-4-cyanophenoxy)-2,2,4,4-tetramethylcyclobutyl)-4-(4-(7-(1-(3-(2,6- |
| | dioxopiperidin-3-yl)-4-oxo-3,4-dihydrobenzo[d][1,2,3]triazin-6-yl)azetidine-3-carbonyl)-7- |
| | azaspiro[3.5]nonan-2-yl)piperazin-1-yl)benzamide |
| 22 | N-((1r,3r)-3-(3-chloro-4-cyanophenoxy)-2,2,4,4-tetramethylcyclobutyl)-4-(4-(7-(3-(2,6- |
| | dioxopiperidin-3-yl)-4-oxo-3,4-dihydrobenzo[d][1,2,3]triazin-5-yl)-7-azaspiro[3.5]nonan-2- |
| | yl)piperazin-1-yl)benzamide |
| 23 | N-((1r,3r)-3-(3-chloro-4-cyanophenoxy)-2,2,4,4-tetramethylcyclobutyl)-4-(4-(7-(3-(2,6- |
| | dioxopiperidin-3-yl)-4-oxo-3,4-dihydrobenzo[d][1,2,3]triazin-6-yl)-7-azaspiro[3.5]nonan-2- |
| | yl)piperazin-1-yl)benzamide |
| 24 | N-((1r,3r)-3-(3-chloro-4-cyanophenoxy)-2,2,4,4-tetramethylcyclobutyl)-4-(4-(2-(1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)ethyl)piperazin-1-yl)benzamide |
| 25 | N-((1r,3r)-3-(3-chloro-4-cyanophenoxy)-2,2,4,4-tetramethylcyclobutyl)-4-(4-(2-(1-((3-(2,6- |
| | dioxopiperidin-3-yl)-4-oxo-3,4-dihydrobenzo[d][1,2,3]triazin-6-yl)glycyl)piperidin-4- |
| | yl)ethyl)piperazin-1-yl)benzamide |
| 26 | N-((1r,3r)-3-(3-chloro-4-cyanophenoxy)-2,2,4,4-tetramethylcyclobutyl)-4-(4-(2-(1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)ethyl)piperazin-1-yl)benzamide |
| 27 | N-((1r,3r)-3-(3-chloro-4-cyanophenoxy)-2,2,4,4-tetramethylcyclobutyl)-4-(4-(2-(1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)ethyl)piperazin-1-yl)benzamide |
| 28 | N-((1r,3r)-3-(3-chloro-4-cyanophenoxy)-2,2,4,4-tetramethylcyclobutyl)-4-(4-(2-(1-(3-(2,6- |
| | dioxopiperidin-3-yl)-4-oxo-3,4-dihydrobenzo[d][1,2,3]triazin-5-yl)piperidin-4- |
| | yl)ethyl)piperazin-1-yl)benzamide |
| 29 | N-((1r,3r)-3-(3-chloro-4-cyanophenoxy)-2,2,4,4-tetramethylcyclobutyl)-4-(4-(2-(1-(3-(2,6- |
| | dioxopiperidin-3-yl)-4-oxo-3,4-dihydrobenzo[d][1,2,3]triazin-6-yl)piperidin-4- |
| | yl)ethyl)piperazin-1-yl)benzamide |
| 30 | N-((1r,3r)-3-(3-chloro-4-cyanophenoxy)-2,2,4,4-tetramethylcyclobutyl)-4-(4-(2-(1-(3-(2,6- |
| | dioxopiperidin-3-yl)-4-oxo-3,4-dihydrobenzo[d][1,2,3]triazin-6-yl)piperidin-4- |
| | yl)ethyl)piperazin-1-yl)benzamide |
| 31 | N-((1r,3r)-3-(3-chloro-4-cyanophenoxy)-2,2,4,4-tetramethylcyclobutyl)-4-(4-(2-((1-((3- |
| | (2,6-dioxopiperidin-3-yl)-4-oxo-3,4-dihydrobenzo[d][1,2,3]triazin-6-yl)glycyl)piperidin-4- |
| | yl)oxy)ethyl)piperazin-1-yl)benzamide |
| 32 | N-((1r,3r)-3-(3-chloro-4-cyanophenoxy)-2,2,4,4-tetramethylcyclobutyl)-4-(4-(2-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)oxy)ethyl)piperazin-1-yl)benzamide |
| 33 | N-((1r,3r)-3-(3-chloro-4-cyanophenoxy)-2,2,4,4-tetramethylcyclobutyl)-4-(4-(2-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)oxy)ethyl)piperazin-1-yl)benzamide |
| 34 | N-((1r,3r)-3-(3-chloro-4-cyanophenoxy)-2,2,4,4-tetramethylcyclobutyl)-4-(4-(2-((1-(3-(2,6- |
| | dioxopiperidin-3-yl)-4-oxo-3,4-dihydrobenzo[d][1,2,3]triazin-6-yl)piperidin-4- |
| | yl)oxy)ethyl)piperazin-1-yl)benzamide |
| 35 | N-((1r,3r)-3-(3-chloro-4-cyanophenoxy)-2,2,4,4-tetramethylcyclobutyl)-4-(4-(3-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)oxy)propyl)piperazin-1-yl)benzamide |
| 36 | N-((1r,3r)-3-(3-chloro-4-cyanophenoxy)-2,2,4,4-tetramethylcyclobutyl)-4-(4-(3-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)oxy)propyl)piperazin-1-yl)benzamide |
| 37 | N-((1r,3r)-3-(3-chloro-4-cyanophenoxy)-2,2,4,4-tetramethylcyclobutyl)-4-(4-(3-((1-((3- |
| | (2,6-dioxopiperidin-3-yl)-4-oxo-3,4-dihydrobenzo[d][1,2,3]triazin-6-yl)glycyl)piperidin-4- |
| | yl)oxy)propyl)piperazin-1-yl)benzamide |
| 38 | N-((1r,3r)-3-(3-chloro-4-cyanophenoxy)-2,2,4,4-tetramethylcyclobutyl)-4-(4-(3-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)oxy)propyl)piperazin-1-yl)benzamide |
| 39 | N-((1r,3r)-3-(3-chloro-4-cyanophenoxy)-2,2,4,4-tetramethylcyclobutyl)-4-(4-(3-((1-(3-(2,6- |
| | dioxopiperidin-3-yl)-4-oxo-3,4-dihydrobenzo[d][1,2,3]triazin-6-yl)piperidin-4- |
| | yl)oxy)propyl)piperazin-1-yl)benzamide |
| 40 | N-((1r,3r)-3-(3-chloro-4-cyanophenoxy)-2,2,4,4-tetramethylcyclobutyl)-4-(4-((3-(2,6- |
| | dioxopiperidin-3-yl)-4-oxo-3,4-dihydrobenzo[d][1,2,3]triazin-6-yl)glycyl)piperazin-1- |
| | yl)benzamide |
| 41 | N-((1r,3r)-3-(3-chloro-4-cyanophenoxy)-2,2,4,4-tetramethylcyclobutyl)-4-(4-(2-((3-(2,6- |
| | dioxopiperidin-3-yl)-4-oxo-3,4-dihydrobenzo[d][1,2,3]triazin-6-yl)oxy)acetyl)piperazin-1- |
| | yl)benzamide |
| 42 | N-((1r,3r)-3-(3-chloro-4-cyanophenoxy)-2,2,4,4-tetramethylcyclobutyl)-4-(4-(1-(3-(2,6- |
| | dioxopiperidin-3-yl)-4-oxo-3,4-dihydrobenzo[d][1,2,3]triazin-6-yl)piperidine-4- |
| | carbonyl)piperazin-1-yl)benzamide |
| 43 | N-((1r,3r)-3-(3-chloro-4-cyanophenoxy)-2,2,4,4-tetramethylcyclobutyl)-4-(4-(1-(3-(2,6- |
| | dioxopiperidin-3-yl)-4-oxo-3,4-dihydrobenzo[d][1,2,3]triazin-6-yl)azetidine-3- |
| | carbonyl)piperazin-1-yl)benzamide |
| 44 | N-((1r,3r)-3-(3-chloro-4-cyanophenoxy)-2,2,4,4-tetramethylcyclobutyl)-4-(4-(1-(3-(2,6- |
| | dioxopiperidin-3-yl)-4-oxo-3,4-dihydrobenzo[d][1,2,3]triazin-6-yl)azetidine-3- |
| | carbonyl)piperazin-1-yl)benzamide |
| 45 | N-((1r,3r)-3-(3-chloro-4-cyanophenoxy)-2,2,4,4-tetramethylcyclobutyl)-4-(4-(3-(1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)propyl)piperazin-1-yl)benzamide |
| 46 | N-((1r,3r)-3-(3-chloro-4-cyanophenoxy)-2,2,4,4-tetramethylcyclobutyl)-4-(4-(3-(1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)propyl)piperazin-1-yl)benzamide |
| 47 | N-((1r,3r)-3-(3-chloro-4-cyanophenoxy)-2,2,4,4-tetramethylcyclobutyl)-4-(4-(3-(1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)propyl)piperazin-1-yl)benzamide |
| 48 | N-((1r,3r)-3-(3-chloro-4-cyanophenoxy)-2,2,4,4-tetramethylcyclobutyl)-4-(4-(3-(1-(3-(2,6- |
| | dioxopiperidin-3-yl)-4-oxo-3,4-dihydrobenzo[d][1,2,3]triazin-6-yl)piperidin-4- |
| | yl)propyl)piperazin-1-yl)benzamide |
| 49 | N-((1r,3r)-3-(3-chloro-4-cyanophenoxy)-2,2,4,4-tetramethylcyclobutyl)-4-(4-((4-(1-(3-(2,6- |
| | dioxopiperidin-3-yl)-4-oxo-3,4-dihydrobenzo[d][1,2,3]triazin-6-yl)piperidine-4- |
| | carbonyl)piperazin-1-yl)methyl)piperidin-1-yl)benzamide |
| 50 | N-((1r,3r)-3-(3-chloro-4-cyanophenoxy)-2,2,4,4-tetramethylcyclobutyl)-4-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)benzamide |
| 51 | N-((1r,3r)-3-(3-chloro-4-cyanophenoxy)-2,2,4,4-tetramethylcyclobutyl)-4-(1-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)benzamide |
| 52 | N-((1r,3r)-3-(3-chloro-4-cyanophenoxy)-2,2,4,4-tetramethylcyclobutyl)-4-(1-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)benzamide |
| 53 | N-((1r,3r)-3-(3-chloro-4-cyanophenoxy)-2,2,4,4-tetramethylcyclobutyl)-4-(4-((1-((3-(2,6- |
| | dioxopiperidin-3-yl)-4-oxo-3,4-dihydrobenzo[d][1,2,3]triazin-6-yl)glycyl)-4- |
| | hydroxypiperidin-4-yl)methyl)piperazin-1-yl)benzamide |
| 54 | N-((1r,3r)-3-(3-chloro-4-cyanophenoxy)-2,2,4,4-tetramethylcyclobutyl)-4-(4-((1-(2-((3- |
| | (2,6-dioxopiperidin-3-yl)-4-oxo-3,4-dihydrobenzo[d][1,2,3]triazin-6-yl)oxy)acetyl)-4- |
| | hydroxypiperidin-4-yl)methyl)piperazin-1-yl)benzamide |
| 55 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)piperidine-4-carbonyl)-4- |
| | hydroxypiperidin-4-yl)methyl)piperazin-1-yl)benzamide |
| 56 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)azetidine-3-carbonyl)-4- |
| | hydroxypiperidin-4-yl)methyl)piperazin-1-yl)benzamide |
| 57 | N-((1r,3r)-3-(3-chloro-4-cyanophenoxy)-2,2,4,4-tetramethylcyclobutyl)-4-(4-(2-(4-(3-(2,6- |
| | dioxopiperidin-3-yl)-4-oxo-3,4-dihydrobenzo[d][1,2,3]triazin-6-yl)piperazin-1- |
| | yl)acetyl)piperazin-1-yl)benzamide |
| 58 | N-((1r,3r)-3-(3-chloro-4-cyanophenoxy)-2,2,4,4-tetramethylcyclobutyl)-4-(4-((1-(2-(4-(3- |
| | (2,6-dioxopiperidin-3-yl)-4-oxo-3,4-dihydrobenzo[d][1,2,3]triazin-6-yl)piperazin-1- |
| | yl)acetyl)piperidin-4-yl)methyl)piperazin-1-yl)benzamide |
| 59 | N-((1r,3r)-3-(3-chloro-4-cyanophenoxy)-2,2,4,4-tetramethylcyclobutyl)-4-(4-((1-(2-(2-((3- |
| | (2,6-dioxopiperidin-3-yl)-4-oxo-3,4-dihydrobenzo[d][1,2,3]triazin-6- |
| | yl)amino)acetamido)ethyl)piperidin-4-yl)methyl)piperazin-1-yl)benzamide |
| 60 | N-((1r,3r)-3-(3-chloro-4-cyanophenoxy)-2,2,4,4-tetramethylcyclobutyl)-4-(4-((1-(2-(2-((3- |
| | (2,6-dioxopiperidin-3-yl)-4-oxo-3,4-dihydrobenzo[d][1,2,3]triazin-6- |
| | yl)oxy)acetamido)ethyl)piperidin-4-yl)methyl)piperazin-1-yl)benzamide |
| 61 | N-((1r,3r)-3-(3-chloro-4-cyanophenoxy)-2,2,4,4-tetramethylcyclobutyl)-4-(4-((1-(3-(2,6- |
| | dioxopiperidin-3-yl)-4-oxo-3,4-dihydrobenzo[d][1,2,3]triazin-6-yl)-4-fluoropiperidin-4- |
| | yl)methyl)piperazin-1-yl)benzamide |
| 62 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)piperidine-4-carbonyl)-4- |
| | fluoropiperidin-4-yl)methyl)piperazin-1-yl)benzamide |
| 63 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)azetidine-3-carbonyl)-4- |
| | fluoropiperidin-4-yl)methyl)piperazin-1-yl)benzamide |
| 64 | N-((1r,3r)-3-(3-chloro-4-cyanophenoxy)-2,2,4,4-tetramethylcyclobutyl)-4-(4-((1-(3-(2,6- |
| | dioxopiperidin-3-yl)-4-oxo-3,4-dihydrobenzo[d][1,2,3]triazin-6-yl)piperidin-4- |
| | ylidene)methyl)piperazin-1-yl)benzamide |
| 65 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-ylidene)methyl)piperazin-1-yl)benzamide |
| 66 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-ylidene)methyl)piperazin-1-yl)benzamide |
| 67 | N-((1r,3r)-3-(3-chloro-4-cyanophenoxy)-2,2,4,4-tetramethylcyclobutyl)-4-(4-((1-((1-(3- |
| | (2,6-dioxopiperidin-3-yl)-4-oxo-3,4-dihydrobenzo[d][1,2,3]triazin-6-yl)azetidin-3- |
| | yl)methyl)piperidin-4-yl)methyl)piperazin-1-yl)benzamide |
| 68 | N-(2-(4-((4-(4-(((1r,3r)-3-(3-chloro-4-cyanophenoxy)-2,2,4,4- |
| | tetramethylcyclobutyl)carbamoyl)phenyl)piperazin-1-yl)methyl)piperidin-1-yl)ethyl)-1-(3- |
| | (2,6-dioxopiperidin-3-yl)-4-oxo-3,4-dihydrobenzo[d][1,2,3]triazin-6-yl)piperidine-4- |
| | carboxamide |
| 69 | N-(2-(4-((4-(4-(((1r,3r)-3-(3-chloro-4-cyanophenoxy)-2,2,4,4- |
| | tetramethylcyclobutyl)carbamoyl)phenyl)piperazin-1-yl)methyl)piperidin-1-yl)ethyl)-1-(3- |
| | (2,6-dioxopiperidin-3-yl)-4-oxo-3,4-dihydrobenzo[d][1,2,3]triazin-6-yl)azetidine-3- |
| | carboxamide |
| 70 | N-((1r,3r)-3-(3-chloro-4-cyanophenoxy)-2,2,4,4-tetramethylcyclobutyl)-4-(4-((1-((1-(3- |
| | (2,6-dioxopiperidin-3-yl)-4-oxo-3,4-dihydrobenzo[d][1,2,3]triazin-6-yl)piperidin-4- |
| | yl)methyl)piperidin-4-yl)methyl)piperazin-1-yl)benzamide |
| 71 | N-((1r,3r)-3-(3-chloro-4-cyanophenoxy)-2,2,4,4-tetramethylcyclobutyl)-4-(5-(7-(1-(3-(2,6- |
| | dioxopiperidin-3-yl)-4-oxo-3,4-dihydrobenzo[d][1,2,3]triazin-6-yl)piperidine-4-carbonyl)-7- |
| | azaspiro[3.5]nonan-2-yl)-2,5-diazabicyclo[2.2.1]heptan-2-yl)benzamide |
| 72 | N-((1r,3r)-3-(3-chloro-4-cyanophenoxy)-2,2,4,4-tetramethylcyclobutyl)-4-(5-((1-(3-(2,6- |
| | dioxopiperidin-3-yl)-4-oxo-3,4-dihydrobenzo[d][1,2,3]triazin-6-yl)piperidin-4-yl)methyl)- |
| | 2,5-diazabicyclo[2.2.1]heptan-2-yl)benzamide |
| 73 | N-((1r,3r)-3-(3-chloro-4-cyanophenoxy)-2,2,4,4-tetramethylcyclobutyl)-4-(5-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)-2,5-diazabicyclo[2.2.1]heptan-2-yl)benzamide |
| 74 | N-((1r,3r)-3-(3-chloro-4-cyanophenoxy)-2,2,4,4-tetramethylcyclobutyl)-4-(1-(2-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)oxy)ethyl)piperidin-4-yl)benzamide |
| 75 | N-((1r,3r)-3-(3-chloro-4-cyanophenoxy)-2,2,4,4-tetramethylcyclobutyl)-4-(1-(2-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)oxy)ethyl)piperidin-4-yl)benzamide |
| 76 | N-((1r,3r)-3-(3-chloro-4-cyanophenoxy)-2,2,4,4-tetramethylcyclobutyl)-4-(1-(2-((1-(3-(2,6- |
| | dioxopiperidin-3-yl)-4-oxo-3,4-dihydrobenzo[d][1,2,3]triazin-6-yl)piperidin-4- |
| | yl)oxy)ethyl)piperidin-4-yl)benzamide |
| 77 | N-((1r,3r)-3-(3-chloro-4-isocyanophenoxy)-2,2,4,4-tetramethylcyclobutyl)-4-(4-(2-(1-(3- |
| | (2,6-dioxopiperidin-3-yl)-4-oxo-3,4-dihydrobenzo[d][1,2,3]triazin-6-yl)-1H-1,2,3-triazol-4- |
| | yl)ethyl)piperazin-1-yl)benzamide |
| 78 | N-((1r,3r)-3-(3-chloro-4-cyanophenoxy)-2,2,4,4-tetramethylcyclobutyl)-6-(4-((1-(3-(2,6- |
| | dioxopiperidin-3-yl)-4-oxo-3,4-dihydrobenzo[d][1,2,3]triazin-6-yl)piperidin-4- |
| | yl)methyl)piperazin-1-yl)nicotinamide |
| 79 | N-((1r,3r)-3-(3-chloro-4-cyanophenoxy)-2,2,4,4-tetramethylcyclobutyl)-5-(4-((1-(3-(2,6- |
| | dioxopiperidin-3-yl)-4-oxo-3,4-dihydrobenzo[d][1,2,3]triazin-6-yl)piperidin-4- |
| | yl)methyl)piperazin-1-yl)pyrazine-2-carboxamide |
| 80 | N-((1r,3r)-3-(3-chloro-4-cyanophenoxy)-2,2,4,4-tetramethylcyclobutyl)-6-(4-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)nicotinamide |
| 81 | N-((1r,3r)-3-(3-chloro-4-cyanophenoxy)-2,2,4,4-tetramethylcyclobutyl)-6-(4-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)nicotinamide |
| 82 | N-((1r,3r)-3-(3-chloro-4-cyanophenoxy)-2,2,4,4-tetramethylcyclobutyl)-4-(1-(1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)benzamide |
| 83 | N-((1r,3r)-3-(3-chloro-4-cyanophenoxy)-2,2,4,4-tetramethylcyclobutyl)-4-(1-(1-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)benzamide |
| 84 | N-((1r,3r)-3-(3-chloro-4-cyanophenoxy)-2,2,4,4-tetramethylcyclobutyl)-4-(1-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)-1H-pyrazol-4-yl)benzamide |
| 85 | N-((1r,3r)-3-(3-chloro-4-cyanophenoxy)-2,2,4,4-tetramethylcyclobutyl)-4-(3-(1-(3-(2,6- |
| | dioxopiperidin-3-yl)-4-oxo-3,4-dihydrobenzo[d][1,2,3]triazin-6-yl)piperidin-4-yl)prop-1-yn- |
| | 1-yl)benzamide |
| 86 | N-((1r,3r)-3-(3-chloro-4-cyanophenoxy)-2,2,4,4-tetramethylcyclobutyl)-4-(3-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)prop-1-yn-1-yl)benzamide |
| 87 | N-((1r,3r)-3-(3-chloro-4-cyanophenoxy)-2,2,4,4-tetramethylcyclobutyl)-4-(3-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)prop-1-yn-1-yl)benzamide |
| 88 | N-((1r,3r)-3-(3-chloro-4-cyanophenoxy)-2,2,4,4-tetramethylcyclobutyl)-4-(3-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)prop-1-yn-1-yl)benzamide |
| 89 | N-((1r,3r)-3-(3-chloro-4-cyanophenoxy)-2,2,4,4-tetramethylcyclobutyl)-4-(3-(1 [symbol] (3-(2,6- |
| | dioxopiperidin-3-yl)-4-oxo-3,4-dihydrobenzo[d][1,2,3]triazin-6-yl)-[1,4 [symbol] bipiperidin]-4- |
| | yl)prop-1-yn-1-yl)benzamide |
| 90 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)-1H-1,2,3-triazol-1-yl)benzamide |
| 91 | N-((1r,3r)-3-(3-chloro-4-cyanophenoxy)-2,2,4,4-tetramethylcyclobutyl)-4-(4-((1-(3-(2,6- |
| | dioxopiperidin-3-yl)-4-oxo-3,4-dihydrobenzo[d][1,2,3]triazin-6-yl)-1H-1,2,3-triazol-4- |
| | yl)methyl)piperazin-1-yl)benzamide |
| 92 | N-((1r,3r)-3-(3-chloro-4-cyanophenoxy)-2,2,4,4-tetramethylcyclobutyl)-4-(2-(5-(3-(2,6- |
| | dioxopiperidin-3-yl)-4-oxo-3,4-dihydrobenzo[d][1,2,3]triazin-6-yl)hexahydropyrrolo[3,4- |
| | c]pyrrol-2(1H)-yl)ethyl)benzamide |
| 93 | N-((1r,3r)-3-(3-chloro-4-cyanophenoxy)-2,2,4,4-tetramethylcyclobutyl)-4-(2-(5-(1-(3-(2,6- |
| | dioxopiperidin-3-yl)-4-oxo-3,4-dihydrobenzo[d][1,2,3]triazin-6-yl)piperidine-4- |
| | carbonyl)hexahydropyrrolo[3,4-c]pyrrol-2(1H)-yl)ethyl)benzamide |
| 94 | N-((1r,3r)-3-(3-chloro-4-cyanophenoxy)-2,2,4,4-tetramethylcyclobutyl)-4-(2-(5-(1-(3-(2,6- |
| | dioxopiperidin-3-yl)-4-oxo-3,4-dihydrobenzo[d][1,2,3]triazin-6-yl)azetidine-3- |
| | carbonyl)hexahydropyrrolo[3,4-c]pyrrol-2(1H)-yl)ethyl)benzamide |
| 95 | N-((1r,3r)-3-(3-chloro-4-cyanophenoxy)-2,2,4,4-tetramethylcyclobutyl)-4-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)ethynyl)benzamide |
| 96 | N-((1r,3r)-3-(3-chloro-4-cyanophenoxy)-2,2,4,4-tetramethylcyclobutyl)-4-((1-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)ethynyl)benzamide |
| 97 | N-((1r,3r)-3-(3-chloro-4-cyanophenoxy)-2,2,4,4-tetramethylcyclobutyl)-4-((1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)benzamide |
| 98 | N-((1r,3r)-3-(3-chloro-4-cyanophenoxy)-2,2,4,4-tetramethylcyclobutyl)-4-((1-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)piperidin-4-yl)ethynyl)benzamide |
| 99 | N-((1r,3r)-3-(3-chloro-4-cyanophenoxy)-2,2,4,4-tetramethylcyclobutyl)-4-((1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)ethynyl)benzamide |
| 100 | N-((1r,3r)-3-(3-chloro-4-cyanophenoxy)-2,2,4,4-tetramethylcyclobutyl)-2-(4-((1-(3-(2,6- |
| | dioxopiperidin-3-yl)-4-oxo-3,4-dihydrobenzo[d][1,2,3]triazin-6-yl)piperidin-4- |
| | yl)methyl)piperazin-1-yl)pyrimidine-5-carboxamide |
| 101 | N-((1r,3r)-3-(3-chloro-4-cyanophenoxy)-2,2,4,4-tetramethylcyclobutyl)-4-(4-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)-1H-1,2,3-triazol-1-yl)benzamide |
| 102 | N-((1r,3r)-3-(3-chloro-4-cyanophenoxy)-2,2,4,4-tetramethylcyclobutyl)-4-(4-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)-1H-1,2,3-triazol-1-yl)benzamide |
| 103 | N-((1r,3r)-3-(3-chloro-4-cyanophenoxy)-2,2,4,4-tetramethylcyclobutyl)-4-(4-((1-(3-(2,6- |
| | dioxopiperidin-3-yl)-4-oxo-3,4-dihydrobenzo[d][1,2,3]triazin-6-yl)piperidin-4-yl)methyl)- |
| | 1H-1,2,3-triazol-1-yl)benzamide |
| 104 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)-1H-1,2,3-triazol-1-yl)benzamide |
| 105 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)piperidin-4-yl)-1H-1,2,3- |
| | triazol-4-yl)methyl)piperazin-1-yl)benzamide |
| 106 | N-((1r,3r)-3-(3-chloro-4-cyanophenoxy)-2,2,4,4-tetramethylcyclobutyl)-4-(4-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)-1H-1,2,3-triazol-1-yl)benzamide |
| 107 | N-((1r,3r)-3-(3-chloro-4-cyanophenoxy)-2,2,4,4-tetramethylcyclobutyl)-4-(4-(1-((3-(2,6- |
| | dioxopiperidin-3-yl)-4-oxo-3,4-dihydrobenzo[d][1,2,3]triazin-6-yl)glycyl)piperidin-4-yl)-1H- |
| | 1,2,3-triazol-1-yl)benzamide |
| 108 | N-((1r,3r)-3-(3-chloro-4-cyanophenoxy)-2,2,4,4-tetramethylcyclobutyl)-4-(2-(4-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)ethyl)benzamide |
| 109 | N-((1r,3r)-3-(3-chloro-4-cyanophenoxy)-2,2,4,4-tetramethylcyclobutyl)-4-(2-(4-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)ethyl)benzamide |
| 110 | N-((1r,3r)-3-(3-chloro-4-cyanophenoxy)-2,2,4,4-tetramethylcyclobutyl)-4-(2-(4-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperazin-1-yl)ethyl)benzamide |
| 111 | N-((1r,3r)-3-(3-chloro-4-cyanophenoxy)-2,2,4,4-tetramethylcyclobutyl)-4-(2-(4-((1-(3-(2,6- |
| | dioxopiperidin-3-yl)-4-oxo-3,4-dihydrobenzo[d][1,2,3]triazin-6-yl)piperidin-4- |
| | yl)methyl)piperazin-1-yl)ethyl)benzamide |
| 112 | N-((1r,3r)-3-(3-chloro-4-cyanophenoxy)-2,2,4,4-tetramethylcyclobutyl)-4-(1-(1-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)benzamide |
| 113 | N-((1r,3r)-3-(3-chloro-4-cyanophenoxy)-2,2,4,4-tetramethylcyclobutyl)-4-(1-(1-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)methyl)piperidin-4-yl)-1H-pyrazol-4-yl)benzamide |
| 114 | N-((1r,3r)-3-(3-chloro-4-cyanophenoxy)-2,2,4,4-tetramethylcyclobutyl)-6-(1-(1-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)nicotinamide |
| 115 | N-((1r,3r)-3-(3-chloro-4-cyanophenoxy)-2,2,4,4-tetramethylcyclobutyl)-6-(1-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)-1H-pyrazol-4-yl)nicotinamide |
| 116 | N-((1r,3r)-3-(3-chloro-4-cyanophenoxy)-2,2,4,4-tetramethylcyclobutyl)-4-(3-(1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)prop-1-yn-1-yl)benzamide |
| 117 | N-((1r,3r)-3-(3-chloro-4-cyanophenoxy)-2,2,4,4-tetramethylcyclobutyl)-4-(3-(1-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)prop-1-yn-1-yl)benzamide |
| 118 | N-((1r,3r)-3-(3-chloro-4-cyanophenoxy)-2,2,4,4-tetramethylcyclobutyl)-4-(3-(1-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperidin-4-yl)prop-1-yn-1-yl)benzamide |
| 119 | N-((1r,3r)-3-(3-chloro-4-cyanophenoxy)-2,2,4,4-tetramethylcyclobutyl)-4-(3-(1-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)methyl)piperidin-4-yl)prop-1-yn-1-yl)benzamide |
| 120 | N-((1r,3r)-3-(3-chloro-4-cyanophenoxy)-2,2,4,4-tetramethylcyclobutyl)-4-(4-((4-(3-(2,6- |
| | dioxopiperidin-3-yl)-4-oxo-3,4-dihydrobenzo[d][1,2,3]triazin-6-yl)piperazin-1-yl)methyl)- |
| | 1H-1,2,3-triazol-1-yl)benzamide |
| 121 | N-((1r,3r)-3-(3-chloro-4-cyanophenoxy)-2,2,4,4-tetramethylcyclobutyl)-4-(4-((4-(1-(3-(2,6- |
| | dioxopiperidin-3-yl)-4-oxo-3,4-dihydrobenzo[d][1,2,3]triazin-6-yl)piperidine-4- |
| | carbonyl)piperazin-1-yl)methyl)-1H-1,2,3-triazol-1-yl)benzamide |
| 122 | N-((1r,3r)-3-(3-chloro-4-cyanophenoxy)-2,2,4,4-tetramethylcyclobutyl)-4-(4-((4-(1-(3-(2,6- |
| | dioxopiperidin-3-yl)-4-oxo-3,4-dihydrobenzo[d][1,2,3]triazin-6-yl)azetidine-3- |
| | carbonyl)piperazin-1-yl)methyl)-1H-1,2,3-triazol-1-yl)benzamide |
| 123 | N-((1r,3r)-3-(3-chloro-4-cyanophenoxy)-2,2,4,4-tetramethylcyclobutyl)-4-(4-((4-((3-(2,6- |
| | dioxopiperidin-3-yl)-4-oxo-3,4-dihydrobenzo[d][1,2,3]triazin-6-yl)glycyl)piperazin-1- |
| | yl)methyl)-1H-1,2,3-triazol-1-yl)benzamide |
| 124 | N-((1r,3r)-3-(3-chloro-4-cyanophenoxy)-2,2,4,4-tetramethylcyclobutyl)-6-(1-(1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)nicotinamide |
| 125 | N-((1r,3r)-3-(3-chloro-4-cyanophenoxy)-2,2,4,4-tetramethylcyclobutyl)-6-(1-(1-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)nicotinamide |
| 126 | N-((1r,3r)-3-(3-chloro-4-cyanophenoxy)-2,2,4,4-tetramethylcyclobutyl)-6-(3-(1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)prop-1-yn-1-yl)nicotinamide |
| 127 | N-((1r,3r)-3-(3-chloro-4-cyanophenoxy)-2,2,4,4-tetramethylcyclobutyl)-6-(3-(1-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)prop-1-yn-1-yl)nicotinamide |
| 128 | N-((1r,3r)-3-(3-chloro-4-cyanophenoxy)-2,2,4,4-tetramethylcyclobutyl)-6-(3-(1-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperidin-4-yl)prop-1-yn-1-yl)nicotinamide |
| 129 | N-((1r,3r)-3-(3-chloro-4-cyanophenoxy)-2,2,4,4-tetramethylcyclobutyl)-6-(3-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)prop-1-yn-1-yl)nicotinamide |
| 130 | N-((1r,3r)-3-(3-chloro-4-cyanophenoxy)-2,2,4,4-tetramethylcyclobutyl)-6-((1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)nicotinamide |
| 131 | N-((1r,3r)-3-(3-chloro-4-cyanophenoxy)-2,2,4,4-tetramethylcyclobutyl)-6-((1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)nicotinamide |
| 132 | N-((1r,3r)-3-(3-chloro-4-cyanophenoxy)-2,2,4,4-tetramethylcyclobutyl)-6-((1-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)piperidin-4-yl)ethynyl)nicotinamide |
| 133 | N-((1r,3r)-3-(3-chloro-4-cyanophenoxy)-2,2,4,4-tetramethylcyclobutyl)-6-((1-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)methyl)piperidin-4-yl)ethynyl)nicotinamide |
| 134 | N-((1r,3r)-3-(3-chloro-4-cyanophenoxy)-2,2,4,4-tetramethylcyclobutyl)-4-(4-(3-(1-(3-(2,6- |
| | dioxopiperidin-3-yl)-4-oxo-3,4-dihydrobenzo[d][1,2,3]triazin-6-yl)-1H-1,2,3-triazol-4- |
| | yl)propyl)piperazin-1-yl)benzamide |
| 135 | N-((1r,3r)-3-(3-chloro-4-cyanophenoxy)-2,2,4,4-tetramethylcyclobutyl)-4-(5-(2-((3-(2,6- |
| | dioxopiperidin-3-yl)-4-oxo-3,4-dihydrobenzo[d][1,2,3]triazin-6- |
| | yl)oxy)acetyl)hexahydropyrrolo[3,4-c]pyrrol-2(1H)-yl)benzamide |
| 136 | N-((1r,3r)-3-(3-chloro-4-cyanophenoxy)-2,2,4,4-tetramethylcyclobutyl)-4-(5-((3-(2,6- |
| | dioxopiperidin-3-yl)-4-oxo-3,4-dihydrobenzo[d][1,2,3]triazin-6- |
| | yl)glycyl)hexahydropyrrolo[3,4-c]pyrrol-2(1H)-yl)benzamide |
| 137 | N-((1r,3r)-3-(3-chloro-4-cyanophenoxy)-2,2,4,4-tetramethylcyclobutyl)-4-(5-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)methyl)hexahydropyrrolo[3,4-c]pyrrol-2(1H)-yl)benzamide |
| 138 | N-((1r,3r)-3-(3-chloro-4-cyanophenoxy)-2,2,4,4-tetramethylcyclobutyl)-4-(5-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)hexahydropyrrolo[3,4-c]pyrrol-2(1H)-yl)benzamide |
| 139 | N-((1r,3r)-3-(3-chloro-4-cyanophenoxy)-2,2,4,4-tetramethylcyclobutyl)-4-(5-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)hexahydropyrrolo[3,4-c]pyrrol-2(1H)-yl)benzamide |
| 140 | N-((1r,3r)-3-(3-chloro-4-cyanophenoxy)-2,2,4,4-tetramethylcyclobutyl)-4-(5-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)hexahydropyrrolo[3,4-c]pyrrol-2(1H)-yl)benzamide |
| 141 | N-((1r,3r)-3-(3-chloro-4-cyanophenoxy)-2,2,4,4-tetramethylcyclobutyl)-4-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)ethynyl)benzamide |
| 142 | N-((1r,3r)-3-(3-chloro-4-cyanophenoxy)-2,2,4,4-tetramethylcyclobutyl)-4-((1-(3-(2,6- |
| | dioxopiperidin-3-yl)-4-oxo-3,4-dihydrobenzo[d][1,2,3]triazin-6-yl)piperidin-4- |
| | yl)ethynyl)benzamide |
| 143 | N-((1r,3r)-3-(3-chloro-4-cyanophenoxy)-2,2,4,4-tetramethylcyclobutyl)-4-((1-((3-(2,6- |
| | dioxopiperidin-3-yl)-4-oxo-3,4-dihydrobenzo[d][1,2,3]triazin-6-yl)glycyl)piperidin-4- |
| | yl)ethynyl)benzamide |
| 144 | N-((1r,3r)-3-(3-chloro-4-cyanophenoxy)-2,2,4,4-tetramethylcyclobutyl)-4-((1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)ethynyl)benzamide |
| 145 | N-((1r,3r)-3-(3-chloro-4-cyanophenoxy)-2,2,4,4-tetramethylcyclobutyl)-2-(4-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)pyrimidine-5-carboxamide |
| 146 | N-((1r,3r)-3-(3-chloro-4-cyanophenoxy)-2,2,4,4-tetramethylcyclobutyl)-2-(4-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)pyrimidine-5-carboxamide |
| 147 | N-((1r,3r)-3-(3-chloro-4-cyanophenoxy)-2,2,4,4-tetramethylcyclobutyl)-2-(4-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)piperazin-1-yl)pyrimidine-5-carboxamide |
| 148 | N-((1r,3r)-3-(3-chloro-4-cyanophenoxy)-2,2,4,4-tetramethylcyclobutyl)-2-(4-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)methyl)piperazin-1-yl)pyrimidine-5-carboxamide |
| 149 | N-((1r,3r)-3-(3-chloro-4-cyanophenoxy)-2,2,4,4-tetramethylcyclobutyl)-4-(4-(((3-(2,6- |
| | dioxopiperidin-3-yl)-4-oxo-3,4-dihydrobenzo[d][1,2,3]triazin-6-yl)amino)methyl)-1H-1,2,3- |
| | triazol-1-yl)benzamide |
| 150 | N-((1-(4-(((1r,3r)-3-(3-chloro-4-cyanophenoxy)-2,2,4,4- |
| | tetramethylcyclobutyl)carbamoyl)phenyl)-1H-1,2,3-triazol-4-yl)methyl)-1-(3-(2,6- |
| | dioxopiperidin-3-yl)-4-oxo-3,4-dihydrobenzo[d][1,2,3]triazin-6-yl)piperidine-4- |
| | carboxamide |
| 151 | N-((1-(4-(((1r,3r)-3-(3-chloro-4-cyanophenoxy)-2,2,4,4- |
| | tetramethylcyclobutyl)carbamoyl)phenyl)-1H-1,2,3-triazol-4-yl)methyl)-1-(3-(2,6- |
| | dioxopiperidin-3-yl)-4-oxo-3,4-dihydrobenzo[d][1,2,3]triazin-6-yl)azetidine-3-carboxamide |
| 152 | N-((1r,3r)-3-(3-chloro-4-cyanophenoxy)-2,2,4,4-tetramethylcyclobutyl)-4-(4-((2-((3-(2,6- |
| | dioxopiperidin-3-yl)-4-oxo-3,4-dihydrobenzo[d][1,2,3]triazin-6- |
| | yl)amino)acetamido)methyl)-1H-1,2,3-triazol-1-yl)benzamide |
| 153 | N-((1r,3r)-3-(3-chloro-4-cyanophenoxy)-2,2,4,4-tetramethylcyclobutyl)-4-(4-(2-((3-(2,6- |
| | dioxopiperidin-3-yl)-4-oxo-3,4-dihydrobenzo[d][1,2,3]triazin-6-yl)amino)ethyl)-1H-1,2,3- |
| | triazol-1-yl)benzamide |
| 154 | N-(2-(1-(4-(((1r,3r)-3-(3-chloro-4-cyanophenoxy)-2,2,4,4- |
| | tetramethylcyclobutyl)carbamoyl)phenyl)-1H-1,2,3-triazol-4-yl)ethyl)-1-(3-(2,6- |
| | dioxopiperidin-3-yl)-4-oxo-3,4-dihydrobenzo[d][1,2,3]triazin-6-yl)piperidine-4- |
| | carboxamide |
| 155 | N-(2-(1-(4-(((1r,3r)-3-(3-chloro-4-cyanophenoxy)-2,2,4,4- |
| | tetramethylcyclobutyl)carbamoyl)phenyl)-1H-1,2,3-triazol-4-yl)ethyl)-1-(3-(2,6- |
| | dioxopiperidin-3-yl)-4-oxo-3,4-dihydrobenzo[d][1,2,3]triazin-6-yl)azetidine-3-carboxamide |
| 156 | N-((1r,3r)-3-(3-chloro-4-cyanophenoxy)-2,2,4,4-tetramethylcyclobutyl)-4-(4-(2-(2-((3-(2,6- |
| | dioxopiperidin-3-yl)-4-oxo-3,4-dihydrobenzo[d][1,2,3]triazin-6-yl)amino)acetamido)ethyl)- |
| | 1H-1,2,3-triazol-1-yl)benzamide |
| 157 | N-(2-(2-(2-(1-(4-(((1r,3r)-3-(3-chloro-4-cyanophenoxy)-2,2,4,4- |
| | tetramethylcyclobutyl)carbamoyl)phenyl)-1H-1,2,3-triazol-4-yl)ethoxy)ethoxy)ethyl)-1-(3- |
| | (2,6-dioxopiperidin-3-yl)-4-oxo-3,4-dihydrobenzo[d][1,2,3]triazin-6-yl)azetidine-3- |
| | carboxamide |
| 158 | N-(2-(2-(2-(1-(4-(((1r,3r)-3-(3-chloro-4-cyanophenoxy)-2,2,4,4- |
| | tetramethylcyclobutyl)carbamoyl)phenyl)-1H-1,2,3-triazol-4-yl)ethoxy)ethoxy)ethyl)-1-(3- |
| | (2,6-dioxopiperidin-3-yl)-4-oxo-3,4-dihydrobenzo[d][1,2,3]triazin-6-yl)piperidine-4- |
| | carboxamide |
| 159 | N-((1r,3r)-3-(3-chloro-4-cyanophenoxy)-2,2,4,4-tetramethylcyclobutyl)-4-(4-(2-(2-(2-(2- |
| | ((3-(2,6-dioxopiperidin-3-yl)-4-oxo-3,4-dihydrobenzo[d][1,2,3]triazin-6- |
| | yl)amino)acetamido)ethoxy)ethoxy)ethyl)-1H-1,2,3-triazol-1-yl)benzamide |
| 160 | N-((1r,4r)-4-(3-chloro-4-cyanophenoxy)cyclohexyl)-6-(4-((4-(3-(2,6-dioxopiperidin-3-yl)-4- |
| | oxo-3,4-dihydrobenzo[d][1,2,3]triazin-6-yl)piperazin-1-yl)methyl)piperidin-1- |
| | yl)nicotinamide |
| 161 | N-((1r,4r)-4-(3-chloro-4-cyanophenoxy)cyclohexyl)-6-(4-(3-((3-(2,6-dioxopiperidin-3-yl)-4- |
| | oxo-3,4-dihydrobenzo[d][1,2,3]triazin-6-yl)oxy)propyl)piperazin-1-yl)nicotinamide |
| 162 | N-((1r,3r)-3-(3-chloro-4-cyanophenoxy)-2,2,4,4-tetramethylcyclobutyl)-6-(4-((4-(3-(2,6- |
| | dioxopiperidin-3-yl)-4-oxo-3,4-dihydrobenzo[d][1,2,3]triazin-6-yl)piperazin-1- |
| | yl)methyl)piperidin-1-yl)nicotinamide |
| 163 | N-((1r,3r)-3-(3-chloro-4-cyanophenoxy)-2,2,4,4-tetramethylcyclobutyl)-6-(4-(5-((3-(2,6- |
| | dioxopiperidin-3-yl)-4-oxo-3,4-dihydrobenzo[d][1,2,3]triazin-6-yl)oxy)pentyl)piperazin-1- |
| | yl)nicotinamide |
| 164 | N-((1r,4r)-4-(3-chloro-4-cyanophenoxy)cyclohexyl)-6-(4-((4-(3-(2,6-dioxopiperidin-3-yl)-4- |
| | oxo-3,4-dihydrobenzo[d][1,2,3]triazin-7-yl)piperazin-1-yl)methyl)piperidin-1- |
| | yl)nicotinamide |
| 165 | N-((1r,3r)-3-(3-chloro-4-cyanophenoxy)-2,2,4,4-tetramethylcyclobutyl)-4-(4-((4-(3-(2,6- |
| | dioxopiperidin-3-yl)-4-oxo-3,4-dihydrobenzo[d][1,2,3]triazin-7-yl)piperazin-1- |
| | yl)methyl)piperidin-1-yl)benzamide |
| 166 | N-((1r,3r)-3-(3-chloro-4-cyanophenoxy)-2,2,4,4-tetramethylcyclobutyl)-4-(4-((4-(3-(2,6- |
| | dioxopiperidin-3-yl)-4-oxo-3,4-dihydrobenzo[d][1,2,3]triazin-6-yl)piperazin-1- |
| | yl)methyl)piperidin-1-yl)benzamide |
| 167 | N-((1r,4r)-4-(3-chloro-4-cyanophenoxy)cyclohexyl)-6-(4-((7-(3-(2,6-dioxopiperidin-3-yl)-4- |
| | oxo-3,4-dihydrobenzo[d][1,2,3]triazin-7-yl)-2,7-diazaspiro[3.5]nonan-2- |
| | yl)methyl)piperidin-1-yl)nicotinamide |
| 168 | N-((1r,4r)-4-(3-chloro-4-cyanophenoxy)cyclohexyl)-6-(4-((7-(3-(2,6-dioxopiperidin-3-yl)-4- |
| | oxo-3,4-dihydrobenzo[d][1,2,3]triazin-6-yl)-2,7-diazaspiro[3.5]nonan-2- |
| | yl)methyl)piperidin-1-yl)nicotinamide |
| 169 | N-((1r,3r)-3-(3-chloro-4-cyanophenoxy)-2,2,4,4-tetramethylcyclobutyl)-6-(4-((7-(3-(2,6- |
| | dioxopiperidin-3-yl)-4-oxo-3,4-dihydrobenzo[d][1,2,3]triazin-7-yl)-2,7- |
| | diazaspiro[3.5]nonan-2-yl)methyl)piperidin-1-yl)nicotinamide |
| 170 | N-((1r,3r)-3-(3-chloro-4-cyanophenoxy)-2,2,4,4-tetramethylcyclobutyl)-6-(4-((7-(3-(2,6- |
| | dioxopiperidin-3-yl)-4-oxo-3,4-dihydrobenzo[d][1,2,3]triazin-6-yl)-2,7- |
| | diazaspiro[3.5]nonan-2-yl)methyl)piperidin-1-yl)nicotinamide |
| 171 | N-((1r,3r)-3-(3-chloro-4-cyanophenoxy)-2,2,4,4-tetramethylcyclobutyl)-6-(4-((2-(3-(2,6- |
| | dioxopiperidin-3-yl)-4-oxo-3,4-dihydrobenzo[d][1,2,3]triazin-7-yl)-2,7- |
| | diazaspiro[3.5]nonan-7-yl)methyl)piperidin-1-yl)nicotinamide |
| 172 | N-((1r,3r)-3-(3-chloro-4-cyanophenoxy)-2,2,4,4-tetramethylcyclobutyl)-6-(4-((2-(3-(2,6- |
| | dioxopiperidin-3-yl)-4-oxo-3,4-dihydrobenzo[d][1,2,3]triazin-6-yl)-2,7- |
| | diazaspiro[3.5]nonan-7-yl)methyl)piperidin-1-yl)nicotinamide |
| 173 | N-((1r,3r)-3-(3-chloro-4-cyanophenoxy)-2,2,4,4-tetramethylcyclobutyl)-6-(2-((1-(3-(2,6- |
| | dioxopiperidin-3-yl)-4-oxo-3,4-dihydrobenzo[d][1,2,3]triazin-7-yl)piperidin-4-yl)methyl)- |
| | 2,7-diazaspiro[3.5]nonan-7-yl)nicotinamide |
| 174 | N-((1r,3r)-3-(3-chloro-4-cyanophenoxy)-2,2,4,4-tetramethylcyclobutyl)-6-(2-((1-(3-(2,6- |
| | dioxopiperidin-3-yl)-4-oxo-3,4-dihydrobenzo[d][1,2,3]triazin-6-yl)piperidin-4-yl)methyl)- |
| | 2,7-diazaspiro[3.5]nonan-7-yl)nicotinamide |
| 175 | N-((1r,3r)-3-(3-chloro-4-cyanophenoxy)-2,2,4,4-tetramethylcyclobutyl)-6-(2-((1-(3-(2,6- |
| | dioxopiperidin-3-yl)-4-oxo-3,4-dihydrobenzo[d][1,2,3]triazin-7-yl)azetidin-3-yl)methyl)- |
| | 2,7-diazaspiro[3.5]nonan-7-yl)nicotinamide |
| 176 | N-((1r,3r)-3-(3-chloro-4-cyanophenoxy)-2,2,4,4-tetramethylcyclobutyl)-6-(2-((1-(3-(2,6- |
| | dioxopiperidin-3-yl)-4-oxo-3,4-dihydrobenzo[d][1,2,3]triazin-6-yl)azetidin-3-yl)methyl)- |
| | 2,7-diazaspiro[3.5]nonan-7-yl)nicotinamide |
| 177 | N-((1r,3r)-3-(3-chloro-4-cyanophenoxy)-2,2,4,4-tetramethylcyclobutyl)-6-(3-(4-(3-(2,6- |
| | dioxopiperidin-3-yl)-4-oxo-3,4-dihydrobenzo[d][1,2,3]triazin-7-yl)piperazin-1-yl)prop-1-yn- |
| | 1-yl)nicotinamide |
| 178 | N-((1r,3r)-3-(3-chloro-4-cyanophenoxy)-2,2,4,4-tetramethylcyclobutyl)-6-(3-(4-(3-(2,6- |
| | dioxopiperidin-3-yl)-4-oxo-3,4-dihydrobenzo[d][1,2,3]triazin-6-yl)piperazin-1-yl)prop-1-yn- |
| | 1-yl)nicotinamide |
| 179 | N-((1r,3r)-3-(3-chloro-4-cyanophenoxy)-2,2,4,4-tetramethylcyclobutyl)-6-(3-(7-(3-(2,6- |
| | dioxopiperidin-3-yl)-4-oxo-3,4-dihydrobenzo[d][1,2,3]triazin-7-yl)-2,7- |
| | diazaspiro[3.5]nonan-2-yl)prop-1-yn-1-yl)nicotinamide |
| 180 | N-((1r,3r)-3-(3-chloro-4-cyanophenoxy)-2,2,4,4-tetramethylcyclobutyl)-6-(3-(7-(3-(2,6- |
| | dioxopiperidin-3-yl)-4-oxo-3,4-dihydrobenzo[d][1,2,3]triazin-6-yl)-2,7- |
| | diazaspiro[3.5]nonan-2-yl)prop-1-yn-1-yl)nicotinamide |
| 181 | N-((1r,3r)-3-(3-chloro-4-cyanophenoxy)-2,2,4,4-tetramethylcyclobutyl)-6-(3-(2-(3-(2,6- |
| | dioxopiperidin-3-yl)-4-oxo-3,4-dihydrobenzo[d][1,2,3]triazin-7-yl)-2,7- |
| | diazaspiro[3.5]nonan-7-yl)prop-1-yn-1-yl)nicotinamide |
| 182 | N-((1r,3r)-3-(3-chloro-4-cyanophenoxy)-2,2,4,4-tetramethylcyclobutyl)-6-(3-(2-(3-(2,6- |
| | dioxopiperidin-3-yl)-4-oxo-3,4-dihydrobenzo[d][1,2,3]triazin-6-yl)-2,7- |
| | diazaspiro[3.5]nonan-7-yl)prop-1-yn-1-yl)nicotinamide |
| 183 | N-((1r,3r)-3-(3-chloro-4-cyanophenoxy)-2,2,4,4-tetramethylcyclobutyl)-6-((1-((1-(3-(2,6- |
| | dioxopiperidin-3-yl)-4-oxo-3,4-dihydrobenzo[d][1,2,3]triazin-7-yl)azetidin-3- |
| | yl)methyl)piperidin-4-yl)ethynyl)nicotinamide |
| 184 | N-((1r,3r)-3-(3-chloro-4-cyanophenoxy)-2,2,4,4-tetramethylcyclobutyl)-6-((1-((1-(3-(2,6- |
| | dioxopiperidin-3-yl)-4-oxo-3,4-dihydrobenzo[d][1,2,3]triazin-6-yl)azetidin-3- |
| | yl)methyl)piperidin-4-yl)ethynyl)nicotinamide |
| 185 | N-((1r,3R)-3-(3-chloro-4-cyanophenoxy)-2,2,4,4-tetramethylcyclobutyl)-6-((3R)-3-((7-(3- |
| | (2,6-dioxopiperidin-3-yl)-4-oxo-3,4-dihydrobenzo[d][1,2,3]triazin-7-yl)-2,7- |
| | diazaspiro[3.5]nonan-2-yl)methyl)pyrrolidin-1-yl)nicotinamide |
| 186 | N-((1r,3R)-3-(3-chloro-4-cyanophenoxy)-2,2,4,4-tetramethylcyclobutyl)-6-((3R)-3-((7-(3- |
| | (2,6-dioxopiperidin-3-yl)-4-oxo-3,4-dihydrobenzo[d][1,2,3]triazin-6-yl)-2,7- |
| | diazaspiro[3.5]nonan-2-yl)methyl)pyrrolidin-1-yl)nicotinamide |
| 187 | N-((1r,3S)-3-(3-chloro-4-cyanophenoxy)-2,2,4,4-tetramethylcyclobutyl)-6-((3S)-3-((7-(3- |
| | (2,6-dioxopiperidin-3-yl)-4-oxo-3,4-dihydrobenzo[d][1,2,3]triazin-7-yl)-2,7- |
| | diazaspiro[3.5]nonan-2-yl)methyl)pyrrolidin-1-yl)nicotinamide |
| 188 | N-((1r,3S)-3-(3-chloro-4-cyanophenoxy)-2,2,4,4-tetramethylcyclobutyl)-6-((3S)-3-((7-(3- |
| | (2,6-dioxopiperidin-3-yl)-4-oxo-3,4-dihydrobenzo[d][1,2,3]triazin-6-yl)-2,7- |
| | diazaspiro[3.5]nonan-2-yl)methyl)pyrrolidin-1-yl)nicotinamide |
| 189 | N-((1r,3R)-3-(3-chloro-4-cyanophenoxy)-2,2,4,4-tetramethylcyclobutyl)-6-((3R)-3-((2-(3- |
| | (2,6-dioxopiperidin-3-yl)-4-oxo-3,4-dihydrobenzo[d][1,2,3]triazin-7-yl)-2,7- |
| | diazaspiro[3.5]nonan-7-yl)methyl)pyrrolidin-1-yl)nicotinamide |
| 190 | N-((1r,3R)-3-(3-chloro-4-cyanophenoxy)-2,2,4,4-tetramethylcyclobutyl)-6-((3R)-3-((6-(3- |
| | (2,6-dioxopiperidin-3-yl)-4-oxo-3,4-dihydrobenzo[d][1,2,3]triazin-7-yl)-2,6- |
| | diazaspiro[3.3]heptan-2-yl)methyl)pyrrolidin-1-yl)nicotinamide |
| 191 | N-((1r,3R)-3-(3-chloro-4-cyanophenoxy)-2,2,4,4-tetramethylcyclobutyl)-6-((3R)-3-((4-(1- |
| | (3-(2,6-dioxopiperidin-3-yl)-4-oxo-3,4-dihydrobenzo[d][1,2,3]triazin-7-yl)piperidin-4- |
| | yl)piperazin-1-yl)methyl)pyrrolidin-1-yl)nicotinamide |
| 192 | N-((1r,3R)-3-(3-chloro-4-cyanophenoxy)-2,2,4,4-tetramethylcyclobutyl)-6-((3R)-3-((4-(1- |
| | (3-(2,6-dioxopiperidin-3-yl)-4-oxo-3,4-dihydrobenzo[d][1,2,3]triazin-6-yl)piperidin-4- |
| | yl)piperazin-1-yl)methyl)pyrrolidin-1-yl)nicotinamide |
| 193 | N-((1r,3r)-3-(3-chloro-4-cyanophenoxy)-2,2,4,4-tetramethylcyclobutyl)-6-(4-((4-(1-(3-(2,6- |
| | dioxopiperidin-3-yl)-4-oxo-3,4-dihydrobenzo[d][1,2,3]triazin-7-yl)piperidin-4-yl)piperazin- |
| | 1-yl)methyl)piperidin-1-yl)nicotinamide |
| 194 | N-((1r,3r)-3-(3-chloro-4-cyanophenoxy)-2,2,4,4-tetramethylcyclobutyl)-6-(4-((4-(1-(3-(2,6- |
| | dioxopiperidin-3-yl)-4-oxo-3,4-dihydrobenzo[d][1,2,3]triazin-6-yl)piperidin-4-yl)piperazin- |
| | 1-yl)methyl)piperidin-1-yl)nicotinamide |
| 195 | N-((1r,3r)-3-(3-chloro-4-cyanophenoxy)-2,2,4,4-tetramethylcyclobutyl)-6-((1-((1-(3-(2,6- |
| | dioxopiperidin-3-yl)-4-oxo-3,4-dihydrobenzo[d][1,2,3]triazin-7-yl)piperidin-4- |
| | yl)methyl)piperidin-4-yl)ethynyl)nicotinamide |
| 196 | N-((1r,3r)-3-(3-chloro-4-cyanophenoxy)-2,2,4,4-tetramethylcyclobutyl)-6-((1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)ethynyl)nicotinamide |
| 197 | N-((1r,3r)-3-(3-chloro-4-cyanophenoxy)-2,2,4,4-tetramethylcyclobutyl)-6-(4-(2-(4-(3-(2,6- |
| | dioxopiperidin-3-yl)-4-oxo-3,4-dihydrobenzo[d][1,2,3]triazin-7-yl)piperazin-1- |
| | yl)ethyl)piperidin-1-yl)nicotinamide |
| 198 | N-((1r,3r)-3-(3-chloro-4-cyanophenoxy)-2,2,4,4-tetramethylcyclobutyl)-6-(4-(2-(4-(3-(2,6- |
| | dioxopiperidin-3-yl)-4-oxo-3,4-dihydrobenzo[d][1,2,3]triazin-6-yl)piperazin-1- |
| | yl)ethyl)piperidin-1-yl)nicotinamide |

10. The compound of claim 9 or a pharmaceutically acceptable salt thereof, wherein the corn und is an one of the following compounds:

| Compound | |
|---|---|
| No. | IUPAC name |
| 11 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)piperazin-1-yl)benzamide |
| 20 | N-((1r,3r)-3-(3-chloro-4-cyanophenoxy)-2,2,4,4-tetramethylcyclobutyl)-4-(4-(7-(1-(3-(2,6- |
| | dioxopiperidin-3-yl)-4-oxo-3,4-dihydrobenzo[d][1,2,3]triazin-6-yl)piperidine-4-carbonyl)-7- |
| | azaspiro[3.5]nonan-2-yl)piperazin-1-yl)benzamide |
| 33 | N-((1r,3r)-3-(3-chloro-4-cyanophenoxy)-2,2,4,4-tetramethylcyclobutyl)-4-(4-(2-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)oxy)ethyl)piperazin-1-yl)benzamide |
| 34 | N-((1r,3r)-3-(3-chloro-4-cyanophenoxy)-2,2,4,4-tetramethylcyclobutyl)-4-(4-(2-((1-(3-(2,6- |
| | dioxopiperidin-3-yl)-4-oxo-3,4-dihydrobenzo[d][1,2,3]triazin-6-yl)piperidin-4- |
| | yl)oxy)ethyl)piperazin-1-yl)benzamide |
| 44 | N-((1r,3r)-3-(3-chloro-4-cyanophenoxy)-2,2,4,4-tetramethylcyclobutyl)-4-(4-(1-(3-(2,6- |
| | dioxopiperidin-3-yl)-4-oxo-3,4-dihydrobenzo[d][1,2,3]triazin-6-yl)azetidine-3- |
| | carbonyl)piperazin-1-yl)benzamide |
| 47 | N-((1r,3r)-3-(3-chloro-4-cyanophenoxy)-2,2,4,4-tetramethylcyclobutyl)-4-(4-(3-(1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)propyl)piperazin-1-yl)benzamide |
| 63 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)azetidine-3-carbonyl)-4- |
| | fluoropiperidin-4-yl)methyl)piperazin-1-yl)benzamide |
| 75 | N-((1r,3r)-3-(3-chloro-4-cyanophenoxy)-2,2,4,4-tetramethylcyclobutyl)-4-(1-(2-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)oxy)ethyl)piperidin-4-yl)benzamide |
| 76 | N-((1r,3r)-3-(3-chloro-4-cyanophenoxy)-2,2,4,4-tetramethylcyclobutyl)-4-(1-(2-((1-(3-(2,6- |
| | dioxopiperidin-3-yl)-4-oxo-3,4-dihydrobenzo[d][1,2,3]triazin-6-yl)piperidin-4- |
| | yl)oxy)ethyl)piperidin-4-yl)benzamide |
| 78 | N-((1r,3r)-3-(3-chloro-4-cyanophenoxy)-2,2,4,4-tetramethylcyclobutyl)-6-(4-((1-(3-(2,6- |
| | dioxopiperidin-3-yl)-4-oxo-3,4-dihydrobenzo[d][1,2,3]triazin-6-yl)piperidin-4- |
| | yl)methyl)piperazin-1-yl)nicotinamide |
| 82 | N-((1r,3r)-3-(3-chloro-4-cyanophenoxy)-2,2,4,4-tetramethylcyclobutyl)-4-(1-(1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)benzamide |
| 83 | N-((1r,3r)-3-(3-chloro-4-cyanophenoxy)-2,2,4,4-tetramethylcyclobutyl)-4-(1-(1-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)benzamide |
| 84 | N-((1r,3r)-3-(3-chloro-4-cyanophenoxy)-2,2,4,4-tetramethylcyclobutyl)-4-(1-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)-1H-pyrazol-4-yl)benzamide |
| 88 | N-((1r,3r)-3-(3-chloro-4-cyanophenoxy)-2,2,4,4-tetramethylcyclobutyl)-4-(3-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)prop-1-yn-1-yl)benzamide |
| 90 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)-1H-1,2,3-triazol-1-yl)benzamide |
| 94 | N-((1r,3r)-3-(3-chloro-4-cyanophenoxy)-2,2,4,4-tetramethylcyclobutyl)-4-(2-(5-(1-(3-(2,6- |
| | dioxopiperidin-3-yl)-4-oxo-3,4-dihydrobenzo[d][1,2,3]triazin-6-yl)azetidine-3- |
| | carbonyl)hexahydropyrrolo[3,4-c]pyrrol-2(1H)-yl)ethyl)benzamide |
| 95 | N-((1r,3r)-3-(3-chloro-4-cyanophenoxy)-2,2,4,4-tetramethylcyclobutyl)-4-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)ethynyl)benzamide |
| 96 | N-((1r,3r)-3-(3-chloro-4-cyanophenoxy)-2,2,4,4-tetramethylcyclobutyl)-4-((1-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)ethynyl)benzamide |
| 97 | N-((1r,3r)-3-(3-chloro-4-cyanophenoxy)-2,2,4,4-tetramethylcyclobutyl)-4-((1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)benzamide |
| 98 | N-((1r,3r)-3-(3-chloro-4-cyanophenoxy)-2,2,4,4-tetramethylcyclobutyl)-4-((1-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)piperidin-4-yl)ethynyl)benzamide |
| 99 | N-((1r,3r)-3-(3-chloro-4-cyanophenoxy)-2,2,4,4-tetramethylcyclobutyl)-4-((1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)ethynyl)benzamide |
| 100 | N-((1r,3r)-3-(3-chloro-4-cyanophenoxy)-2,2,4,4-tetramethylcyclobutyl)-2-(4-((1-(3-(2,6- |
| | dioxopiperidin-3-yl)-4-oxo-3,4-dihydrobenzo[d][1,2,3]triazin-6-yl)piperidin-4- |
| | yl)methyl)piperazin-1-yl)pyrimidine-5-carboxamide |
| 101 | N-((1r,3r)-3-(3-chloro-4-cyanophenoxy)-2,2,4,4-tetramethylcyclobutyl)-4-(4-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)-1H-1,2,3-triazol-1-yl)benzamide |
| 102 | N-((1r,3r)-3-(3-chloro-4-cyanophenoxy)-2,2,4,4-tetramethylcyclobutyl)-4-(4-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)-1H-1,2,3-triazol-1-yl)benzamide |
| 103 | N-((1r,3r)-3-(3-chloro-4-cyanophenoxy)-2,2,4,4-tetramethylcyclobutyl)-4-(4-((1-(3-(2,6- |
| | dioxopiperidin-3-yl)-4-oxo-3,4-dihydrobenzo[d][1,2,3]triazin-6-yl)piperidin-4-yl)methyl)- |
| | 1H-1,2,3-triazol-1-yl)benzamide |
| 104 | N-((1r,3r)-3-(3-chloro-4-cyanophenoxy)-2,2,4,4-tetramethylcyclobutyl)-4-(4-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)-1H-1,2,3-triazol-1-yl)benzamide |
| 106 | N-((1r,3r)-3-(3-chloro-4-cyanophenoxy)-2,2,4,4-tetramethylcyclobutyl)-4-(4-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)-1H-1,2,3-triazol-1-yl)benzamide |
| 107 | N-((1r,3r)-3-(3-chloro-4-cyanophenoxy)-2,2,4,4-tetramethylcyclobutyl)-4-(4-(1-((3-(2,6- |
| | dioxopiperidin-3-yl)-4-oxo-3,4-dihydrobenzo[d][1,2,3]triazin-6-yl)glycyl)piperidin-4-yl)-1H- |
| | 1,2,3-triazol-1-yl)benzamide |
| 108 | N-((1r,3r)-3-(3-chloro-4-cyanophenoxy)-2,2,4,4-tetramethylcyclobutyl)-4-(2-(4-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperazin-1-yl)ethyl)benzamide |
| 110 | N-((1r,3r)-3-(3-chloro-4-cyanophenoxy)-2,2,4,4-tetramethylcyclobutyl)-4-(2-(4-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperazin-1-yl)ethyl)benzamide |
| 111 | N-((1r,3r)-3-(3-chloro-4-cyanophenoxy)-2,2,4,4-tetramethylcyclobutyl)-4-(2-(4-((1-(3-(2,6- |
| | dioxopiperidin-3-yl)-4-oxo-3,4-dihydrobenzo[d][1,2,3]triazin-6-yl)piperidin-4- |
| | yl)methyl)piperazin-1-yl)ethyl)benzamide |
| 112 | N-((1r,3r)-3-(3-chloro-4-cyanophenoxy)-2,2,4,4-tetramethylcyclobutyl)-4-(1-(1-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)benzamide |
| 114 | N-((1r,3r)-3-(3-chloro-4-cyanophenoxy)-2,2,4,4-tetramethylcyclobutyl)-6-(1-(1-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)nicotinamide |
| 116 | N-((1r,3r)-3-(3-chloro-4-cyanophenoxy)-2,2,4,4-tetramethylcyclobutyl)-4-(3-(1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)prop-1-yn-1-yl)benzamide |
| 117 | N-((1r,3r)-3-(3-chloro-4-cyanophenoxy)-2,2,4,4-tetramethylcyclobutyl)-4-(3-(1-((1-(1-(3- |
| | (2,6-dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)prop-1-yn-1-yl)benzamide |
| 118 | N-((1r,3r)-3-(3-chloro-4-cyanophenoxy)-2,2,4,4-tetramethylcyclobutyl)-4-(3-(1-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperidin-4-yl)prop-1-yn-1-yl)benzamide |
| 121 | N-((1r,3r)-3-(3-chloro-4-cyanophenoxy)-2,2,4,4-tetramethylcyclobutyl)-4-(4-((4-(1-(3-(2,6- |
| | dioxopiperidin-3-yl)-4-oxo-3,4-dihydrobenzo[d][1,2,3]triazin-6-yl)piperidine-4- |
| | carbonyl)piperazin-1-yl)methyl)-1H-1,2,3-triazol-1-yl)benzamide |
| 122 | N-((1r,3r)-3-(3-chloro-4-cyanophenoxy)-2,2,4,4-tetramethylcyclobutyl)-4-(4-((4-(1-(3-(2,6- |
| | dioxopiperidin-3-yl)-4-oxo-3,4-dihydrobenzo[d][1,2,3]triazin-6-yl)azetidine-3- |
| | carbonyl)piperazin-1-yl)methyl)-1H-1,2,3-triazol-1-yl)benzamide |
| 124 | N-((1r,3r)-3-(3-chloro-4-cyanophenoxy)-2,2,4,4-tetramethylcyclobutyl)-6-(1-(1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)nicotinamide |
| 125 | N-((1r,3r)-3-(3-chloro-4-cyanophenoxy)-2,2,4,4-tetramethylcyclobutyl)-6-(1-(1-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)nicotinamide |
| 128 | N-((1r,3r)-3-(3-chloro-4-cyanophenoxy)-2,2,4,4-tetramethylcyclobutyl)-6-(3-(1-((1-((3- |
| | (2,6-dioxopiperidin-3-yl)-4-oxo-3,4-dihydrobenzo[d][1,2,3]triazin-6-yl)glycyl)piperidin-4- |
| | yl)methyl)piperidin-4-yl)prop-1-yn-1-yl)nicotinamide |
| 129 | N-((1r,3r)-3-(3-chloro-4-cyanophenoxy)-2,2,4,4-tetramethylcyclobutyl)-6-(3-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)prop-1-yn-1-yl)nicotinamide |
| 130 | N-((1r,3r)-3-(3-chloro-4-cyanophenoxy)-2,2,4,4-tetramethylcyclobutyl)-6-((1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)nicotinamide |
| 131 | N-((1r,3r)-3-(3-chloro-4-cyanophenoxy)-2,2,4,4-tetramethylcyclobutyl)-6-((1-((1-(1-(3- |
| | (2,6-dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)ethynyl)nicotinamide |
| 132 | N-((1r,3r)-3-(3-chloro-4-cyanophenoxy)-2,2,4,4-tetramethylcyclobutyl)-6-((1-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)piperidin-4-yl)ethynyl)nicotinamide |
| 133 | N-((1r,3r)-3-(3-chloro-4-cyanophenoxy)-2,2,4,4-tetramethylcyclobutyl)-6-((1-((1-(2-((3- |
| | (2,6-dioxopiperidin-3-yl)-4-oxo-3,4-dihydrobenzo[d][1,2,3]triazin-6-yl)oxy)acetyl)piperidin- |
| | 4-yl)methyl)piperidin-4-yl)ethynyl)nicotinamide |
| 142 | N-((1r,3r)-3-(3-chloro-4-cyanophenoxy)-2,2,4,4-tetramethylcyclobutyl)-4-((1-(3-(2,6- |
| | dioxopiperidin-3-yl)-4-oxo-3,4-dihydrobenzo[d][1,2,3]triazin-6-yl)piperidin-4- |
| | yl)ethynyl)benzamide |
| 143 | N-((1r,3r)-3-(3-chloro-4-cyanophenoxy)-2,2,4,4-tetramethylcyclobutyl)-4-((1-((3-(2,6- |
| | dioxopiperidin-3-yl)-4-oxo-3,4-dihydrobenzo[d][1,2,3]triazin-6-yl)glycyl)piperidin-4- |
| | yl)ethynyl)benzamide |
| 150 | N-((1-(4-(((1r,3r)-3-(3-chloro-4-cyanophenoxy)-2,2,4,4- |
| | tetramethylcyclobutyl)carbamoyl)phenyl)-1H-1,2,3-triazol-4-yl)methyl)-1-(3-(2,6- |
| | dioxopiperidin-3-yl)-4-oxo-3,4-dihydrobenzo[d][1,2,3]triazin-6-yl)piperidine-4- |
| | carboxamide |
| 165 | N-((1r,3r)-3-(3-chloro-4-cyanophenoxy)-2,2,4,4-tetramethylcyclobutyl)-4-(4-((4-(3-(2,6- |
| | dioxopiperidin-3-yl)-4-oxo-3,4-dihydrobenzo[d][1,2,3]triazin-7-yl)piperazin-1- |
| | yl)methyl)piperidin-1-yl)benzamide |
| 166 | N-((1r,3r)-3-(3-chloro-4-cyanophenoxy)-2,2,4,4-tetramethylcyclobutyl)-4-(4-((4-(3-(2,6- |
| | dioxopiperidin-3-yl)-4-oxo-3,4-dihydrobenzo[d][1,2,3]triazin-6-yl)piperazin-1- |
| | yl)methyl)piperidin-1-yl)benzamide |
| 169 | N-((1r,3r)-3-(3-chloro-4-cyanophenoxy)-2,2,4,4-tetramethylcyclobutyl)-6-(4-((7-(3-(2,6- |
| | dioxopiperidin-3-yl)-4-oxo-3,4-dihydrobenzo[d][1,2,3]triazin-7-yl)-2,7- |
| | diazaspiro[3.5]nonan-2-yl)methyl)piperidin-1-yl)nicotinamide |
| 170 | N-((1r,3r)-3-(3-chloro-4-cyanophenoxy)-2,2,4,4-tetramethylcyclobutyl)-6-(4-((7-(3-(2,6- |
| | dioxopiperidin-3-yl)-4-oxo-3,4-dihydrobenzo[d][1,2,3]triazin-6-yl)-2,7- |
| | diazaspiro[3.5]nonan-2-yl)methyl)piperidin-1-yl)nicotinamide |
| 171 | N-((1r,3r)-3-(3-chloro-4-cyanophenoxy)-2,2,4,4-tetramethylcyclobutyl)-6-(4-((2-(3-(2,6- |
| | dioxopiperidin-3-yl)-4-oxo-3,4-dihydrobenzo[d][1,2,3]triazin-7-yl)-2,7- |
| | diazaspiro[3.5]nonan-7-yl)methyl)piperidin-1-yl)nicotinamide |
| 172 | N-((1r,3r)-3-(3-chloro-4-cyanophenoxy)-2,2,4,4-tetramethylcyclobutyl)-6-(4-((2-(3-(2,6- |
| | dioxopiperidin-3-yl)-4-oxo-3,4-dihydrobenzo[d][1,2,3]triazin-6-yl)-2,7- |
| | diazaspiro[3.5]nonan-7-yl)methyl)piperidin-1-yl)nicotinamide |
| 173 | N-((1r,3r)-3-(3-chloro-4-cyanophenoxy)-2,2,4,4-tetramethylcyclobutyl)-6-(2-((1-(3-(2,6- |
| | dioxopiperidin-3-yl)-4-oxo-3,4-dihydrobenzo[d][1,2,3]triazin-7-yl)piperidin-4-yl)methyl)- |
| | 2,7-diazaspiro[3.5]nonan-7-yl)nicotinamide |
| 174 | N-((1r,3r)-3-(3-chloro-4-cyanophenoxy)-2,2,4,4-tetramethylcyclobutyl)-6-(2-((1-(3-(2,6- |
| | dioxopiperidin-3-yl)-4-oxo-3,4-dihydrobenzo[d][1,2,3]triazin-6-yl)piperidin-4-yl)methyl)- |
| | 2,7-diazaspiro[3.5]nonan-7-yl)nicotinamide |
| 175 | N-((1r,3r)-3-(3-chloro-4-cyanophenoxy)-2,2,4,4-tetramethylcyclobutyl)-6-(2-((1-(3-(2,6- |
| | dioxopiperidin-3-yl)-4-oxo-3,4-dihydrobenzo[d][1,2,3]triazin-7-yl)azetidin-3-yl)methyl)- |
| | 2,7-diazaspiro[3.5]nonan-7-yl)nicotinamide |
| 176 | N-((1r,3r)-3-(3-chloro-4-cyanophenoxy)-2,2,4,4-tetramethylcyclobutyl)-6-(2-((1-(3-(2,6- |
| | dioxopiperidin-3-yl)-4-oxo-3,4-dihydrobenzo[d][1,2,3]triazin-7-yl)azetidin-3-yl)methyl)- |
| | 2,7-diazaspiro[3.5]nonan-7-yl)nicotinamide |
| 183 | N-((1r,3r)-3-(3-chloro-4-cyanophenoxy)-2,2,4,4-tetramethylcyclobutyl)-6-((1-((1-(3-(2,6- |
| | dioxopiperidin-3-yl)-4-oxo-3,4-dihydrobenzo[d][1,2,3]triazin-7-yl)azetidin-3- |
| | yl)methyl)piperidin-4-yl)ethynyl)nicotinamide |
| 184 | N-((1r,3r)-3-(3-chloro-4-cyanophenoxy)-2,2,4,4-tetramethylcyclobutyl)-6-((1-((1-(3-(2,6- |
| | dioxopiperidin-3-yl)-4-oxo-3,4-dihydrobenzo[d][1,2,3]triazin-6-yl)azetidin-3- |
| | yl)methyl)piperidin-4-yl)ethynyl)nicotinamide |
| 185 | N-((1r,3R)-3-(3-chloro-4-cyanophenoxy)-2,2,4,4-tetramethylcyclobutyl)-6-((3R)-3-((7-(3- |
| | (2,6-dioxopiperidin-3-yl)-4-oxo-3,4-dihydrobenzo[d][1,2,3]triazin-7-yl)-2,7- |
| | diazaspiro[3.5]nonan-2-yl)methyl)pyrrolidin-1-yl)nicotinamide |
| 186 | N-((1r,3R)-3-(3-chloro-4-cyanophenoxy)-2,2,4,4-tetramethylcyclobutyl)-6-((3R)-3-((7-(3- |
| | (2,6-dioxopiperidin-3-yl)-4-oxo-3,4-dihydrobenzo[d][1,2,3]triazin-6-yl)-2,7- |
| | diazaspiro[3.5]nonan-2-yl)methyl)pyrrolidin-1-yl)nicotinamide |
| 193 | N-((1r,3r)-3-(3-chloro-4-cyanophenoxy)-2,2,4,4-tetramethylcyclobutyl)-6-(4-((4-(1-(3-(2,6- |
| | dioxopiperidin-3-yl)-4-oxo-3,4-dihydrobenzo[d][1,2,3]triazin-7-yl)piperidin-4-yl)piperazin- |
| | 1-yl)methyl)piperidin-1-yl)nicotinamide |
| 194 | N-((1r,3r)-3-(3-chloro-4-cyanophenoxy)-2,2,4,4-tetramethylcyclobutyl)-6-(4-((4-(1-(3-(2,6- |
| | dioxopiperidin-3-yl)-4-oxo-3,4-dihydrobenzo[d][1,2,3]triazin-6-yl)piperidin-4-yl)piperazin- |
| | 1-yl)methyl)piperidin-1-yl)nicotinamide |
| 195 | N-((1r,3r)-3-(3-chloro-4-cyanophenoxy)-2,2,4,4-tetramethylcyclobutyl)-6-((1-((1-(3-(2,6- |
| | dioxopiperidin-3-yl)-4-oxo-3,4-dihydrobenzo[d][1,2,3]triazin-7-yl)piperidin-4- |
| | yl)methyl)piperidin-4-yl)ethynyl)nicotinamide |
| 196 | N-((1r,3r)-3-(3-chloro-4-cyanophenoxy)-2,2,4,4-tetramethylcyclobutyl)-6-((1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)ethynyl)nicotinamide |

11. A composition comprising a compound of claim 1 or a pharmaceutically acceptable salt thereof, and a pharmaceutically acceptable carrier.

12.-14. (canceled)

15. A method for treating or preventing an androgen receptor-related disease, comprising:
administering to a subject in need thereof a therapeutically effective amount of a compound of claim 1 or pharmaceutically acceptable salt thereof.

16. The method of claim 15, wherein the androgen receptor-related disease is cancer, Kennedy® disease, acne, alopecia, cutaneous wound, or hirsutism.

17. The method of claim 16, wherein the androgen receptor-related disease is prostate cancer.
